SEQUENCE LISTING (288 sequences)

SEQ ID NO: 1 - protein, 413 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 2 - protein, 426 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHH

SEQ ID NO: 3 - protein, 442 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHHGGLNDIFEAQKIEWHE

SEQ ID NO: 4 - protein, 418 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 5 - protein, 504 aa, synthetic construct
MSRSVALAVLALLSLSGLEAGILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 6 - protein, 418 aa, synthetic construct; identical to SEQ ID NO: 4

SEQ ID NO: 7 - protein, 431 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHH

SEQ ID NO: 8 - protein, 447 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHHGGLNDIFEAQKIEWHE

SEQ ID NO: 9 - protein, 415 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 10 - protein, 428 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 11 - protein, 415 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 12 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 13 - protein, 415 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 14 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 15 - protein, 444 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHHGGLNDIFEAQKIEWHE

SEQ ID NO: 16 - protein, 396 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPS

SEQ ID NO: 17 - protein, 480 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 18 - protein, 391 aa, synthetic construct
IQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 19 - protein, 504 aa, synthetic construct
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 20 - protein, 118 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 21 - protein, 118 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 22 - protein, 120 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 23 - protein, 120 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 24 - protein, 123 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 25 - protein, 123 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 26 - protein, 118 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 27 - protein, 118 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 28 - protein, 118 aa, synthetic construct
SYRPGTVCLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 29 - protein, 118 aa, synthetic construct
SRRYCNVAYGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 30 - protein, 413 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 31 - protein, 426 aa, synthetic construct
GILGFVFTLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEENLYFQGHHHHHH

SEQ ID NO: 32 - protein, 415 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 33 - protein, 428 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 34 - protein, 415 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 35 - protein, 428 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 36 - protein, 415 aa, synthetic construct
RYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 37 - protein, 428 aa, synthetic construct
RYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 38 - protein, 412 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 39 - protein, 425 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 40 - protein, 548 aa, synthetic construct
MSRSVALAVLALLSLSGLEARYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPMGREENFLUORESCENTPROTEIN

SEQ ID NO: 41 - protein, 412 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 42 - protein, 425 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 43 - protein, 412 aa, synthetic construct
RYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 44 - protein, 425 aa, synthetic construct
RYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 45 - protein, 423 aa, synthetic construct
RYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 46 - protein, 423 aa, synthetic construct; identical to SEQ ID NO: 45

SEQ ID NO: 47 - protein, 410 aa, synthetic construct
RYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 48 - protein, 423 aa, synthetic construct
RYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 49 - protein, 410 aa, synthetic construct
RYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 50 - protein, 423 aa, synthetic construct
RYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 51 - protein, 415 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 52 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 53 - protein, 415 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 54 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 55 - protein, 415 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 56 - protein, 428 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 57 - protein, 412 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 58 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 59 - protein, 548 aa, synthetic construct
MSRSVALAVLALLSLSGLEASYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPMGREENFLUORESCENTPROTEIN

SEQ ID NO: 60 - protein, 412 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 61 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 62 - protein, 412 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 63 - protein, 425 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 64 - protein, 410 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 65 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 66 - protein, 410 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 67 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 68 - protein, 410 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 69 - protein, 423 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 70 - protein, 415 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 71 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 72 - protein, 444 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHHGGLNDIFEAQKIEWHE

SEQ ID NO: 73 - protein, 415 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 74 - protein, 428 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 75 - protein, 444 aa, synthetic construct
SYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 76 - protein, 415 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 77 - protein, 428 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 78 - protein, 444 aa, synthetic construct
SYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 79 - protein, 412 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 80 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 81 - protein, 441 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHHGGLNDIFEAQKIEWHE

SEQ ID NO: 82 - protein, 548 aa, synthetic construct
MSRSVALAVLALLSLSGLEASYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPMGREENFLUORESCENTPROTEIN

SEQ ID NO: 83 - protein, 412 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 84 - protein, 441 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 85 - protein, 425 aa, synthetic construct
SYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 86 - protein, 412 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 87 - protein, 425 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 88 - protein, 441 aa, synthetic construct
SYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 89 - protein, 410 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 90 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 91 - protein, 439 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHHGGLNDIFEAQKIEWHE

SEQ ID NO: 92 - protein, 410 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 93 - protein, 423 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 94 - protein, 439 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 95 - protein, 410 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 96 - protein, 423 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 97 - protein, 439 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 98 - protein, 412 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 99 - protein, 432 aa, synthetic construct
MSRSVALAVLALLSLSGLEARYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 100 - protein, 525 aa, synthetic construct
MSRSVALAVLALLSLSGLEARYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSCSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 101 - protein, 415 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 102 - protein, 551 aa, synthetic construct
MSRSVALAVLALLSLSGLEARYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPMGREENFLUORESCENTPROTEIN

SEQ ID NO: 103 - protein, 415 aa, synthetic construct
RYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 104 - protein, 528 aa, synthetic construct
MSRSVALAVLALLSLSGLEARYRPGTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 105 - protein, 415 aa, synthetic construct
RYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 106 - protein, 528 aa, synthetic construct
MSRSVALAVLALLSLSGLEARYRPGTVALGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 107 - protein, 412 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 108 - protein, 525 aa, synthetic construct
MSRSVALAVLALLSLSGLEARYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 109 - protein, 412 aa, synthetic construct
RYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 110 - protein, 525 aa, synthetic construct
MSRSVALAVLALLSLSGLEARYRPGTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 111 - protein, 412 aa, synthetic construct
RYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 112 - protein, 525 aa, synthetic construct
MSRSVALAVLALLSLSGLEARYRPGTVALGCGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 113 - protein, 410 aa, synthetic construct
RYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 114 - protein, 523 aa, synthetic construct
MSRSVALAVLALLSLSGLEARYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 115 - protein, 410 aa, synthetic construct
RYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 116 - protein, 523 aa, synthetic construct
MSRSVALAVLALLSLSGLEARYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 117 - protein, 410 aa, synthetic construct
RYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 118 - protein, 523 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 119 - protein, 407 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVETRPAGDGTFQKWAAVVVPSGQEQRYTCHMQHEGLQEPLTLSWEPS

SEQ ID NO: 120 - protein, 406 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVTRPAGDGTFQKWAAVVVPSGQEQRYTCHMQHEGLQEPLTLSWEPS

SEQ ID NO: 121 - protein, 410 aa, synthetic construct
SYRPGTEALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 122 - protein, 410 aa, synthetic construct
SYRPGTVELGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 123 - protein, 409 aa, synthetic construct
SYRPGTVALGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 124 - protein, 415 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 125 - protein, 410 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 126 - protein, 410 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 127 - protein, 410 aa, synthetic construct
SYRPGTVCLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 128 - protein, 410 aa, synthetic construct
SRRYCNVAYGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 129 - protein, 405 aa, synthetic construct
SYRPGTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 130 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPS

SEQ ID NO: 131 - protein, 290 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHH

SEQ ID NO: 132 - protein, 306 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSENLYFQGHHHHHHGGLNDIFEAQKIEWHE

SEQ ID NO: 133 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 134 - protein, 290 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 135 - protein, 306 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 136 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 137 - protein, 290 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHH

SEQ ID NO: 138 - protein, 306 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSENLYFQGHHHHHHGGLNDIFEAQKIEWHE

SEQ ID NO: 139 - protein, 277 aa, synthetic construct
GSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 140 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 141 - protein, 277 aa, synthetic construct
GSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 142 - protein, 415 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGAAAAAAAAGGGSGGGGGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 143 - protein, 410 aa, synthetic construct
SWRYCNVAYGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 144 - protein, 410 aa, synthetic construct
SRRYCQVAYGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 145 - protein, 410 aa, synthetic construct
SRRYEMCAMGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 146 - protein, 410 aa, synthetic construct
SRRYLMCAYGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 147 - protein, 410 aa, synthetic construct
SRRWLMCAYGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 148 - protein, 410 aa, synthetic construct
SRRMEMVCIGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 149 - protein, 410 aa, synthetic construct
SRRMWMVCIGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 150 - protein, 410 aa, synthetic construct
SRRMEMVCRGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 151 - protein, 407 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVETRPAGDGTFQKWAAVVVPSGQEQRYTCHMQHEGLQEPLTLSWEPS

SEQ ID NO: 152 - protein, 406 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVTRPAGDGTFQKWAAVVVPSGQEQRYTCHMQHEGLQEPLTLSWEPS

SEQ ID NO: 153 - protein, 410 aa, synthetic construct
SYRPGTEALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 154 - protein, 410 aa, synthetic construct
SYRPGTVELGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 155 - protein, 409 aa, synthetic construct
SYRPGTVALGCGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 156 - protein, 405 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 157 - protein, 415 aa, synthetic construct
SYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 158 - protein, 9 aa, Influenza A virus
GILGFVFTL

SEQ ID NO: 159 - protein, 9 aa, Homo sapiens
RYRPGTVAL

SEQ ID NO: 160 - protein, 9 aa, synthetic construct
SYRPGTVAL

SEQ ID NO: 161 - protein, 9 aa, synthetic construct
SRRYCNVAY

SEQ ID NO: 162 - protein, 9 aa, synthetic construct
SYRPCTVAL

SEQ ID NO: 163 - protein, 9 aa, synthetic construct
SYRPGTCAL

SEQ ID NO: 164 - protein, 9 aa, synthetic construct
SYRPGTVCL

SEQ ID NO: 165 - protein, 9 aa, synthetic construct
SYRPGTEAL

SEQ ID NO: 166 - protein, 9 aa, synthetic construct
SYRPGTVEL

SEQ ID NO: 167 - protein, 9 aa, synthetic construct
SWRYCNVAY

SEQ ID NO: 168 - protein, 9 aa, synthetic construct
SRRYCQVAY

SEQ ID NO: 169 - protein, 9 aa, synthetic construct
SRRYEMCAM

SEQ ID NO: 170 - protein, 9 aa, synthetic construct
SRRYLMCAY

SEQ ID NO: 171 - protein, 9 aa, synthetic construct
SRRWLMCAY

SEQ ID NO: 172 - protein, 9 aa, synthetic construct
SRRMEMVCI

SEQ ID NO: 173 - protein, 9 aa, synthetic construct
SRRMWMVCI

SEQ ID NO: 174 - protein, 9 aa, synthetic construct
SRRMEMVCR

SEQ ID NO: 175 - protein, 10 aa, synthetic construct
GGGGSGGGGS

SEQ ID NO: 176 - protein, 10 aa, synthetic construct
GCGGSGGGGS

SEQ ID NO: 177 - protein, 12 aa, synthetic construct
GGGGSGGGGSGS

SEQ ID NO: 178 - protein, 12 aa, synthetic construct
GCGGSGGGGSGS

SEQ ID NO: 179 - protein, 15 aa, synthetic construct
GGGGSGGGGSGGGGS

SEQ ID NO: 180 - protein, 15 aa, synthetic construct
GCGGSGGGGSGGGGS

SEQ ID NO: 181 - protein, 9 aa, synthetic construct
GGGSGGGGS

SEQ ID NO: 182 - protein, 9 aa, synthetic construct
GCGSGGGGS

SEQ ID NO: 183 - protein, 15 aa, synthetic construct
GGGGSGGGGSGGGGS

SEQ ID NO: 184 - protein, 20 aa, synthetic construct
GGGGSGGGGSGGGGSGGGGS

SEQ ID NO: 185 - protein, 10 aa, synthetic construct
GGGGSGGGGS

SEQ ID NO: 186 - protein, 20 aa, synthetic construct
GGGGAAAAAAAAGGGSGGGG

SEQ ID NO: 187 - protein, 151 aa, synthetic construct
QFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTLEVLFQGPSSHHHHHHSSGLNDIFEAQKIEWHE

SEQ ID NO: 188 - protein, 152 aa, synthetic construct
SQFRVSPLDRTWNLGETVELKCQVLLSNPTSGSSWLFQPRGAAASPTFLLYLSQNKPKAAEGLDTQRFSGKRLGDTFVLTLSDFRRENEGYYFCSALSNSIMYFSHFVPVFLPAKPTTTLEVLFQGPSSHHHHHHSSGLNDIFEAQKIEWHE

SEQ ID NO: 189 - protein, 152 aa, synthetic construct
SQFRVSPLDRTWNLGETVELKCQVLLSNPTSGASWLFQPRGAAASPTFLLYLSQNKPKAAEGLDTQRFSGKRLGDTFVLTLSDFRRENEGYYFCSALSNSIMYFSHFVPVFLPAKPTTTLEVLFQGPSSHHHHHHSSGLNDIFEAQKIEWHE

SEQ ID NO: 190 - protein, 240 aa, synthetic construct
MHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHECRFSAGPKVNGTFQADFPLGPATHGGTYRCFGSFRDSPYEWSNSSDPLLVSVIGNPSNSLEVLFQGPSSHHHHHHSSGLNDIFEAQKIEWHE

SEQ ID NO: 191 - protein, 240 aa, synthetic construct
MHEGVHRKPSLLAHPGRLVKSEETVILQCWSDVRFEHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHEARFSAGPKVNGTFQADFPLGPATHGGTYRCFGSFRDSPYEWSNSSDPLLVSVIGNPSNSLEVLFQGPSSHHHHHHSSGLNDIFEAQKIEWHE

SEQ ID NO: 192 - protein, 240 aa, synthetic construct
MHEGVHRKPSLLAHPGPLVKSEETVILQCWSDVRFQHFLLHREGKFKDTLHLIGEHHDGVSKANFSIGPMMQDLAGTYRCYGSVTHSPYQLSAPSDPLDIVITGLYEKPSLSAQPGPTVLAGESVTLSCSSRSSYDMYHLSREGEAHERRFSAGPKVNGTFQADFPLGPATHGGTYRCFGSFRDSPYEWSNSSDPLLVSVTGNPSNSLEVLFQGPSSHHHHHHSSGLNDIFEAQKIEWHE

SEQ ID NO: 193 - protein, 229 aa, synthetic construct
GHLPKPTLWAEPGSVITQGSPVTLRCQGGQETQEYRLYREKKTALWITRIPQELVKKGQFPIPSITWEHAGRYRCYYGSDTAGRSESSDPLELVVTGAYIKPTLSAQPSPVVNSGGNVILQCDSQVAFDGFSLCKEGEDEHPQCLNSQPHARGSSRAIFSVGPVSPSRRWWYRCYAYDSNSPYEWSLPSDLLELLVLGLEVLFQGPSSHHHHHHGLNDIFEAQKIEWHE

SEQ ID NO: 194 - protein, 278 aa, synthetic construct
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSS

SEQ ID NO: 195 - protein, 100 aa, synthetic construct [sequence omitted]

SEQ ID NO: 196 - protein, 5 aa, synthetic construct
VRAKR

SEQ ID NO: 197 - protein, 18 aa, synthetic construct
EGRGSLLTCGDVEENPGP

SEQ ID NO: 198 - protein, 4 aa, synthetic construct
SGSG

SEQ ID NO: 199 - protein, 123 aa, synthetic construct
SYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 200 - protein, 123 aa, synthetic construct
SYRPGTCALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 201 - protein, 120 aa, synthetic construct
SYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 202 - protein, 120 aa, synthetic construct
SYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 203 - protein, 397 aa, synthetic construct
SYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGFYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 204 - protein, 397 aa, synthetic construct
SYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGLYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 205 - protein, 397 aa, synthetic construct
SYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGFYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 206 - protein, 397 aa, synthetic construct
SYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGLYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 207 - protein, 523 aa, synthetic construct
MSRSVALAVLALLSLSGLEARYRPGTEALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 208 - protein, 523 aa, synthetic construct
MSRSVALAVLALLSLSGLEARYRPGTVELGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 209 - protein, 477 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACS

SEQ ID NO: 210 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 211 - protein, 528 aa, synthetic construct
MSRSVALAVLALLSLSGLEARYRPGTCALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 212 - protein, 528 aa, synthetic construct
MSRSVALAVLALLSLSGLEARYRPGTVCLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 213 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 214 - protein, 525 aa, synthetic construct
MSRSVALAVLALLSLSGLEARYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 215 - protein, 525 aa, synthetic construct
MSRSVALAVLALLSLSGLEARYRPGTVCLGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 216 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 217 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 218 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 219 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 220 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQVTQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 221 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 222 - protein, 528 aa, synthetic construct
MSRSVALAVLALLSLSGLEARYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 223 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 224 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 225 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 226 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 227 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQVTQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 228 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 229 - protein, 525 aa, synthetic construct
MSRSVALAVLALLSLSGLEARYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 230 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 231 - protein, 523 aa, synthetic construct
MSRSVALAVLALLSLSGLEARYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 232 - protein, 523 aa, synthetic construct
MSRSVALAVLALLSLSGLEARYRPGTVCLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQCDRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 233 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 234 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 235 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 236 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 237 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQVTQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 238 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 239 - protein, 523 aa, synthetic construct
MSRSVALAVLALLSLSGLEARYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 240 - protein, 522 aa, synthetic construct
MSRSVALAVLALLSLSGLEARYRPCTVALGGGGSGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQVTQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 241 - protein, 521 aa, synthetic construct
MSRSVALAVLALLSLSGLEARYRPCTVALGGGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQVTQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 242 - protein, 400 aa, synthetic construct
SYRPCTVALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 243 - protein, 400 aa, synthetic construct
SYRPGTCALGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 244 - protein, 397 aa, synthetic construct
SYRPCTVALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 245 - protein, 397 aa, synthetic construct
SYRPGTCALGGGGSGGGGSGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 246 - protein, 395 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 247 - protein, 395 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 248 - protein, 395 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 249 - protein, 395 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 250 - protein, 395 aa, synthetic construct
SYRPCTVALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 251 - protein, 395 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 252 - protein, 395 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGFYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 253 - protein, 395 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGLYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 254 - protein, 395 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGFYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 255 - protein, 395 aa, synthetic construct
SYRPGTCALGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGSHSMRYFDTAVSRPGRGEPRFISVGYVDDTQFVRFDSDAASPRGEPRAPWVEQEGPEYWDRETQKYKRQAQADRVSLRNLRGLYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 256 - protein, 415 aa, synthetic construct; identical to SEQ ID NO: 142

SEQ ID NO: 257 - protein, 410 aa, synthetic construct; identical to SEQ ID NO: 143

SEQ ID NO: 258 - protein, 410 aa, synthetic construct; identical to SEQ ID NO: 144

SEQ ID NO: 259 - protein, 410 aa, synthetic construct; identical to SEQ ID NO: 145

SEQ ID NO: 260 - protein, 410 aa, synthetic construct; identical to SEQ ID NO: 146

SEQ ID NO: 261 - protein, 410 aa, synthetic construct; identical to SEQ ID NO: 147

SEQ ID NO: 262 - protein, 410 aa, synthetic construct; identical to SEQ ID NO: 148

SEQ ID NO: 263 - protein, 410 aa, synthetic construct; identical to SEQ ID NO: 149

SEQ ID NO: 264 - protein, 410 aa, synthetic construct; identical to SEQ ID NO: 150

SEQ ID NO: 265 - protein, 407 aa, synthetic construct; identical to SEQ ID NO: 151

SEQ ID NO: 266 - protein, 406 aa, synthetic construct; identical to SEQ ID NO: 152

SEQ ID NO: 267 - protein, 410 aa, synthetic construct; identical to SEQ ID NO: 153

SEQ ID NO: 268 - protein, 410 aa, synthetic construct; identical to SEQ ID NO: 154

SEQ ID NO: 269 - protein, 409 aa, synthetic construct; identical to SEQ ID NO: 155

SEQ ID NO: 270 - protein, 405 aa, synthetic construct; identical to SEQ ID NO: 156

SEQ ID NO: 271 - protein, 415 aa, synthetic construct; identical to SEQ ID NO: 157

SEQ ID NO: 272 - protein, 523 aa, synthetic construct
MSRSVALAVLALLSLSGLEARYRPGTEALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 273 - protein, 523 aa, synthetic construct
MSRSVALAVLALLSLSGLEARYRPGTVELGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 274 - protein, 520 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 275 - protein, 519 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTELVTRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 276 - protein, 518 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 277 - protein, 528 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 278 - protein, 523 aa, synthetic construct
MSRSVALAVLALLSLSGLEARYRPGTVALGCGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFDTAVSRPGRGEPRFISVGYVDDTQFVRFDSDAASPRGEPRAPWVEQEGPEYWDRETQKYKRQAQADRVSLRNLRGCYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWEPSSQPTIPIMGIVAGLAVLVVLAVLGAVVTAMMCRRKSSGGKGGSCSQAACSNSAQGSDESLITCKAVRAKRSGSGEGRGSLLTCGDVEENPGPM

SEQ ID NO: 279 - protein, 277 aa, synthetic construct
GSHSMRYFDTAVSRPGRGEPRFISVGYVDDTQFVRFDSDAASPRGEPRAPWVEQEGPEYWDRETQKYKCQAQADRVSLRNLRGAYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 280 - protein, 277 aa, synthetic construct
GSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 281 - protein, 277 aa, synthetic construct
GSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 282 - protein, 277 aa, synthetic construct
GSHSMRYFDTAVSRPGRGEPRFISVGYVDDTQFVRFDSDAASPRGEPRAPWVEQEGPEYWDRETQKYKCQAQADRVSLRNLRGFYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 283 - protein, 277 aa, synthetic construct
GSHSMRYFDTAVSRPGRGEPRFISVGYVDDTQFVRFDSDAASPRGEPRAPWVEQEGPEYWDRETQKYKCQAQADRVSLRNLRGLYNQSEDGSHTLQRMSGCDLGPDGRLLRGYDQSAYDGKDYIALNEDLRSWTAADTAAQATQRKLEAARAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 284 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGAYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 285 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGFYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 286 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGLYNQSEDGSHTLQRMSGCDLGPDGRLLRGYDQSAYDGKDYIALNEDLRSWTAADTAAQITQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 287 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGFYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

SEQ ID NO: 288 - protein, 277 aa, synthetic construct
GSHSMRYFDTAVSRPGRGEPRFISVGYVDDTQFVRFDSDAASPRGEPRAPWVEQEGPEYWDRETQKYKRQAQADRVSLRNLRGLYNQSEDGSHTLQRMSGCDLGPDGRLLRGYDQSAYDGKDYIALNEDLRSWTAADTAAQATQRKLEACRAAEQLRAYLEGTCVEWLRRYLENGKETLQRAEPPKTHVTHHPLSDHEATLRCWALGFYPAEITLTWQRDGEDQTAKTELVETRPAGDGTFQKWAAVVVPSGQEQRYTCHMQHEGLQEPLTLSWEPS

(19) United States
(12) Patent Application Publication
(10) Pub. No.: US 2025/0270283 A1
(43) Pub. Date: Aug. 28, 2025

(54) METHODS AND COMPOSITIONS FOR CELLULAR THERAPY

(71) Applicant: REPLAY HOLDINGS, INC., San Diego, CA (US)

(72) Inventors: Adrian WOOLFSON, San Diego, CA (US); Herman WALDMANN, San Diego, CA (US); Ashley BUCKLE, San Diego, CA (US)

(21) Appl. No.: 18/858,247

(22) PCT Filed: Apr. 19, 2023
(86) PCT No.: PCT/US2023/065977

§ 371 Date: Oct. 18, 2024

(30) Foreign Application Priority Data
Apr. 20, 2022 (GB) 2205771.5

(51) Int. Cl. C07K 14/74; C12N 5/0735; C12N 5/074; C12N 5/0775; C12N 5/0789; C12N 15/62; C12N 15/85

(52) U.S. Cl. CPC C07K 14/70539; C12N 5/0606; C12N 5/0647; C12N 5/0663; C12N 5/0696; C12N 15/625; C12N 15/85; C07K 2319/02; C07K 2319/70; C12N 2510/00; C12N 2820/60

(57) ABSTRACT

Provided herein are synthetic constructs (synHLA) comprising one or more human leukocyte antigens, a targeting moiety, and linker regions, wherein said constructs do not elicit an immune response. Also provided are a nucleic acid molecule encoding said construct, an immune incompetent stem cell comprising said construct or said nucleic acid molecule, and a method of treating a disease or disorder comprising administering said construct, or said nucleic acid molecule.

### CROSS-REFERENCE

This application claims the benefit of UK Patent Application No 2205771.5, filed Apr. 20, 2022, which application is incorporated herein by reference in its entirety.

### SEQUENCE LISTING

The instant application contains a Sequence Listing which has been submitted electronically in XML format and is hereby incorporated by reference in its entirety. Said XML copy, created on Apr. 18, 2023, is named 62600-718_601_SL.xml and is 352,190 bytes in size.

### BACKGROUND

Cellular therapies hold great promise for combating previously intractable diseases. While the use of autologous cells is generally optimal, this approach can be cost-prohibitive and burdensome. Although allogeneic pluripotent stem cells (PSCs) provide greater scalability and cost savings, their utility is limited by the need to match human leukocyte antigen (HLA) class 1 alleles, the most genetically polymorphic region in the human genome. Mismatches in HLA class 1 haplotypes lead to the “self versus non-self” immune response that can result in the body's rejection of transplanted therapeutic cells. The general utility of recent efforts to engineer HLA constructs that are blocked from triggering a T cell-activated immune response has been limited by the failure of these constructs to successfully engage the killer-cell immunoglobulin-like receptor (KIR), resulting in a “missing self” immune response. Consequently, there remains an unmet need for the development of engineered pluripotent stem cells that circumvent both T cell- and KIR-mediated immune responses.

### SUMMARY

An aspect of the present disclosure provides a construct comprising: One or more targeting moieties; One or more major histocompatibility complex (MHC) regions, wherein at least one of the one or more MHC regions comprise a cluster of differentiation 8 (CD8) binding site; and One or more linker regions. In some embodiments, the CD8 binding site comprises one or more mutations. In some embodiments, the construct further comprises one or more disulfide staple pairs. In some embodiments, the one or more MHC regions are inhibited from eliciting a T cell or NK response when the construct is interrogated by one or more T cells. In some embodiments, the one or more MHC regions comprise one or more mutated residues relative to the wild-type version of the one or more MHC regions, wherein the one or more mutated residues are located at a corresponding position to the tyrosine 84 (Y84) residue of the human leukocyte antigen (HLA) protein HLA-C. In some embodiments, the one or more mutated residues comprise alanine. In some embodiments, the one or more mutated residues comprise cysteine. In some embodiments, the CD8 binding site comprises a mutation to the residue corresponding to the Q226 residue of HLA-C. In some embodiments, the CD8 binding site comprises a mutation to the residue corresponding to the D227K residue of HLA-C. In some embodiments, the T225 residue of the CD8 binding site is deleted. In some embodiments, wherein the Q226 residue of the CD8 binding site is deleted. In some embodiments, the T225 residue of the CD8 binding site is deleted. In some embodiments, the E232 residue of the CD8 binding site is deleted. In some embodiments, the one or more MHC regions further comprise one or more mutations to the residue corresponding to the C1 residue of HLA-C. In some embodiments, the residue is a glycine. In some embodiments, the disulfide staple pair is formed between the residue of the one or more MHC regions corresponding to the Y84 residue of an HLA-C and a residue in the one or more linker regions. In some embodiments, the disulfide staple pair is formed between the residue of the one or more MHC regions corresponding to the R69 residue of an HLA-C and a residue in the one or more targeting moieties. In some embodiments, the disulfide staple pair is formed between the residue of the one or more MHC regions corresponding to the A150 residue of an HLA-C and a residue in the one or more targeting moieties. In some embodiments, the disulfide staple pair is formed between the residue of the one or more MHC regions corresponding to the A73 residue of an HLA-C and a residue in the one or more targeting moieties. In some embodiments, the construct is soluble. In some embodiments, the construct is insoluble. In some embodiments, the construct comprises a beta-2 microglobulin (B2M) leader sequence. In some embodiments, the construct further comprises an N-terminal signal sequence. In some embodiments, the construct further comprises a C-terminal signal sequence. In some embodiments, the construct is a single chain trimer (SCT). In some embodiments, the construct is a single chain dimer (SCD). In some embodiments, the one or more MHC regions comprise one or more human HLA class 1 heavy chain sequences. In some embodiments, the one or more human HLA class 1 heavy chain sequences are derived from HLA-A, HLA-B, HLA-C, HLA-D, HLA-E, HLA-F, HLA-G, or some combination thereof. In some embodiments, the one or more targeting moieties comprises a peptide. In some embodiments, the peptide comprises a sequence at least about 50%, 60%, 70%, 80%, 90%, 95%, or 99% identical to any one of SEQ ID NOs: 158-174. In some embodiments, a linker region of the one or more linker regions comprises a sequence at least about 50%, 60%, 70%, 80%, 90%, 95%, or 99% identical to any one of SEQ ID NOs 175-186: In some embodiments, the construct further comprises a first linker region and a second linker region, wherein the first linker region comprises a sequence at least about 50%, 60%, 70%, 80%, 90%, 95%, or 99% identical to any one of SEQ ID NOs: 175-186; and wherein the second linker region comprises a sequence at least about 50%, 60%, 70%, 80%, 90%, 95%, or 99% identical to any one of SEQ ID NOs: 175-186. In some embodiments, the construct further comprises a sequence at least 50%, 60%, 70%, 80%, 90%, 95%, or 99% identical to any one of SEQ ID NOs: 30-129 and 142-157. In some embodiments, the construct comprises in N-terminus to C-terminus order: a targeting moiety of the one or more targeting moieties; a first linker of the one or more linkers; and a MHC region of the one or more MHC regions; and a disulfide stable pair configured to associate the targeting moiety and the MHC region or configured to associate the first linker and the MHC region. In some embodiments, the targeting moiety of the one or more targeting moieties comprises a sequence at least about 50%, 60%, 70%, 80%, 90%, 95%, or 99% identical to any one of SEQ ID NOs: 158-174. In some embodiments, the first linker comprises a sequence at least about 50%, 60%, 70%, 80%, 90%, 95%, or 99% identical to any one of SEQ ID NOs: 175-186. In some embodiments, the MHC region comprises a sequence at least about 50%, 60%, 70%, 80%, 90%, 95%, or 99% identical to any one of SEQ ID NOs: 130-141. In some embodiments, the construct further comprises a B2M leader sequence between the first linker and the MHC region. In some embodiments, the construct further comprises a second linker between the B2M leader sequence and the MHC region. In some embodiments, the second linker comprises a sequence at least about 50%, 60%, 70%, 80%, 90%, 95%, or 99% identical to any one of SEQ ID NOs: 175-186.

Another aspect of the present disclosure provides a hypo-immunogenic pluripotent stem cell comprising a construct comprising: one or more targeting moieties; one or more major histocompatibility complex (MHC) regions, wherein at least one of the one or more MHC regions comprise a cluster of differentiation 8 (CD8) binding site; and one or more linker regions; In some embodiments, the CD8 binding site comprises one or more mutations. In some embodiments, the hypo-immunogenic pluripotent stem cell further comprises one or more disulfide staple pairs. In some embodiments, the one or more MHC regions are inhibited from eliciting a T cell response when the complex is interrogated by one or more T cells. In some embodiments, the one or more MHC regions comprise one or more mutated residues relative to the wild-type version of the one or more MHC regions, wherein the one or more mutated residues are located at a corresponding position to the tyrosine 84 (Y84) residue of the human leukocyte antigen (HLA) protein HLA-C. In some embodiments, the one or more mutated residues comprise alanine. In some embodiments, the one or more mutated residues comprise cysteine. In some embodiments, the CD8 binding site comprises a mutation to the residue corresponding to the Q226 residue of HLA-C. In some embodiments, the CD8 binding site comprises a mutation to the residue corresponding to the D227K residue of HLA-C. In some embodiments, the T225 residue of the CD8 binding site is deleted. In some embodiments, the Q226 residue of the CD8 binding site is deleted. In some embodiments, the T22D2275 residue of the CD8 binding site is deleted. In some embodiments, the E232 residue of the CD8 binding site is deleted. In some embodiments, the one or more MHC regions further comprise one or more mutations to the residue corresponding to the C1 residue of HLA-C. In some embodiments, the residue is a glycine. In some embodiments, the disulfide staple pair is formed between the residue of the one or more MHC regions corresponding to the Y84 residue of an HLA-C and a residue in the one or more linker regions. In some embodiments, the disulfide staple pair is formed between the residue of the one or more MHC regions corresponding to the R69 residue of an HLA-C and a residue in the one or more targeting moieties. In some embodiments, the disulfide staple pair is formed between the residue of the one or more MHC regions corresponding to the A150 residue of an HLA-C and a residue in the one or more targeting moieties. In some embodiments, the disulfide staple pair is formed between the residue of the one or more MHC regions corresponding to the A73 residue of an HLA-C and a residue in the one or more targeting moieties. In some embodiments, the construct is soluble. In some embodiments, the construct is insoluble. In some embodiments, the construct comprises a beta-2 microglobulin (B2M) leader sequence. In some embodiments, the construct further comprises an N-terminal signal sequence. In some embodiments, the construct further comprises a C-terminal signal sequence. In some embodiments, the construct is a single chain trimer (SCT). In some embodiments, the construct is a single chain dimer (SCD). In some embodiments, the one or more MHC regions comprise one or more human HLA class 1 heavy chain sequences. In some embodiments, the one or more human HLA class 1 heavy chain sequences are derived from HLA-A, HLA-B, HLA-C, HLA-D, HLA-E, HLA-F, HLA-G, or some combination thereof. In some embodiments, the one or more targeting moieties comprises a peptide. In some embodiments, the peptide comprises a sequence at least about 50%, 60%, 70%, 80%, 90%, 95%, or 99% identical to any one of SEQ ID NOs: 158-174. In some embodiments, a linker region of the one or more linker regions comprises a sequence at least about 50%, 60%, 70%, 80%, 90%, 95%, or 99% identical to any one of SEQ ID NOs: 175-186. In some embodiments, the hypo-immunogenic pluripotent stem cell further comprises a first linker region and a second linker region, wherein the first linker region comprises a sequence at least about 50%, 60%, 70%, 80%, 90%, 95%, or 99% identical to any one of SEQ ID NOs: 175-186; and wherein the second linker region comprises a sequence at least about 50%, 60%, 70%, 80%, 90%, 95%, or 99% identical to any one of SEQ ID NOs: 175-186. In some embodiments, the hypo-immunogenic pluripotent stem cell further comprises a sequence at least 50%, 60%, 70%, 80%, 90%, 95%, or 99% identical to any one of SEQ ID NOs: 30-129 and 142-157. In some embodiments, the construct comprises in N-terminus to C-terminus order: a targeting moiety of the one or more targeting moieties; a first linker of the one or more linkers; and a MHC region of the one or more MHC regions; and a disulfide stable pair configured to associate the targeting moiety and the MHC region or configured to associate the first linker and the MHC region. In some embodiments, the targeting moiety of the one or more targeting moieties comprises a sequence at least about 50%, 60%, 70%, 80%, 90%, 95%, or 99% identical to any one of SEQ ID NOs: 158-174. In some embodiments, the first linker comprises a sequence at least about 50%, 60%, 70%, 80%, 90%, 95%, or 99% identical to any one of SEQ ID NOs: 175-186. In some embodiments, the MHC region comprises a sequence at least about 50%, 60%, 70%, 80%, 90%, 95%, or 99% identical to any one of SEQ ID NOs: 130-141. In some embodiments, the hypo-immunogenic pluripotent stem cell further comprises a B2M leader sequence between the first linker and the MHC region. In some embodiments, the hypo-immunogenic pluripotent stem cell further comprises a second linker between the B2M leader sequence and the MHC region. In some embodiments, the second linker comprises a sequence at least about 50%, 60%, 70%, 80%, 90%, 95%, or 99% identical to any one of SEQ ID NOs: 175-186. A method of generating a hypo-immunogenic pluripotent stem cell comprising: Generating a construct comprising one or more targeting moieties, one or more linker regions, and one or more major histocompatibility complex (MHC) regions, wherein at least one of the one or more MHC regions comprise a cluster of differentiation 8 (CD8) binding site; Providing the construct in a pluripotent stem cell (PSC); and Expressing the construct in the (PSC). In some embodiments, the CD8 binding site comprises one or more mutations In some embodiments, the one or more MHC regions are inhibited from eliciting a T cell response when the complex is interrogated by one or more T cells. In some embodiments, the one or more MHC regions comprise one or more mutated residues relative to the wild-type version of the one or more MHC regions, wherein the one or more mutated residues are located at a corresponding position to the tyrosine 84 (Y84) residue of the human leukocyte antigen (HLA) protein HLA-C. In some embodiments, the one or more mutated residues comprise alanine. In some embodiments, the one or more mutated residues comprise cysteine. In some embodiments, the CD8 binding site comprises a mutation to the residue corresponding to the Q226 residue of HLA-C. In some embodiments, the CD8 binding site comprises a mutation to the residue corresponding to the D227K residue of HLA-C. In some embodiments, the T225 residue of the CD8 binding site is deleted. In some embodiments, the Q226 residue of the CD8 binding site is deleted. In some embodiments, the T22D2275 residue of the CD8 binding site is deleted. In some embodiments, the E232 residue of the CD8 binding site is deleted. In some embodiments, the one or more MHC regions further comprise one or more mutations to the residue corresponding to the C1 residue of HLA-C. In some embodiments, the residue is a glycine. In some embodiments, the disulfide staple pair is formed between the residue of the one or more MHC regions corresponding to the Y84 residue of an HLA-C and a residue in the one or more linker regions. In some embodiments, the disulfide staple pair is formed between the residue of the one or more MHC regions corresponding to the R69 residue of an HLA-C and a residue in the one or more targeting moieties. In some embodiments, the disulfide staple pair is formed between the residue of the one or more MHC regions corresponding to the A150 residue of an HLA-C and a residue in the one or more targeting moieties. In some embodiments, the disulfide staple pair is formed between the residue of the one or more MHC regions corresponding to the A73 residue of an HLA-C and a residue in the one or more targeting moieties. In some embodiments, the construct is soluble. In some embodiments, the construct is insoluble. In some embodiments, the construct comprises a beta-2 microglobulin (B2M) leader sequence. In some embodiments, the construct further comprises an N-terminal signal sequence. In some embodiments, the construct further comprises a C-terminal signal sequence. In some embodiments, the construct is a single chain trimer (SCT). In some embodiments, the construct is a single chain dimer (SCD). In some embodiments, the one or more MHC regions comprise one or more human HLA class 1 heavy chain sequences. In some embodiments, the one or more human HLA class 1 heavy chain sequences are derived from HLA-A, HLA-B, HLA-C, HLA-D, HLA-E, HLA-F, HLA-G, or some combination thereof. In some embodiments, the one or more targeting moieties comprises a peptide. In some embodiments, the peptide comprises a sequence at least about 50%, 60%, 70%, 80%, 90%, 95%, or 99% identical to any one of SEQ ID NOs: 158-174. In some embodiments, a linker region of the one or more linker regions comprises a sequence at least about 50%, 60%, 70%, 80%, 90%, 95%, or 99% identical to any one of SEQ ID NOs 175-186: In some embodiments, the method further comprises a first linker region and a second linker region, wherein the first linker region comprises a sequence at least about 50%, 60%, 70%, 80%, 90%, 95%, or 99% identical to any one of SEQ ID NOs: 175-186; and wherein the second linker region comprises a sequence at least about 50%, 60%, 70%, 80%, 90%, 95%, or 99% identical to any one of SEQ ID NOs: 175-186. In some embodiments, the method further comprises a sequence at least 50%, 60%, 70%, 80%, 90%, 95%, or 99% identical to any one of SEQ ID NOs: 30-129 and 142-157. In some embodiments, the construct comprises in N-terminus to C-terminus order: a targeting moiety of the one or more targeting moieties; a first linker of the one or more linkers; and a MHC region of the one or more MHC regions; and a disulfide stable pair configured to associate the targeting moiety and the MHC region or configured to associate the first linker and the MHC region. In some embodiments, the targeting moiety of the one or more targeting moieties comprises a sequence at least about 50%, 60%, 70%, 80%, 90%, 95%, or 99% identical to any one of SEQ ID NOs: 158-174. In some embodiments, the first linker comprises a sequence at least about 50%, 60%, 70%, 80%, 90%, 95%, or 99% identical to any one of SEQ ID NOs: 175-186. In some embodiments, the MHC region comprises a sequence at least about 50%, 60%, 70%, 80%, 90%, 95%, or 99% identical to any one of SEQ ID NOs: 130-141. In some embodiments, the method further comprises a B2M leader sequence between the first linker and the MHC region. In some embodiments, the method further comprises a second linker between the B2M leader sequence and the MHC region. In some embodiments, the second linker comprises a sequence at least about 50%, 60%, 70%, 80%, 90%, 95%, or 99% identical to any one of SEQ ID NOs: 175-186.

Another aspect of the present disclosure provides for a construct comprising: a targeting moiety; a major histocompatibility complex (MHC) region; and a linker region disposed between the targeting moiety and the MHC region; wherein one of the targeting moiety, the MHC region, and the linker region comprises a first cysteine residue and another of the targeting moiety, the MHC region, and the linker region comprises a second cysteine residue, wherein the first cysteine residue and the second cysteine residue are configured to form a disulfide bond with one another when the construct is expressed on a surface of a cell.

In some embodiments, the MHC region comprises an MHC class I heavy chain. In some embodiments, the MHC region is derived from an HLA-A, HLA-B, or HLA-C sequence. In some embodiments, the MHC class I heavy chain comprises a sequence at least about 50%, 60%, 70%, 80%, 90%, 95%, or 99% identical to any one of SEQ ID NOs 130-141 and 279-288. In some embodiments, the MHC class I heavy chain comprises a mutation corresponding to the C1 residue of HLA-C (e.g., SEQ ID NO: 194). In some embodiments, the mutation comprises a glycine residue. In some embodiments, the construct further comprises a second MHC region. In some embodiments, the second MHC region is derived from an HLA-A, HLA-B, HLA-C, HLA-E, HLA-F, or HLA-G sequence. In some embodiments, the construct further comprises a beta-2 microglobulin (B2M) region. In some embodiments, the B2M region is disposed between the targeting moiety and the MHC region. In some embodiments, the B2M region comprises a sequence at least about 50%, 60%, 70%, 80%, 90%, 95%, or 99% identical to SEQ ID NO: 195. In some embodiments, the construct further comprises a second linker region disposed between the B2M region and the targeting moiety or the MHC region. In some embodiments, the linker region is disposed between the targeting moiety and the B2M region and the second linker region is disposed between the B2M region and the MHC region. In some embodiments, the linker region is less than fifteen amino acid residues in length. In some embodiments, the linker region is less than fourteen amino acid residues in length. In some embodiments, the linker region is less than thirteen amino acid residues in length. In some embodiments, the linker region is at least eight amino acid residues in length. In some embodiments, the linker region comprises at least one cysteine residue. In some embodiments, the linker region comprises amino acids selected from the group consisting of: glycine, serine, and cysteine. In some embodiments, the linker region comprises a sequence that is at least about 50%, 60%, 70%, 80%, 90%, 95%, or 99% identical to any one of SEQ ID NOs 175-186. In some embodiments, the linker region comprises a sequence selected from SEQ ID NOs: 175-186 and 198. In some embodiments, the second linker region comprises a sequence that is at least about 50%, 60%, 70%, 80%, 90%, 95%, or 99% identical to any one of SEQ ID NOs 175-186. In some embodiments, the second linker region comprises a sequence selected from SEQ ID NOs: 175-186 and 198. In some embodiments, the linker region and the second linker region are independently selected from SEQ ID NOs: 175-186 and 198. In some embodiments, the linker region comprises SEQ ID NO: 175 or 176 and the second linker region comprises SEQ ID NO: 183. In some embodiments, the targeting moiety comprises the first cysteine residue and the MHC region comprises the second cysteine residue. In some embodiments, the first cysteine residue is located any one of positions 1 9 of the targeting moiety. In some embodiments, the first cysteine residue is a C5, C7, or C8 residue of the targeting moiety. In some embodiments, the second cysteine residue corresponds to a Y84 residue an HLA-C heavy chain. In some embodiments, the second cysteine residue corresponds to an R69 residue an HLA-C heavy chain. In some embodiments, the second cysteine residue corresponds to an A73 residue of an HLA-C heavy chain. In some embodiments, the second cysteine residue corresponds to an A150 residue of an HLA-C heavy chain. In some embodiments, the linker region comprises the first cysteine and the MHC region comprises the second cysteine residue. In some embodiments, the first cysteine residue is a C2 of the linker. In some embodiments, the targeting moiety comprises a peptide configured to form a complex with the MHC region. In some embodiments, the peptide comprises a sequence at least about 50%, about 60%, about 70%, about 80%, about 90%, about 95%, or about 99% identical to any one of SEQ ID NOs: 158-174. In some embodiments, the peptide comprises a second amino acid residue selected form L, M, S, I, F, T, V, and Y. In some embodiments, the second amino acid residue is selected from T, V, and Y. In some embodiments, the peptide comprises a last amino acid residue selected from V, I, F, W, Y, L, R, and K. In some embodiments, the last amino acid residue is selected from Y, L, R, and K. In some embodiments, the peptide comprises a second amino acid residue selected from E, P, L, Q, A, R, H, S, T, V, M, D, and K. In some embodiments, the second amino acid residue is selected from E, P, L, Q, A, R, and H. In some embodiments, the peptide comprises a last amino acid residue selected from V, L, F, A, I, Y, M, W, P, and R. In some embodiments, the last amino acid residue is selected from V, L, and F. In some embodiments, the peptide comprises a second amino acid residue selected from A, Y, S, T, V, I, L, F, Q, R, N, and W. In some embodiments, the second amino acid residue is selected from A and Y. In some embodiments, the peptide comprises a last amino acid residue selected from L, V, M, F, Y, and I. In some embodiments, the last amino acid residue is L. In some embodiments, the last amino acid residue is a ninth, tenth, eleventh, twelfth reside of the peptide. In some embodiments, the construct comprises a sequence at least about 50%, 60%, 70%, 80%, 90%, 95%, or 99% identical to any one of SEQ ID NOs 30-129, 142-157, and 203-278. In some embodiments, the construct is inhibited form eliciting an NK cell response when the construct is interrogated by one or more NK cells.

A construct comprising: a targeting moiety; a major histocompatibility complex (MHC) region; and a linker region disposed between the targeting moiety and the MHC region; wherein the linker region comprises fewer than fifteen amino acid residues.

In some embodiments, MHC region comprises an MHC class 1 heavy chain. In some embodiments, the MHC class I heavy chain is derived from an HLA-A, HLA-B, or HLA-C sequence. In some embodiments, the MCH region comprises a sequence at least about 50%, 60%, 70%, 80%, 90%, 95%, or 99% identical to any one of SEQ ID NOs 130-141 and 279-288. In some embodiments, the MHC class I heavy chain comprises a mutation corresponding to the C1 residue of HLA-C (e.g., SEQ ID NO: 194). In some embodiments, the mutation comprises a glycine residue. In some embodiments, the construct further comprises a second MHC region. In some embodiments, the second MHC region is derived from an HLA-A, BLA-B, HLA-C, HLAE, HLA-F, or HLA-G sequence. In some embodiments, the construct further comprises a beta-2 microglobulin (B2M) region. In some embodiments, the B2M region is disposed between the targeting moiety and the MHC region. In some embodiments, the B2M region comprises a sequence at least about 50%, 60%, 70%, 80%, 90%, 95%, or 99% identical to SEQ ID NO: 195. In some embodiments, the construct further comprises a second linker region disposed between the B2M region and the targeting moiety or the MHC region. In some embodiments, the linker region is disposed between the targeting moiety and the B2M region and the second linker region is disposed between the B2M region and the MHC region. In some embodiments, the linker region is less than fourteen amino acid residues in length. In some embodiments, the linker region is less than thirteen amino acid residues in length. In some embodiments, the linker region is at least eight amino acid residues in length. In some embodiments, the linker comprises a first cysteine residue configured to form a disulfide staple pair with a second cysteine residue of the MHC region. In some embodiments, the first cysteine residue is a C2 of the linker. In some embodiments, the linker comprises amino acids selected from the group consisting of: glycine, serine, and cysteine. In some embodiments, the linker comprises a sequence that is at least about 50%, 60%, 70%, 80%, 90%, 95%, or 99% identical to any one of SEQ ID NOs 175-186 and 198. In some embodiments, the linker comprises a sequence selected from SEQ ID NOs: 175-186 and 198. In some embodiments, the second linker region comprises a sequence that is at least about 50%, 60%, 70%, 80%, 90%, 95%, or 99% identical to any one of SEQ ID NOs 175-186. In some embodiments, the second linker region comprises a sequence selected from SEQ ID NOs: 175-186 and 198. In some embodiments, the linker region and the second linker region are independently selected from SEQ ID NOs: 175-186 and 198. In some embodiments, the linker region comprises SEQ ID NO: 175 or 176 and the second linker region comprises SEQ ID NO: 183. In some embodiments, the targeting moiety comprises a peptide configured to form a complex with the MHC region. In some embodiments, the peptide comprises a sequence at least about 50%, about 60%, about 70%, about 80%, about 90%, about 95%, or about 99% identical to any one of SEQ ID NOs: 158-174. In some embodiments, the peptide comprises a second amino acid residue selected form L, M, S, I, F, T, V, and Y. In some embodiments, the second amino acid residue is selected from T, V, and Y. In some embodiments, the peptide comprises a last amino acid residue selected from V, I, F, W, Y, L, R, and K. In some embodiments, the last amino acid residue is selected from Y, L, R, and K. In some embodiments, the peptide comprises a second amino acid residue selected from E, P, L, Q, A, R, H, S, T, V, M, D, and K. In some embodiments, the second amino acid residue is selected from E, P, L, Q, A, R, and H. In some embodiments, the peptide comprises a last amino acid residue selected from V, L, F, A, I, Y, M, W, P, and R. In some embodiments, the last amino acid residue is selected from V, L, and F. In some embodiments, the peptide comprises a second amino acid residue selected from A, Y, S, T, V, I, L, F, Q, R, N, and W. In some embodiments, the second amino acid residue is selected from A and Y. In some embodiments, the peptide comprises a last amino acid residue selected from L, V, M, F, Y, and I. In some embodiments, the last amino acid residue is L. In some embodiments, the last amino acid residue is a ninth, tenth, eleventh, twelfth reside of the peptide.

Another aspect of the present disclosure provides for a nucleic acid encoding any of the constructs disclosed herein.

Another aspect of the present disclosure provides for an engineered vector encoding any of the nucleic acids disclosed herein.

In some embodiments, the vector is a plasmid, a minicircle, a CELiD, an adeno-associated virus (AAV) derived virion, a lentivirus, an adenovirus, or a herpes simplex virus (HSV).

Another aspect of the present disclosure provides for a method of generating a hypo-immunogenic cell comprising administering to a cell any vector disclosed herein.

Another aspect of the present disclosure provides for a hypo-immunogenic cell comprising any construct disclosed herein.

In some embodiments, the cell is a stem cell. In some embodiments, the stem cell is an embryonic stem cell (ESC), a mesenchymal stem cell (MSC), an induced pluripotent stem cell (iPSC), or a hematopoietic stem cell (HSC).

### INCORPORATION BY REFERENCE

All publications, patents, and patent applications mentioned in this specification and appendences attached hereto, are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a domain view of the synthetic human leukocyte antigen (synHLA) construct provided herein.

FIG. 2 shows a three-dimensional view of the construct architecture of the synthetic human leukocyte antigen (synHLA) construct provided herein.

FIG. 3 shows an HLA-bound immunogenic peptide engaging with a T-cell receptor, resulting in T cell activation.

FIG. 4 shows the synthetic human leukocyte antigen (synHLA) construct provided herein engaging with a T-cell receptor, resulting in failed T cell activation.

FIG. 5 shows a single-chain trimer (SCT) in construct with killer-cell immunoglobulin-like receptor (KIR), resulting in a blocked KIR interaction and a “missing self” immune signal.

FIG. 6 shows the synthetic human leukocyte antigen (synHLA) construct provided herein in construct with killer-cell immunoglobulin-like receptor (KIR), resulting in a successful KIR interaction and no “missing self” immune signal.

FIG. 7 shows an overlay of a single-chain trimer (SCT) in construct with killer-cell immunoglobulin-like receptor (KIR) and the synthetic human leukocyte antigen (synHLA) construct provided herein in construct with killer-cell immunoglobulin-like receptor (KIR).

FIG. 8 shows the synthetic human leukocyte antigen (synHLA) construct provided herein engaging with CD8.

FIG. 9 shows the immune incompetent cell provided herein.

FIG. 10 shows an SDS-PAGE gel of synHLA constructs as described herein expressed recombinantly in bacteria.

FIG. 11 shows an SDS-PAGE of a synHLA construct as described herein expressed recombinantly in bacteria.

FIG. 12A shows a raw thermal melt curve of SYNC4-1 and SYNC4-1+KIR2DL2.

FIG. 12B shows the first derivative of a thermal melt curve of SYNC4-1 and SYNC4-1+KIR2DL2.

FIG. 13A shows a raw thermal melt curve of SYNC4-1, SYNC4-1+KIR2DL2, and KIR2DL2.

FIG. 13B shows the first derivative of a thermal melt curve of SYNC4-1, SYNC4-1+KIR2DL2, and KIR2DL2.

FIG. 14A shows a raw thermal melt curve of SYNA1-1, SYNA1-1+KIR2DL2, and KIR2DL2.

FIG. 14B shows the first derivative of a thermal melt curve of SYNA1-1, SYNA1-1+KIR2DL2, and KIR2DL2.

FIG. 15 shows a domain view of a synthetic human leukocyte antigen (synHLA) construct provided herein as designed for expression in bacteria.

FIG. 16 shows an SDS-PAGE gel of a synHLA construct as described herein expressed recombinantly in bacteria.

FIG. 17 shows an SDS-PAGE gel of a synHLA construct as described herein expressed recombinantly in bacteria.

FIG. 18 shows a mass spectrum of a synHLA construct as described herein as measured by TOF-MS.

FIGS. 19A-19F show results of dynamic light scattering (DLS) experiments as performed on constructs described herein.

FIG. 20 shows an SDS-PAGE gel of a synHLA construct as described herein expressed recombinantly in bacteria.

FIG. 21 shows a domain view of a synHLA construct provided herein as designed for expression in bacteria by multiple transcription units.

FIG. 22 shows an SDS-PAGE gel of a synHLA construct as described herein expressed recombinantly in bacteria.

FIG. 23 shows representative surface plasmon resonance (SPR) sensorgrams for binding of representative HLA proteins as disclosed herein to immobilized killer-cell immunoglobulin-like receptors (KIRs).

FIG. 24 shows relative binding of HLA proteins as disclosed herein to KIRs as determined by SPR.

FIG. 25 illustrates representative flow cytometric data showing generation of beta-2 microglobulin (B2M) deficient EBV cell lines.

FIG. 26 illustrates representative histograms from flow cytometry experiments showing expression of synthetic HLA proteins as described herein on the surface of B2M deficient EBV cells. Expression of the HLA-A2 on the surface of β2M null EBV 9031 cells was examined by staining with an antibody specific for HLA-A2 (BB7.2). The plot shows the staining profile of negative control samples, either transfected with an irrelevant protein or untransfected (as arrowed). HLA-A2 expressed on the surface of cells after transfection with the SCTA2-M1 construct or the SCDA1-M1 construct (as arrowed) is shown. All transfected cells were grown in the presence of puromycin for at least 7 days to select the transfected cells. MFI, mean fluorescent intensities.

FIG. 27 depicts a schematic illustration of multi-gene expression vector products. HLA proteins were fused to a GFP reporter in a multi-gene expression vector. The anticipated dominant products are shown schematically, assuming furin cleavage of the T2A peptide and ribosome skipping at the 2A ‘break’ site. Furin cleavage site, VRAKR (SEQ ID NO: 196); T2A peptide, EGRGSLLTCGDVEENPGP (SEQ ID NO: 197); SGSG linker (SEQ ID NO: 198); GFP, green fluorescent protein.

FIG. 28 depicts a table summarizing synthetic HLA constructs and resultant HLA-C expression. The proportion of HLA-C positive cells within the GFP+ population is reported for two experiments. HLA-C staining is reported after subtraction of staining from the isotype control.

FIG. 29 depicts histograms measuring the surface expression of HLA-C after transfecting B2M deficient EBV cells with different synthetic HLA constructs. The B2M deficient EBV transformed B-cell line, 9031, was transfected with a derivative of the pCE plasmid which encodes the expression of HLA-C single chain trimer variants fused, via a T2A peptide, to green fluorescent protein (GFP). For analysis the GFP+ population was gated (far left plot ‘starting population’), from this population the % of positive cells after staining with an isotype control (clone MPC-11 AlexaFluor 647) was determined and then the % of HLA-C expressing cells (after staining with the mAb DT-9 AlexaFluor 647) was determined. The number refers to the % of cells falling within the black box from the entire plot. Unstained, GFP-ve cells fall within the lower left quadrant.

FIG. 30 shows a graph summarizing the proportion of cells expressing GFP and presenting synthetic HLA proteins at the cell surface The B2M deficient EBV transformed B-cell line, 9031 was transfected with a derivative of the pCE plasmid which encodes the expression of synthetic HLA variants fused, via a T2A peptide, to green fluorescent protein (GFP). For analysis the GFP+ population was gated (far left plot ‘starting population’), from this population the % of positive cells after staining with an isotype control (clone MPC-11 AlexaFluor 647) was determined and then the % of HLA-C expressing cells (after staining with the mAb DT-9 AlexaFluor 647) was determined.

FIG. 31 illustrates dynamics of surface expression of HLA-C after transfecting B2M deficient EBV cells with different constructs. The B2M deficient EBV transformed B-cell line, 9031 was transfected with a derivative of the pCE plasmid which encodes the expression of HLA-C single chain trimer variants fused, via a T2A peptide, to green fluorescent protein (GFP). For analysis the percentage of GFP⁺HLA-C+, detected by staining with the mAb DT-9 AlexaFluor 647, minus the % of positive cells after staining with an isotype control (clone MPC-11 AlexaFluor 647) was determined for each construct at 1, 2, 5, 9 and 13 days after transfection.

FIG. 32 depicts a gel showing expression of GFP in transfected cells. Cells (2.0×10⁵) were lysed in RIPA buffer (Sigma, P0278-50ML) with Complete protease inhibitor cocktail (Sigma, P8340-1ML) and run on a reducing 4-12% SDS PAGE gel (Thermo Fisher Scientific, NW04127BOX). The proteins were transferred to Amersham Protran 0.45 mm NC nitrocellulose membrane (Sigma, GE10600008) and probed with a mouse anti-GFP mAb (clone B34, Biolegend cat #902601) which was detected using a goat anti-mouse IgG (H+L) antibody conjugated to DyLight 800 (Thermo Fisher Scientific, SA5-35521). The membrane was imaged using an Odyssey CLx imaging system. The highest molecular weight band is consistent with GFP linked to HLA-C, and ˜62 kDa band is consistent with human immunoglobulin (Hu-Ig). GFP (˜27 kDa) is predominantly observed in a free form.

FIG. 33 depicts flow cytometry results illustrating NK cell expression. NK cells were purified from peripheral blood mononuclear cells (PBMC) of healthy volunteer donors using a Miltenyi NK Cell Isolation Kit (Cat No. 130-092-657). Purified NK cells were expanded using a bead bound antibodies from a Miltenyi NK Activation/expansion kit (cat No. 130-094-483) in Miltenyi's NK media, supplemented with 5% pooled human serum and 500 U/ml of IL-2. Expanded NK cells were used in functional assays at least 7-10 days after the start of the culture.

FIG. 34 depicts thermostability data for constructs described herein.

FIG. 35 depicts representative surface plasmon resonance (SPR) sensorgrams characterizing the interaction between killer-cell immunoglobulin-like receptors (KIRs) and constructs described herein.

FIG. 36 depicts the results of a chromium-release assay demonstrating protection against NK-cell cytotoxicity provided by constructs described herein.

FIG. 37A depicts a schematic representation of synergistic inhibition of NK cells by co-expression of HLA-E and constructs described herein.

FIG. 37B depicts a schematic representation of an interaction between HLA-E and an NKG2A receptor.

FIG. 38 depicts the results of a chromium-release assay demonstrating synergistic protection against NK-cell cytotoxicity provided by constructs described herein in combination with HLA-E.

FIG. 39 depicts part of a crystal structure of a complex between a KIR2DL2 receptor and a construct as described herein.

FIG. 40 illustrates the results of a luciferase-based assay for cytotoxicity as described herein

### BRIEF DESCRIPTION OF THE SEQUENCE LISTING

The Sequence Listing filed herewith provides example polynucleotide and polypeptide sequences for use in the methods, compositions, and systems according to the disclosure. Below are representative descriptions of sequences therein.

SEQ ID NOs: 1-15 show representative amino acid sequences of single chain trimer (SCT) constructs as described herein.

SEQ ID NOs: 16-29 and 199-202 show representative amino acid sequences of single chain dimer (SCD) constructs as described herein.

SEQ ID NOs: 30-129, 142-157, and 203-278 show full-length amino acid sequences of representative synthetic HLA (synHLA) constructs as described herein.

SEQ ID NOs: 130-141 and 279-288 show representative amino acid sequences of HLA heavy chain (HHC) constructs as described herein.

SEQ ID NOs: 158-174 show amino acid sequences of representative peptides configured to weaken or inhibit HLA activity as described herein.

SEQ ID NOs: 175-186 and 198 show amino acid sequences of representative linkers between inhibitory amino acids and HLA sequences or between B2M regions and HLA sequences as described herein.

SEQ ID NOs: 187-189 show amino acid sequences of representative CD8-α chains as expressed and described herein.

SEQ ID NOs: 190-192 show amino acid sequences of killer Ig-like receptor (KIR) domain as expressed and described herein.

SEQ ID NO: 193 shows an amino acid sequence of a leukocyte immunoglobulin-like receptor (LILR) domain as expressed and described herein.

SEQ ID NO: 194 show an amino acid sequence of a representative HLA-C allele.

SEQ ID NO: 195 shows an amino acid sequence of a representative beta-2 microglobulin (B2M) sequence as described herein.

SEQ ID NO: 196 shows an amino acid sequence of a representative furin cleavage site.

SEQ ID NO: 197 shows an amino acid sequence of a representative T2A peptide.

### DETAILED DESCRIPTION OF THE INVENTION

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

As used herein, a “T cell” generally refers to a cell comprising a T-cell receptor.

As used herein, a “peptide” is a chain of between two and fifty amino acid residues.

A “pharmaceutically acceptable carrier” generally refers to an ingredient in a pharmaceutical formulation, other than an active ingredient, which is nontoxic to a subject. A pharmaceutically acceptable carrier includes, but is not limited to, a buffer, excipient, stabilizer, or preservative, such as those known in the art, for example, described in Remington's Pharmaceutical Sciences 16th edition, Osol, A. Ed. (1980).

As used herein, “treatment” or “treating” is an approach for obtaining beneficial or desired results including and preferably clinical results. For example, beneficial or desired clinical results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals.

As used herein, an “effective dosage” or “effective amount” of construct, nucleic acid molecule, immune incompetent cell, or pharmaceutical composition thereof generally refers to an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (e.g., slow to some extent and preferably stop) cancer cell infiltration into peripheral organs; inhibit (e.g., slow to some extent and preferably stop) tumor metastasis; inhibiting, to some extent, tumor growth; and/or relieving to some extent one or more of the symptoms associated with the disorder. An effective dosage can be administered in one or more administrations. For purposes of this invention, an effective dosage of construct, nucleic acid molecule, immune incompetent cell, or pharmaceutical composition thereof is an amount sufficient to accomplish prophylactic or therapeutic treatment either directly or indirectly. As is understood in the clinical context, an effective dosage of a construct, nucleic acid molecule, immune incompetent cell, or pharmaceutical composition thereof may or may not be achieved in conjunction with another construct, nucleic acid molecule, immune incompetent cell, or pharmaceutical composition thereof. Thus, an “effective dosage” may be considered in the context of administering one or more therapeutic agents, and a single agent may be considered to be given in an effective amount if, in conjunction with one or more other agents, a desirable result may be or is achieved.

As defined herein, the term “inhibition”, “inhibit”, “inhibiting” and the like in reference to a protein-inhibitor interaction generally means negatively affecting (e.g. decreasing) the activity or function of the protein relative to the activity or function of the protein in the absence of the inhibitor. Inhibition may refer to reduction of a disease or symptoms of disease. Inhibition may refer to a reduction in the activity of a particular protein or nucleic acid target. The protein may be deoxycytidine kinase. Thus, inhibition includes, at least in part, partially or totally blocking stimulation, decreasing, preventing, or delaying activation, or inactivating, desensitizing, or down-regulating signal transduction or enzymatic activity or the amount of a protein.

The term “modulator” generally refers to a composition that increases or decreases the level of a target molecule or the function of a target molecule or the physical state of the target of the molecule.

The term “modulate” is used in accordance with its plain ordinary meaning and generally refers to the act of changing or varying one or more properties. “Modulation” refers to the process of changing or varying one or more properties. For example, a modulator of a target protein changes by increasing or decreasing a property or function of the target molecule or the amount of the target molecule. A modulator of a disease decreases a symptom, cause, or characteristic of the targeted disease.

“Pharmaceutically acceptable excipient” and “pharmaceutically acceptable carrier” generally refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the constructs, nucleic acid molecules, or immune incompetent cells of the invention. One of skill in the art will recognize that other pharmaceutical excipients are useful in the present invention.

As used herein, the term “administering” generally includes oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration can be by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

“Patient,” “subject,” “patient in need thereof,” and “subject in need thereof” are herein used interchangeably and generally refer to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human. A “cancer-patient” is a patient suffering from, or prone to developing cancer.

Unless clearly indicated otherwise, the term “individual” as used herein generally refers to a mammal, including but not limited to, bovine, horse, feline, rabbit, canine, rodent, or primate (e.g., human). In some embodiments, an individual is a human. In some embodiments, an individual is a non-human primate such as chimpanzees and other apes and monkey species. In some embodiments, an individual is a farm animal such as cattle, horses, sheep, goats and swine; pets such as rabbits, dogs and cats; laboratory animals including rodents, such as rats, mice, and guinea pigs; and the like. In some embodiments, the invention find use in both human medicine and in the veterinary context.

“Disease” or “condition” generally refer to a state of being or health status of a patient or subject capable of being treated with the constructs, nucleic acid molecules, immune incompetent cells, or methods provided herein. In some embodiments, the disease as used herein refers to cancer.

As used herein, “immune checkpoint modulator” generally refers to an agent which results in the activation or inhibition of one or more immune checkpoint proteins. For example, immune checkpoint modulators may include, but are not limited to, CD47, PD-L1, A2AR, B7-H3, B7-H4, BTLA, CTLA-4, IDO, KIR, LAG3, NOX2, PD-1, TIM-3, VISTA, and SIGLEC7.

As used herein, “mutation” generally refers to an alteration in the sequence of a nucleic acid molecule. Mutations include, but are not limited to, insertions, deletions, and substitutions.

As used herein, the abbreviations for amino acids are conventional and can be as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Other amino acids include citrulline (Cit); homocysteine (Hey); hydroxyproline (Hyp); ornithine (Orn); and thyroxine (Thx). Examples of amino acids that are not charged at physiological pH include, but are not limited to, alanine, asparagine, cysteine, glutamine, glycine, isoleucine, leucine, methionine, phenylalanine, proline, serine, threonine, tryptophan, tyrosine, and valine.

As used herein, an “anchor residue” of a peptide generally refers to a conserved amino acid residue that plays a role in binding the peptide into the groove of a given HLA allele.

As used herein and in the appended claims, the singular forms “a,” “an,” and “the” include plural reference unless the context clearly indicates otherwise.

It is understood that aspect and variations of the invention described herein include “consisting” and/or “consisting essentially of” aspects and variations.

Synthetic Human Leukocyte Antigen (synHLA) Constructs

Provided herein, in one aspect, is a construct comprising one or more human leukocyte antigens (HLAs). In some embodiments, the one or more HLAs are inhibited from eliciting a T cell response when the construct is interrogated by one or more T cells. In some embodiments, the one or more HLAs are inhibited from eliciting a natural killer (NK)-cell response when the construct is interrogated by one or more NK cells. In some embodiments, the one or more HLAs are inhibited from eliciting a T cell response when interrogated by one or more T cells and from eliciting an NK cell response when interrogated by one or more NK cells.

In some embodiments, the construct comprises, in N-terminus to C-terminus order, a segment comprising a peptide and a segment comprising a beta-2 microglobulin (B2M) sequence. In some embodiments, the construct comprises, in N-terminus to C-terminus order, a segment comprising a peptide and a segment comprising a human HLA class 1 heavy chain sequence. In some embodiments, the construct comprises, in N-terminus to C-terminus order, a segment comprising a peptide, a segment comprising a B2M sequence, and a sequence comprising a human HLA class 1 heavy chain sequence.

In some embodiments, the human HLA class 1 heavy chain sequence comprises one or more class 1 HLAs. In some embodiments, the human HLA class 1 heavy chain sequence comprises HLA-A, HLA-B, HLA-C, or any combination thereof. In some embodiments, the human HLA class 1 heavy chain sequence comprises HLA-A. In some embodiments, the human HLA class 1 heavy chain sequence comprises HLA-B. In some embodiments, the human HLA class 1 heavy chain sequence comprises HLA-C. In some embodiments, the human HLA class 1 heavy chain sequence comprises HLA-A and HLA-B. In some embodiments, the human HLA class 1 heavy chain sequence comprises HLA-A and HLA-C. In some embodiments, the human HLA class 1 heavy chain sequence comprises HLA-B and HLA-C. In some embodiments, the human HLA class 1 heavy chain sequence comprises HLA-A, HLA-B, and HLA-C. In some embodiments, the human HLA class 1 heavy chain sequence comprise multiple versions of HLA-A, HLA-B, HLA-C, or any combination thereof. In some embodiments, the human HLA class 1 heavy chain sequence comprise multiple versions of HLA-A. In some embodiments, the human HLA class 1 heavy chain sequence comprise multiple versions of HLA-B. In some embodiments, the human HLA class 1 heavy chain sequence comprise multiple versions of HLA-C. In some embodiments, the human HLA class 1 heavy chain sequence comprise multiple versions of HLA-A and HLA-B. In some embodiments, the human HLA class 1 heavy chain sequence comprise multiple versions of HLA-A and HLA-C. In some embodiments, the human HLA class 1 heavy chain sequence comprise multiple versions of HLA-B and HLA-C. In some embodiments, the human HLA class 1 heavy chain sequence comprise multiple versions of HLA-A, HLA-B, and HLA-C. In some embodiments, the human HLA class 1 heavy chain sequence comprises the HLA-A, wherein the HLA-A is displaced between the HLA-B and the HLA-C.

In some embodiments, the construct further comprises one or more immune checkpoint modulators. In some embodiments, the one or more immune checkpoint modulators comprise CD47, PD-L1, A2AR, B7-H3, B7-H4, BTLA, CTLA-4, IDO, KIR, LAG3, NOX2, PD-1, TIM-3, VISTA, SIGLEC7, or any combination thereof. In some embodiments, the construct comprises CD47. In some embodiments, the construct comprises PD-L1. In some embodiments, the construct comprises A2AR. In some embodiments, the construct comprises B7-H3. In some embodiments, the construct comprises B7-H4. In some embodiments, the construct comprises BTLA. In some embodiments, the construct comprises CTLA-4. In some embodiments, the construct comprises IDO. In some embodiments, the construct comprises KIR. In some embodiments, the construct comprises LAG3. In some embodiments, the construct comprises NOX2. In some embodiments, the construct comprises PD-1. In some embodiments, the construct comprises TIM-3. In some embodiments, the construct comprises VISTA. In some embodiments, the construct comprises SIGLEC7.

In some embodiments, the human HLA class 1 heavy chain sequence comprises HLA-E or a fragment thereof, HLA-F or a fragment thereof, HLA-G or a fragment thereof, or any combination thereof. In some embodiments, the human HLA class 1 heavy chain sequence comprises HLA-E or a fragment thereof. In some embodiments, the human HLA class 1 heavy chain sequence comprises HLA-F or a fragment thereof. In some embodiments, the human HLA class 1 heavy chain sequence comprises HLA-G or a fragment thereof. In some embodiments, the human HLA class 1 heavy chain sequence comprises HLA-E or a fragment thereof and HLA-F or a fragment thereof. In some embodiments, the human HLA class 1 heavy chain sequence comprises HLA-E or a fragment thereof and HLA-G or a fragment thereof. In some embodiments, the human HLA class 1 heavy chain sequence comprises HLA-F or a fragment thereof and HLA-G or a fragment thereof. In some embodiments, the human HLA class 1 heavy chain sequence comprises HLA-E or a fragment thereof, HLA-F or a fragment thereof, and HLA-G or a fragment thereof. In some embodiments, at least one of the HLA-E or the fragment thereof, HLA-F or the fragment thereof, HLA-G or the fragment thereof, or any combination thereof is inhibited from eliciting a T cell response when the construct is interrogated by one or more T cells. In some embodiments, HLA-E or the fragment thereof is inhibited from eliciting a T cell response when the construct is interrogated by one or more T cells. In some embodiments, HLA-F or the fragment thereof is inhibited from eliciting a T cell response when the construct is interrogated by one or more T cells. In some embodiments, HLA-G or the fragment thereof is inhibited from eliciting a T cell response when the construct is interrogated by one or more T cells. In some embodiments, HLA-E or the fragment thereof and HLA-F or the fragment thereof are inhibited from eliciting a T cell response when the construct is interrogated by one or more T cells. In some embodiments, HLA-E or the fragment thereof and HLA-G or the fragment thereof are inhibited from eliciting a T cell response when the construct is interrogated by one or more T cells. In some embodiments, HLA-F or the fragment thereof and HLA-G or the fragment thereof are inhibited from eliciting a T cell response when the construct is interrogated by one or more T cells. In some embodiments, HLA-E or the fragment thereof, HLA-F or the fragment thereof, and HLA-G or the fragment thereof are inhibited from eliciting a T cell response when the construct is interrogated by one or more T cells.

In some embodiments, the construct further comprises an epitope configured to allow for detection of the construct. In some embodiments, the epitope comprises 3,5-dinitrosalicylic acid.

In some embodiments, the construct comprises a human beta-2 microglobulin (B2M) sequence. In some embodiments, the human B2M sequence is a wild-type human B2M sequence. In some embodiments, the B2M sequence comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to SEQ ID NO: 195.

In some embodiments, the construct comprises, in N-terminus to C-terminus order,
a. the peptide;
b. a first linker of the one or more linkers;
c. the human B2M sequence;
d. a second linker of the one or more linkers; and
e. the human HLA class 1 heavy chain sequence.

Provided herein, in another aspect, is a construct comprising one or more human leukocyte antigens (HLAs). In some embodiments, the construct comprises, in N-terminus to C-terminus order,
a. a peptide, wherein the peptide is incapable of activating the one or more T cells;
b. a first linker; and
c. a segment comprising a human HLA class 1 heavy chain sequence;
   wherein the first linker comprises a conformation configured to not block one or more killer-cell immunoglobulin-like receptor (KIR) binding sites on the human HLA class 1 heavy chain sequence. In some embodiments, the conformation is further configured to resist proteolytic cleavage.

In some embodiments, the one or more HLAs are inhibited from eliciting a T cell response when the construct is interrogated by one or more T cells. In some embodiments, the one or more HLAs are inhibited from eliciting an NK cell response when the construct is interrogated by one or more NK cells. In some embodiments, the construct comprises one or more targeting moieties (e.g., peptides); one or more linker regions; one or more major histocompatibility complex (MHC) regions (such as a HLA class 1 heavy chain sequence), wherein at least one of the one or more MHC regions comprises a cluster of differentiation 8 (CD8) binding site and wherein the CD8 binding site comprises one or more mutations; and one or more disulfide staple pairs.

The one or more targeting moieties may comprise a peptide incapable of activating one or more T cells as described elsewhere herein (e.g., as listed in Table 2) when associated with an MHC region (e.g., an HLA class I heavy chain sequence). The targeting moieties may comprise a peptide incapable of activating one or more NK cells as described elsewhere herein (e.g., as listed in Table 2) when associated with an HLA class I heavy chain sequence.

The one or more MHC regions may comprise one or more human HLA class I heavy chain sequences. In some cases, the human HLA class 1 heavy chain sequence is derived from HLA-A, HLA-B, HLA-C, HLA-D, HLA-E, HLA-F, HLA-G, or any combination thereof. In some cases, the human HLA class 1 heavy chain sequence comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 130-141 and 279-288.

The one or more MHC regions may be inhibited from eliciting a T cell response when the construct is interrogated by one or more T cells. The one or more MHC regions may comprise one or more mutated residues relative to a wild-type version of the one or more MHC regions. In some embodiments, the mutation is at a site corresponding to tyrosine 84 (Y84) residue of a human leukocyte antigen (HLA) protein HLA-C (e.g., SEQ ID NO:194). The mutation may be to any suitable amino acid. In some embodiments, the mutation comprises an alanine residue (e.g., a Y84A mutation). In some embodiments, the mutation comprises a cysteine residue (e.g., a Y84C mutation).

The CD8 binding site may comprise a mutation at any residue to any other amino acid or a deletion at any residue. In some embodiments, the mutation may comprise an insertion before or after a residue. In some embodiments, the CD8 binding site may comprise a mutation at the position corresponding to glutamine 226 (Q226) of HLA-C (e.g., SEQ ID NO:194). In some embodiments, the CD8 binding site may comprise a mutation to the residue corresponding to aspartate 227 (D227) of HLA-C. In some embodiments, the mutation at D227 of HLA-C is to a lysine residue (e.g., a D227K mutation). In some embodiments, the CD8 binding site may comprise a deletion of the residue corresponding to threonine 225 (T225) of HLA-C. In some embodiments, the CD8 binding site may comprise a deletion of the Q226 residue. In some embodiments, the CD8 binding site may comprise a deletion of the D227 residue. In some embodiments, the CD8 binding site may comprise a deletion of the residue corresponding to glutamate 232 (E232) of HLA-C.

In some embodiments, the construct may further comprise a mutation to the residue corresponding to the cysteine 1 (C1) residue of HLA-C (e.g., SEQ ID NO: 194). In some embodiments, the mutation is to a glycine (e.g., a CIG mutation).

In some embodiments, the construct comprises a single chain trimer (SCT). A single chain trimer may comprise a targeting peptide (e.g., moiety), a beta-2 microglobulin (B2M), and an HLA heavy chain, optionally connected by one or more linkers. In some embodiments, the SCT construct comprises a peptide comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 30-129, 142-157, and 203-278. In some embodiments, the SCT construct comprises a B2M region comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to SEQ ID NO: 195. In some embodiments, the SCT construct comprises an HLA heavy chain comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 130-141 and 279-288. In some embodiments, the SCT construct comprises a linker between the peptide and the B2M region comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 175-186 and 198. In some embodiments, the SCT construct comprises a linker between the B2M region and the HLA heavy chain comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 175-186 and 198. In some embodiments, the SCT construct comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 1-15.

In some embodiments, the construct comprises a single chain dimer (SCD). A single chain dimer may comprise a targeting moiety (e.g., peptide) and a beta-2 microglobulin (B2M). The single chain dimer may comprise a targeting moiety (e.g., peptide) and a B2M region connected by a linker. In some embodiments, the SCD construct comprises a peptide comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 16-29 and 199-202. In some embodiments, the SCD construct comprises a B2M region comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to SEQ ID NO: 195. In some embodiments, the SCD construct can be combined with an HLA heavy chain comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 130-141 and 279-288. In some embodiments, the SCD construct comprises a linker between the peptide and the B2M region comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 175-186 and 198. In some embodiments, the SCD construct comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 16-29 and 199-202.

In some embodiments, constructs of the disclosure comprise SCD constructs combined with HLA heavy chain constructs as described herein. In an example, a SCD construct comprises as targeting moiety (e.g., peptide) as described herein connected to a B2M sequence as described herein, optionally by a linker. The SCD construct may then be combined with an HLA heavy chain to provide a complex comprising a targeting moiety, a B2M sequence, and one or more HLA heavy chain sequences. In some embodiments, the SCD construct and the HLA heavy chain construct each comprise one part of a disulfide staple pair as described herein configured to form a disulfide bond. In some embodiments, the SCD comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 16-29 and 199-202. In some embodiments, the HLA heavy chain comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to any one of SEQ ID NOs: 130-141 and 279-288.

In some embodiments, the construct comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to a sequence listed in Table 1 below.

TABLE 1
HLA construct sequences

| | | SEQ ID |
|---|---|---|
| Description | Sequence | NO |
| Single | GILGFVFTLGGGGSGGGGSGGGGSIQRTPKIQVYSRHPAENGK | 1 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGSHSMRYFFTSVSRPGRGEPRFIAVGYVDDT | |
| | QFVRFDSDAASQRMEPRAPWIEQEGPEYWDGETRKVKAHSQ | |
| | THRVDLGTLRGYYNQSEAGSHTVQRMYGCDVGSDWRFLRG | |
| | YHQYAYDGKDYIALKEDLRSWTAADMAAQTTKHKWEAAHV | |
| | AEQLRAYLEGTCVEWLRRYLENGKETLQRTDAPKTHMTHHA | |
| | VSDHEATLRCWALSFYPAEITLTWQRDGEDQTQDTELVETRP | |
| | AGDGTFQKWAAVVVPSGQEQRYTCHVQHEGLPKPLTLRWE | |
| Single | GILGFVFTLGGGGSGGGGSGGGGSIQRTPKIQVYSRHPAENGK | 2 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGSHSMRYFFTSVSRPGRGEPRFIAVGYVDDT | |
| | QFVRFDSDAASQRMEPRAPWIEQEGPEYWDGETRKVKAHSQ | |
| | THRVDLGTLRGYYNQSEAGSHTVQRMYGCDVGSDWRFLRG | |
| | YHQYAYDGKDYIALKEDLRSWTAADMAAQTTKHKWEAAHV | |
| | AEQLRAYLEGTCVEWLRRYLENGKETLQRTDAPKTHMTHHA | |
| | VSDHEATLRCWALSFYPAEITLTWQRDGEDQTQDTELVETRP | |
| | AGDGTFQKWAAVVVPSGQEQRYTCHVQHEGLPKPLTLRWEE | |
| | NLYFQGHHHHHH | |
| Single | GILGFVFTLGGGGSGGGGSGGGGSIQRTPKIQVYSRHPAENGK | 3 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGSHSMRYFFTSVSRPGRGEPRFIAVGYVDDT | |
| | QFVRFDSDAASQRMEPRAPWIEQEGPEYWDGETRKVKAHSQ | |
| | THRVDLGTLRGYYNQSEAGSHTVQRMYGCDVGSDWRFLRG | |
| | YHQYAYDGKDYIALKEDLRSWTAADMAAQTTKHKWEAAHV | |
| | AEQLRAYLEGTCVEWLRRYLENGKETLQRTDAPKTHMTHHA | |
| | VSDHEATLRCWALSFYPAEITLTWQRDGEDQTQDTELVETRP | |
| | AGDGTFQKWAAVVVPSGQEQRYTCHVQHEGLPKPLTLRWEE | |
| | NLYFQGHHHHHHGGLNDIFEAQKIEWHE | |
| Single | GILGFVFTLGGGGSGGGGSGGGGSIQRTPKIQVYSRHPAENGK | 4 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGGGGSGSHSMRYFFTSVSRPGRGEPRFIAVG | |
| | YVDDTQFVRFDSDAASQRMEPRAPWIEQEGPEYWDGETRKV | |
| | KAHSQTHRVDLGTLRGYYNQSEAGSHTVQRMYGCDVGSDW | |
| | RFLRGYHQYAYDGKDYIALKEDLRSWTAADMAAQTTKHKW | |
| | EAAHVAEQLRAYLEGTCVEWLRRYLENGKETLQRTDAPKTH | |
| | MTHHAVSDHEATLRCWALSFYPAEITLTWQRDGEDQTQDTE | |
| | LVETRPAGDGTFQKWAAVVVPSGQEQRYTCHVQHEGLPKPL | |
| | TLRWE | |
| Single | MSRSVALAVLALLSLSGLEAGILGFVFTLGGGGSGGGGSGGG | 5 |
| chain | GSIQRTPKIQVYSRHPAENGKSNFLNCYVSGFHPSDIEVDLLKN | |
| trimer | GERIEKVEHSDLSFSKDWSFYLLYYTEFTPTEKDEYACRVNHV | |
| (SCT) | TLSQPKIVKWDRDMGGGGSGGGGSGGGGSGGGGSGSHSMRY | |
| | FFTSVSRPGRGEPRFIAVGYVDDTQFVRFDSDAASQRMEPRAP | |
| | WIEQEGPEYWDGETRKVKAHSQTHRVDLGTLRGYYNQSEAG | |
| | SHTVQRMYGCDVGSDWRFLRGYHQYAYDGKDYIALKEDLRS | |
| | WTAADMAAQTTKHKWEAAHVAEQLRAYLEGTCVEWLRRY | |
| | LENGKETLQRTDAPKTHMTHHAVSDHEATLRCWALSFYPAEI | |
| | TLTWQRDGEDQTQDTELVETRPAGDGTFQKWAAVVVPSGQE | |
| | QRYTCHVQHEGLPKPLTLRWEPSSQPTIPIVGIIAGLVLFGAVIT | |
| | GAVVAAVMWRRKSSDRKGGSYSQAASSDSAQGSDVSLTACK | |
| | V | |
| Single | GILGFVFTLGGGGSGGGGSGGGGSIQRTPKIQVYSRHPAENGK | 6 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGGGGSGSHSMRYFFTSVSRPGRGEPRFIAVG | |
| | YVDDTQFVRFDSDAASQRMEPRAPWIEQEGPEYWDGETRKV | |
| | KAHSQTHRVDLGTLRGYYNQSEAGSHTVQRMYGCDVGSDW | |
| | RFLRGYHQYAYDGKDYIALKEDLRSWTAADMAAQTTKHKW | |
| | EAAHVAEQLRAYLEGTCVEWLRRYLENGKETLQRTDAPKTH | |
| | MTHHAVSDHEATLRCWALSFYPAEITLTWQRDGEDQTQDTE | |
| | LVETRPAGDGTFQKWAAVVVPSGQEQRYTCHVQHEGLPKPL | |
| | TLRWE | |
| Single | GILGFVFTLGGGGSGGGGSGGGGSIQRTPKIQVYSRHPAENGK | 7 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGGGGSGSHSMRYFFTSVSRPGRGEPRFIAVG | |
| | YVDDTQFVRFDSDAASQRMEPRAPWIEQEGPEYWDGETRKV | |
| | KAHSQTHRVDLGTLRGYYNQSEAGSHTVQRMYGCDVGSDW | |
| | RFLRGYHQYAYDGKDYIALKEDLRSWTAADMAAQTTKHKW | |
| | EAAHVAEQLRAYLEGTCVEWLRRYLENGKETLQRTDAPKTH | |
| | MTHHAVSDHEATLRCWALSFYPAEITLTWQRDGEDQTQDTE | |
| | LVETRPAGDGTFQKWAAVVVPSGQEQRYTCHVQHEGLPKPL | |
| | TLRWEENLYFQGHHHHHH | |
| Single | GILGFVFTLGGGGSGGGGSGGGGSIQRTPKIQVYSRHPAENGK | 8 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSGGGGSGSHSMRYFFTSVSRPGRGEPRFIAVG | |
| | YVDDTQFVRFDSDAASQRMEPRAPWIEQEGPEYWDGETRKV | |
| | KAHSQTHRVDLGTLRGYYNQSEAGSHTVQRMYGCDVGSDW | |
| | RFLRGYHQYAYDGKDYIALKEDLRSWTAADMAAQTTKHKW | |
| | EAAHVAEQLRAYLEGTCVEWLRRYLENGKETLQRTDAPKTH | |
| | MTHHAVSDHEATLRCWALSFYPAEITLTWQRDGEDQTQDTE | |
| | LVETRPAGDGTFQKWAAVVVPSGQEQRYTCHVQHEGLPKPL | |
| | TLRWEENLYFQGHHHHHHGGLNDIFEAQKIEWHE | |
| Single | GILGFVFTLGGGGSGGGGSGGGGSIQRTPKIQVYSRHPAENGK | 9 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSCSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGYYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTQDTELVETRPAGD | |
| | GTFQKWAAVVVPSGQEQRYTCHMQHEGLQEPLTLSWEPS | |
| Single | GILGFVFTLGGGGSGGGGSGGGGSIQRTPKIQVYSRHPAENGK | 10 |
| chain | SNFLNCYVSGFHPSDIEVDLLKNGERIEKVEHSDLSFSKDWSF | |
| trimer | YLLYYTEFTPTEKDEYACRVNHVTLSQPKIVKWDRDMGGGG | |
| (SCT) | SGGGGSGGGGSCSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGYYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTQDTELVETRPAGD | |
| | GTFQKWAAVVVPSGQEQRYTCHMQHEGLQEPLTLSWEPSEN | |
| | LYFQGHHHHHH | |
| Single | SYRPGTVALGGGGSGGGGSGGGGSIQRTPKIQVYSRHPAENG | 11 |
| chain | KSNFLNCYVSGFHPSDIEVDLLKNGERIEKVEHSDLSFSKDWS | |
| trimer | FYLLYYTEFTPTEKDEYACRVNHVTLSQPKIVKWDRDMGGG | |
| (SCT) | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGYYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPS | |
| Single | SYRPGTVALGGGGSGGGGSGGGGSIQRTPKIQVYSRHPAENG | 12 |
| chain | KSNFLNCYVSGFHPSDIEVDLLKNGERIEKVEHSDLSFSKDWS | |
| trimer | FYLLYYTEFTPTEKDEYACRVNHVTLSQPKIVKWDRDMGGG | |
| (SCT) | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGYYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHH | |
| Single | SYRPGTVALGGGGSGGGGSGGGGSIQRTPKIQVYSRHPAENG | 13 |
| chain | KSNFLNCYVSGFHPSDIEVDLLKNGERIEKVEHSDLSFSKDWS | |
| trimer | FYLLYYTEFTPTEKDEYACRVNHVTLSQPKIVKWDRDMGGG | |
| (SCT) | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGYYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPS | |
| Single | SYRPGTVALGGGGSGGGGSGGGGSIQRTPKIQVYSRHPAENG | 14 |
| chain | KSNFLNCYVSGFHPSDIEVDLLKNGERIEKVEHSDLSFSKDWS | |
| trimer | FYLLYYTEFTPTEKDEYACRVNHVTLSQPKIVKWDRDMGGG | |
| (SCT) | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGYYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHH | |
| Single | SYRPGTVALGGGGSGGGGSGGGGSIQRTPKIQVYSRHPAENG | 15 |
| chain | KSNFLNCYVSGFHPSDIEVDLLKNGERIEKVEHSDLSFSKDWS | |
| trimer | FYLLYYTEFTPTEKDEYACRVNHVTLSQPKIVKWDRDMGGG | |
| (SCT) | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGYYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHHGGLNDIFEAQKIEWHE | |
| Single | IQRTPKIQVYSRHPAENGKSNFLNCYVSGFHPSDIEVDLLKNG | 16 |
| chain | ERIEKVEHSDLSFSKDWSFYLLYYTEFTPTEKDEYACRVNHVT | |
| dimer | LSQPKIVKWDRDMGGGGSGGGGSGGGGSGGGGSGSHSMRYF | |
| (SCD) | FTSVSRPGRGEPRFIAVGYVDDTQFVRFDSDAASQRMEPRAP | |
| | WIEQEGPEYWDGETRKVKAHSQTHRVDLGTLRGYYNQSEAG | |
| | SHTVQRMYGCDVGSDWRFLRGYHQYAYDGKDYIALKEDLRS | |
| | WTAADMAAQTTKHKWEAAHVAEQLRAYLEGTCVEWLRRY | |
| | LENGKETLQRTDAPKTHMTHHAVSDHEATLRCWALSFYPAEI | |
| | TLTWQRDGEDQTQDTELVETRPAGDGTFQKWAAVVVPSGQE | |
| | QRYTCHVQHEGLPKPLTLRWEPS | |
| Single | MSRSVALAVLALLSLSGLEAIQRTPKIQVYSRHPAENGKSNFL | 17 |
| chain | NCYVSGFHPSDIEVDLLKNGERIEKVEHSDLSFSKDWSFYLLY | |
| dimer | YTEFTPTEKDEYACRVNHVTLSQPKIVKWDRDMGGGGSGGG | |
| (SCD) | GSGGGGSGGGGSGSHSMRYFFTSVSRPGRGEPRFIAVGYVDD | |
| | TQFVRFDSDAASQRMEPRAPWIEQEGPEYWDGETRKVKAHS | |
| | QTHRVDLGTLRGYYNQSEAGSHTVQRMYGCDVGSDWRFLR | |
| | GYHQYAYDGKDYIALKEDLRSWTAADMAAQTTKHKWEAAH | |
| | VAEQLRAYLEGTCVEWLRRYLENGKETLQRTDAPKTHMTHH | |
| | AVSDHEATLRCWALSFYPAEITLTWQRDGEDQTQDTELVETR | |
| | PAGDGTFQKWAAVVVPSGQEQRYTCHVQHEGLPKPLTLRWE | |
| | PSSQPTIPIVGIIAGLVLFGAVITGAVVAAVMWRRKSSDRKGGS | |
| | YSQAASSDSAQGSDVSLTACKV | |
| Single | IQRTPKIQVYSRHPAENGKSNFLNCYVSGFHPSDIEVDLLKNG | 18 |
| chain | ERIEKVEHSDLSFSKDWSFYLLYYTEFTPTEKDEYACRVNHVT | |
| dimer | LSQPKIVKWDRDMGGGGSGGGGSGGGGSCSHSMRYFDTAVS | |
| (SCD) | RPGRGEPRFISVGYVDDTQFVRFDSDAASPRGEPRAPWVEQE | |
| | GPEYWDRETQKYKRQAQADRVSLRNLRGYYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPS | |
| Single | MSRSVALAVLALLSLSGLEAIQRTPKIQVYSRHPAENGKSNFL | 19 |
| chain | NCYVSGFHPSDIEVDLLKNGERIEKVEHSDLSFSKDWSFYLLY | |
| dimer | YTEFTPTEKDEYACRVNHVTLSQPKIVKWDRDMGGGGSGGG | |
| (SCD) | GSGGGGSCSHSMRYFDTAVSRPGRGEPRFISVGYVDDTQFVR | |
| | FDSDAASPRGEPRAPWVEQEGPEYWDRETQKYKRQAQADRV | |
| | SLRNLRGYYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTQDTELVETRPAGDGTFQ | |
| | KWAAVVVPSGQEQRYTCHMQHEGLQEPLTLSWEPSSQPTIPI | |
| | MGIVAGLAVLVVLAVLGAVVTAMMCRRKSSGGKGGSCSQA | |
| | ACSNSAQGSDESLITCKAVRAKRSGSGEGRGSLLTCGDVEENP | |
| | GPM | |
| Single | SYRPGTVALGGGGSGGGGSIQRTPKIQVYSRHPAENGKSNFLN | 20 |
| chain | CYVSGFHPSDIEVDLLKNGERIEKVEHSDLSFSKDWSFYLLYY | |
| dimer | TEFTPTEKDEYACRVNHVTLSQPKIVKWDRDM | |
| (SCD) | | |
| Single | SYRPGTVALGCGGSGGGGSIQRTPKIQVYSRHPAENGKSNFLN | 21 |
| chain | CYVSGFHPSDIEVDLLKNGERIEKVEHSDLSFSKDWSFYLLYY | |
| dimer | TEFTPTEKDEYACRVNHVTLSQPKIVKWDRDM | |
| (SCD) | | |
| Single | SYRPGTVALGGGGSGGGGSGSIQRTPKIQVYSRHPAENGKSNF | 22 |
| chain | LNCYVSGFHPSDIEVDLLKNGERIEKVEHSDLSFSKDWSFYLL | |
| dimer | YYTEFTPTEKDEYACRVNHVTLSQPKIVKWDRDM | |
| (SCD) | | |
| Single | SYRPGTVALGCGGSGGGGSGSIQRTPKIQVYSRHPAENGKSNF | 23 |
| chain | LNCYVSGFHPSDIEVDLLKNGERIEKVEHSDLSFSKDWSFYLL | |
| dimer | YYTEFTPTEKDEYACRVNHVTLSQPKIVKWDRDM | |
| (SCD) | | |
| Single | SYRPGTVALGGGGSGGGGSGGGGSIQRTPKIQVYSRHPAENG | 24 |
| chain | KSNFLNCYVSGFHPSDIEVDLLKNGERIEKVEHSDLSFSKDWS | |
| dimer | FYLLYYTEFTPTEKDEYACRVNHVTLSQPKIVKWDRDM | |
| (SCD) | | |
| Single | SYRPGTVALGCGGSGGGGSGGGGSIQRTPKIQVYSRHPAENG | 25 |
| chain | KSNFLNCYVSGFHPSDIEVDLLKNGERIEKVEHSDLSFSKDWS | |
| dimer | FYLLYYTEFTPTEKDEYACRVNHVTLSQPKIVKWDRDM | |
| (SCD) | | |
| Single | SYRPCTVALGGGGSGGGGSIQRTPKIQVYSRHPAENGKSNFLN | 26 |
| chain | CYVSGFHPSDIEVDLLKNGERIEKVEHSDLSFSKDWSFYLLYY | |
| dimer | TEFTPTEKDEYACRVNHVTLSQPKIVKWDRDM | |
| (SCD) | | |
| Single | SYRPGTCALGGGGSGGGGSIQRTPKIQVYSRHPAENGKSNFLN | 27 |
| chain | CYVSGFHPSDIEVDLLKNGERIEKVEHSDLSFSKDWSFYLLYY | |
| dimer | TEFTPTEKDEYACRVNHVTLSQPKIVKWDRDM | |
| (SCD) | | |
| Single | SYRPGTVCLGGGGSGGGGSIQRTPKIQVYSRHPAENGKSNFLN | 28 |
| chain | CYVSGFHPSDIEVDLLKNGERIEKVEHSDLSFSKDWSFYLLYY | |
| dimer | TEFTPTEKDEYACRVNHVTLSQPKIVKWDRDM | |
| (SCD) | | |
| Single | SRRYCNVAYGGGGSGGGGSIQRTPKIQVYSRHPAENGKSNFL | 29 |
| chain | NCYVSGFHPSDIEVDLLKNGERIEKVEHSDLSFSKDWSFYLLY | |
| dimer | YTEFTPTEKDEYACRVNHVTLSQPKIVKWDRDM | |
| (SCD) | | |
| Single | SYRPCTVALGGGGSGGGGSGGGGSIQRTPKIQVYSRHPAENG | 199 |
| chain | KSNFLNCYVSGFHPSDIEVDLLKNGERIEKVEHSDLSFSKDWS | |
| dimer | FYLLYYTEFTPTEKDEYACRVNHVTLSQPKIVKWDRDM | |
| (SCD) | | |
| Single | SYRPGTCALGGGGSGGGGSGGGGSIQRTPKIQVYSRHPAENG | 200 |
| chain | KSNFLNCYVSGFHPSDIEVDLLKNGERIEKVEHSDLSFSKDWS | |
| dimer | FYLLYYTEFTPTEKDEYACRVNHVTLSQPKIVKWDRDM | |
| (SCD) | | |
| Single | SYRPCTVALGGGGSGGGGSGSIQRTPKIQVYSRHPAENGKSNF | 201 |
| chain | LNCYVSGFHPSDIEVDLLKNGERIEKVEHSDLSFSKDWSFYLL | |
| dimer | YYTEFTPTEKDEYACRVNHVTLSQPKIVKWDRDM | |
| (SCD) | | |
| Single | SYRPGTCALGGGGSGGGGSGSIQRTPKIQVYSRHPAENGKSNF | 202 |
| chain | LNCYVSGFHPSDIEVDLLKNGERIEKVEHSDLSFSKDWSFYLL | |
| dimer | YYTEFTPTEKDEYACRVNHVTLSQPKIVKWDRDM | |
| (SCD) | | |
| Synthetic | GILGFVFTLGGGGSGGGGSGGGGSIQRTPKIQVYSRHPAENGK | 30 |
| HLA | SNFLNCYVSGFHPSDIEVDLLKNGERIEKVEHSDLSFSKDWSF | |
| (synHLA) | YLLYYTEFTPTEKDEYACRVNHVTLSQPKIVKWDRDMGGGG | |
| | SGGGGSGGGGSGSHSMRYFFTSVSRPGRGEPRFIAVGYVDDT | |
| | QFVRFDSDAASQRMEPRAPWIEQEGPEYWDGETRKVKAHSQ | |
| | THRVDLGTLRGAYNQSEAGSHTVQRMYGCDVGSDWRFLRG | |
| | YHQYAYDGKDYIALKEDLRSWTAADMAAQTTKHKWEAAHV | |
| | AEQLRAYLEGTCVEWLRRYLENGKETLQRTDAPKTHMTHHA | |
| | VSDHEATLRCWALSFYPAEITLTWQRDGEDQTQDTELVETRP | |
| | AGDGTFQKWAAVVVPSGQEQRYTCHVQHEGLPKPLTLRWE | |
| Synthetic | GILGFVFTLGGGGSGGGGSGGGGSIQRTPKIQVYSRHPAENGK | 31 |
| HLA | SNFLNCYVSGFHPSDIEVDLLKNGERIEKVEHSDLSFSKDWSF | |
| (synHLA) | YLLYYTEFTPTEKDEYACRVNHVTLSQPKIVKWDRDMGGGG | |
| | SGGGGSGGGGSGSHSMRYFFTSVSRPGRGEPRFIAVGYVDDT | |
| | QFVRFDSDAASQRMEPRAPWIEQEGPEYWDGETRKVKAHSQ | |
| | THRVDLGTLRGAYNQSEAGSHTVQRMYGCDVGSDWRFLRG | |
| | YHQYAYDGKDYIALKEDLRSWTAADMAAQTTKHKWEAAHV | |
| | AEQLRAYLEGTCVEWLRRYLENGKETLQRTDAPKTHMTHHA | |
| | VSDHEATLRCWALSFYPAEITLTWQRDGEDQTQDTELVETRP | |
| | AGDGTFQKWAAVVVPSGQEQRYTCHVQHEGLPKPLTLRWEE | |
| | NLYFQGHHHHHH | |
| Synthetic | RYRPGTVALGGGGSGGGGSGGGGSIQRTPKIQVYSRHPAENG | 32 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPS | |
| Synthetic | RYRPGTVALGGGGSGGGGGGGGSIQRTPKIQVYSRHPAENG | 33 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHH | |
| Synthetic | RYRPGTVALGGGGSGGGGSGGGGSIQRTPKIQVYSRHPAENG | 34 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | RYRPGTVALGGGGSGGGGSGGGGSIQRTPKIQVYSRHPAENG | 35 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHH | |
| Synthetic | RYRPGTVALGCGGSGGGGSGGGGSIQRTPKIQVYSRHPAENG | 36 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | RYRPGTVALGCGGSGGGGSGGGGSIQRTPKIQVYSRHPAENG | 37 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHH | |
| Synthetic | RYRPGTVALGGGGSGGGGSGSIQRTPKIQVYSRHPAENGKSNF | 38 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPS | |
| Synthetic | RYRPGTVALGGGGSGGGGSGSIQRTPKIQVYSRHPAENGKSNF | 39 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPSENLYF | |
| | QGHHHHHH | |
| Synthetic | MSRSVALAVLALLSLSGLEARYRPGTVALGGGGSGGGGSGSI | 40 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSCSHSMRYFDTAVSR | |
| | PGRGEPRFISVGYVDDTQFVRFDSDAASPRGEPRAPWVEQEGP | |
| | EYWDRETQKYKRQAQADRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM-green fluorescent protein | |
| Synthetic | RYRPGTVALGGGGSGGGGSGSIQRTPKIQVYSRHPAENGKSNF | 41 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | RYRPGTVALGGGGSGGGGSGSIQRTPKIQVYSRHPAENGKSNF | 42 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHH | |
| Synthetic | RYRPGTVALGCGGSGGGGSGSIQRTPKIQVYSRHPAENGKSNF | 43 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | RYRPGTVALGCGGSGGGGSGSIQRTPKIQVYSRHPAENGKSNF | 44 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHH | |
| Synthetic | RYRPGTVALGGGGSGGGGSIQRTPKIQVYSRHPAENGKSNFL | 45 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSCSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTQDTELVETRPAGDGTFQ | |
| | KWAAVVVPSGQEQRYTCHMQHEGLQEPLTLSWEPSENLYFQ | |
| | GHHHHHH | |
| Synthetic | RYRPGTVALGGGGSGGGGSIQRTPKIQVYSRHPAENGKSNFL | 46 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSCSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTQDTELVETRPAGDGTFQ | |
| | KWAAVVVPSGQEQRYTCHMQHEGLQEPLTLSWEPSENLYFQ | |
| | GHHHHHH | |
| Synthetic | RYRPGTVALGGGGSGGGGSIQRTPKIQVYSRHPAENGKSNFL | 47 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSCSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | RYRPGTVALGGGGSGGGGSIQRTPKIQVYSRHPAENGKSNFL | 48 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSCSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPSENLYFQ | |
| | GHHHHHH | |
| Synthetic | RYRPGTVALGCGGSGGGGSIQRTPKIQVYSRHPAENGKSNFLN | 49 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | RYRPGTVALGCGGSGGGGSIQRTPKIQVYSRHPAENGKSNFLN | 50 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHH | |
| Synthetic | SYRPGTVALGGGGSGGGGSGGGGSIQRTPKIQVYSRHPAENG | 51 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPS | |
| Synthetic | SYRPGTVALGGGGSGGGGSGGGGSIQRTPKIQVYSRHPAENG | 52 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHH | |
| Synthetic | SYRPGTVALGGGGSGGGGSGGGGSIQRTPKIQVYSRHPAENG | 53 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | SYRPGTVALGGGGSGGGGSGGGGSIQRTPKIQVYSRHPAENG | 54 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHH | |
| Synthetic | SYRPGTVALGCGGSGGGGSGGGGSIQRTPKIQVYSRHPAENG | 55 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | SYRPGTVALGCGGSGGGGSGGGGSIQRTPKIQVYSRHPAENG | 56 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSCSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHH | |
| Synthetic | SYRPGTVALGGGGSGGGGSGSIQRTPKIQVYSRHPAENGKSNF | 57 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPS | |
| Synthetic | SYRPGTVALGGGGSGGGGSGSIQRTPKIQVYSRHPAENGKSNF | 58 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPSENLYF | |
| | QGHHHHHH | |
| Synthetic | MSRSVALAVLALLSLSGLEASYRPGTVALGGGGSGGGGSGSI | 59 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSCSHSMRYFDTAVSR | |
| | PGRGEPRFISVGYVDDTQFVRFDSDAASPRGEPRAPWVEQEGP | |
| | EYWDRETQKYKRQAQADRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM-green fluorescent protein | |
| Synthetic | SYRPGTVALGGGGSGGGGSGSIQRTPKIQVYSRHPAENGKSNF | 60 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | SYRPGTVALGGGGSGGGGSGSIQRTPKIQVYSRHPAENGKSNF | 61 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHH | |
| Synthetic | SYRPGTVALGCGGSGGGGSGSIQRTPKIQVYSRHPAENGKSNF | 62 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | SYRPGTVALGCGGSGGGGSGSIQRTPKIQVYSRHPAENGKSNF | 63 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHH | |
| Synthetic | SYRPGTVALGGGGSGGGGSIQRTPKIQVYSRHPAENGKSNFLN | 64 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTQDTELVETRPAGDGTFQK | |
| | WAAVVVPSGQEQRYTCHMQHEGLQEPLTLSWEPS | |
| Synthetic | SYRPGTVALGGGGSGGGGSIQRTPKIQVYSRHPAENGKSNFLN | 65 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTQDTELVETRPAGDGTFQK | |
| | WAAVVVPSGQEQRYTCHMQHEGLQEPLTLSWEPSENLYFQG | |
| | HHHHHH | |
| Synthetic | SYRPGTVALGGGGSGGGGSIQRTPKIQVYSRHPAENGKSNFLN | 66 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGGGGSGGGGSIQRTPKIQVYSRHPAENGKSNFLN | 67 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHH | |
| Synthetic | SYRPGTVALGCGGSGGGGSIQRTPKIQVYSRHPAENGKSNFLN | 68 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGCGGSGGGGSIQRTPKIQVYSRHPAENGKSNFLN | 69 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSCSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHH | |
| Synthetic | SYRPGTVALGGGGSGGGGSGGGGSIQRTPKIQVYSRHPAENG | 70 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPS | |
| Synthetic | SYRPGTVALGGGGSGGGGSGGGGSIQRTPKIQVYSRHPAENG | 71 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHH | |
| Synthetic | SYRPGTVALGGGGSGGGGSGGGGSIQRTPKIQVYSRHPAENG | 72 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPSE | |
| | NLYFQGHHHHHHGGLNDIFEAQKIEWHE | |
| Synthetic | SYRPGTVALGGGGSGGGGSGGGGSIQRTPKIQVYSRHPAENG | 73 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | SYRPGTVALGGGGSGGGGSGGGGSIQRTPKIQVYSRHPAENG | 74 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHH | |
| Synthetic | SYRPGTVALGGGGSGGGGSGGGGSIQRTPKIQVYSRHPAENG | 75 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHHGGLNDIFEAQKIEWHE | |
| Synthetic | SYRPGTVALGCGGSGGGGSGGGGSIQRTPKIQVYSRHPAENG | 76 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | SYRPGTVALGCGGSGGGGSGGGGSIQRTPKIQVYSRHPAENG | 77 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHH | |
| Synthetic | SYRPGTVALGCGGSGGGGSGGGGSIQRTPKIQVYSRHPAENG | 78 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPSE | |
| | NLYFQGHHHHHHGGLNDIFEAQKIEWHE | |
| Synthetic | SYRPGTVALGGGGSGGGGSGSIQRTPKIQVYSRHPAENGKSNF | 79 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPS | |
| Synthetic | SYRPGTVALGGGGSGGGGSGSIQRTPKIQVYSRHPAENGKSNF | 80 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPSENLYF | |
| | QGHHHHHH | |
| Synthetic | SYRPGTVALGGGGSGGGGSGSIQRTPKIQVYSRHPAENGKSNF | 81 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPSENLYF | |
| | QGHHHHHHGGLNDIFEAQKIEWHE | |
| Synthetic | MSRSVALAVLALLSLSGLEASYRPGTVALGGGGSGGGGSGSI | 82 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGGGGGSGGGGSGSHSMRYFDTAVSR | |
| | PGRGEPRFISVGYVDDTQFVRFDSDAASPRGEPRAPWVEQEGP | |
| | EYWDRETQKYKRQAQADRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM-green fluorescent protein | |
| Synthetic | SYRPGTVALGGGGSGGGGSGSIQRTPKIQVYSRHPAENGKSNF | 83 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | SYRPGTVALGGGGSGGGGSGSIQRTPKIQVYSRHPAENGKSNF | 84 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHHGGLNDIFEAQKIEWHE | |
| Synthetic | SYRPGTVALGGGGSGGGGSGSIQRTPKIQVYSRHPAENGKSNF | 85 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHH | |
| Synthetic | SYRPGTVALGCGGSGGGGSGSIQRTPKIQVYSRHPAENGKSNF | 86 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | SYRPGTVALGCGGSGGGGSGSIQRTPKIQVYSRHPAENGKSNF | 87 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHH | |
| Synthetic | SYRPGTVALGCGGSGGGGSGSIQRTPKIQVYSRHPAENGKSNF | 88 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPSENLYF | |
| | QGHHHHHHGGLNDIFEAQKIEWHE | |
| Synthetic | SYRPGTVALGGGGSGGGGSIQRTPKIQVYSRHPAENGKSNFLN | 89 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTQDTELVETRPAGDGTFQK | |
| | WAAVVVPSGQEQRYTCHMQHEGLQEPLTLSWEPS | |
| Synthetic | SYRPGTVALGGGGSGGGGSIQRTPKIQVYSRHPAENGKSNFLN | 90 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTQDTELVETRPAGDGTFQK | |
| | WAAVVVPSGQEQRYTCHMQHEGLQEPLTLSWEPSENLYFQG | |
| | HHHHHH | |
| Synthetic | SYRPGTVALGGGGSGGGGSIQRTPKIQVYSRHPAENGKSNFLN | 91 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTQDTELVETRPAGDGTFQK | |
| | WAAVVVPSGQEQRYTCHMQHEGLQEPLTLSWEPSENLYFQG | |
| | HHHHHHGGLNDIFEAQKIEWHE | |
| Synthetic | SYRPGTVALGGGGSGGGGSIQRTPKIQVYSRHPAENGKSNFLN | 92 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGGGGSGGGGSIQRTPKIQVYSRHPAENGKSNFLN | 93 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHH | |
| Synthetic | SYRPGTVALGGGGSGGGGSIQRTPKIQVYSRHPAENGKSNFLN | 94 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHHGGLNDIFEAQKIEWHE | |
| Synthetic | SYRPGTVALGCGGSGGGGSIQRTPKIQVYSRHPAENGKSNFLN | 95 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGCGGSGGGGSIQRTPKIQVYSRHPAENGKSNFLN | 96 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHH | |
| Synthetic | SYRPGTVALGCGGSGGGGSIQRTPKIQVYSRHPAENGKSNFLN | 97 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPSENLYFQG | |
| | HHHHHHGGLNDIFEAQKIEWHE | |
| Synthetic | RYRPGTVALGGGGSGGGGSGSIQRTPKIQVYSRHPAENGKSNF | 98 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSCSHSMRYFDTAVSRPGRGEPRFISVGYVDDTQFV | |
| | RFDSDAASPRGEPRAPWVEQEGPEYWDRETQKYKRQAQADR | |
| | VSLRNLRGYYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPS | |
| Synthetic | MSRSVALAVLALLSLSGLEARYRPGTVALGGGGSGGGGSGSI | 99 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSCSHSMRYFDTAVSR | |
| | PGRGEPRFISVGYVDDTQFVRFDSDAASPRGEPRAPWVEQEGP | |
| | EYWDRETQKYKRQAQADRVSLRNLRGYYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPS | |
| Synthetic | MSRSVALAVLALLSLSGLEARYRPGTVALGGGGSGGGGSGSI | 100 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSCSHSMRYFDTAVSR | |
| | PGRGEPRFISVGYVDDTQFVRFDSDAASPRGEPRAPWVEQEGP | |
| | EYWDRETQKYKRQAQADRVSLRNLRGYYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | RYRPGTVALGGGGSGGGGSGGGGSIQRTPKIQVYSRHPAENG | 101 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTQDTELVETRPAG | |
| | DGTFQKWAAVVVPSGQEQRYTCHMQHEGLQEPLTLSWEPS | |
| Synthetic | MSRSVALAVLALLSLSGLEARYRPGTVALGGGGSGGGGSGG | 102 |
| HLA | GGSIQRTPKIQVYSRHPAENGKSNFLNCYVSGFHPSDIEVDLLK | |
| (synHLA) | NGERIEKVEHSDLSFSKDWSFYLLYYTEFTPTEKDEYACRVNH | |
| | VTLSQPKIVKWDRDMGGGGSGGGGSGGGGSGSHSMRYFDTA | |
| | VSRPGRGEPRFISVGYVDDTQFVRFDSDAASPRGEPRAPWVEQ | |
| | EGPEYWDRETQKYKRQAQADRVSLRNLRGAYNQSEDGSHTL | |
| | QRMSGCDLGPDGRLLRGYDQSAYDGKDYIALNEDLRSWTAA | |
| | DTAAQITQRKLEAARAAEQLRAYLEGTCVEWLRRYLENGKE | |
| | TLQRAEPPKTHVTHHPLSDHEATLRCWALGFYPAEITLTWQR | |
| | DGEDQTQDTELVETRPAGDGTFQKWAAVVVPSGQEQRYTCH | |
| | MQHEGLQEPLTLSWEPSSQPTIPIMGIVAGLAVLVVLAVLGAV | |
| | VTAMMCRRKSSGGKGGSCSQAACSNSAQGSDESLITCKAVRA | |
| | KRSGSGEGRGSLLTCGDVEENPGPM-green fluorescent protein | |
| Synthetic | RYRPGTVALGGGGSGGGGGGGGSIQRTPKIQVYSRHPAENG | 103 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGAYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | MSRSVALAVLALLSLSGLEARYRPGTVALGGGGSGGGGSGG | 104 |
| HLA | GGSIQRTPKIQVYSRHPAENGKSNFLNCYVSGFHPSDIEVDLLK | |
| (synHLA) | NGERIEKVEHSDLSFSKDWSFYLLYYTEFTPTEKDEYACRVNH | |
| | VTLSQPKIVKWDRDMGGGGSGGGGSGGGGSGSHSMRYFDTA | |
| | VSRPGRGEPRFISVGYVDDTQFVRFDSDAASPRGEPRAPWVEQ | |
| | EGPEYWDRETQKYKRQAQADRVSLRNLRGAYNQSEDGSHTL | |
| | QRMSGCDLGPDGRLLRGYDQSAYDGKDYIALNEDLRSWTAA | |
| | DTAAQITQRKLEAARAAEQLRAYLEGTCVEWLRRYLENGKE | |
| | TLQRAEPPKTHVTHHPLSDHEATLRCWALGFYPAEITLTWQR | |
| | DGEDQTAKTELVETRPAGDGTFQKWAAVVVPSGQEQRYTCH | |
| | MQHEGLQEPLTLSWEPSSQPTIPIMGIVAGLAVLVVLAVLGAV | |
| | VTAMMCRRKSSGGKGGSCSQAACSNSAQGSDESLITCKAVRA | |
| | KRSGSGEGRGSLLTCGDVEENPGPM | |
| Synthetic | RYRPGTVALGCGGSGGGGSGGGGSIQRTPKIQVYSRHPAENG | 105 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGGG | |
| | GSGGGGSGGGGSGSHSMRYFDTAVSRPGRGEPRFISVGYVDD | |
| | TQFVRFDSDAASPRGEPRAPWVEQEGPEYWDRETQKYKRQA | |
| | QADRVSLRNLRGCYNQSEDGSHTLQRMSGCDLGPDGRLLRG | |
| | YDQSAYDGKDYIALNEDLRSWTAADTAAQITQRKLEAARAA | |
| | EQLRAYLEGTCVEWLRRYLENGKETLQRAEPPKTHVTHHPLS | |
| | DHEATLRCWALGFYPAEITLTWQRDGEDQTAKTELVETRPAG | |
| | DGTFQKWAAVVVPSGQEQRYTCHMQHEGLQEPLTLSWEPS | |
| Synthetic | MSRSVALAVLALLSLSGLEARYRPGTVALGCGGSGGGGSGGG | 106 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKRQAQADRVSLRNLRGCYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | RYRPGTVALGGGGSGGGGSGSIQRTPKIQVYSRHPAENGKSNF | 107 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTQDTELVETRPAGDGTF | |
| | QKWAAVVVPSGQEQRYTCHMQHEGLQEPLTLSWEPS | |
| Synthetic | MSRSVALAVLALLSLSGLEARYRPGTVALGGGGSGGGGSGSI | 108 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKRQAQADRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTQDTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | RYRPGTVALGGGGSGGGGSGSIQRTPKIQVYSRHPAENGKSNF | 109 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGAYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | MSRSVALAVLALLSLSGLEARYRPGTVALGGGGSGGGGSGSI | 110 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKRQAQADRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | RYRPGTVALGCGGSGGGGSGSIQRTPKIQVYSRHPAENGKSNF | 111 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGGGGSGG | |
| | GGSGGGGSGSHSMRYFDTAVSRPGRGEPRFISVGYVDDTQFV | |
| | RFDSDAASPRGEPRAPWVEQEGPEYWDRETQKYKRQAQADR | |
| | VSLRNLRGCYNQSEDGSHTLQRMSGCDLGPDGRLLRGYDQS | |
| | AYDGKDYIALNEDLRSWTAADTAAQITQRKLEAARAAEQLR | |
| | AYLEGTCVEWLRRYLENGKETLQRAEPPKTHVTHHPLSDHEA | |
| | TLRCWALGFYPAEITLTWQRDGEDQTAKTELVETRPAGDGTF | |
| | QKWAAVVVPSGQEQRYTCHMQHEGLQEPLTLSWEPS | |
| Synthetic | MSRSVALAVLALLSLSGLEARYRPGTVALGCGGSGGGGSGSI | 112 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKRQAQADRVSLRNLRGCYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | RYRPGTVALGGGGSGGGGSIQRTPKIQVYSRHPAENGKSNFL | 113 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTQDTELVETRPAGDGTFQ | |
| | KWAAVVVPSGQEQRYTCHMQHEGLQEPLTLSWEPS | |
| Synthetic | MSRSVALAVLALLSLSGLEARYRPGTVALGGGGSGGGGSIQR | 114 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGAYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TQDTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | RYRPGTVALGGGGSGGGGSIQRTPKIQVYSRHPAENGKSNFL | 115 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | MSRSVALAVLALLSLSGLEARYRPGTVALGGGGSGGGGSIQR | 116 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGAYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | RYRPGTVALGCGGSGGGGSIQRTPKIQVYSRHPAENGKSNFLN | 117 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | MSRSVALAVLALLSLSGLEARYRPGTVALGCGGSGGGGSIQR | 118 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | SYRPGTVALGGGGSGGGGSIQRTPKIQVYSRHPAENGKSNFLN | 119 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTELVETRPAGDGTFQKWA | |
| | AVVVPSGQEQRYTCHMQHEGLQEPLTLSWEPS | |
| Synthetic | SYRPGTVALGGGGSGGGGSIQRTPKIQVYSRHPAENGKSNFLN | 120 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTELVTRPAGDGTFQKWAA | |
| | VVVPSGQEQRYTCHMQHEGLQEPLTLSWEPS | |
| Synthetic | SYRPGTEALGGGGSGGGGSIQRTPKIQVYSRHPAENGKSNFLN | 121 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVELGGGGSGGGGSIQRTPKIQVYSRHPAENGKSNFLN | 122 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGGGSGGGGSIQRTPKIQVYSRHPAENGKSNFLNC | 123 |
| HLA | YVSGFHPSDIEVDLLKNGERIEKVEHSDLSFSKDWSFYLLYYT | |
| (synHLA) | EFTPTEKDEYACRVNHVTLSQPKIVKWDRDMGGGGSGGGGS | |
| | GGGGSGSHSMRYFDTAVSRPGRGEPRFISVGYVDDTQFVRFD | |
| | SDAASPRGEPRAPWVEQEGPEYWDRETQKYKRQAQADRVSL | |
| | RNLRGAYNQSEDGSHTLQRMSGCDLGPDGRLLRGYDQSAYD | |
| | GKDYIALNEDLRSWTAADTAAQITQRKLEAARAAEQLRAYLE | |
| | GTCVEWLRRYLENGKETLQRAEPPKTHVTHHPLSDHEATLRC | |
| | WALGFYPAEITLTWQRDGEDQTAKTELVETRPAGDGTFQKW | |
| | AAVVVPSGQEQRYTCHMQHEGLQEPLTLSWEPS | |
| Synthetic | SYRPGTVALGGGGSGGGGSIQRTPKIQVYSRHPAENGKSNFLN | 124 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGGGGSGSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGAYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTAKTELVETRPAGD | |
| | GTFQKWAAVVVPSGQEQRYTCHMQHEGLQEPLTLSWEPS | |
| Synthetic | SYRPCTVALGGGGSGGGGSIQRTPKIQVYSRHPAENGKSNFLN | 125 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKCQAQADRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTCALGGGGSGGGGSIQRTPKIQVYSRHPAENGKSNFLN | 126 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGAYNQSEDGSHTLQRMSGCDLGPDGRLLRGYDQSAY | |
| | DGKDYIALNEDLRSWTAADTAAQITQRKLEACRAAEQLRAYL | |
| | EGTCVEWLRRYLENGKETLQRAEPPKTHVTHHPLSDHEATLR | |
| | CWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVCLGGGGSGGGGSIQRTPKIQVYSRHPAENGKSNFLN | 127 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQCDRVS | |
| | LRNLRGAYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SRRYCNVAYGGGGSGGGGSIQRTPKIQVYSRHPAENGKSNFL | 128 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKCQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SYRPGTVALGGGGSGGGGSIQRTPKIQVYSRHPAENGKSNFLN | 129 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGSHSMRYFDTAVSRPGRGEPRFISVGYVDDTQFVRFDSDAAS | |
| | PRGEPRAPWVEQEGPEYWDRETQKYKRQAQADRVSLRNLRG | |
| | AYNQSEDGSHTLQRMSGCDLGPDGRLLRGYDQSAYDGKDYI | |
| | ALNEDLRSWTAADTAAQITQRKLEAARAAEQLRAYLEGTCV | |
| | EWLRRYLENGKETLQRAEPPKTHVTHHPLSDHEATLRCWAL | |
| | GFYPAEITLTWQRDGEDQTAKTELVETRPAGDGTFQKWAAV | |
| | VVPSGQEQRYTCHMQHEGLQEPLTLSWEPS | |
| HLA heavy | GSHSMRYFDTAVSRPGRGEPRFISVGYVDDTQFVRFDSDAASP | 130 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTQDTELVETRPAGDGTFQKWAAVVV | |
| | PSGQEQRYTCHMQHEGLQEPLTLSWEPS | |
| HLA heavy | GSHSMRYFDTAVSRPGRGEPRFISVGYVDDTQFVRFDSDAASP | 131 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTQDTELVETRPAGDGTFQKWAAVVV | |
| | PSGQEQRYTCHMQHEGLQEPLTLSWEPSENLYFQGHHHHHH | |
| HLA heavy | GSHSMRYFDTAVSRPGRGEPRFISVGYVDDTQFVRFDSDAASP | 132 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTQDTELVETRPAGDGTFQKWAAVVV | |
| | PSGQEQRYTCHMQHEGLQEPLTLSWEPSENLYFQGHHHHHH | |
| | GGLNDIFEAQKIEWHE | |
| HLA heavy | GSHSMRYFDTAVSRPGRGEPRFISVGYVDDTQFVRFDSDAASP | 133 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPS | |
| HLA heavy | GSHSMRYFDTAVSRPGRGEPRFISVGYVDDTQFVRFDSDAASP | 134 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPSENLYFQGHHHHHH | |
| HLA heavy | GSHSMRYFDTAVSRPGRGEPRFISVGYVDDTQFVRFDSDAASP | 135 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPSENLYFQGHHHHHH | |
| | GGLNDIFEAQKIEWHE | |
| HLA heavy | GSHSMRYFDTAVSRPGRGEPRFISVGYVDDTQFVRFDSDAASP | 136 |
| chain | RGEPRAPWVEQEGPEYWDRETQKYKRQAQADRVSLRNLRGC | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPS | |
| HLA heavy | GSHSMRYFDTAVSRPGRGEPRFISVGYVDDTQFVRFDSDAASP | 137 |
| chain | RGEPRAPWVEQEGPEYWDRETQKYKRQAQADRVSLRNLRGC | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPSENLYFQGHHHHHH | |
| HLA heavy | GSHSMRYFDTAVSRPGRGEPRFISVGYVDDTQFVRFDSDAASP | 138 |
| chain | RGEPRAPWVEQEGPEYWDRETQKYKRQAQADRVSLRNLRGC | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPSENLYFQGHHHHHH | |
| | GGLNDIFEAQKIEWHE | |
| HLA heavy | GSHSMRYFDTAVSRPGRGEPRFISVGYVDDTQFVRFDSDAASP | 139 |
| chain | RGEPRAPWVEQEGPEYWDRETQKYKCQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPS | |
| HLA heavy | GSHSMRYFDTAVSRPGRGEPRFISVGYVDDTQFVRFDSDAASP | 140 |
| chain | RGEPRAPWVEQEGPEYWDRETQKYKRQAQADRVSLRNLRGA | |
| (HHC) | YNQSEDGSHTLQRMSGCDLGPDGRLLRGYDQSAYDGKDYIA | |
| | LNEDLRSWTAADTAAQITQRKLEACRAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPS | |
| HLA heavy | GSHSMRYFDTAVSRPGRGEPRFISVGYVDDTQFVRFDSDAASP | 141 |
| chain | RGEPRAPWVEQEGPEYWDRETQKYKRQAQCDRVSLRNLRGA | |
| (HHC) | YNQSEDGSHTLQRMSGCDLGPDGRLLRGYDQSAYDGKDYIA | |
| | LNEDLRSWTAADTAAQITQRKLEAARAAEQLRAYLEGTCVE | |
| | WLRRYLENGKETLQRAEPPKTHVTHHPLSDHEATLRCWALGF | |
| | YPAEITLTWQRDGEDQTAKTELVETRPAGDGTFQKWAAVVV | |
| | PSGQEQRYTCHMQHEGLQEPLTLSWEPS | |
| HLA heavy | GSHSMRYFDTAVSRPGRGEPRFISVGYVDDTQFVRFDSDAASPRGEPRAPWVEQEG | 279 |
| chain | PEYWDRETQKYKCQAQADRVSLRNLRGAYNQSEDGSHTLQRMSGCDLGPDGRLLRG | |
| (HHC) | YDQSAYDGKDYIALNEDLRSWTAADTAAQATQRKLEAARAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 280 |
| chain | PEYWDRETQKYKCQAQADRVSLRNLRGFYNQSEDGSHTLQRMSGCDLGPDGRLLRG | |
| (HHC) | YDQSAYDGKDYIALNEDLRSWTAADTAAQITQRKLEAARAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 281 |
| chain | PEYWDRETQKYKCQAQADRVSLRNLRGLYNQSEDGSHTLQRMSGCDLGPDGRLLRG | |
| (HHC) | YDQSAYDGKDYIALNEDLRSWTAADTAAQITQRKLEAARAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 282 |
| chain | PEYWDRETQKYKCQAQADRVSLRNLRGFYNQSEDGSHTLQRMSGCDLGPDGRLLRG | |
| (HHC) | YDQSAYDGKDYIALNEDLRSWTAADTAAQATQRKLEAARAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 283 |
| chain | PEYWDRETQKYKCQAQADRVSLRNLRGLYNQSEDGSHTLQRMSGCDLGPDGRLLRG | |
| (HHC) | YDQSAYDGKDYIALNEDLRSWTAADTAAQATQRKLEAARAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 284 |
| chain | PEYWDRETQKYKRQAQADRVSLRNLRGAYNQSEDGSHTLQRMSGCDLGPDGRLLRG | |
| (HHC) | YDQSAYDGKDYIALNEDLRSWTAADTAAQATQRKLEACRAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 285 |
| chain | PEYWDRETQKYKRQAQADRVSLRNLRGFYNQSEDGSHTLQRMSGCDLGPDGRLLRG | |
| (HHC) | YDQSAYDGKDYIALNEDLRSWTAADTAAQITQRKLEACRAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 286 |
| chain | PEYWDRETQKYKRQAQADRVSLRNLRGLYNQSEDGSHTLQRMSGCDLGPDGRLLRG | |
| (HHC) | YDQSAYDGKDYIALNEDLRSWTAADTAAQITQRKLEACRAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 287 |
| chain | PEYWDRETQKYKRQAQADRVSLRNLRGFYNQSEDGSHTLQRMSGCDLGPDGRLLRG | |
| (HHC) | YDQSAYDGKDYIALNEDLRSWTAADTAAQATQRKLEACRAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| HLA heavy | GSHSMRYFDTAVSRPGRGEPRFISVGYVDDTQFVRFDSDAASPRGEPRAPWVEQEG | 288 |
| chain | PEYWDRETQKYKRQAQADRVSLRNLRGLYNQSEDGSHTLQRMSGCDLGPDGRLLRG | |
| (HHC) | YDQSAYDGKDYIALNEDLRSWTAADTAAQATQRKLEACRAAEQLRAYLEGTCVEWL | |
| | RRYLENGKETLQRAEPPKTHVTHHPLSDHEATLRCWALGFYPAEITLTWQRDGEDQ | |
| | TAKTELVETRPAGDGTFQKWAAVVVPSGQEQRYTCHMQHEGLQEPLTLSWEPSENL | |
| | YFQGHHHHHHGLNDIFEAQKIEWHE | |
| Synthetic | SYRPGTVALGCGGSGGGGSIQRTPKIQVYSRHPAENGKSNFLN | 142 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGAAAAA | |
| | AAAGGGSGGGGGSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGCYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTAKTELVETRPAGD | |
| | GTFQKWAAVVVPSGQEQRYTCHMQHEGLQEPLTLSWEPS | |
| Synthetic | SWRYCNVAYGGGGSGGGGSIQRTPKIQVYSRHPAENGKSNFL | 143 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKCQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRYCQVAYGGGGSGGGGSIQRTPKIQVYSRHPAENGKSNFL | 144 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKCQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRYEMCAMGGGGSGGGGSIQRTPKIQVYSRHPAENGKSNFL | 145 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEACRAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRYLMCAYGGGGSGGGGSIQRTPKIQVYSRHPAENGKSNFL | 146 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEACRAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRWLMCAYGGGGSGGGGSIQRTPKIQVYSRHPAENGKSNFL | 147 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEACRAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRMEMVCIGGGGSGGGGSIQRTPKIQVYSRHPAENGKSNFL | 148 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQCDRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRMWMVCIGGGGSGGGGSIQRTPKIQVYSRHPAENGKSNFL | 149 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQCDRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRMEMVCRGGGGSGGGGSIQRTPKIQVYSRHPAENGKSNFL | 150 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQCDRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SYRPGTVALGCGGSGGGGSIQRTPKIQVYSRHPAENGKSNFLN | 151 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQ--- | |
| | TELVETRPAGDGTFQKWAAVVVPSGQEQRYTCHMQHEGLQE | |
| | PLTLSWEPS | |
| Synthetic | SYRPGTVALGCGGSGGGGSIQRTPKIQVYSRHPAENGKSNFLN | 152 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTELVTRPAGDGTFQKWAA | |
| | VVVPSGQEQRYTCHMQHEGLQEPLTLSWEPS | |
| Synthetic | SYRPGTEALGCGGSGGGGSIQRTPKIQVYSRHPAENGKSNFLN | 153 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVELGCGGSGGGGSIQRTPKIQVYSRHPAENGKSNFLN | 154 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGCGSGGGGSIQRTPKIQVYSRHPAENGKSNFLNC | 155 |
| HLA | YVSGFHPSDIEVDLLKNGERIEKVEHSDLSFSKDWSFYLLYYT | |
| (synHLA) | EFTPTEKDEYACRVNHVTLSQPKIVKWDRDMGGGGSGGGGS | |
| | GGGGSGSHSMRYFDTAVSRPGRGEPRFISVGYVDDTQFVRFD | |
| | SDAASPRGEPRAPWVEQEGPEYWDRETQKYKRQAQADRVSL | |
| | RNLRGCYNQSEDGSHTLQRMSGCDLGPDGRLLRGYDQSAYD | |
| | GKDYIALNEDLRSWTAADTAAQITQRKLEAARAAEQLRAYLE | |
| | GTCVEWLRRYLENGKETLQRAEPPKTHVTHHPLSDHEATLRC | |
| | WALGFYPAEITLTWQRDGEDQTAKTELVETRPAGDGTFQKW | |
| | AAVVVPSGQEQRYTCHMQHEGLQEPLTLSWEPS | |
| Synthetic | SYRPGTVALGCGGSGGGGSIQRTPKIQVYSRHPAENGKSNFLN | 156 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGSHSMRYFDTAVSRPGRGEPRFISVGYVDDTQFVRFDSDAAS | |
| | PRGEPRAPWVEQEGPEYWDRETQKYKRQAQADRVSLRNLRG | |
| | CYNQSEDGSHTLQRMSGCDLGPDGRLLRGYDQSAYDGKDYI | |
| | ALNEDLRSWTAADTAAQITQRKLEAARAAEQLRAYLEGTCV | |
| | EWLRRYLENGKETLQRAEPPKTHVTHHPLSDHEATLRCWAL | |
| | GFYPAEITLTWQRDGEDQTAKTELVETRPAGDGTFQKWAAV | |
| | VVPSGQEQRYTCHMQHEGLQEPLTLSWEPS | |
| Synthetic | SYRPGTVALGCGGSGGGGSIQRTPKIQVYSRHPAENGKSNFLN | 157 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGGGGSGSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGCYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTAKTELVETRPAGD | |
| | GTFQKWAAVVVPSGQEQRYTCHMQHEGLQEPLTLSWEPS | |
| Synthetic | SYRPGTCALGGGGSGGGGSGSIQRTPKIQVYSRHPAENGKSNF | 203 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGSHSMRY | |
| | FDTAVSRPGRGEPRFISVGYVDDTQFVRFDSDAASPRGEPRAP | |
| | WVEQEGPEYWDRETQKYKRQAQADRVSLRNLRGFYNQSED | |
| | GSHTLQRMSGCDLGPDGRLLRGYDQSAYDGKDYIALNEDLRS | |
| | WTAADTAAQITQRKLEACRAAEQLRAYLEGTCVEWLRRYLE | |
| | NGKETLQRAEPPKTHVTHHPLSDHEATLRCWALGFYPAEITLT | |
| | WQRDGEDQTAKTELVETRPAGDGTFQKWAAVVVPSGQEQR | |
| | YTCHMQHEGLQEPLTLSWEPS | |
| Synthetic | SYRPGTCALGGGGSGGGGSGSIQRTPKIQVYSRHPAENGKSNF | 204 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGSHSMRY | |
| | FDTAVSRPGRGEPRFISVGYVDDTQFVRFDSDAASPRGEPRAP | |
| | WVEQEGPEYWDRETQKYKRQAQADRVSLRNLRGLYNQSED | |
| | GSHTLQRMSGCDLGPDGRLLRGYDQSAYDGKDYIALNEDLRS | |
| | WTAADTAAQITQRKLEACRAAEQLRAYLEGTCVEWLRRYLE | |
| | NGKETLQRAEPPKTHVTHHPLSDHEATLRCWALGFYPAEITLT | |
| | WQRDGEDQTAKTELVETRPAGDGTFQKWAAVVVPSGQEQR | |
| | YTCHMQHEGLQEPLTLSWEPS | |
| Synthetic | SYRPGTCALGGGGSGGGGSGSIQRTPKIQVYSRHPAENGKSNF | 205 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGSHSMRY | |
| | FDTAVSRPGRGEPRFISVGYVDDTQFVRFDSDAASPRGEPRAP | |
| | WVEQEGPEYWDRETQKYKRQAQADRVSLRNLRGFYNQSED | |
| | GSHTLQRMSGCDLGPDGRLLRGYDQSAYDGKDYIALNEDLRS | |
| | WTAADTAAQATQRKLEACRAAEQLRAYLEGTCVEWLRRYLE | |
| | NGKETLQRAEPPKTHVTHHPLSDHEATLRCWALGFYPAEITLT | |
| | WQRDGEDQTAKTELVETRPAGDGTFQKWAAVVVPSGQEQR | |
| | YTCHMQHEGLQEPLTLSWEPS | |
| Synthetic | SYRPGTCALGGGGSGGGGSGSIQRTPKIQVYSRHPAENGKSNF | 206 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGSHSMRY | |
| | FDTAVSRPGRGEPRFISVGYVDDTQFVRFDSDAASPRGEPRAP | |
| | WVEQEGPEYWDRETQKYKRQAQADRVSLRNLRGLYNQSED | |
| | GSHTLQRMSGCDLGPDGRLLRGYDQSAYDGKDYIALNEDLRS | |
| | WTAADTAAQATQRKLEACRAAEQLRAYLEGTCVEWLRRYLE | |
| | NGKETLQRAEPPKTHVTHHPLSDHEATLRCWALGFYPAEITLT | |
| | WQRDGEDQTAKTELVETRPAGDGTFQKWAAVVVPSGQEQR | |
| | YTCHMQHEGLQEPLTLSWEPS | |
| Synthetic | MSRSVALAVLALLSLSGLEARYRPGTEALGGGGSGGGGSIQR | 207 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVELGGGGSGGGGSIQR | 208 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVALGCGGSGGGGSIQR | 209 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | ___TELVETRPAGDGTFQKWAAVVVPSGQEQRYTCHMQHEGL | |
| | QEPLTLSWEPSSQPTIPIMGIVAGLAVLVVLAVLGAVVTAMM | |
| | CRRKSSGGKGGSCSQAACS | |
| Synthetic | MSRSVALAVLALLSLSGLEARYRPCTVALGGGGSGGGGSGGG | 210 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGAYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPGTCALGGGGSGGGGSGGG | 211 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKRQAQADRVSLRNLRGAYNQSEDGSHTLQ | |
| | RMSGCDLGPDGRLLRGYDQSAYDGKDYIALNEDLRSWTAAD | |
| | TAAQITQRKLEACRAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPGTVCLGGGGSGGGGSGGG | 212 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKRQAQCDRVSLRNLRGAYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 213 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPGTCALGGGGSGGGGSGSI | 214 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKRQAQADRVSLRNLRGAYNQSEDGSHTLQR | |
| | MSGCDLGPDGRLLRGYDQSAYDGKDYIALNEDLRSWTAADT | |
| | AAQITQRKLEACRAAEQLRAYLEGTCVEWLRRYLENGKETLQ | |
| | RAEPPKTHVTHHPLSDHEATLRCWALGFYPAEITLTWQRDGE | |
| | DQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPGTVCLGGGGSGGGGSGSI | 215 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKRQAQCDRVSLRNLRGAYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 216 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGYYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 217 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGFYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 218 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGLYNQSEDGSHTLQ | |
| | RMSGCDLGPDGRLLRGYDQSAYDGKDYIALNEDLRSWTAAD | |
| | TAAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 219 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGAYNQSEDGSHTLQ | |
| | RMSGCDLGPDGRLLRGYDQSAYDGKDYIALNEDLRSWTAAD | |
| | TAAQATQRKLEAARAAEQLRAYLEGTCVEWLRRYLENGKET | |
| | LQRAEPPKTHVTHHPLSDHEATLRCWALGFYPAEITLTWQRD | |
| | GEDQTAKTELVETRPAGDGTFQKWAAVVVPSGQEQRYTCHM | |
| | QHEGLQEPLTLSWEPSSQPTIPIMGIVAGLAVLVVLAVLGAVV | |
| | TAMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 220 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGAYNQSEDGSHTLQ | |
| | RMSGCDLGPDGRLLRGYDQSAYDGKDYIALNEDLRSWTAAD | |
| | TAAQVTQRKLEAARAAEQLRAYLEGTCVEWLRRYLENGKET | |
| | LQRAEPPKTHVTHHPLSDHEATLRCWALGFYPAEITLTWQRD | |
| | GEDQTAKTELVETRPAGDGTFQKWAAVVVPSGQEQRYTCHM | |
| | QHEGLQEPLTLSWEPSSQPTIPIMGIVAGLAVLVVLAVLGAVV | |
| | TAMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 22 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGFYNQSEDGSHTLQ | |
| | RMSGCDLGPDGRLLRGYDQSAYDGKDYIALNEDLRSWTAAD | |
| | TAAQATQRKLEAARAAEQLRAYLEGTCVEWLRRYLENGKET | |
| | LQRAEPPKTHVTHHPLSDHEATLRCWALGFYPAEITLTWQRD | |
| | GEDQTAKTELVETRPAGDGTFQKWAAVVVPSGQEQRYTCHM | |
| | QHEGLQEPLTLSWEPSSQPTIPIMGIVAGLAVLVVLAVLGAVV | |
| | TAMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGGG | 222 |
| HLA | GSIQRTPKIQVYSRHPAENGKSNFLNCYVSGFHPSDIEVDLLKN | |
| (synHLA) | GERIEKVEHSDLSFSKDWSFYLLYYTEFTPTEKDEYACRVNHV | |
| | TLSQPKIVKWDRDMGGGGSGGGGSGGGGSGSHSMRYFDTAV | |
| | SRPGRGEPRFISVGYVDDTQFVRFDSDAASPRGEPRAPWVEQE | |
| | GPEYWDRETQKYKCQAQADRVSLRNLRGLYNQSEDGSHTLQ | |
| | RMSGCDLGPDGRLLRGYDQSAYDGKDYIALNEDLRSWTAAD | |
| | TAAQATQRKLEAARAAEQLRAYLEGTCVEWLRRYLENGKET | |
| | LQRAEPPKTHVTHHPLSDHEATLRCWALGFYPAEITLTWQRD | |
| | GEDQTAKTELVETRPAGDGTFQKWAAVVVPSGQEQRYTCHM | |
| | QHEGLQEPLTLSWEPSSQPTIPIMGIVAGLAVLVVLAVLGAVV | |
| | TAMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 223 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGYYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 224 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGFYNQSEDGSHTLQRM | |
| | SGCDLGPDGRLLRGYDQSAYDGKDYIALNEDLRSWTAADTA | |
| | AQITQRKLEAARAAEQLRAYLEGTCVEWLRRYLENGKETLQR | |
| | AEPPKTHVTHHPLSDHEATLRCWALGFYPAEITLTWQRDGED | |
| | QTAKTELVETRPAGDGTFQKWAAVVVPSGQEQRYTCHMQHE | |
| | GLQEPLTLSWEPSSQPTIPIMGIVAGLAVLVVLAVLGAVVTAM | |
| | MCRRKSSGGKGGSCSQAACSNSAQGSDESLITCKAVRAKRSG | |
| | SGEGRGSLLTCGDVEENPGPM | |
| Synthetic | MSRSVALAVLALLSLSGLEARYRPCTVALGGGGSGGGGSGSI | 225 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGLYNQSEDGSHTLQR | |
| | MSGCDLGPDGRLLRGYDQSAYDGKDYIALNEDLRSWTAADT | |
| | AAQITQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 226 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGAYNQSEDGSHTLQR | |
| | MSGCDLGPDGRLLRGYDQSAYDGKDYIALNEDLRSWTAADT | |
| | AAQATQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 227 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGAYNQSEDGSHTLQR | |
| | MSGCDLGPDGRLLRGYDQSAYDGKDYIALNEDLRSWTAADT | |
| | AAQVTQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 228 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGFYNQSEDGSHTLQRM | |
| | SGCDLGPDGRLLRGYDQSAYDGKDYIALNEDLRSWTAADTA | |
| | AQATQRKLEAARAAEQLRAYLEGTCVEWLRRYLENGKETLQ | |
| | RAEPPKTHVTHHPLSDHEATLRCWALGFYPAEITLTWQRDGE | |
| | DQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSGSI | 229 |
| HLA | QRTPKIQVYSRHPAENGKSNFLNCYVSGFHPSDIEVDLLKNGE | |
| (synHLA) | RIEKVEHSDLSFSKDWSFYLLYYTEFTPTEKDEYACRVNHVTL | |
| | SQPKIVKWDRDMGGGGSGGGGSGGGGSGSHSMRYFDTAVSR | |
| | PGRGEPRFISVGYVDDTQFVRFDSDAASPRGEPRAPWVEQEGP | |
| | EYWDRETQKYKCQAQADRVSLRNLRGLYNQSEDGSHTLQR | |
| | MSGCDLGPDGRLLRGYDQSAYDGKDYIALNEDLRSWTAADT | |
| | AAQATQRKLEAARAAEQLRAYLEGTCVEWLRRYLENGKETL | |
| | QRAEPPKTHVTHHPLSDHEATLRCWALGFYPAEITLTWQRDG | |
| | EDQTAKTELVETRPAGDGTFQKWAAVVVPSGQEQRYTCHMQ | |
| | HEGLQEPLTLSWEPSSQPTIPIMGIVAGLAVLVVLAVLGAVVT | |
| | AMMCRRKSSGGKGGSCSQAACSNSAQGSDESLITCKAVRAK | |
| | RSGSGEGRGSLLTCGDVEENPGPM | |
| Synthetic | MSRSVALAVLALLSLSGLEARYRPCTVALGGGGSGGGGSIQR | 230 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGAYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTCALGGGGSGGGGSIQR | 231 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGAYNQSEDGSHTLQRMS | |
| | GCDLGPDGRLLRGYDQSAYDGKDYIALNEDLRSWTAADTAA | |
| | QITQRKLEACRAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVCLGGGGSGGGGSIQR | 232 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQCDRVSLRNLRGAYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPCTVALGGGGSGGGGSIQR | 233 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGYYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPCTVALGGGGSGGGGSIQR | 234 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGFYNQSEDGSHTLQRMSG | |
| | CDLGPDGRLLRGYDQSAYDGKDYIALNEDLRSWTAADTAAQ | |
| | ITQRKLEAARAAEQLRAYLEGTCVEWLRRYLENGKETLQRAE | |
| | PPKTHVTHHPLSDHEATLRCWALGFYPAEITLTWQRDGEDQT | |
| | AKTELVETRPAGDGTFQKWAAVVVPSGQEQRYTCHMQHEGL | |
| | QEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPCTVALGGGGSGGGGSIQR | 235 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGLYNQSEDGSHTLQRMSG | |
| | CDLGPDGRLLRGYDQSAYDGKDYIALNEDLRSWTAADTAAQ | |
| | ITQRKLEAARAAEQLRAYLEGTCVEWLRRYLENGKETLQRAE | |
| | PPKTHVTHHPLSDHEATLRCWALGFYPAEITLTWQRDGEDQT | |
| | AKTELVETRPAGDGTFQKWAAVVVPSGQEQRYTCHMQHEGL | |
| | QEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPCTVALGGGGSGGGGSIQR | 236 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGAYNQSEDGSHTLQRMS | |
| | GCDLGPDGRLLRGYDQSAYDGKDYIALNEDLRSWTAADTAA | |
| | QATQRKLEAARAAEQLRAYLEGTCVEWLRRYLENGKETLQR | |
| | AEPPKTHVTHHPLSDHEATLRCWALGFYPAEITLTWQRDGED | |
| | QTAKTELVETRPAGDGTFQKWAAVVVPSGQEQRYTCHMQHE | |
| | GLQEPLTLSWEPSSQPTIPIMGIVAGLAVLVVLAVLGAVVTAM | |
| | MCRRKSSGGKGGSCSQAACSNSAQGSDESLITCKAVRAKRSG | |
| | SGEGRGSLLTCGDVEENPGPM | |
| Synthetic | MSRSVALAVLALLSLSGLEARYRPCTVALGGGGSGGGGSIQR | 237 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGAYNQSEDGSHTLQRMS | |
| | GCDLGPDGRLLRGYDQSAYDGKDYIALNEDLRSWTAADTAA | |
| | QVTQRKLEAARAAEQLRAYLEGTCVEWLRRYLENGKETLQR | |
| | AEPPKTHVTHHPLSDHEATLRCWALGFYPAEITLTWQRDGED | |
| | QTAKTELVETRPAGDGTFQKWAAVVVPSGQEQRYTCHMQHE | |
| | GLQEPLTLSWEPSSQPTIPIMGIVAGLAVLVVLAVLGAVVTAM | |
| | MCRRKSSGGKGGSCSQAACSNSAQGSDESLITCKAVRAKRSG | |
| | SGEGRGSLLTCGDVEENPGPM | |
| Synthetic | MSRSVALAVLALLSLSGLEARYRPCTVALGGGGSGGGGSIQR | 238 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGFYNQSEDGSHTLQRMSG | |
| | CDLGPDGRLLRGYDQSAYDGKDYIALNEDLRSWTAADTAAQ | |
| | ATQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPCTVALGGGGSGGGGSIQR | 239 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKCQAQADRVSLRNLRGLYNQSEDGSHTLQRMSG | |
| | CDLGPDGRLLRGYDQSAYDGKDYIALNEDLRSWTAADTAAQ | |
| | ATQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPCTVALGGGGSGGGSIQRTP | 240 |
| HLA | KIQVYSRHPAENGKSNFLNCYVSGFHPSDIEVDLLKNGERIEK | |
| (synHLA) | VEHSDLSFSKDWSFYLLYYTEFTPTEKDEYACRVNHVTLSQP | |
| | KIVKWDRDMGGGGSGGGGSGGGGSGSHSMRYFDTAVSRPGR | |
| | GEPRFISVGYVDDTQFVRFDSDAASPRGEPRAPWVEQEGPEY | |
| | WDRETQKYKCQAQADRVSLRNLRGAYNQSEDGSHTLQRMS | |
| | GCDLGPDGRLLRGYDQSAYDGKDYIALNEDLRSWTAADTAA | |
| | QVTQRKLEAARAAEQLRAYLEGTCVEWLRRYLENGKETLQR | |
| | AEPPKTHVTHHPLSDHEATLRCWALGFYPAEITLTWQRDGED | |
| | QTAKTELVETRPAGDGTFQKWAAVVVPSGQEQRYTCHMQHE | |
| | GLQEPLTLSWEPSSQPTIPIMGIVAGLAVLVVLAVLGAVVTAM | |
| | MCRRKSSGGKGGSCSQAACSNSAQGSDESLITCKAVRAKRSG | |
| | SGEGRGSLLTCGDVEENPGPM | |
| Synthetic | MSRSVALAVLALLSLSGLEARYRPCTVALGGGGSGGSIQRTPK | 241 |
| HLA | IQVYSRHPAENGKSNFLNCYVSGFHPSDIEVDLLKNGERIEKV | |
| (synHLA) | EHSDLSFSKDWSFYLLYYTEFTPTEKDEYACRVNHVTLSQPKI | |
| | VKWDRDMGGGGSGGGGSGGGGSGSHSMRYFDTAVSRPGRG | |
| | EPRFISVGYVDDTQFVRFDSDAASPRGEPRAPWVEQEGPEYW | |
| | DRETQKYKCQAQADRVSLRNLRGAYNQSEDGSHTLQRMSGC | |
| | DLGPDGRLLRGYDQSAYDGKDYIALNEDLRSWTAADTAAQV | |
| | TQRKLEAARAAEQLRAYLEGTCVEWLRRYLENGKETLQRAE | |
| | PPKTHVTHHPLSDHEATLRCWALGFYPAEITLTWQRDGEDQT | |
| | AKTELVETRPAGDGTFQKWAAVVVPSGQEQRYTCHMQHEGL | |
| | QEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | SYRPCTVALGGGGSGGGGSGGGGSIQRTPKIQVYSRHPAENG | 242 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGSHS | |
| | MRYFDTAVSRPGRGEPRFISVGYVDDTQFVRFDSDAASPRGEP | |
| | RAPWVEQEGPEYWDRETQKYKCQAQADRVSLRNLRGAYNQ | |
| | SEDGSHTLQRMSGCDLGPDGRLLRGYDQSAYDGKDYIALNE | |
| | DLRSWTAADTAAQITQRKLEAARAAEQLRAYLEGTCVEWLR | |
| | RYLENGKETLQRAEPPKTHVTHHPLSDHEATLRCWALGFYPA | |
| | EITLTWQRDGEDQTAKTELVETRPAGDGTFQKWAAVVVPSG | |
| | QEQRYTCHMQHEGLQEPLTLSWEPS | |
| Synthetic | SYRPGTCALGGGGSGGGGSGGGGSIQRTPKIQVYSRHPAENG | 243 |
| HLA | KSNFLNCYVSGFHPSDIEVDLLKNGERIEKVEHSDLSFSKDWS | |
| (synHLA) | FYLLYYTEFTPTEKDEYACRVNHVTLSQPKIVKWDRDMGSHS | |
| | MRYFDTAVSRPGRGEPRFISVGYVDDTQFVRFDSDAASPRGEP | |
| | RAPWVEQEGPEYWDRETQKYKRQAQADRVSLRNLRGAYNQ | |
| | SEDGSHTLQRMSGCDLGPDGRLLRGYDQSAYDGKDYIALNE | |
| | DLRSWTAADTAAQITQRKLEACRAAEQLRAYLEGTCVEWLR | |
| | RYLENGKETLQRAEPPKTHVTHHPLSDHEATLRCWALGFYPA | |
| | EITLTWQRDGEDQTAKTELVETRPAGDGTFQKWAAVVVPSG | |
| | QEQRYTCHMQHEGLQEPLTLSWEPS | |
| Synthetic | SYRPCTVALGGGGSGGGGSGSIQRTPKIQVYSRHPAENGKSNF | 244 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGSHSMRY | |
| | FDTAVSRPGRGEPRFISVGYVDDTQFVRFDSDAASPRGEPRAP | |
| | WVEQEGPEYWDRETQKYKCQAQADRVSLRNLRGAYNQSED | |
| | GSHTLQRMSGCDLGPDGRLLRGYDQSAYDGKDYIALNEDLRS | |
| | WTAADTAAQITQRKLEAARAAEQLRAYLEGTCVEWLRRYLE | |
| | NGKETLQRAEPPKTHVTHHPLSDHEATLRCWALGFYPAEITLT | |
| | WQRDGEDQTAKTELVETRPAGDGTFQKWAAVVVPSGQEQR | |
| | YTCHMQHEGLQEPLTLSWEPS | |
| Synthetic | SYRPGTCALGGGGSGGGGSGSIQRTPKIQVYSRHPAENGKSNF | 245 |
| HLA | LNCYVSGFHPSDIEVDLLKNGERIEKVEHSDLSFSKDWSFYLL | |
| (synHLA) | YYTEFTPTEKDEYACRVNHVTLSQPKIVKWDRDMGSHSMRY | |
| | FDTAVSRPGRGEPRFISVGYVDDTQFVRFDSDAASPRGEPRAP | |
| | WVEQEGPEYWDRETQKYKRQAQADRVSLRNLRGAYNQSED | |
| | GSHTLQRMSGCDLGPDGRLLRGYDQSAYDGKDYIALNEDLRS | |
| | WTAADTAAQITQRKLEACRAAEQLRAYLEGTCVEWLRRYLE | |
| | NGKETLQRAEPPKTHVTHHPLSDHEATLRCWALGFYPAEITLT | |
| | WQRDGEDQTAKTELVETRPAGDGTFQKWAAVVVPSGQEQR | |
| | YTCHMQHEGLQEPLTLSWEPS | |
| Synthetic | SYRPCTVALGGGGSGGGGSIQRTPKIQVYSRHPAENGKSNFLN | 246 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKCQAQADRVSLRNLRGAYNQSEDGSHT | |
| | LQRMSGCDLGPDGRLLRGYDQSAYDGKDYIALNEDLRSWTA | |
| | ADTAAQATQRKLEAARAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPCTVALGGGGSGGGGSIQRTPKIQVYSRHPAENGKSNFLN | 247 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKCQAQADRVSLRNLRGFYNQSEDGSHT | |
| | LQRMSGCDLGPDGRLLRGYDQSAYDGKDYIALNEDLRSWTA | |
| | ADTAAQITQRKLEAARAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPCTVALGGGGSGGGGSIQRTPKIQVYSRHPAENGKSNFLN | 248 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKCQAQADRVSLRNLRGLYNQSEDGSHT | |
| | LQRMSGCDLGPDGRLLRGYDQSAYDGKDYIALNEDLRSWTA | |
| | ADTAAQITQRKLEAARAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPCTVALGGGGSGGGGSIQRTPKIQVYSRHPAENGKSNFLN | 249 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKCQAQADRVSLRNLRGFYNQSEDGSHT | |
| | LQRMSGCDLGPDGRLLRGYDQSAYDGKDYIALNEDLRSWTA | |
| | ADTAAQATQRKLEAARAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPCTVALGGGGSGGGGSIQRTPKIQVYSRHPAENGKSNFLN | 250 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKCQAQADRVSLRNLRGLYNQSEDGSHT | |
| | LQRMSGCDLGPDGRLLRGYDQSAYDGKDYIALNEDLRSWTA | |
| | ADTAAQATQRKLEAARAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPGTCALGGGGSGGGGSIQRTPKIQVYSRHPAENGKSNFLN | 251 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKRQAQADRVSLRNLRGAYNQSEDGSHT | |
| | LQRMSGCDLGPDGRLLRGYDQSAYDGKDYIALNEDLRSWTA | |
| | ADTAAQATQRKLEACRAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPGTCALGGGGSGGGGSIQRTPKIQVYSRHPAENGKSNFLN | 252 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKRQAQADRVSLRNLRGFYNQSEDGSHT | |
| | LQRMSGCDLGPDGRLLRGYDQSAYDGKDYIALNEDLRSWTA | |
| | ADTAAQITQRKLEACRAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPGTCALGGGGSGGGGSIQRTPKIQVYSRHPAENGKSNFLN | 253 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKRQAQADRVSLRNLRGLYNQSEDGSHT | |
| | LQRMSGCDLGPDGRLLRGYDQSAYDGKDYIALNEDLRSWTA | |
| | ADTAAQITQRKLEACRAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPGTCALGGGGSGGGGSIQRTPKIQVYSRHPAENGKSNFLN | 254 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKRQAQADRVSLRNLRGFYNQSEDGSHT | |
| | LQRMSGCDLGPDGRLLRGYDQSAYDGKDYIALNEDLRSWTA | |
| | ADTAAQATQRKLEACRAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPGTCALGGGGSGGGGSIQRTPKIQVYSRHPAENGKSNFLN | 255 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGSHSMRYFDT | |
| | AVSRPGRGEPRFISVGYVDDTQFVRFDSDAASPRGEPRAPWVE | |
| | QEGPEYWDRETQKYKRQAQADRVSLRNLRGLYNQSEDGSHT | |
| | LQRMSGCDLGPDGRLLRGYDQSAYDGKDYIALNEDLRSWTA | |
| | ADTAAQATQRKLEACRAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPS | |
| Synthetic | SYRPGTVALGCGGSGGGGSIQRTPKIQVYSRHPAENGKSNFLN | 256 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGAAAAA | |
| | AAAGGGSGGGGGSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGCYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTAKTELVETRPAGD | |
| | GTFQKWAAVVVPSGQEQRYTCHMQHEGLQEPLTLSWEPS | |
| Synthetic | SWRYCNVAYGGGGSGGGGSIQRTPKIQVYSRHPAENGKSNFL | 257 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKCQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRYCQVAYGGGGSGGGGSIQRTPKIQVYSRHPAENGKSNFL | 258 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKCQAQADRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRYEMCAMGGGGSGGGGSIQRTPKIQVYSRHPAENGKSNFL | 259 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEACRAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRYLMCAYGGGGSGGGGSIQRTPKIQVYSRHPAENGKSNFL | 260 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEACRAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRWLMCAYGGGGSGGGGSIQRTPKIQVYSRHPAENGKSNFL | 26 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQADRV | |
| | SLRNLRGAYNQSEDGSHTLQRMSGCDLGPDGRLLRGYDQSA | |
| | YDGKDYIALNEDLRSWTAADTAAQITQRKLEACRAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRMEMVCIGGGGSGGGGSIQRTPKIQVYSRHPAENGKSNFL | 262 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQCDRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRMWMVCIGGGGSGGGGSIQRTPKIQVYSRHPAENGKSNFL | 263 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQCDRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SRRMEMVCRGGGGSGGGGSIQRTPKIQVYSRHPAENGKSNFL | 264 |
| HLA | NCYVSGFHPSDIEVDLLKNGERIEKVEHSDLSFSKDWSFYLLY | |
| (synHLA) | YTEFTPTEKDEYACRVNHVTLSQPKIVKWDRDMGGGGSGGG | |
| | GSGGGGSGSHSMRYFDTAVSRPGRGEPRFISVGYVDDTQFVR | |
| | FDSDAASPRGEPRAPWVEQEGPEYWDRETQKYKRQAQCDRV | |
| | SLRNLRGAYNQSEDGSHTLQRMSGCDLGPDGRLLRGYDQSA | |
| | YDGKDYIALNEDLRSWTAADTAAQITQRKLEAARAAEQLRA | |
| | YLEGTCVEWLRRYLENGKETLQRAEPPKTHVTHHPLSDHEAT | |
| | LRCWALGFYPAEITLTWQRDGEDQTAKTELVETRPAGDGTFQ | |
| | KWAAVVVPSGQEQRYTCHMQHEGLQEPLTLSWEPS | |
| Synthetic | SYRPGTVALGCGGSGGGGSIQRTPKIQVYSRHPAENGKSNFLN | 265 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQ--- | |
| | TELVETRPAGDGTFQKWAAVVVPSGQEQRYTCHMQHEGLQE | |
| | PLTLSWEPS | |
| Synthetic | SYRPGTVALGCGGSGGGGSIQRTPKIQVYSRHPAENGKSNFLN | 266 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTELVTRPAGDGTFQKWAA | |
| | VVVPSGQEQRYTCHMQHEGLQEPLTLSWEPS | |
| Synthetic | SYRPGTEALGCGGSGGGGSIQRTPKIQVYSRHPAENGKSNFLN | 267 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVELGCGGSGGGGSIQRTPKIQVYSRHPAENGKSNFLN | 268 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGSHSMRYFDTAVSRPGRGEPRFISVGYVDDTQFVRF | |
| | DSDAASPRGEPRAPWVEQEGPEYWDRETQKYKRQAQADRVS | |
| | LRNLRGCYNQSEDGSHTLQRMSGCDLGPDGRLLRGYDQSAY | |
| | DGKDYIALNEDLRSWTAADTAAQITQRKLEAARAAEQLRAY | |
| | LEGTCVEWLRRYLENGKETLQRAEPPKTHVTHHPLSDHEATL | |
| | RCWALGFYPAEITLTWQRDGEDQTAKTELVETRPAGDGTFQK | |
| | WAAVVVPSGQEQRYTCHMQHEGLQEPLTLSWEPS | |
| Synthetic | SYRPGTVALGCGSGGGGSIQRTPKIQVYSRHPAENGKSNFLNC | 269 |
| HLA | YVSGFHPSDIEVDLLKNGERIEKVEHSDLSFSKDWSFYLLYYT | |
| (synHLA) | EFTPTEKDEYACRVNHVTLSQPKIVKWDRDMGGGGSGGGGS | |
| | GGGGSGSHSMRYFDTAVSRPGRGEPRFISVGYVDDTQFVRFD | |
| | SDAASPRGEPRAPWVEQEGPEYWDRETQKYKRQAQADRVSL | |
| | RNLRGCYNQSEDGSHTLQRMSGCDLGPDGRLLRGYDQSAYD | |
| | GKDYIALNEDLRSWTAADTAAQITQRKLEAARAAEQLRAYLE | |
| | GTCVEWLRRYLENGKETLQRAEPPKTHVTHHPLSDHEATLRC | |
| | WALGFYPAEITLTWQRDGEDQTAKTELVETRPAGDGTFQKW | |
| | AAVVVPSGQEQRYTCHMQHEGLQEPLTLSWEPS | |
| Synthetic | SYRPGTVALGCGGSGGGGSIQRTPKIQVYSRHPAENGKSNFLN | 270 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGSHSMRYFDTAVSRPGRGEPRFISVGYVDDTQFVRFDSDAAS | |
| | PRGEPRAPWVEQEGPEYWDRETQKYKRQAQADRVSLRNLRG | |
| | CYNQSEDGSHTLQRMSGCDLGPDGRLLRGYDQSAYDGKDYI | |
| | ALNEDLRSWTAADTAAQITQRKLEAARAAEQLRAYLEGTCV | |
| | EWLRRYLENGKETLQRAEPPKTHVTHHPLSDHEATLRCWAL | |
| | GFYPAEITLTWQRDGEDQTAKTELVETRPAGDGTFQKWAAV | |
| | VVPSGQEQRYTCHMQHEGLQEPLTLSWEPS | |
| Synthetic | SYRPGTVALGCGGSGGGGSIQRTPKIQVYSRHPAENGKSNFLN | 271 |
| HLA | CYVSGFHPSDIEVDLLKNGERIEKVEHSDLSFSKDWSFYLLYY | |
| (synHLA) | TEFTPTEKDEYACRVNHVTLSQPKIVKWDRDMGGGGSGGGG | |
| | SGGGGSGGGGSGSHSMRYFDTAVSRPGRGEPRFISVGYVDDT | |
| | QFVRFDSDAASPRGEPRAPWVEQEGPEYWDRETQKYKRQAQ | |
| | ADRVSLRNLRGCYNQSEDGSHTLQRMSGCDLGPDGRLLRGY | |
| | DQSAYDGKDYIALNEDLRSWTAADTAAQITQRKLEAARAAE | |
| | QLRAYLEGTCVEWLRRYLENGKETLQRAEPPKTHVTHHPLSD | |
| | HEATLRCWALGFYPAEITLTWQRDGEDQTAKTELVETRPAGD | |
| | GTFQKWAAVVVPSGQEQRYTCHMQHEGLQEPLTLSWEPS | |
| Synthetic | MSRSVALAVLALLSLSGLEARYRPGTEALGCGGSGGGGSIQR | 272 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVELGCGGSGGGGSIQR | 273 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TAKTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVALGCGGSGGGGSIQR | 274 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | ___TELVETRPAGDGTFQKWAAVVVPSGQEQRYTCHMQHEGL | |
| | QEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVALGCGGSGGGGSIQR | 275 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | ___TELV_TRPAGDGTFQKWAAVVVPSGQEQRYTCHMQHEGL | |
| | QEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |
| Synthetic | MSRSVALAVLALLSLSGLEARYRPGTVALGCGGSGGGGSIQR | 276 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGSHSMRYFDTAVSRPGRGEPR | |
| | FISVGYVDDTQFVRFDSDAASPRGEPRAPWVEQEGPEYWDRE | |
| | TQKYKRQAQADRVSLRNLRGCYNQSEDGSHTLQRMSGCDLG | |
| | PDGRLLRGYDQSAYDGKDYIALNEDLRSWTAADTAAQITQR | |
| | KLEAARAAEQLRAYLEGTCVEWLRRYLENGKETLQRAEPPKT | |
| | HVTHHPLSDHEATLRCWALGFYPAEITLTWQRDGEDQTAKTE | |
| | LVETRPAGDGTFQKWAAVVVPSGQEQRYTCHMQHEGLQEPL | |
| | TLSWEPSSQPTIPIMGIVAGLAVLVVLAVLGAVVTAMMCRRK | |
| | SSGGKGGSCSQAACSNSAQGSDESLITCKAVRAKRSGSGEGR | |
| | GSLLTCGDVEENPGPM | |
| Synthetic | MSRSVALAVLALLSLSGLEARYRPGTVALGCGGSGGGGSIQR | 277 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGGGGSGSHSMRYFDT | |
| | AVSRPGRGEPRFISVGYVDDTQFVRFDSDAASPRGEPRAPWVE | |
| | QEGPEYWDRETQKYKRQAQADRVSLRNLRGCYNQSEDGSHT | |
| | LQRMSGCDLGPDGRLLRGYDQSAYDGKDYIALNEDLRSWTA | |
| | ADTAAQITQRKLEAARAAEQLRAYLEGTCVEWLRRYLENGK | |
| | ETLQRAEPPKTHVTHHPLSDHEATLRCWALGFYPAEITLTWQ | |
| | RDGEDQTAKTELVETRPAGDGTFQKWAAVVVPSGQEQRYTC | |
| | HMQHEGLQEPLTLSWEPSSQPTIPIMGIVAGLAVLVVLAVLGA | |
| | VVTAMMCRRKSSGGKGGSCSQAACSNSAQGSDESLITCKAVR | |
| | AKRSGSGEGRGSLLTCGDVEENPGPM | |
| Synthetic | MSRSVALAVLALLSLSGLEARYRPGTVALGCGGSGGGGSIQR | 278 |
| HLA | TPKIQVYSRHPAENGKSNFLNCYVSGFHPSDIEVDLLKNGERIE | |
| (synHLA) | KVEHSDLSFSKDWSFYLLYYTEFTPTEKDEYACRVNHVTLSQ | |
| | PKIVKWDRDMGGGGSGGGGSGGGGSGSHSMRYFDTAVSRPG | |
| | RGEPRFISVGYVDDTQFVRFDSDAASPRGEPRAPWVEQEGPEY | |
| | WDRETQKYKRQAQADRVSLRNLRGCYNQSEDGSHTLQRMS | |
| | GCDLGPDGRLLRGYDQSAYDGKDYIALNEDLRSWTAADTAA | |
| | QITQRKLEAARAAEQLRAYLEGTCVEWLRRYLENGKETLQRA | |
| | EPPKTHVTHHPLSDHEATLRCWALGFYPAEITLTWQRDGEDQ | |
| | TQDTELVETRPAGDGTFQKWAAVVVPSGQEQRYTCHMQHEG | |
| | LQEPLTLSWEPSSQPTIPIMGIVAGLAVLVVLAVLGAVVTAMM | |
| | CRRKSSGGKGGSCSQAACSNSAQGSDESLITCKAVRAKRSGS | |
| | GEGRGSLLTCGDVEENPGPM | |

### Targeting Moieties

Provided herein, in one aspect, are targeting moieties. In some embodiments, the targeting moiety comprises a peptide. In some embodiments, the peptide is configured to weaken or inhibit HLA activity. In some embodiments, the peptide is configured to weaken or inhibit T cell activity. In some embodiments, the peptide is configured to weaken or inhibit NK cell activity.

In some embodiments, the peptide does not elicit a T cell response when the peptide (or a complex comprising the peptide) is interrogated by one or more T cells. In some embodiments, the peptide is incapable of activating the one or more T cells. In some embodiments, the peptide is capable of binding to a receptor of the one or more T cells, and wherein the binding is insufficient to activate the one or more T cells. In some embodiments, the peptide does not elicit an NK cell response when the peptide (or a complex comprising the peptide) is interrogated by one or more NK cells). In some embodiments, the peptide is incapable of activating the one or more NK cells. In some embodiments, the peptide is capable of binding to a receptor of the one or more NK cells, and wherein the binding is insufficient to activate the one or more NK cells.

In some embodiments, a peptide is configured to covalently bind to HLA. In some embodiments, the peptide is configured to bind to the N-terminus of the beta chain of HLA Class II or the N-terminus of beta-2 microglobulin (B2M) chain of HLA Class 1. In some embodiments, the peptide is configured to be specific for the MHC binding groove but does not comprise the correct TCR-facing/solvent exposed amino acids required for recognition by the T-cell receptor (TCR). The presence of specific “anchor residues” in the MHC binding groove of HLA act to anchor the bound peptide.

In some embodiments, residues in the peptide can be altered in order to configure the peptide so that a TCR does not recognize and/or bind to the peptide-bound MHC (pMHC,) (e.g., a peptide bound to a human HLA class 1 heavy chain). Although the TCR can interact with residues in the MHC, 1-3 TCR-facing, solvent-exposed residues from the peptide also contribute directly to the TCR interaction. In some embodiments, the TCR-facing residues of the peptide are configured to antagonize TCR interaction.

In some embodiments, the peptide binds to one or more HLA binding groove domain residues of the human HLA class 1 heavy chain sequence. In some embodiments, the peptide modulates a conformation of the human HLA class 1 heavy chain sequence. In some embodiments, the conformation prevents the one or more T cells from binding to the human HLA class 1 heavy chain sequence.

In some embodiments, the sequence of the bound peptide can affect the intrinsic flexibility of the pMHC. In some embodiments, the conformational flexibility of the pMHC facilitates TCR interaction. In some embodiments, the peptide configured to bind to HLA is further configured to increase the conformational variability of pMHC and to prevent TCR engagement.

In some embodiments, the peptide is about 8 amino acids in length to about 15 amino acids in length. In some embodiments, the peptide is about 8 amino acids in length to about 9 amino acids in length, about 8 amino acids in length to about 10 amino acids in length, about 8 amino acids in length to about 11 amino acids in length, about 8 amino acids in length to about 12 amino acids in length, about 8 amino acids in length to about 13 amino acids in length, about 8 amino acids in length to about 14 amino acids in length, about 8 amino acids in length to about 15 amino acids in length, about 9 amino acids in length to about 10 amino acids in length, about 9 amino acids in length to about 11 amino acids in length, about 9 amino acids in length to about 12 amino acids in length, about 9 amino acids in length to about 13 amino acids in length, about 9 amino acids in length to about 14 amino acids in length, about 9 amino acids in length to about 15 amino acids in length, about 10 amino acids in length to about 11 amino acids in length, about 10 amino acids in length to about 12 amino acids in length, about 10 amino acids in length to about 13 amino acids in length, about 10 amino acids in length to about 14 amino acids in length, about 10 amino acids in length to about 15 amino acids in length, about 11 amino acids in length to about 12 amino acids in length, about 11 amino acids in length to about 13 amino acids in length, about 11 amino acids in length to about 14 amino acids in length, about 11 amino acids in length to about 15 amino acids in length, about 12 amino acids in length to about 13 amino acids in length, about 12 amino acids in length to about 14 amino acids in length, about 12 amino acids in length to about 15 amino acids in length, about 13 amino acids in length to about 14 amino acids in length, about 13 amino acids in length to about 15 amino acids in length, or about 14 amino acids in length to about 15 amino acids in length. In some embodiments, the peptide is about 8 amino acids in length, about 9 amino acids in length, about 10 amino acids in length, about 11 amino acids in length, about 12 amino acids in length, about 13 amino acids in length, about 14 amino acids in length, or about 15 amino acids in length. In some embodiments, the peptide is at least about 8 amino acids in length, about 9 amino acids in length, about 10 amino acids in length, about 11 amino acids in length, about 12 amino acids in length, about 13 amino acids in length, or about 14 amino acids in length. In some embodiments, the peptide is at most about 9 amino acids in length, about 10 amino acids in length, about 11 amino acids in length, about 12 amino acids in length, about 13 amino acids in length, about 14 amino acids in length, or about 15 amino acids in length. In some embodiments, the peptide comprises greater than 14 amino acids.

In some embodiments, the use of unusually long peptides to bind to the MHC binding groove of HLA inhibits HLA activity. MHC-I can bind peptides 8-10 amino acids in length, but can also bind non-canonical, longer peptides (e.g. 13 amino acids). The ends of such a long peptide bind to the MHC binding groove at the anchor residues, creating a “bulge” at the center of the peptide binding site. In such pMHC-TCR constructs, the TCR makes relatively few contacts with the MHC (typically with canonical, short peptides in such constructs the MHC heavy chain dominates the interface with TCR) and instead the interaction with TCR is dominated by the peptide directly. The bulged peptide also represents a steric challenge for TCR engagement. Given the dominance of peptide-TCR interactions in such a system, by selecting the peptide sequence at the bulge, it is possible to prevent TCR-binding. In some embodiments, the peptide is configured to block and/or silence the amino acids of HLA required for molecular contacts with TCR and/or the peptide does not comprise amino acid residues sufficient for TCR binding and/or activity. In some embodiments, the peptide is configured to increase the conformational heterogeneity of HLA in this region as to render the HLA incapable of TCR binding and/or activity. In some embodiments, the peptide is configured to do any combination of the functions described above.

In some embodiments, the peptide is coupled to the construct by a disulfide bond. The peptide can additionally be covalently linked to the rest of the construct. The disulfide bond may connect a disulfide staple pair. The disulfide staple pair may be located or distributed across appropriate part or parts of the construct, such as on the peptide and on the human HLA class I heavy chain or on the peptide and on a linker.

In some embodiments, the peptide comprises or is otherwise derived from an Influenza A virus M1 peptide (e.g., SEQ ID NO: 158) or mutant thereof. In some embodiments, the peptide comprises or is otherwise derived from a Histone H3 peptide (e.g., SEQ ID NO: 159) or mutant thereof.

In some embodiments, the construct further comprises a regulatory peptide. In some embodiments, the regulatory peptide is an apoptosis-inducing peptide. In some embodiments, the apoptosis-inducing peptide acts as a “kill switch” for the construct.

In some embodiments, the targeting moiety comprises a peptide comprising a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to a sequence listed in Table 2 below.

TABLE 2
Peptides to weaken or inhibit HLA activity

| | Peptide | | SEQ ID |
|---|---|---|---|
| Peptide name | sequence | Description | NO |
| GL9 | GILGFVFTL | Influenza A virus M1(58-66) peptide | 158 |
| RL9 | RYRPGTVAL | Histone H3 peptide | 159 |
| RL2(R1S) | SYRPGTVAL | Histone H3 peptide. R1S to facilitate | 160 |
| | | initiating Met cleavage | |
| RL9 AM1 | SRRYCNVAY | Affinity matured RL9 peptide | 161 |
| [R1S, G5C] | | | |
| RL9[R1S, G5C] | SYRPCTVAL | Cys mutated to pair with R69C mutation | 162 |
| RL9[R1S, V7C] | SYRPGTCAL | Cys mutated to pair with A150C | 163 |
| | | mutation | |
| RL9[R1S, A8C] | SYRPGTVCL | Cys mutated to pair with A73C mutation | 164 |
| RL9[R1S, V7E] | SYRPGTEAL | Predicted to block KIR binding | 165 |
| RL9[R1S, A8E] | SYRPGTVEL | Predicted to block KIR binding | 166 |
| RL9 AM2 | SWRYCNVAY | Affinity matured RL9 peptide; Cys | 167 |
| [R1S, G5C] | | mutation to pair with R69C mutation | |
| RL9 AM3 | SRRYCQVAY | Affinity matured RL9 peptide; Cys | 168 |
| [R1S, G5C] | | mutation to pair with R69C mutation | |
| RL9 AM4 | SRRYEMCAM | Affinity matured RL9 peptide; Cys | 169 |
| [R1S, V7C] | | mutation to pair with A150C mutation | |
| RL9 AM5 | SRRYLMCAY | Affinity matured RL9 peptide; Cys | 170 |
| [R1S, V7C] | | mutation to pair with A150C mutation | |
| RL9 AM6 | SRRWLMCAY | Affinity matured RL9 peptide; Cys | 171 |
| [R1S, V7C] | | mutation to pair with A150C mutation | |
| RL9 AM7 | SRRMEMVCI | Affinity matured RL9 peptide; Cys | 172 |
| [R1S, A8C] | | mutation to pair with A73C mutation | |
| RL9 AM8 | SRRMWMVCI | Affinity matured RL9 peptide; Cys | 173 |
| [R1S, A8C] | | mutation to pair with A73C mutation | |
| RL9 AM9 | SRRMEMVCR | Affinity matured RL9 peptide; Cys | 174 |
| [R1S, A8C] | | mutation to pair with A73C mutation | |

### Linkers

In some embodiments, the construct comprises one or more linkers between the targeting moiety (e.g., peptide) and an MHC region (e.g., a human HLA class 1 heavy chain sequence). In some embodiments, the one or more linkers are configured to resist proteolytic cleavage. In some embodiments, the one or more linkers comprise a conformation configured to not block one or more killer-cell immunoglobulin-like receptor (KIR) binding sites on the human HLA class 1 heavy chain sequence. In some embodiments, the one or more linkers are structurally stable. In some embodiments, the one or more linkers are rigid. In some embodiments, the one or more linkers possess limited flexibility. In some embodiments, the structural stability, rigidity, and limited flexibility of the one or more linkers increase resistance to proteolytic degradation. In some embodiments, the one or more linkers comprise a cysteine which is part of a disulfide staple pair as described herein.

In some embodiments, a linker of the one or more linkers is disposed between the targeting moiety (e.g., peptide) and the human beta-2 microglobulin (B2M) sequence, between the human B2M sequence and the human HLA class 1 heavy chain sequence, or both. In some embodiments, a linker of the one or more linkers is disposed between the peptide and the human B2M sequence. In some embodiments, a linker of the one or more linkers is disposed between the human B2M sequence and the human HLA class 1 heavy chain sequence. In some embodiments, a first linker of the one or more linkers is disposed between the peptide and the human B2M sequence and a second linker of the one or more linkers is disposed between the human B2M sequence and the human HLA class 1 heavy chain sequence. In some embodiments, the second linker comprises a conformation configured to resist proteolytic cleavage. In some embodiments, the second linker is further configured to not block one or more killer-cell immunoglobulin-like receptor (KIR) binding sites on the human HLA class 1 heavy chain sequence. In some embodiments, the conformation of the second linker allows for KIR binding to the human HLA class 1 heavy chain sequence, preventing a “missing self” immune response. In some embodiments, the conformation of the second linker prevents attack by one or more natural killer cells.

A linker may be configured to impart a certain secondary, tertiary, or quaternary structure when the construct sequence is arranged in three-dimensional space, such as when expressed in a host cell. The secondary, tertiary, or quaternary structure may be determined from experimental structural biology data (e.g., X-ray crystallographic data, cryogenic electron microscopy data, nuclear magnetic resonance data), biochemical data (e.g., mass spectrometry data, chromatographic data, electrophoretic data), or computer simulation or modeling data (e.g., molecular dynamics simulations, de novo or ab initio prediction, homology modeling, fragment assembly, secondary structure prediction). Alternatively or additionally, the linker may be configured to impart a certain functional consequence on the expressed construct. In some embodiments, the linker enhances expression (e.g., cell surface expression) of the contrast, enhance stability of the construct, prevent exchange of the peptide, or some combination thereof. In some embodiments, the linker enhances expression of the construct.

In some embodiments, the linker may be configured to increase expression in a host cell (e.g., relative to a wild-type or other construct lacking the linker). In some embodiments, the expression is cell-surface expression. The expression may be measured by, for example, flow cytometry, fluorescence microscopy, mass spectrometry, or any other suitable quantification method. In some embodiments, the presence of a linker increases expression of the construct in a host cell relative to a reference (e.g., wild-type) construct. In some embodiments, expression is increased by at least about 1%, about 5%, about 10%, about 20%, about 30%, about 40%, about 50%, about 60%, about 70%, about 80%, about 90%, or about 100%, or more. In some embodiments, expression is increased by about 1% to about 100%. In some embodiments, expression is increased by about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, expression is increased by about 1%, about 5%, about 10%, about 20%, about 30%, about 40%, about 50%, about 60%, about 70%, about 80%, about 90%, or about 100%. In some embodiments, expression is increased by at least about 1%, about 5%, about 10%, about 20%, about 30%, about 40%, about 50%, about 60%, about 70%, about 80%, or about 90%. In some embodiments, expression is increased by at most about 5%, about 10%, about 20%, about 30%, about 40%, about 50%, about 60%, about 70%, about 80%, about 90%, or about 100%.

In some embodiments, the linker may be configured to increase stability of the construct (e.g., as compared to a construct not comprising the linker, such as a wild-type construct). The presence of the linker may increase stability by restricting the conformational flexibility of a construct to help the construct fold into and retain a particular secondary, tertiary, and/or quaternary structure. The stability of a construct may be measured by differential scanning calorimetry (DSC), pulse-chase assays (such as bleach-chase and cycloheximide-chase assays), thermal shift assays, circular dichroism (CD) spectroscopy, UV-vis spectroscopy, nuclear magnetic resonance (NMR), gel filtration, isothermal calorimetry, light scattering, or any other suitable assay or instrument. In some embodiments the stability of a construct is expressed in relative terms (e.g., percent or fold change in stability with respect to a reference, such as a wild-type construct). In some embodiments, the stability of a construct is expressed in absolute terms (e.g., in terms of thermodynamic coordinates such as melting or other phase transition temperatures or a free energy of folding).

In some embodiments, the presence of a linker increases the stability of a construct at least about 1%, about 5%, about 10%, about 20%, about 30%, about 40%, about 50%, about 60%, about 70%, about 80%, about 90%, or about 100%, or more, relative to a reference (e.g., construct not comprising the linker, such as a wild-type construct). In some embodiments, stability is increased by about 1% to about 100%. In some embodiments, stability is increased by about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, stability is increased by about 1%, about 5%, about 10%, about 20%, about 30%, about 40%, about 50%, about 60%, about 70%, about 80%, about 90%, or about 100%. In some embodiments, stability is increased by at least about 1%, about 5%, about 10%, about 20%, about 30%, about 40%, about 50%, about 60%, about 70%, about 80%, or about 90%. In some embodiments, stability is increased by at most about 5%, about 10%, about 20%, about 30%, about 40%, about 50%, about 60%, about 70%, about 80%, about 90%, or about 100%.

In some embodiments, the presence of a linker increases the stability of a construct by about 2-fold, about 4-fold, about 5-fold, about 6-fold, about 7-fold, about 8-fold, about 9-fold, about 10-fold, about 50-fold, about 100-fold, about 1000, or more, relative to a refence (e.g., construct not comprising the linker, such as a wild-type construct). In some embodiments, stability is increased by about 1-fold to about 1,000-fold. In some embodiments, stability is increased by about 1-fold to about 2-fold, about 1-fold to about 3-fold, about 1-fold to about 4-fold, about 1-fold to about 5-fold, about 1-fold to about 10-fold, about 1-fold to about 100-fold, about 1-fold to about 1,000-fold, about 2-fold to about 3-fold, about 2-fold to about 4-fold, about 2-fold to about 5-fold, about 2-fold to about 10-fold, about 2-fold to about 100-fold, about 2-fold to about 1,000-fold, about 3-fold to about 4-fold, about 3-fold to about 5-fold, about 3-fold to about 10-fold, about 3-fold to about 100-fold, about 3-fold to about 1,000-fold, about 4-fold to about 5-fold, about 4-fold to about 10-fold, about 4-fold to about 100-fold, about 4-fold to about 1,000-fold, about 5-fold to about 10-fold, about 5-fold to about 100-fold, about 5-fold to about 1,000-fold, about 10-fold to about 100-fold, about 10-fold to about 1,000-fold, or about 100-fold to about 1,000-fold. In some embodiments, stability is increased by about 1-fold, about 2-fold, about 3-fold, about 4-fold, about 5-fold, about 10-fold, about 100-fold, or about 1,000-fold. In some embodiments, stability is increased by at least about 1-fold, about 2-fold, about 3-fold, about 4-fold, about 5-fold, about 10-fold, or about 100-fold. In some embodiments, stability is increased by at most about 2-fold, about 3-fold, about 4-fold, about 5-fold, about 10-fold, about 100-fold, or about 1,000-fold.

In some embodiments, a construct as described herein comprising a linker may show a higher melting temperature (Tₘ) (e.g., as determined by a thermodynamic technique, such as DSC) relative to a reference (e.g., a construct that does not comprise the linker, such as a wild-type construct). In some embodiments, the Tₘ is increased by about 1° C., about 2° C., about 3° C., about 4° C., about 5° C., about 6° C., about 7° C., about 8° C., about 9° C., about 10° C., about 20° C., about 30° C., or more, relative to the reference. In some embodiments, the Tₘ is increased by about 1° C. to about 30° C. In some embodiments, the Tₘ is increased by about 1° C. to about 2° C., about 1° C. to about 3° C., about 1° C. to about 4° C., about 1° C. to about 5° C., about 1° C. to about 6° C., about 1° C. to about 7° C., about 1° C. to about 8° C., about 1° C. to about 9° C., about 1° C. to about 10° C., about 1° C. to about 20° C., about 1° C. to about 30° C., about 2° C. to about 3° C., about 2° C. to about 4° C., about 2° C. to about 5° C., about 2° C. to about 6° C., about 2° C. to about 7° C., about 2° C. to about 8° C., about 2° C. to about 9° C., about 2° C. to about 10° C., about 2° C. to about 20° C., about 2° C. to about 30° C., about 3° C. to about 4° C., about 3° C. to about 5° C., about 3° C. to about 6° C., about 3° C. to about 7° C., about 3° C. to about 8° C., about 3° C. to about 9° C., about 3° C. to about 10° C., about 3° C. to about 20° C., about 3° C. to about 30° C., about 4° C. to about 5° C., about 4° C. to about 6° C., about 4° C. to about 7° C., about 4° C. to about 8° C., about 4° C. to about 9° C., about 4° C. to about 10° C., about 4° C. to about 20° C., about 4° C. to about 30° C., about 5° C. to about 6° C., about 5° C. to about 7° C., about 5° C. to about 8° C., about 5° C. to about 9° C., about 5° C. to about 10° C., about 5° C. to about 20° C., about 5° C. to about 30° C., about 6° C. to about 7° C., about 6° C. to about 8° C., about 6° C. to about 9° C., about 6° C. to about 10° C., about 6° C. to about 20° C., about 6° C. to about 30° C., about 7° C. to about 8° C., about 7° C. to about 9° C., about 7° C. to about 10° C., about 7° C. to about 20° C., about 7° C. to about 30° C., about 8° C. to about 9° C., about 8° C. to about 10° C., about 8° C. to about 20° C., about 8° C. to about 30° C., about 9° C. to about 10° C., about 9° C. to about 20° C., about 9° C. to about 30° C., about 10° C. to about 20° C., about 10° C. to about 30° C., or about 20° C. to about 30° C. In some embodiments, the Tₘ is increased by about 1° C., about 2° C., about 3° C., about 4° C., about 5° C., about 6° C., about 7° C., about 8° C., about 9° C., about 10° C., about 20° C., or about 30° C. In some embodiments, the Tₘ is increased by at least about 1° C., about 2° C., about 3° C., about 4° C., about 5° C., about 6° C., about 7° C., about 8° C., about 9° C., about 10° C., or about 20° C. In some embodiments, the Tₘ is increased by at most about 2° C., about 3° C., about 4° C., about 5° C., about 6° C., about 7° C., about 8° C., about 9° C., about 10° C., about 20° C., or about 30° C.

In some embodiments, a linker may be configured not to block a killer-cell immunoglobulin-like receptor (KIR) receptor. The linker can impart to the construct a particular secondary, tertiary, and/or quaternary structure that does not block binding of an (e.g., inhibitory) KIR to the construct). In some embodiments, the linker does not block KIR binding when expressed on the surface of a cell. Accordingly, when a cell (e.g., NK cell) comprising the KIR interrogates the construct, the KIR is not activated or is activated less than it is when interacting with a reference (e.g., wild-type) construct. In some embodiments, the KIR comprises one or more of KIR2DL1, KIR2DL2, KIR2DL3, KIR3DL1, KIR3DL2, KIR3DL3, KIR2DL5A, or KIR2DL5B. A linker's ability to permit the binding of KIR to the construct may be measured by assaying the binding affinity between the construct (e.g., either soluble form or cell-surface form) and/or by a functional assay. In some embodiments, the functional assay is an NK-cell killing assay. In some embodiments, the NK-cell killing assay is a chromium-release assay.

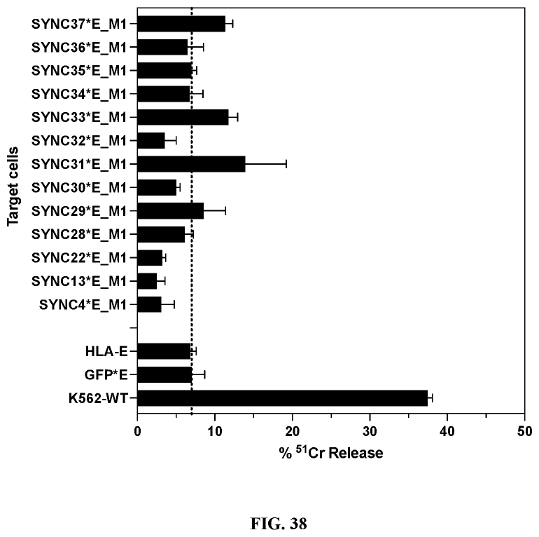

An example of a linker configured not to block a KIR is illustrated in FIG. 39. FIG. 39 shows a 2.35 Å resolution X-ray crystal structure of an example synthetic human leukocyte antigen (synHLA) construct 3901 as described herein (SEQ ID NO: 125). in complex with killer-cell immunoglobulin-like receptor (KIR2DL2, 3902), resulting in a successful KIR interaction and no “missing self” immune signal. Linker 1 3901b does not block the KIR2DL2 interaction. The targeting moiety 3901a (illustrated here as a peptide) is shown in black spheres in complex with the HLA heavy chain of construct 3901 and KIR2DL2 3902.

In some embodiments, a construct comprising the linker is characterized by a higher affinity of the construct for a (e.g., inhibitory) KIR than a refence (e.g., a construct not comprising the linker). In some embodiments, the presence of a linker increases the affinity of a KIR for the construct at least about 1%, about 5%, about 10%, about 20%, about 30%, about 40%, about 50%, about 60%, about 70%, about 80%, about 90%, or about 100%, or more, relative to a reference (e.g., construct not comprising the linker, such as a wild-type construct). In some embodiments, stability is increased by about 1% to about 100%. In some embodiments, stability is increased by about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, stability is increased by about 1%, about 5%, about 10%, about 20%, about 30%, about 40%, about 50%, about 60%, about 70%, about 80%, about 90%, or about 100%. In some embodiments, stability is increased by at least about 1%, about 5%, about 10%, about 20%, about 30%, about 40%, about 50%, about 60%, about 70%, about 80%, or about 90%. In some embodiments, stability is increased by at most about 5%, about 10%, about 20%, about 30%, about 40%, about 50%, about 60%, about 70%, about 80%, about 90%, or about 100%.

In some embodiments, the presence of a linker increases the affinity of a KIR for the construct by about 2-fold, about 4-fold, about 5-fold, about 6-fold, about 7-fold, about 8-fold, about 9-fold, about 10-fold, about 50-fold, about 100-fold, about 1000, or more, relative to a refence (e.g., construct not comprising the linker, such as a wild-type construct). In some embodiments, stability is increased by about 1-fold to about 1,000-fold. In some embodiments, stability is increased by about 1-fold to about 2-fold, about 1-fold to about 3-fold, about 1-fold to about 4-fold, about 1-fold to about 5-fold, about 1-fold to about 10-fold, about 1-fold to about 100-fold, about 1-fold to about 1,000-fold, about 2-fold to about 3-fold, about 2-fold to about 4-fold, about 2-fold to about 5-fold, about 2-fold to about 10-fold, about 2-fold to about 100-fold, about 2-fold to about 1,000-fold, about 3-fold to about 4-fold, about 3-fold to about 5-fold, about 3-fold to about 10-fold, about 3-fold to about 100-fold, about 3-fold to about 1,000-fold, about 4-fold to about 5-fold, about 4-fold to about 10-fold, about 4-fold to about 100-fold, about 4-fold to about 1,000-fold, about 5-fold to about 10-fold, about 5-fold to about 100-fold, about 5-fold to about 1,000-fold, about 10-fold to about 100-fold, about 10-fold to about 1,000-fold, or about 100-fold to about 1,000-fold. In some embodiments, stability is increased by about 1-fold, about 2-fold, about 3-fold, about 4-fold, about 5-fold, about 10-fold, about 100-fold, or about 1,000-fold. In some embodiments, stability is increased by at least about 1-fold, about 2-fold, about 3-fold, about 4-fold, about 5-fold, about 10-fold, or about 100-fold. In some embodiments, stability is increased by at most about 2-fold, about 3-fold, about 4-fold, about 5-fold, about 10-fold, about 100-fold, or about 1,000-fold.

In some embodiments, a construct comprising the linker is characterized by a lower amount of NK-cell killing (e.g., as determined by a chromium-release assay) than a refence (e.g., a construct not comprising a linker). In some embodiments, the presence of a linker reduced NK-cell killing by at least about 1%, about 5%, about 10%, about 20%, about 30%, about 40%, about 50%, about 60%, about 70%, about 80%, about 90%, or about 100%, or more, relative to a reference (e.g., construct not comprising the linker, such as a wild-type construct). In some embodiments, NK-cell is reduced by about 1% to about 100%. In some embodiments, NK-cell killing is reduced by about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, NK-cell killing is reduced by about 1%, about 5%, about 10%, about 20%, about 30%, about 40%, about 50%, about 60%, about 70%, about 80%, about 90%, or about 100%. In some embodiments, NK-cell killing is reduced by at least about 1%, about 5%, about 10%, about 20%, about 30%, about 40%, about 50%, about 60%, about 70%, about 80%, or about 90%. In some embodiments, NK-cell killing is reduced by at most about 5%, about 10%, about 20%, about 30%, about 40%, about 50%, about 60%, about 70%, about 80%, about 90%, or about 100%.

In some embodiments, the presence of a reduced NK-cell killing of a cell expressing the construct by about 2-fold, about 4-fold, about 5-fold, about 6-fold, about 7-fold, about 8-fold, about 9-fold, about 10-fold, about 50-fold, about 100-fold, about 1000, or more, relative to a refence (e.g., construct not comprising the linker, such as a wild-type construct). In some embodiments, NK-cell killing is decreased by about 1-fold to about 1,000-fold. In some embodiments, NK-cell killing is decreased by about 1-fold to about 2-fold, about 1-fold to about 3-fold, about 1-fold to about 4-fold, about 1-fold to about 5-fold, about 1-fold to about 10-fold, about 1-fold to about 100-fold, about 1-fold to about 1,000-fold, about 2-fold to about 3-fold, about 2-fold to about 4-fold, about 2-fold to about 5-fold, about 2-fold to about 10-fold, about 2-fold to about 100-fold, about 2-fold to about 1,000-fold, about 3-fold to about 4-fold, about 3-fold to about 5-fold, about 3-fold to about 10-fold, about 3-fold to about 100-fold, about 3-fold to about 1,000-fold, about 4-fold to about 5-fold, about 4-fold to about 10-fold, about 4-fold to about 100-fold, about 4-fold to about 1,000-fold, about 5-fold to about 10-fold, about 5-fold to about 100-fold, about 5-fold to about 1,000-fold, about 10-fold to about 100-fold, about 10-fold to about 1,000-fold, or about 100-fold to about 1,000-fold. In some embodiments, NK-cell killing is decreased by about 1-fold, about 2-fold, about 3-fold, about 4-fold, about 5-fold, about 10-fold, about 100-fold, or about 1,000-fold. In some embodiments, NK-cell killing is decreased by at least about 1-fold, about 2-fold, about 3-fold, about 4-fold, about 5-fold, about 10-fold, or about 100-fold. In some embodiments, NK-cell killing is decreased by at most about 2-fold, about 3-fold, about 4-fold, about 5-fold, about 10-fold, about 100-fold, or about 1,000-fold.

In some embodiments, a linker of the one or more linkers comprises a sequence at least about 50%, about 60%, about 70%, about 80%, about 85%, about 86%, about 87%, about 88%, about 89%, about 90%, about 91%, about 92%, about 93%, about 94%, about 95%, about 96%, about 97%, about 98%, about 99%, about 99.9%, or about 100% identical to a sequence listed in Table 3 below.

TABLE 3
Linker sequences

| | | SEQ |
|---|---|---|
| | | ID |
| Linker name | Sequence | NO |
| L1 short | GGGGSGGGGS | 175 |
| L1 short (diS staple) | GCGGSGGGGS | 176 |
| L1 medium | GGGGSGGGGSGS | 177 |
| L1 medium (diS staple) | GCGGSGGGGSGS | 178 |
| L1 long | GGGGSGGGGSGGGGS | 179 |
| L1 long (diS staple) | GCGGSGGGGSGGGGS | 180 |
| Shorter L1 (L1-1) | GGGSGGGGS | 181 |
| Shorter L1 (L1-1) | GCGSGGGGS | 182 |
| (diS staple) | | |
| L2 long | (GGGGS)3 | 183 |
| L2 extra long | (GGGGS)4 | 184 |
| L2 short | GGGGSGGGGS | 185 |
| L2 extra-long ala | GGGGAAAAAAAAGGGSGGGG | 186 |
| rich | | |
| SGSG linker | SGSG | 198 |

In some embodiments, the one or more human leukocyte antigens (HLAs) comprise one or more mutations, wherein the one or more mutations inhibit the one or more HLAs from eliciting a T cell response when the construct is interrogated by one or more cluster of differentiation 8 (CD8) cells. In some embodiments, the one or more mutations may be located in a CD8 binding site. In some embodiments, the one or more mutations comprises a mutation of one or more of amino acid residues 84, 115, 122, 128, 194, 197, 198, 212, 214, 222, 223, 224, 225, 226, 227, 228, 229, 230, 231, 232, 233, 243, 245, 248, 262, or any combination thereof. In some embodiments, the one or more mutations comprises a deletion of one or more of amino acid residues 84, 115, 122, 128, 194, 197, 198, 212, 214, 222, 223, 224, 225, 226, 227, 228, 229, 230, 231, 232, 233, 243, 245, 248, 262, or any combination thereof. In some embodiments, the one or more mutations comprises a mutation of amino acid residue 84. In some embodiments, the one or more mutations comprises a mutation of amino acid residue 115. In some embodiments, the one or more mutations comprises a mutation of amino acid residue 122. In some embodiments, the one or more mutations comprises a mutation of amino acid residue 128. In some embodiments, the one or more mutations comprises a mutation of amino acid residue 194. In some embodiments, the one or more mutations comprises a mutation of amino acid residue 197. In some embodiments, the one or more mutations comprises a mutation of amino acid residue 198. In some embodiments, the one or more mutations comprises a mutation of amino acid residue 212. In some embodiments, the one or more mutations comprises a mutation of amino acid residue 214. In some embodiments, the one or more mutations comprises a mutation of amino acid residue 222. In some embodiments, the one or more mutations comprises a mutation of amino acid residue 223. In some embodiments, the one or more mutations comprises a mutation of amino acid residue 224. In some embodiments, the one or more mutations comprises a mutation of amino acid residue 225. In some embodiments, the one or more mutations comprises a mutation of amino acid residue 226. In some embodiments, the one or more mutations comprises a mutation of amino acid residue 227. In some embodiments, the one or more mutations comprises a mutation of amino acid residue 228. In some embodiments, the one or more mutations comprises a mutation of amino acid residue 229. In some embodiments, the one or more mutations comprises a mutation of amino acid residue 230. In some embodiments, the one or more mutations comprises a mutation of amino acid residue 231. In some embodiments, the one or more mutations comprises a mutation of amino acid residue 232. In some embodiments, the one or more mutations comprises a mutation of amino acid residue 233. In some embodiments, the one or more mutations comprises a mutation of amino acid residue 243. In some embodiments, the one or more mutations comprises a mutation of amino acid residue 245. In some embodiments, the one or more mutations comprises a mutation of amino acid residue 248. In some embodiments, the one or more mutations comprises a mutation of amino acid residue 262.

In some embodiments, the construct further comprises one or more proteins or fragments thereof that inhibit an immune response by the complement system. In some embodiments, the one or more proteins or fragments thereof are selected from CD48, CD59, or a combination thereof. In some embodiments, the one or more proteins or fragments thereof is CD48. In some embodiments, the one or more proteins or fragments thereof is CD59. In some embodiments, the one or more proteins or fragments thereof are CD48 and CD59.

In some embodiments, the peptide comprises a second amino acid residue selected from L, M, S, I, F, T, V, and Y. In some embodiments, the second amino acid residue is selected from T, V, and Y. In some embodiments, the peptide comprises a last amino acid residue selected from V, I, F, W, Y, L, R, and K. In some embodiments, the last amino acid residue is selected from Y, L, R, and K.

In some embodiments, the peptide comprises a second amino acid residue selected from E, P, L, Q, A, R, H, S, T, V, M, D, and K. In some embodiments, the second amino acid residue is selected from E, P, L, Q, A, R, and H. In some embodiments, the peptide comprises a last amino acid residue selected from V, L, F, A, I, Y, M, W, P, and R. In some embodiments, the last amino acid residue is selected from V, L, and F.

In some embodiments, the peptide comprises a second amino acid residue selected from A, Y, S, T, V, I, L, F, Q, R, N, and W. In some embodiments, the second amino acid residue is selected from A and Y. In some embodiments, the peptide comprises a last amino acid residue selected from L, V, M, F, Y, and I. In some embodiments, the last amino acid residue is L.

### Disulfide Staple Pairs

In some embodiments, the construct may comprise one or more disulfide staple pairs. A disulfide staple pair may comprise two cysteine residues that are configured to form a disulfide bond under suitable conditions (e.g., oxidizing conditions, such as in a cellular compartment). The two cysteine residues may be located at any suitable position on the construct. In some embodiments, the disulfide staple pair is distributed across two parts of the construct, such as across the targeting moiety (e.g., peptide) and the MHC region (e.g., HLA class I heavy chain) or across the peptide and a linker, or across the peptide and the HLA class I heavy chain. In some embodiments, the disulfide staple pair is wholly contained within one region of the construct, such as wholly within a linker, wholly within the HLA class I heavy chain, or wholly within the peptide. In some embodiments, the one or more disulfide staple pairs are introduced to the construct by engineering (e.g., mutated, such as by site-directed mutagenesis). Alternatively or additionally, the one or more disulfide staple pairs are wholly or partially present in a wild type or unmutated sequence.

The disulfide staple pairs may be configured to impart a certain secondary, tertiary, or quaternary structure when the construct sequence is arranged in three-dimensional space, such as when expressed in a host cell. The secondary, tertiary, or quaternary structure may be determined from experimental structural biology data (e.g., X-ray crystallographic data, cryogenic electron microscopy data, nuclear magnetic resonance data), biochemical data (e.g., mass spectrometry data, chromatographic data, electrophoretic data), or computer simulation or modeling data (e.g., molecular dynamics simulations, de novo or ab initio prediction, homology modeling, fragment assembly, secondary structure prediction). Alternatively or additionally, the one or more disulfide staple pairs may be configured to impart a certain functional consequence on the expressed construct. In some embodiments, the one or more disulfide staple pairs enhances expression (e.g., cell surface expression) of the contrast, enhance stability of the construct, prevent exchange of the peptide, or some combination thereof. In some embodiments, the one or more disulfide staple pairs enhance expression of the construct. In some embodiments, the one or more disulfide staple pairs enhance stability of the construct. In some embodiments, the one or more disulfide staple pairs prevent exchange of the peptide.

In some embodiments, the disulfide staple pair may be configured to increase expression in a host cell (e.g., relative to a wild-type or other construct lacking the disulfide staple pair). In some embodiments, the expression is cell-surface expression. The expression may be measured by, for example, flow cytometry, fluorescence microscopy, mass spectrometry, or any other suitable quantification method. In some embodiments, the presence of a disulfide staple pair increases expression of the construct in a host cell relative to a reference (e.g., wild-type) construct. In some embodiments, expression is increased by at least about 1%, about 5%, about 10%, about 20%, about 30%, about 40%, about 50%, about 60%, about 70%, about 80%, about 90%, or about 100%, or more. In some embodiments, expression is increased by about 1% to about 100%. In some embodiments, expression is increased by about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, expression is increased by about 1%, about 5%, about 10%, about 20%, about 30%, about 40%, about 50%, about 60%, about 70%, about 80%, about 90%, or about 100%. In some embodiments, expression is increased by at least about 1%, about 5%, about 10%, about 20%, about 30%, about 40%, about 50%, about 60%, about 70%, about 80%, or about 90%. In some embodiments, expression is increased by at most about 5%, about 10%, about 20%, about 30%, about 40%, about 50%, about 60%, about 70%, about 80%, about 90%, or about 100%.

In some embodiments, the disulfide staple pair may be configured to increase stability of the construct (e.g., as compared to a construct not comprising the disulfide staple pair, such as a wild-type construct). The presence of the disulfide staple pair may increase stability by allowing for the formation of an additional disulfide bond to help the construct retain a particular secondary, tertiary, and/or quaternary structure even in the presence of forces or conditions (e.g., chaotropic agents, increased temperature) that tend to unfold proteins. The stability of a construct may be measured by differential scanning calorimetry (DSC), pulse-chase assays (such as bleach-chase and cycloheximide-chase assays), thermal shift assays, circular dichroism (CD) spectroscopy, UV-vis spectroscopy, nuclear magnetic resonance (NMR), gel filtration, isothermal calorimetry, light scattering, or any other suitable assay or instrument. In some embodiments the stability of a construct is expressed in relative terms (e.g., percent or fold change in stability with respect to a reference, such as a wild-type construct). In some embodiments, the stability of a construct is expressed in absolute terms (e.g., in terms of thermodynamic coordinates such as melting or other phase transition temperatures or a free energy of folding).

In some embodiments, the presence of a disulfide staple pair increases the stability of a construct at least about 1%, about 5%, about 10%, about 20%, about 30%, about 40%, about 50%, about 60%, about 70%, about 80%, about 90%, or about 100%, or more, relative to a reference (e.g., construct not comprising the disulfide pair, such as a wild-type construct). In some embodiments, stability is increased by about 1% to about 100%. In some embodiments, stability is increased by about 1% to about 5%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 100%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 100%, about 10% to about 20%, about 10% to about 30%, about 10% to about 40%, about 10% to about 50%, about 10% to about 60%, about 10% to about 70%, about 10% to about 80%, about 10% to about 90%, about 10% to about 100%, about 20% to about 30%, about 20% to about 40%, about 20% to about 50%, about 20% to about 60%, about 20% to about 70%, about 20% to about 80%, about 20% to about 90%, about 20% to about 100%, about 30% to about 40%, about 30% to about 50%, about 30% to about 60%, about 30% to about 70%, about 30% to about 80%, about 30% to about 90%, about 30% to about 100%, about 40% to about 50%, about 40% to about 60%, about 40% to about 70%, about 40% to about 80%, about 40% to about 90%, about 40% to about 100%, about 50% to about 60%, about 50% to about 70%, about 50% to about 80%, about 50% to about 90%, about 50% to about 100%, about 60% to about 70%, about 60% to about 80%, about 60% to about 90%, about 60% to about 100%, about 70% to about 80%, about 70% to about 90%, about 70% to about 100%, about 80% to about 90%, about 80% to about 100%, or about 90% to about 100%. In some embodiments, stability is increased by about 1%, about 5%, about 10%, about 20%, about 30%, about 40%, about 50%, about 60%, about 70%, about 80%, about 90%, or about 100%. In some embodiments, stability is increased by at least about 1%, about 5%, about 10%, about 20%, about 30%, about 40%, about 50%, about 60%, about 70%, about 80%, or about 90%. In some embodiments, stability is increased by at most about 5%, about 10%, about 20%, about 30%, about 40%, about 50%, about 60%, about 70%, about 80%, about 90%, or about 100%.

In some embodiments, the presence of a disulfide staple pair increases the stability of a construct by about 2-fold, about 4-fold, about 5-fold, about 6-fold, about 7-fold, about 8-fold, about 9-fold, about 10-fold, about 50-fold, about 100-fold, about 1000, or more, relative to a refence (e.g., construct not comprising the disulfide staple pair, such as a wild-type construct). In some embodiments, stability is increased by about 1-fold to about 1,000-fold. In some embodiments, stability is increased by about 1-fold to about 2-fold, about 1-fold to about 3-fold, about 1-fold to about 4-fold, about 1-fold to about 5-fold, about 1-fold to about 10-fold, about 1-fold to about 100-fold, about 1-fold to about 1,000-fold, about 2-fold to about 3-fold, about 2-fold to about 4-fold, about 2-fold to about 5-fold, about 2-fold to about 10-fold, about 2-fold to about 100-fold, about 2-fold to about 1,000-fold, about 3-fold to about 4-fold, about 3-fold to about 5-fold, about 3-fold to about 10-fold, about 3-fold to about 100-fold, about 3-fold to about 1,000-fold, about 4-fold to about 5-fold, about 4-fold to about 10-fold, about 4-fold to about 100-fold, about 4-fold to about 1,000-fold, about 5-fold to about 10-fold, about 5-fold to about 100-fold, about 5-fold to about 1,000-fold, about 10-fold to about 100-fold, about 10-fold to about 1,000-fold, or about 100-fold to about 1,000-fold. In some embodiments, stability is increased by about 1-fold, about 2-fold, about 3-fold, about 4-fold, about 5-fold, about 10-fold, about 100-fold, or about 1,000-fold. In some embodiments, stability is increased by at least about 1-fold, about 2-fold, about 3-fold, about 4-fold, about 5-fold, about 10-fold, or about 100-fold. In some embodiments, stability is increased by at most about 2-fold, about 3-fold, about 4-fold, about 5-fold, about 10-fold, about 100-fold, or about 1,000-fold.

In some embodiments, a construct as described herein comprising a disulfide staple pair may show a higher melting temperature (Tₘ) (e.g., as determined by a thermodynamic technique, such as DSC) relative to a reference (e.g., a construct that does not comprise the disulfide staple pair, such as a wild-type construct). In some embodiments, the Tₘ is increased by about 1° C., about 2° C., about 3° C., about 4° C., about 5° C., about 6° C., about 7° C., about 8° C., about 9° C., about 10° C., about 20° C., about 30° C., or more, relative to the reference. In some embodiments, the Tₘ is increased by about 1° C. to about 30° C. In some embodiments, the Tₘ is increased by about 1° C. to about 2° C., about 1° C. to about 3° C., about 1° C. to about 4° C., about 1° C. to about 5° C., about 1° C. to about 6° C., about 1° C. to about 7° C., about 1° C. to about 8° C., about 1° C. to about 9° C., about 1° C. to about 10° C., about 1° C. to about 20° C., about 1° C. to about 30° C., about 2° C. to about 3° C., about 2° C. to about 4° C., about 2° C. to about 5° C., about 2° C. to about 6° C., about 2° C. to about 7° C., about 2° C. to about 8° C., about 2° C. to about 9° C., about 2° C. to about 10° C., about 2° C. to about 20° C., about 2° C. to about 30° C., about 3° C. to about 4° C., about 3° C. to about 5° C., about 3° C. to about 6° C., about 3° C. to about 7° C., about 3° C. to about 8° C., about 3° C. to about 9° C., about 3° C. to about 10° C., about 3° C. to about 20° C., about 3° C. to about 30° C., about 4° C. to about 5° C., about 4° C. to about 6° C., about 4° C. to about 7° C., about 4° C. to about 8° C., about 4° C. to about 9° C., about 4° C. to about 10° C., about 4° C. to about 20° C., about 4° C. to about 30° C., about 5° C. to about 6° C., about 5° C. to about 7° C., about 5° C. to about 8° C., about 5° C. to about 9° C., about 5° C. to about 10° C., about 5° C. to about 20° C., about 5° C. to about 30° C., about 6° C. to about 7° C., about 6° C. to about 8° C., about 6° C. to about 9° C., about 6° C. to about 10° C., about 6° C. to about 20° C., about 6° C. to about 30° C., about 7° C. to about 8° C., about 7° C. to about 9° C., about 7° C. to about 10° C., about 7° C. to about 20° C., about 7° C. to about 30° C., about 8° C. to about 9° C., about 8° C. to about 10° C., about 8° C. to about 20° C., about 8° C. to about 30° C., about 9° C. to about 10° C., about 9° C. to about 20° C., about 9° C. to about 30° C., about 10° C. to about 20° C., about 10° C. to about 30° C., or about 20° C. to about 30° C. In some embodiments, the Tₘ is increased by about 1° C., about 2° C., about 3° C., about 4° C., about 5° C., about 6° C., about 7° C., about 8° C., about 9° C., about 10° C., about 20° C., or about 30° C. In some embodiments, the Tₘ is increased by at least about 1° C., about 2° C., about 3° C., about 4° C., about 5° C., about 6° C., about 7° C., about 8° C., about 9° C., about 10° C., or about 20° C. In some embodiments, the Tₘ is increased by at most about 2° C., about 3° C., about 4° C., about 5° C., about 6° C., about 7° C., about 8° C., about 9° C., about 10° C., about 20° C., or about 30° C.

In some embodiments, the construct may not comprise one or more disulfide staple pairs. In some embodiments, the construct may be engineered (e.g., mutated, such as by site-directed mutagenesis) to ablate one or more disulfide staple pairs. Such ablation may be performed to reduce the likelihood of alternative or undesired arrangements of construct component from forming under certain conditions (e.g., when expressed in a host cell).

In some embodiments, disulfide staple pairs are introduced by mutation of one or more residues to cysteine. In some embodiments, the mutation is in a targeting moiety (e.g., peptide) as described herein. In some embodiments, the mutation is in an HLA class I heavy chain as described herein. In some embodiments, the disulfide staple pair comprises a residue corresponding to the Y84 residue of an HLA-C (e.g., SEQ ID NO: 194). In some embodiments, the disulfide staple pair comprises a residue corresponding to the R69 residue of an HLA-C. In some embodiments, the disulfide staple pair comprises a residue corresponding to the A150 residue of an HLA-C. In some embodiments, the disulfide staple pair comprises a residue corresponding to the A73 residue of an HLA-C. In some embodiments, the disulfide staple pair comprises a cysteine in any one of positions 1-9 of a targeting moiety (e.g., peptide) as described herein. In some embodiments, the disulfide staple pair comprises a C5 residue of a peptide. In some embodiments, the disulfide staple pair comprises a C7 residue of a peptide. In some embodiments, the disulfide staple pair comprises a C8 residue of a peptide. In some embodiments, the disulfide staple pair comprises a cysteine in a peptide as listed in Table 2. In some embodiments, the disulfide staple pair comprises a cysteine in a linker as listed in Table 3. In some embodiments, the disulfide staple pair comprises a residue corresponding to the Y84 residue of an HLA-C and a residue in a linker region. In some embodiments, the disulfide staple pair comprises a residue corresponding to the R69 residue of an HLA-C and a residue in a targeting moiety (e.g., peptide). In some embodiments, the disulfide staple pair comprises a residue corresponding to the R69 residue of an HLA-C and a residue in a linker region. In some embodiments, the disulfide staple pair comprises a residue corresponding to the A150 residue of an HLA-C and a targeting moiety (e.g., peptide). In some embodiments, the disulfide staple pair comprises a residue corresponding to the A150 residue of an HLA-C and a residue in a linker region. In some embodiments, the disulfide staple pair comprises a residue corresponding to the A73 residue of an HLA-C and a targeting moiety (e.g., peptide). In some embodiments, the disulfide staple pair comprises a residue corresponding to the A73 residue of an HLA-C and a residue in a linker region.

### N- and C-Terminal Additions

In some embodiments, constructs as described herein comprise one or more N- or C-terminal additions. The N- or C-terminal additions may be added, for example, for purposes of purification or targeting to a particular cellular component (e.g., nucleus). In some embodiments, the construct comprises an N-terminal nuclear localization signal (NLS). In some embodiments, the construct comprises a purification tag, such as a hexa-his tag. The purification tag may be located at the N-terminus or the C-terminus of the construct. In some embodiments, the N- or C-terminal addition comprises a Tobacco Etch Virus (TEV) protease cleavage site. In some embodiments, the tag comprises a peptide sequence comprising one part of a cognate binding pair. In some embodiments, the cognate binding pair comprise an avidin-biotin binding pair or a streptavidin-biotin binding pair. In such embodiments, the tag may comprise a sequence configured to be biotinylated such that the biotinylated construct may be bound to a streptavidin or avidin moiety. In some embodiments, the N- or C-terminal addition comprises a furin cleavage site. In some embodiments, the N- or C-terminal addition comprises a 2A self-cleaving peptide (2A peptide). In some embodiments, the 2A peptide comprises a T2A peptide, P2A peptide, E2A peptide, or F2A peptide. In some embodiments, an SGSG linker is disposed between the 2A peptide and the rest of the construct (e.g., the HLA class 1 heavy chain domain).

### Nucleic Acid Molecules

Provided herein, in another aspect, is a nucleic acid molecule encoding constructs as provided herein.

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion comprises a deletion in the endogenous HLA-A locus. In some embodiments, the deletion comprises a deletion in the endogenous HLA-B locus. In some embodiments, the deletion comprises a deletion in the endogenous HLA-C locus. In some embodiments, the deletion comprises a deletion in the endogenous HLA-A locus and the HLA-B locus. In some embodiments, the deletion comprises a deletion in the endogenous HLA-A locus and the HLA-C locus. In some embodiments, the deletion comprises a deletion in the endogenous HLA-B locus and the HLA-C locus. In some embodiments, the deletion comprises a deletion in the endogenous HLA-A locus, the HLA-B locus, and the HLA-C locus. In some embodiments, the deletion is complete deletion of the endogenous HLA locus.

In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises an HLA-A sequence. In some embodiments, the sequence encoding a human HLA class 1 heavy chain sequence comprises an HLA-B sequence. In some embodiments, the sequence encoding a human HLA class 1 heavy chain sequence comprises an HLA-C sequence. In some embodiments, the sequence encoding a human HLA class 1 heavy chain sequence comprises an HLA-A sequence and an HLA-B sequence. In some embodiments, the sequence encoding a human HLA class 1 heavy chain sequence comprises an HLA-A sequence and an HLA-C sequence. In some embodiments, the sequence encoding a human HLA class 1 heavy chain sequence comprises an HLA-B sequence and an HLA-C sequence. In some embodiments, the sequence encoding a human HLA class 1 heavy chain sequence comprises an HLA-A sequence, an HLA-B sequence, and an HLA-C sequence.

In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises multiple alleles of an HLA-A sequence. In some embodiments, the sequence encoding a human HLA class 1 heavy chain sequence comprises multiple alleles of an HLA-B sequence. In some embodiments, the sequence encoding a human HLA class 1 heavy chain sequence comprises multiple alleles of an HLA-C sequence. In some embodiments, the sequence encoding a human HLA class 1 heavy chain sequence comprises multiple alleles of an HLA-A sequence and multiple alleles of an HLA-B sequence. In some embodiments, the sequence encoding a human HLA class 1 heavy chain sequence comprises multiple alleles of an HLA-A sequence and multiple alleles of an HLA-C sequence. In some embodiments, the sequence encoding a human HLA class 1 heavy chain sequence comprises multiple alleles of an HLA-B sequence and multiple alleles of an HLA-C sequence. In some embodiments, the sequence encoding a human HLA class 1 heavy chain sequence comprises multiple alleles of an HLA-A sequence, multiple alleles of an HLA-B sequence, and multiple alleles of an HLA-C sequence.

In some embodiments, the alleles of an HLA-A sequence are selected from HLA-A*02:01, HLA-A*01:01, HLA-A*03:01, HLA-A*11:01, HLA-A*24:02, HLA-A*29:02, HLA-A*26:01, HLA-A*32:01, HLA-A*23:01, HLA-A*68:02, HLA-A*30:01, HLA-A*30:02, HLA-A*34:02, HLA-A*31:01, HLA-A*33:03, HLA-A*02:07, HLA-A*02:06, and HLA-A*02:03.

In some embodiments, the alleles of an HLA-B sequence are selected from HLA-B*44:02, HLA-B*07:02, HLA-B*08:01, HLA-B*40:01, HLA-B*35:01, HLA-B*51:01, HLA-B*15:01, HLA-B*53:01, HLA-B*15:03, HLA-B*58:01, HLA-B*45:01, HLA-B*42:01, HLA-B*44:03, HLA-B*18:01, HLA-B*52:01, HLA-B*14:02, HLA-B*46:01, HLA-B*38:02, and HLA-B*15:02.

In some embodiments, the alleles of an HLA-C sequence are selected from HLA-C*07:01, HLA-C*07:02, HLA-C*04:01, HLA-C*05:01, HLA-C*03:04, HLA-C*06:02, HLA-C*03:03, HLA-C*12:03, HLA-C*08:02, HLA-C*02:02, HLA-C*16:01, HLA-C*17:01, HLA-C*01:02, HLA-C*02:01, HLA-C*08:01, HLA-C*03:02, and HLA-C*14:02.

In some embodiments, the sequence encoding a human HLA class 1 heavy chain sequence comprises an HLA-A sequence, wherein the HLA-A sequence is displaced between the HLA-B sequence and the HLA-C sequence.

In some embodiments, the sequence encoding a human HLA class 1 heavy chain sequence comprises fewer than 1700 base pairs (bp). In some embodiments, the sequence encoding a human HLA class 1 heavy chain sequence comprises fewer than 1600 base pairs (bp). In some embodiments, the sequence encoding a human HLA class 1 heavy chain sequence comprises fewer than 1500 base pairs (bp). In some embodiments, the sequence encoding a human HLA class 1 heavy chain sequence comprises fewer than 1400 base pairs (bp). In some embodiments, the sequence encoding a human HLA class 1 heavy chain sequence comprises fewer than 1300 base pairs (bp). In some embodiments, the sequence encoding a human HLA class 1 heavy chain sequence comprises fewer than 1200 base pairs (bp). In some embodiments, the sequence encoding a human HLA class 1 heavy chain sequence comprises fewer than 1100 base pairs (bp). In some embodiments, the sequence encoding a human HLA class 1 heavy chain sequence comprises fewer than 1000 base pairs (bp). In some embodiments, the sequence encoding a human HLA class 1 heavy chain sequence comprises fewer than 900 base pairs (bp). In some embodiments, the sequence encoding a human HLA class 1 heavy chain sequence comprises fewer than 800 base pairs (bp). In some embodiments, the sequence encoding a human HLA class 1 heavy chain sequence comprises fewer than 700 base pairs (bp). In some embodiments, the sequence encoding a human HLA class 1 heavy chain sequence comprises fewer than 600 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 450 bp. In some embodiments, the sequence encoding a human HLA class 1 heavy chain sequence comprises fewer than 400 bp. In some embodiments, the sequence encoding a human HLA class 1 heavy chain sequence comprises fewer than 350 bp. In some embodiments, the sequence encoding a human HLA class 1 heavy chain sequence comprises fewer than 300 bp. In some embodiments, the sequence encoding a human HLA class 1 heavy chain sequence comprises fewer than 250 bp. In some embodiments, the sequence encoding a human HLA class 1 heavy chain sequence comprises fewer than 200 bp. In some embodiments, the sequence encoding a human HLA class 1 heavy chain sequence comprises fewer than 150 bp. In some embodiments, the sequence encoding a human HLA class 1 heavy chain sequence comprises fewer than 100 bp. In some embodiments, the sequence encoding a human HLA class 1 heavy chain sequence comprises fewer than 50 bp.

In some embodiments, the HLA-A sequence, HLA-B sequence, HLA-C sequences, or combination thereof comprises one or more flanking sequences. In some embodiments, the HLA-A sequence comprises one or more flanking sequences. In some embodiments, the HLA-B sequence comprises one or more flanking sequences. In some embodiments, the HLA-C sequence comprises one or more flanking sequences. In some embodiments, the HLA-A sequence and the HLA-B sequence comprise one or more flanking sequences. In some embodiments, the HLA-A sequence and the HLA-C sequence comprise one or more flanking sequences. In some embodiments, the HLA-B sequence and the HLA-C sequence comprise one or more flanking sequences. In some embodiments, the HLA-A sequence, the HLA-B sequence, and the HLA-C comprise one or more flanking sequences.

In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof. In some embodiments, the HLA-A sequence comprises an endogenous HLA-A promoter. In some embodiments, the HLA-B sequence comprises an endogenous HLA-B promoter. In some embodiments, the HLA-C sequence comprises an endogenous HLA-C promoter. In some embodiments, the HLA-A sequence comprises an endogenous HLA-A promoter and the HLA-B sequence comprises an endogenous HLA-B promoter. In some embodiments, the HLA-A sequence comprises an endogenous HLA-A promoter and the HLA-C sequence comprises an endogenous HLA-C promoter. In some embodiments, the HLA-B sequence comprises an endogenous HLA-B promoter and the HLA-C sequence comprises an endogenous HLA-C promoter. In some embodiments, the HLA-A sequence comprises an endogenous HLA-A promoter, the HLA-B sequence comprises an endogenous HLA-B promoter, and the HLA-C sequence comprises an endogenous HLA-C promoter.

In some embodiments, the sequence encoding a human HLA class 1 heavy chain sequence does not comprise at least a portion of the HLA-A sequence, HLA-B sequence, HLA-C sequence, or combination thereof. In some embodiments, the sequence encoding a human HLA class 1 heavy chain sequence does not comprise at least a portion of the HLA-A sequence. In some embodiments, the sequence encoding a human HLA class 1 heavy chain sequence does not comprise at least a portion of the HLA-B sequence. In some embodiments, the sequence encoding a human HLA class 1 heavy chain sequence does not comprise at least a portion of the HLA-C sequence. In some embodiments, the sequence encoding a human HLA class 1 heavy chain sequence does not comprise at least a portion of the HLA-A sequence or the HLA-B sequence. In some embodiments, the sequence encoding a human HLA class 1 heavy chain sequence does not comprise at least a portion of the HLA-A sequence or the HLA-C sequence. In some embodiments, the sequence encoding a human HLA class 1 heavy chain sequence does not comprise at least a portion of the HLA-B sequence or the HLA-C sequence. In some embodiments, the sequence encoding a human HLA class 1 heavy chain sequence does not comprise at least a portion of the HLA-A sequence, the HLA-B sequence, or the HLA-C sequence.

In some embodiments, the nucleic acid molecule further comprises a sequence encoding a human beta-2 microglobulin (B2M) peptide. In some embodiments, the nucleic acid molecule further comprises a sequence encoding an endogenous human beta-2 microglobulin peptide.

In some embodiments, the nucleic acid molecule further comprises a sequence encoding a targeting moiety (e.g., a peptide). The peptide may comprise any sequence or feature disclosed herein.

In some embodiments, the nucleic acid molecule further comprises one or more sequences encoding one or more linkers between the sequence encoding the peptide and the sequence encoding the human HLA class 1 heavy chain sequence. In some embodiments, a sequence of the one or more sequences encoding one or more linkers is displaced between the sequence encoding the peptide and the sequence encoding the human beta-2 microglobulin peptide, between the sequence encoding the human beta-2 microglobulin peptide and the sequence encoding the human HLA class 1 heavy chain sequence, or both. In some embodiments, a sequence of the one or more sequences encoding one or more linkers is displaced between the sequence encoding the peptide and the sequence encoding the human beta-2 microglobulin peptide. In some embodiments, a sequence of the one or more sequences encoding one or more linkers is displaced between the sequence encoding the human beta-2 microglobulin peptide and the sequence encoding the human HLA class 1 heavy chain sequence. In some embodiments, a first sequence of the one or more sequences encoding one or more linkers is displaced between the sequence encoding the peptide and the sequence encoding the human beta-2 microglobulin peptide, and a second sequence of the one or more sequences encoding one or more linkers is displaced between the sequence encoding the human beta-2 microglobulin peptide and the sequence encoding the human HLA class 1 heavy chain sequence.

In some embodiments, the nucleic acid molecule further comprises a sequence encoding one or more immune checkpoint modulators. In some embodiments, the nucleic acid further comprises a sequence encoding CD8. In some embodiments, the nucleic acid molecule further comprises a sequence encoding CD47. In some embodiments, the nucleic acid molecule further comprises a sequence encoding PD-L1. In some embodiments, the nucleic acid molecule further comprises a sequence encoding A2AR. In some embodiments, the nucleic acid molecule further comprises a sequence encoding B7-H3. In some embodiments, the nucleic acid molecule further comprises a sequence encoding B7-H4. In some embodiments, the nucleic acid molecule further comprises a sequence encoding BTLA. In some embodiments, the nucleic acid molecule further comprises a sequence encoding CTLA-4. In some embodiments, the nucleic acid molecule further comprises a sequence encoding IDO. In some embodiments, the nucleic acid molecule further comprises a sequence encoding KIR. In some embodiments, the nucleic acid molecule further comprises a sequence encoding LAG3. In some embodiments, the nucleic acid molecule further comprises a sequence encoding NOX2. In some embodiments, the nucleic acid molecule further comprises a sequence encoding PD-1. In some embodiments, the nucleic acid molecule further comprises a sequence encoding TIM-3. In some embodiments, the nucleic acid molecule further comprises a sequence encoding VISTA. In some embodiments, the nucleic acid molecule further comprises a sequence encoding SIGLEC7.

In some embodiments, the nucleic acid molecule further comprises a sequence encoding one or more knocked out proteins corresponding to a receptor of the one or more immune checkpoint modulators. In some embodiments, the nucleic acid molecule further comprises a sequence encoding a knocked out CD47 receptor. In some embodiments, the nucleic acid molecule further comprises a sequence encoding a knocked-out PD-L1 receptor. In some embodiments, the nucleic acid molecule further comprises a sequence encoding a knocked out A2AR receptor. In some embodiments, the nucleic acid molecule further comprises a sequence encoding a knocked out B7-H3 receptor. In some embodiments, the nucleic acid molecule further comprises a sequence encoding a knocked out B7-H4 receptor. In some embodiments, the nucleic acid molecule further comprises a sequence encoding a knocked out BTLA receptor. In some embodiments, the nucleic acid molecule further comprises a sequence encoding a knocked out CTLA-4 receptor. In some embodiments, the nucleic acid molecule further comprises a sequence encoding a knocked-out IDO receptor. In some embodiments, the nucleic acid molecule further comprises a sequence encoding a knocked-out KIR receptor. In some embodiments, the nucleic acid molecule further comprises a sequence encoding a knocked out LAG3 receptor. In some embodiments, the nucleic acid molecule further comprises a sequence encoding a knocked out NOX2 receptor. In some embodiments, the nucleic acid molecule further comprises a sequence encoding a knocked-out PD-1 receptor. In some embodiments, the nucleic acid molecule further comprises a sequence encoding a knocked-out TIM-3 receptor. In some embodiments, the nucleic acid molecule further comprises a sequence encoding a knocked-out VISTA receptor. In some embodiments, the nucleic acid molecule further comprises a sequence encoding a knocked out SIGLEC7 receptor.

In some embodiments, the sequence encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, the sequence encoding the human HLA class 1 heavy chain sequence comprises an HLA-E sequence or a fragment thereof. In some embodiments, the sequence encoding the human HLA class 1 heavy chain sequence comprises an HLA-F sequence or a fragment thereof. In some embodiments, the sequence encoding the human HLA class 1 heavy chain sequence comprises an HLA-G sequence or a fragment thereof. In some embodiments, the sequence encoding the human HLA class 1 heavy chain sequence comprises an HLA-E sequence or a fragment thereof and an HLA-F sequence or a fragment thereof. In some embodiments, the sequence encoding the human HLA class 1 heavy chain sequence comprises an HLA-E sequence or a fragment thereof and an HLA-G sequence or a fragment thereof. In some embodiments, the sequence encoding the human HLA class 1 heavy chain sequence comprises an HLA-F sequence or a fragment thereof and an HLA-G sequence or a fragment thereof. In some embodiments, the sequence encoding the human HLA class 1 heavy chain sequence comprises an HLA-E sequence or a fragment thereof, an HLA-F sequence or a fragment thereof, and an HLA-G sequence or a fragment thereof.

In some embodiments, at least one of the HLA-E sequence or the fragment thereof, HLA-F sequence or the fragment thereof, HLA-G sequence or the fragment thereof, or any combination thereof is inhibited from eliciting a T cell response when the construct is interrogated by one or more T cells. In some embodiments, the HLA-E sequence or the fragment thereof is inhibited from inhibited from eliciting a T cell response when the construct is interrogated by one or more T cells. In some embodiments, the HLA-F sequence or the fragment thereof is inhibited from eliciting a T cell response when the construct is interrogated by one or more T cells. In some embodiments, the HLA-G sequence or the fragment thereof is inhibited from eliciting a T cell response when the construct is interrogated by one or more T cells. In some embodiments, the HLA-E sequence or the fragment thereof and the HLA-F sequence or the fragment thereof are inhibited from eliciting a T cell response when the construct is interrogated by one or more T cells. In some embodiments, the HLA-E sequence or the fragment thereof and the HLA-G sequence or the fragment thereof are inhibited from eliciting a T cell response when the construct is interrogated by one or more T cells. In some embodiments, the HLA-F sequence or the fragment thereof and the HLA-G sequence or the fragment thereof are inhibited from eliciting a T cell response when the construct is interrogated by one or more T cells. In some embodiments, the HLA-E sequence or the fragment thereof, the HLA-F sequence or the fragment thereof, and the HLA-G sequence or the fragment thereof are inhibited from eliciting a T cell response when the construct is interrogated by one or more T cells.

In some embodiments, the nucleic acid molecule further comprises a sequence encoding one or more knocked out proteins corresponding class II, major histocompatibility complex, transactivator (CIITA). In some embodiments, the entire class II, major histocompatibility complex, transactivator (CIITA) locus is knocked out.

In some embodiments, the nucleic acid molecule further comprises a sequence encoding a regulatory peptide. In some embodiments, the nucleic acid molecule further comprises a sequence encoding an apoptosis-inducing peptide. In some embodiments, the nucleic acid molecule further comprises a sequence encoding an apoptosis-inducing peptide to act as a “kill switch.”

In some embodiments, the nucleic acid molecule further comprises a sequence encoding an epitope configured to allow for detection of the construct. In some embodiments, the nucleic acid molecule further comprises a sequence encoding an epitope comprising 3,5-dinitrosalicylic acid.

In some embodiments, the nucleic acid molecule further comprises a sequence encoding one or more knocked out proteins. In some embodiments, the one or more knocked out proteins are selected from blood group A antigen and blood group B antigen. In some embodiments, the nucleic acid molecule further comprises a sequence encoding knocked out blood group A antigen. In some embodiments, the nucleic acid molecule further comprises a sequence encoding knocked out blood group B antigen.

In some embodiments, the nucleic acid molecule comprises,
a. the sequence encoding the peptide;
b. a first sequence encoding a first linker of the one or more sequences encoding one or more linkers;
c. the sequence encoding the human beta-2 microglobulin peptide;
d. a second sequence encoding a second linker of the one or more sequences encoding one or more linkers; and
e. the sequence encoding the human HLA class 1 heavy chain sequence.

Provided herein, in another aspect, is a nucleic acid molecule comprising a sequence encoding a construct comprising one or more Class 1 human leukocyte antigen (HLA) proteins, wherein the one or more Class 1 HLA proteins are inhibited from eliciting a T cell response when the construct is interrogated by one or more T cells, and wherein the nucleic acid molecule comprises,
a. a sequence encoding a peptide, wherein the peptide is incapable of activating the one or more T cells;
b. a first sequence encoding a first linker; and
c. a sequence encoding one or more Class 1 HLA proteins;
   wherein the first linker comprises a conformation configured to not block one or more killer-cell immunoglobulin-like receptor (KIR) binding sites on the human HLA class 1 heavy chain sequence, and wherein the conformation is further configured to resist proteolytic cleavage.

In some embodiments, the nucleic acid molecule further comprises a sequence encoding a human beta-2 microglobulin peptide between the sequence encoding the linker and the sequence encoding the human HLA class 1 heavy chain sequence. In some embodiments, the nucleic acid molecule further comprises a sequence encoding an endogenous human beta-2 microglobulin peptide between the sequence encoding the linker and the sequence encoding the human HLA class 1 heavy chain sequence.

In some embodiments, the sequence encoding a human HLA class 1 heavy chain sequence comprises one or more mutations, wherein the one or more mutations inhibit the human HLA class 1 heavy chain sequence from eliciting a T cell response when the human HLA class 1 heavy chain sequence is interrogated by one or more CD8 cells. In some embodiments, the one or more mutations comprises a mutation of one or more of amino acid residues 84, 115, 122, 128, 194, 197, 198, 212, 214, 222, 223, 224, 225, 226, 227, 228, 229, 230, 231, 232, 233, 243, 245, 248, 262, or any combination thereof. In some embodiments, the one or more mutations comprises a mutation of amino acid residue 84. In some embodiments, the one or more mutations comprises a mutation of amino acid residue 115. In some embodiments, the one or more mutations comprises a mutation of amino acid residue 122. In some embodiments, the one or more mutations comprises a mutation of amino acid residue 128. In some embodiments, the one or more mutations comprises a mutation of amino acid residue 194. In some embodiments, the one or more mutations comprises a mutation of amino acid residue 197. In some embodiments, the one or more mutations comprises a mutation of amino acid residue 198. In some embodiments, the one or more mutations comprises a mutation of amino acid residue 212. In some embodiments, the one or more mutations comprises a mutation of amino acid residue 214. In some embodiments, the one or more mutations comprises a mutation of amino acid residue 222. In some embodiments, the one or more mutations comprises a mutation of amino acid residue 223. In some embodiments, the one or more mutations comprises a mutation of amino acid residue 224. In some embodiments, the one or more mutations comprises a mutation of amino acid residue 225. In some embodiments, the one or more mutations comprises a mutation of amino acid residue 226. In some embodiments, the one or more mutations comprises a mutation of amino acid residue 227. In some embodiments, the one or more mutations comprises a mutation of amino acid residue 228. In some embodiments, the one or more mutations comprises a mutation of amino acid residue 229. In some embodiments, the one or more mutations comprises a mutation of amino acid residue 230. In some embodiments, the one or more mutations comprises a mutation of amino acid residue 231. In some embodiments, the one or more mutations comprises a mutation of amino acid residue 232. In some embodiments, the one or more mutations comprises a mutation of amino acid residue 233. In some embodiments, the one or more mutations comprises a mutation of amino acid residue 243. In some embodiments, the one or more mutations comprises a mutation of amino acid residue 245. In some embodiments, the one or more mutations comprises a mutation of amino acid residue 248. In some embodiments, the one or more mutations comprises a mutation of amino acid residue 262.

In some embodiments, the nucleic acid molecule further comprises a sequence encoding one or more proteins or fragments thereof that inhibit an immune response by the complement system. In some embodiments, the one or more proteins or fragments thereof are selected from CD48, CD59, or a combination thereof. In some embodiments, the one or more proteins or fragments thereof is CD48. In some embodiments, the one or more proteins or fragments thereof is CD59. In some embodiments, the one or more proteins or fragments thereof are CD48 and CD59.

Provided herein, in another aspect, is a method for generating the nucleic acid molecule provided herein, comprising displacing a sequence encoding a region configured to receive a sequence comprising the deletion in the HLA locus, a sequence encoding the human HLA class 1 heavy chain sequence, or any combination thereof. In some embodiments, the method comprises displacing a sequence encoding a region configured to receive a sequence comprising the deletion in the HLA locus. In some embodiments, the method comprises displacing a sequence encoding a region configured to receive a sequence encoding the human HLA class 1 heavy chain sequence. In some embodiments, the method comprises displacing a sequence encoding a region configured to receive a sequence comprising the deletion in the HLA locus and a sequence encoding the human HLA class 1 heavy chain sequence.

### Immune Incompetent Cells

Provided herein, in another aspect, is a method of making an immune incompetent cell, comprising administering the construct provided herein or the nucleic acid molecule provided herein to a cell.

In some embodiments, the nucleic acid molecule is delivered to the cell's genome. In some embodiments, the cell is incubated with the construct.

In some embodiments, the cell is a stem cell. In some embodiments, the stem cell is an Induced Pluripotent stem cell (iPSC). In some embodiments, the stem cell is an embryonic stem cell (ESC). In some embodiments, the stem cell is a mesenchymal stem cell (MSC). In some embodiments, the stem cell is a hematopoietic stem cell (HSC). In some embodiments, the cell is a chimeric antigen receptor (CAR) T cell. In some embodiments, the cell is a chimeric antigen receptor macrophage (CAR-M) cell. In some embodiments, the cell is a chimeric antigen receptor natural killer (CAR-NK) cell.

In some embodiments, the immune incompetent cells are suitable for use in cellular therapy. In some embodiments, the immune incompetent cells are suitable for administration to a subject without causing an immune response.

### Other Methods of Gene Therapy/Writing

### Vectors and Nucleic Acids

A variety of nucleic acids may be introduced into cells, for knockout purposes, or to obtain expression of a gene for other purposes. Nucleic acid constructs that can be used to produce transgenic cells including a target nucleic acid sequence. As used herein, the term nucleic acid or “nucleic acid molecule” includes DNA, RNA, and nucleic acid analogs, and nucleic acids that are double-stranded or single-stranded (i.e., a sense or an antisense single strand). Nucleic acid analogs can be modified at the base moiety, sugar moiety, or phosphate backbone to improve, for example, stability, hybridization, or solubility of the nucleic acid. Modifications at the base moiety include deoxyuridine for deoxythymidine, and 5-methyl-2′-deoxycytidine and 5-bromo-2′-doxycytidine for deoxycytidine. Modifications of the sugar moiety include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six membered, morpholino ring, or peptide nucleic acids, in which the deoxyphosphate backbone is replaced by a pseudopeptide backbone and the four bases are retained. See, Summerton and Weller (1997) Antisense Nucleic Acid Drug Dev. 7 (3): 187; and Hyrup et al. (1996) Bioorgan. Med. Chem. 4:5. In addition, the deoxyphosphate backbone can be replaced with, for example, a phosphorothioate or phosphorodithioate backbone, a phosphoroamidite, or an alkyl phosphotriester backbone.

The target nucleic acid sequence can be operably linked to a regulatory region such as a promoter. Regulatory regions can be from any species. As used herein, operably linked refers to positioning of a regulatory region relative to a nucleic acid sequence in such a way as to permit or facilitate transcription of the target nucleic acid.

Any type of promoter can be operably linked to a target nucleic acid sequence. Examples of promoters include, without limitation, tissue-specific promoters, constitutive promoters, and promoters responsive or unresponsive to a particular stimulus. Suitable tissue specific promoters can result in preferential expression of a nucleic acid transcript in beta cells and include, for example, the human insulin promoter. Other tissue specific promoters can result in preferential expression in, for example, hepatocytes or heart tissue and can include the albumin or alpha-myosin heavy chain promoters, respectively. In other embodiments, a promoter that facilitates the expression of a nucleic acid molecule without significant tissue- or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther. 12:563; and Kiwaki et al. (1996) Hum. Gene Ther. 7:821.

An example of an inducible promoter is the tetracycline (tet)-on promoter system, which can be used to regulate transcription of the nucleic acid. In this system, a mutated Tet repressor (TetR) is fused to the activation domain of herpes simplex virus VP 16 trans-activator protein to create a tetracycline-controlled transcriptional activator ((TA), which is regulated by tet or doxycycline (dox). In the absence of antibiotic, transcription is minimal, while in the presence of tet or dox, transcription is induced. Alternative inducible systems include the ecdysone or rapamycin systems. Ecdysone is an insect molting hormone whose production is controlled by a heterodimer of the ecdysone receptor and the product of the ultraspiracle gene (USP).

Expression is induced by treatment with ecdysone or an analog of ecdysone such as muristerone A. The agent that is administered to the subject to trigger the inducible system is referred to as an induction agent.

Additional regulatory regions that may be useful in nucleic acid constructs, include, but are not limited to, polyadenylation sequences, translation control sequences (e.g., an internal ribosome entry segment, IRES), enhancers, inducible elements, or introns. Such regulatory regions may not be necessary, although they may increase expression by affecting transcription, stability of the mRNA, translational efficiency, or the like. Such regulatory regions can be included in a nucleic acid construct as desired to obtain optimal expression of the nucleic acids in the cell(s). Sufficient expression, however, can sometimes be obtained without such additional elements.

A nucleic acid construct may be used that encodes signal peptides or selectable markers. Signal peptides can be used such that an encoded polypeptide is directed to a particular cellular location (e.g., the cell surface). Non-limiting examples of selectable markers include puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture. Other selectable markers include fluorescent polypeptides, such as green fluorescent protein or yellow fluorescent protein.

In some embodiments, a sequence encoding a selectable marker can be flanked by recognition sequences for a recombinase such as, e.g., Cre or Flp. For example, the selectable marker can be flanked by loxP recognition sites (34-bp recognition sites recognized by the Cre recombinase) or FRT recognition sites such that the selectable marker can be excised from the construct. See, Orban, et al., Proc. Natl. Acad. Sci. (1992) 89:6861, for a review of Cre/lox technology, and Brand and Dymecki, Dev. Cell (2004) 6:7. A transposon containing a Cre- or Flp-activatable transgene interrupted by a selectable marker gene also can be used to obtain transgenic cells with conditional expression of a transgene.

In some embodiments, the target nucleic acid encodes a polypeptide. A nucleic acid sequence encoding a polypeptide can include a tag sequence that encodes a “tag” designed to facilitate subsequent manipulation of the encoded polypeptide (e.g., to facilitate localization or detection). Tag sequences can be inserted in the nucleic acid sequence encoding the polypeptide such that the encoded tag is located at either the carboxyl or amino terminus of the polypeptide. Non-limiting examples of encoded tags include glutathione S transferase (GST) and FLAG™ tag (Kodak, New Haven, Conn.).

In other embodiments, the target nucleic acid sequence induces RNA interference against a target nucleic acid such that expression of the target nucleic acid is reduced. For example, the target nucleic acid sequence can induce RNA interference against a nucleic acid encoding a cystic fibrosis transmembrane conductance regulatory (CFTR) polypeptide. For example, double-stranded small interfering RNA (siRNA) or short hairpin RNA (shRNA) homologous to a CFTR DNA can be used to reduce expression of that DNA. Constructs for siRNA can be produced as described, for example, in Fire et al. (1998) Nature 391:806; Romano and Masino (1992) Mol. Microbiol. 6:3343; Cogoni et al. (1996) EMBO J. 15:3153; Cogoni and Masino (1999) Nature 399:166; Misquitta and Paterson (1999) Proc. Natl. Acad. Sci. USA 96:1451; and Kennerdell and Carthew (1998) Cell 95:1017. Constructs for shRNA can be produced as described by McIntyre and Fanning (2006) BMC Biotechnology 6:1. In general, shRNAs are transcribed as a single-stranded RNA molecule containing complementary regions, which can anneal and form short hairpins.

Nucleic acid constructs can be introduced into embryonic, fetal, or adult cells of any type, including, for example, germ cells such as an oocyte or an egg, a progenitor cell, an adult or embryonic stem cell, a hematopoietic stem cell, a mesenchymal stem cell, a primordial germ cell, a kidney cell such as a PK-15 cell, an islet cell, a beta cell, a liver cell, or a fibroblast such as a dermal fibroblast, using a variety of techniques. Non-limiting examples of techniques include the use of transposon systems, recombinant viruses that can infect cells, or liposomes or other non-viral methods such as electroporation, microinjection, or calcium phosphate precipitation, that are capable of delivering nucleic acids to cells.

In transposon systems, the transcriptional unit of a nucleic acid construct, i.e., the regulatory region operably linked to a target nucleic acid sequence, is flanked by an inverted repeat of a transposon. Several transposon systems, including, for example, Sleeping Beauty (see, U.S. Pat. No. 6,613,752 and U.S. Publication No. 2005/0003542); Frog Prince (Miskey et al. (2003) Nucleic Acids Res. 31:6873); Tol2 (Kawakami (2007) Genome Biology 8(Suppl. 1):S7; Minos (Pavlopoulos et al. (2007) Genome Biology 8(Suppl. 1):S2); Hsmarl (Miskey et al. (2007)) Mol Cell Biol. 27:4589); and Passport have been developed to introduce nucleic acids into cells. The Sleeping Beauty and Passport transposon is particularly useful. A transposase can be delivered as a protein, encoded on the same nucleic acid construct as the target nucleic acid, can be introduced on a separate nucleic acid construct, or provided as an mRNA (e.g., an in vitro-transcribed and capped mRNA).

Insulator elements also can be included in a nucleic acid construct to maintain expression of the target nucleic acid and to inhibit the unwanted transcription of host genes. See, for example, U.S. Publication No. 2004/0203158. Typically, an insulator element flanks each side of the transcriptional unit and is internal to the inverted repeat of the transposon. Non-limiting examples of insulator elements include the matrix attachment region-(MAR) type insulator elements and border-type insulator elements. See, for example, U.S. Pat. Nos. 6,395,549, 5,731,178, 6,100,448 and 5,610,053, and U.S. Publication No. 2004/0203158.

Nucleic acids can be incorporated into vectors. A vector is a broad term that includes any specific DNA segment that is designed to move from a carrier into a target DNA. A vector may be referred to as an expression vector, or a vector system, which is a set of components needed to bring about DNA insertion into a genome or other targeted DNA sequence such as an episome, plasmid, or even virus/phage DNA segment. Vector systems such as viral vectors (e.g., retroviruses, adeno-associated virus and integrating phage viruses), and non-viral vectors (e.g., transposons) used for gene delivery in subjects have two basic components: 1) a vector comprised of DNA (or RNA that is reverse transcribed into a cDNA) and 2) a transposase, recombinase, or other integrase enzyme that recognizes both the vector and a DNA target sequence and inserts the vector into the target DNA sequence. Vectors most often contain one or more expression cassettes that comprise one or more expression control sequences, wherein an expression control sequence is a DNA sequence that controls and regulates the transcription and/or translation of another DNA sequence or mRNA, respectively.

Many different types of vectors are known. For example, plasmids and viral vectors, e.g., retroviral vectors, are known. Mammalian expression plasmids typically have an origin of replication, a suitable promoter and optional enhancer, and also any necessary ribosome binding sites, a polyadenylation site, splice donor and acceptor sites, transcriptional termination sequences, and 5′ flanking non-transcribed sequences. Examples of vectors include: plasmids (which may also be a carrier of another type of vector), adenovirus, adeno-associated virus (AAV), lentivirus (e.g., HIV-1, SIV or FIV), retrovirus (e.g., ASV, ALV or MoMLV), herpes simplex virus (HSV), and transposons (e.g., Sleeping Beauty, P-elements, Tol-2, Frog Prince, piggy Bac).

Provided herein, in other aspects, are additional methods of delivering a nucleic acid molecule encoding one or more human leukocyte antigens (HLAs) to a cell. In some embodiments, the method comprises delivery of a nucleic acid molecule encoding one or more HLAs via a viral vector. In some embodiments, the method comprises delivery of a nucleic acid molecule encoding one or more HLAs via a non-viral vector. In some embodiments, the viral vector is derived from a lentivirus.

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion is complete deletion of the endogenous HLA locus.

In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises an HLA-A sequence, wherein the HLA-A sequence is displaced between the HLA-B sequence and the HLA-C sequence.

In some embodiments, the sequence encoding a human HLA class 1 heavy chain sequence comprises fewer than 1700 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 250 bp. In some embodiments, the sequence encoding a human HLA class 1 heavy chain sequence comprises fewer than 150 bp.

In some embodiments, the HLA-A sequence, HLA-B sequence, HLA-C sequences, or combination thereof comprises one or more flanking sequences. In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof.

In some embodiments, the sequence encoding a human HLA class 1 heavy chain sequence does not comprise at least a portion of the HLA-A sequence, HLA-B sequence, HLA-C sequence, or combination thereof.

In some embodiments, the nucleic acid molecule encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, at least one of the HLA-E sequence or the fragment thereof, HLA-F sequence or the fragment thereof, HLA-G sequence or the fragment thereof, or any combination thereof is inhibited from eliciting a T cell response when the construct is interrogated by one or more T cells.

In some embodiments, the nucleic acid molecule encoding a human HLA class 1 heavy chain sequence comprises one or more mutations, wherein a cell comprising the mutated human HLA class 1 heavy chain sequence comprising the one or more mutations does not elicit an immune response when the cell is interrogated by one or more CD8 cells. In some embodiments, the mutated human HLA class 1 heavy chain sequence encodes an HLA comprising one or more mutations at one or more of amino acid residues 115, 122, 128, 194, 197, 198, 212, 214, 222, 223, 224, 225, 226, 227, 228, 229, 230, 231, 232, 233, 243, 245, 248, 262, or any combination thereof.

### Templated and Non-Templated Repairs

Targeted endonuclease technologies, such as zinc finger nucleases (ZFNs), TAL effector nucleases (TALENs) and clustered regularly interspaced short palindromic repeats/CRISPR associated endonuclease cas9 (CRISPR/Cas9) can be utilized to disrupt gene function by introducing insertions and/or deletions (indels) into genomes of species, such as by non-homologous end-joining (NHEJ). However, indels introduced by NHEJ are variable in size and sequence which makes screening for functionally disrupted clones arduous and does not enable precise alterations. TALEN or CRISPR/Cas9 mediated homology-directed repair (HDR) supports the introduction of defined nucleotide changes in eukaryotic cells.

A subject may be modified using TALENs, zinc finger nucleases, or other genetic engineering tools, including various vectors that are known. A genetic modification made by such tools may comprise inactivation of a gene. The term inactivation of a gene refers to preventing the formation of a functional gene product. A gene product is functional only if it fulfills its normal (wild-type) functions. Materials and methods of genetically modifying subjects are further detailed in U.S. Ser. No. 13/404,662 filed Feb. 24, 2012, Ser. No. 13/467,588 filed May 9, 2012, and Ser. No. 12/622,886 filed Nov. 10, 2009 which are hereby incorporated herein by reference for all purposes; in case of conflict, the instant specification is controlling. The term trans-acting refers to processes acting on a target gene from a different molecule (i.e., intermolecular). A trans-acting element is usually a DNA sequence that contains a gene. This gene codes for a protein (or microRNA or other diffusible molecule) that is used in the regulation of the target gene. The trans-acting gene may be on the same chromosome as the target gene, but the activity is via the intermediary protein or RNA that it encodes. Inactivation of a gene using a dominant negative generally involves a trans-acting element. The term cis-regulatory or cis-acting means an action without coding for protein or RNA; in the context of gene inactivation, this generally means inactivation of the coding portion of a gene, or a promoter and/or operator that is necessary for expression of the functional gene.

Various techniques known in the art can be used to introduce nucleic acid constructs into non-humans and humans to produce founder lines, in which the nucleic acid construct is integrated into the genome. Such techniques include, without limitation, pronuclear microinjection (U.S. Pat. No. 4,873,191), retrovirus mediated gene transfer into germ lines (Van der Putten et al. (1985) Proc. Natl. Acad. Sci. USA 82, 6148-1652), gene targeting into embryonic stem cells (Thompson et al. (1989) Cell 56, 313-321), electroporation of embryos (Lo (1983) Mol. Cell. Biol. 3, 1803-1814), sperm-mediated gene transfer (Lavitrano et al. (2002) Proc. Natl. Acad. Sci. USA 99, 14230-14235; Lavitrano et al. (2006) Reprod. Fert. Develop. 18, 19-23), and in vitro transformation of somatic cells, such as cumulus or mammary cells, or adult, fetal, or embryonic stem cells, followed by nuclear transplantation (Wilmut et al. (1997) Nature 385, 810-813; and Wakayama et al. (1998) Nature 394, 369-374). Pronuclear microinjection, sperm mediated gene transfer, and somatic cell nuclear transfer are particularly useful techniques, as well as cytoplasmic injection, primordial germ cell transplantation (Brinster), and blastocyst chimera production whereby a germ cell is propagated in an embryo.

TALENs, zinc finger nucleases, CRISPR nuclease (e.g., CRISPR/Cas9) and recombinase fusion proteins may be used with or without a template. A template is an exogenous DNA added to the cell for cellular repair machinery to use as a guide (template) to repair double stranded breaks (DSB) in DNA. This process is generally referred to as homology directed repair (HDR). Processes without a template involve making DSBs and providing for cellular machinery to make repairs that are often less than perfect, so that an insertion or deletion (an indel) is made. The cellular pathway referred to as non-homologous end joining (NHEJ) typically mediates non-templated repairs of DSBs. The term NHEJ is commonly used to refer to all such non-templated repairs regardless of whether the NHEJ was involved, or an alternative cellular pathway.

### Targeted Nuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a RGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, embodiments for making indels with precision at an intended residue and placement of the allele that is being introgressed at the DNA-binding site.

### Zinc Finger Nucleases (ZFNs)

Zinc-finger nucleases (ZFNs) are artificial restriction enzymes generated by fusing a zinc finger DNA-binding domain to a DNA-cleavage domain. Zinc finger domains can be engineered to target desired DNA sequences and this enables zinc-finger nucleases to target unique sequences within construct genomes. By taking advantage of endogenous DNA repair machinery, these reagents can be used to alter the genomes of higher organisms. ZFNs may be used in methods for inactivating genes.

A zinc finger DNA-binding domain has about 30 amino acids and folds into a stable structure. Each finger primarily binds to a triplet within the DNA substrate. Amino acid residues at key positions contribute to most of the sequence-specific interactions with the DNA site. These amino acids can be changed while maintaining the remaining amino acids to preserve the necessary structure. Binding to longer DNA sequences is achieved by linking several domains in tandem. Other functionalities like non-specific FokI cleavage domain (N), transcription activator domains (A), transcription repressor domains (R) and methylases (M) can be fused to a ZFPs to form ZFNs respectively, zinc finger transcription activators (ZFA), zinc finger transcription repressors (ZFR, and zinc finger methylases (ZFM).

### Transcription Activator-Like Effector Nucleases (TALENs)

The term TALEN, as used herein, is broad and includes a monomeric TALEN that can cleave double stranded DNA without assistance from another TALEN, e.g., as in Beurdeley, M. et al. Compact designer TALENs for efficient genome engineering. Nat. Commun. 4:1762 doi: 10.1038/ncomms2782 (2013). The term TALEN is also used to refer to one or both members of a pair of TALENs that are engineered to work together to cleave DNA at the same site. TALENs that work together may be referred to as a left-TALEN and a right-TALEN, which references the handedness of DNA or a TALEN-pair.

In some embodiments, a monomeric TALEN can be used. TALENs typically function as dimers across a bipartite recognition site with a spacer, such that two TAL effector domains are each fused to a catalytic domain of the FokI restriction enzyme, the DNA-recognition sites for each resulting TALEN are separated by a spacer sequence, and binding of each TALEN monomer to the recognition site allows FokI to dimerize and create a double-strand break within the spacer. Monomeric TALENs also can be constructed, however, such that single TAL effectors are fused to a nuclease that does not require dimerization to function. One such nuclease, for example, is a single-chain variant of FokI in which the two monomers are expressed as a single polypeptide. Other naturally occurring or engineered monomeric nucleases also can serve this role. The DNA recognition domain used for a monomeric TALEN can be derived from a naturally occurring TAL effector. Alternatively, the DNA recognition domain can be engineered to recognize a specific DNA target. Engineered single-chain TALENs may be easier to construct and deploy, as they require only one engineered DNA recognition domain. A dimeric DNA sequence-specific nuclease can be generated using two different DNA binding domains (e.g., one TAL effector binding domain and one binding domain from another type of molecule). TALENs may function as dimers across a bipartite recognition site with a spacer. This nuclease architecture also can be used for target-specific nucleases generated from, for example, one TALEN monomer and one zinc finger nuclease monomer. In such cases, the DNA recognition sites for the TALEN and zinc finger nuclease monomers can be separated by a spacer of appropriate length. Binding of the two monomers can allow FokI to dimerize and create a double-strand break within the spacer sequence. DNA binding domains other than zinc fingers, such as homeodomains, myb repeats or leucine zippers, also can be fused to FokI and serve as a partner with a TALEN monomer to create a functional nuclease.

In some embodiments, a TAL effector can be used to target other protein domains (e.g., non-nuclease protein domains) to specific nucleotide sequences. For example, a TAL effector can be linked to a protein domain from, without limitation, a DNA 20 interacting enzyme (e.g., a methylase, a topoisomerase, an integrase, a transposase, or a ligase), a transcription activators or repressor, or a protein that interacts with or modifies other proteins such as histones. Applications of such TAL effector fusions include, for example, creating or modifying epigenetic regulatory elements, making site-specific insertions, deletions, or repairs in DNA, controlling gene expression, and modifying chromatin structure.

The spacer of the target sequence can be selected or varied to modulate TALEN specificity and activity. The flexibility in spacer length indicates that spacer length can be chosen to target particular sequences with high specificity. Further, the variation in activity has been observed for different spacer lengths indicating that spacer length can be chosen to achieve a desired level of TALEN activity.

Alternative embodiments use alternative mRNA polymerases and cognate binding sites such as T7 or SP6. Other embodiments relate to the use of any of several alterations of the UTR sequences; these could benefit translation of the mRNA. Some examples are: addition of a cytoplasmic polyadenylation element binding site in the 3′ UTR, or exchanging the Xenopus β-globin UTRs with UTR sequences from human, pig, cow, sheep, goat, zebrafish, from genes including B-globin. UTRs from genes may be selected for regulation of expression in embryonic development or in cells. Some examples of UTRs that may be useful include β-actin, DEAH (SEQ ID NO: 527), TPT1, ZF42, SKP1, TKT, TP3, DDX5, EIF3A, DDX39, GAPDH, CDK1, Hsp90ab1, Ybx1 fEif4b Rps27a Stra13, Myc, Paf1 and Foxo1, or CHUK. Such vector or mRNA improvements could be used to direct special or temporal expression of ectopic TALENs for study of gene depletion at desired stages of development.

In some embodiments, a monomeric TALEN can be used. TALEN typically function as dimers across a bipartite recognition site with a spacer, such that two TAL effector domains are each fused to a catalytic domain of the FokI restriction enzyme, the DNA-recognition sites for each resulting TALEN are separated by a spacer sequence, and binding of each TALEN monomer to the recognition site allows FokI to dimerize and create a double-strand break within the spacer. Monomeric TALENs also can be constructed, however, such that single TAL effectors are fused to a nuclease that does not require dimerization to function. One such nuclease, for example, is a single-chain variant of FokI in which the two monomers are expressed as a single polypeptide. Other naturally occurring or engineered monomeric nucleases also can serve this role. The DNA recognition domain used for a monomeric TALEN can be derived from a naturally occurring TAL effector. Alternatively, the DNA recognition domain can be engineered to recognize a specific DNA target. Engineered single-chain TALENs may be easier to construct and deploy, as they require only one engineered DNA recognition domain. A dimeric DNA sequence-specific nuclease can be generated using two different DNA binding domains (e.g., one TAL effector binding domain and one binding domain from another type of molecule). TALENs may function as dimers across a bipartite recognition site with a spacer. This nuclease architecture also can be used for target-specific nucleases generated from, for example, one TALEN monomer and one zinc finger nuclease monomer. In such cases, the DNA recognition sites for the TALEN and zinc finger nuclease monomers can be separated by a spacer of appropriate length. Binding of the two monomers can allow FokI to dimerize and create a double-strand break within the spacer sequence. DNA binding domains other than zinc fingers, such as homeodomains, myb repeats or leucine zippers, also can be fused to FokI and serve as a partner with a TALEN monomer to create a functional nuclease.

The term nuclease includes exonucleases and endonucleases. The term endonuclease refers to any wild-type or variant enzyme capable of catalyzing the hydrolysis (cleavage) of bonds between nucleic acids within a DNA or RNA molecule, preferably a DNA molecule. Non-limiting examples of endonucleases include type II restriction endonucleases such as FokI, HhaI, HindIII, NotI, BbvCl, EcoRI, BglII, and AhwI. Endonucleases comprise also rare-cutting endonucleases when having typically a polynucleotide recognition site of about 12-45 basepairs (bp) in length, more preferably of 14-45 bp. Rare-cutting endonucleases induce DNA double-strand breaks (DSBs) at a defined locus. Rare-cutting endonucleases can for example be a homing endonuclease, a chimeric Zinc-Finger nuclease (ZFN) resulting from the fusion of engineered zinc-finger domains with the catalytic domain of a restriction enzyme such as FokI or a chemical endonuclease. In chemical endonucleases, a chemical or peptidic cleaver is conjugated either to a polymer of nucleic acids or to another DNA recognizing a specific target sequence, thereby targeting the cleavage activity to a specific sequence. Chemical endonucleases also encompass synthetic nucleases like conjugates of orthophenanthroline, a DNA cleaving molecule, and triplex-forming oligonucleotides (TFOs), known to bind specific DNA sequences. Such chemical endonucleases are comprised in the term “endonuclease” according to the present invention. Examples of such endonuclease include I-See I, I-Chu L I-Cre I, I-Csm I, PI-See L PI-Tti L PI-Mtu I, I-Ceu I, I-See IL 1-See III, HO, PI-Civ I, PI-Ctr L PI-Aae I, PI-Bsu I, PI-Dha I, PI-Dra L PI-May L PI-Meh I, PI-Mfu L PI-Mfl I, PI-Mga L PI-Mgo I, PI-Min L PI-Mka L PI-Mle I, PI-Mma I, PI-30 Msh L PI-Msm I, PI-Mth I, PI-Mtu I, PI-Mxe I, PI-Npu I, PI-Pfu L PI-Rma I, PI-Spb I, PI-Ssp L PI-Fae L PI-Mja I, PI-Pho L PI-Tag L PI-Thy I, PI-Tko I, PI-Tsp I, I-Msol.

A genetic modification made by TALENs or other tools may be, for example, chosen from the list consisting of an insertion, a deletion, insertion of an exogenous nucleic acid fragment, and a substitution. The term “insertion” is used broadly to mean either literal insertion into the chromosome or use of the exogenous sequence as a template for repair. In general, a target DNA site is identified and a TALEN-pair is created that will specifically bind to the site. The TALEN is delivered to the cell or embryo, e.g., as a protein, mRNA or by a vector that encodes the TALEN. The TALEN cleaves the DNA to make a double-strand break that is then repaired, often resulting in the creation of an indel, or incorporating sequences or polymorphisms contained in an accompanying exogenous nucleic acid that is either inserted into the chromosome or serves as a template for repair of the break with a modified sequence. This template-driven repair is a useful process for changing a chromosome, and provides for effective changes to cellular chromosomes.

The term exogenous nucleic acid means a nucleic acid that is added to the cell or embryo, regardless of whether the nucleic acid is the same or distinct from nucleic acid sequences naturally in the cell. In some cases, the exogenous nucleic acid differs in sequence from any nucleic acid sequence that occurs naturally within the cell. The term nucleic acid fragment is broad and includes a chromosome, expression cassette, gene, DNA, RNA, mRNA, or portion thereof.

Genetic modification of cells may also include insertion of a reporter. The reporter may be, e.g., a florescent marker, e.g., green fluorescent protein and yellow fluorescent protein. The reporter may be a selection marker, e.g., puromycin, ganciclovir, adenosine deaminase (ADA), aminoglycoside phosphotransferase (neo, G418, APH), dihydrofolate reductase (DHFR), hygromycin-B-phosphtransferase, thymidine kinase (TK), or xanthin-guanine phosphoribosyltransferase (XGPRT). Vectors for the reporter, selection marker, and/or one or more TALEN may be a plasmid,

TALENs may be directed to a plurality of DNA sites. The sites may be separated by several thousand or many thousands of base pairs. The DNA can be rejoined by cellular machinery to thereby cause the deletion of the entire region between the sites. Embodiments include, for example, sites separated by a distance between 1-5 megabases or between 50% and 80% of a chromosome, or between about 100 and about 1,000,000 basepairs; artisans will immediately appreciate that all the ranges and values within the explicitly stated ranges are contemplated, e.g., from about 1,000 to about 10,000 basepairs or from about 500 to about 500,000 basepairs. Alternatively, exogenous DNA may be added to the cell or embryo for insertion of the exogenous DNA, or template-driven repair of the DNA between the sites. Modification at a plurality of sites may be used to make genetically modified cells, embryos, artiodactyls, and livestock. One or more genes may be chosen for complete or at least partial deletion, including a sexual maturation gene or a cis-acting factor thereof.

### Recombinases

Embodiments of the invention include administration of a TALEN or TALENs with a recombinase or other DNA-binding protein associated with DNA recombination. A recombinase forms a filament with a nucleic acid fragment and, in effect, searches cellular DNA to find a DNA sequence substantially homologous to the sequence. An embodiment of a TALEN-recombinase embodiment comprises combining a recombinase with a nucleic acid sequence that serves as a template for HDR. The HDR template sequence has substantial homology to a site that is targeted for cutting by the TALEN/TALEN pair. As described herein, the HDR template provides for a change to the native DNA, by placement of an allele, creation of an indel, insertion of exogenous DNA, or with other changes. The TALEN is placed in the cell or embryo by methods described herein as a protein, mRNA, or by use of a vector. The recombinase is combined with the HDR template to form a filament and placed into the cell. The recombinase and/or HDR template that combines with the recombinase may be placed in the cell or embryo as a protein, an mRNA, or with a vector that encodes the recombinase. The term recombinase refers to a genetic recombination enzyme that enzymatically catalyzes, in a cell, the joining of relatively short pieces of DNA between two relatively longer DNA strands. Recombinases include Cre recombinase, Hin recombinase, RecA, RAD51, Cre, and FLP. Cre recombinase is a Type I topoisomerase from P1 bacteriophage that catalyzes site-specific recombination of DNA between loxP sites. Hin recombinase is a 21 kD protein composed of 198 amino acids that is found in the bacteria Salmonella. Hin belongs to the serine recombinase family of DNA invertases in which it relies on the active site serine to initiate DNA cleavage and recombination. RAD51 is a human gene. The protein encoded by this gene is a member of the RAD51 protein family which assist in repair of DNA double strand breaks. RAD51 family members are homologous to the bacterial RecA and yeast Rad51 genes. Cre recombinase is an enzyme that is used in experiments to delete specific sequences that are flanked by loxP sites. FLP refers to Flippase recombination enzyme (FLP or Flp) derived from the 2μ plasmid of the baker's yeast Saccharomyces cerevisiae.

Herein, “RecA” or “RecA protein” refers to a family of RecA-like recombination proteins having essentially all or most of the same functions, particularly: (i) the ability to position properly oligonucleotides or polynucleotides on their homologous targets for subsequent extension by DNA polymerases; (ii) the ability topologically to prepare duplex nucleic acid for DNA synthesis; and, (iii) the ability of RecA/oligonucleotide or RecA/polynucleotide constructs efficiently to find and bind to complementary sequences. The best characterized RecA protein is from E. coli; in addition to the original allelic form of the protein a number of mutant RecA-like proteins have been identified, for example, RecA803. Further, many organisms have RecA-like strand-transfer proteins including, for example, yeast, Drosophila, mammals including humans, and plants. These proteins include, for example, Rec1, Rec2, Rad51, Rad51B, Rad51C, Rad51D, Rad51E, XRCC2 and DMC1. An embodiment of the recombination protein is the RecA protein of E. coli. Alternatively, the RecA protein can be the mutant RecA-803 protein of E. coli, a RecA protein from another bacterial source or a homologous recombination protein from another organism.

RecA is known for its recombinase activity to catalyze strand exchange during the repair of double-strand breaks by homologous recombination (McGrew and Knight, 2003) Radding, et al., 1981; Seitz et al., 1998). RecA has also been shown to catalyze proteolysis, e.g., of the LexA and X repressor proteins, and to possess DNA-dependent ATPase activity. After a double-strand break occurs from ionizing radiation or some other insult, exonucleases chew back the DNA ends 5′ to 3′, thereby exposing one strand of the DNA (Cox, 1999; McGrew and Knight, 2003). The single-stranded DNA becomes stabilized by single-strand binding protein (SSB). After binding of SSB, RecA binds the single-stranded (ss) DNA and forms a helical nucleoprotein filament (referred to as a filament or a presynaptic filament). During DNA repair, the homology-searching functions of RecA direct the filament to homologous DNA and catalyze homologous base pairing and strand exchange. This results in the formation of DNA heteroduplex. After strand invasion, DNA polymerase elongates the ssDNA based on the homologous DNA template to repair the DNA break, and crossover structures or Holliday junctions are formed. RecA also shows a motor function that participates in the migration of the crossover structures (Campbell and Davis, 1999).

Recombinase activity comprises a number of different functions. For example, polypeptide sequences having recombinase activity are able to bind in a non-sequence-specific fashion to single-stranded DNA to form a nucleoprotein filament. Such recombinase-bound nucleoprotein filaments are able to interact in a non-sequence-specific manner with a double-stranded DNA molecule, search for sequences in the double-stranded molecule that are homologous to sequences in the filament, and, when such sequences are found, displace one of the strands of the double-stranded molecule to allow base-pairing between sequences in the filament and complementary sequences in one of the strands of the double stranded molecule. Such steps are collectively denoted “synapsis.”

Thus recombinase activities include, but are not limited to, single-stranded DNA-binding, synapsis, homology searching, duplex invasion by single-stranded DNA, heteroduplex formation, ATP hydrolysis and proteolysis. The prototypical recombinase is the RecA protein from E. coli. See, for example, U.S. Pat. No. 4,888,274. Prokaryotic RecA-like proteins have also been described in Salmonella, Bacillus and Proteus species. A thermostable RecA protein, from Thermus aquaticus, has been described in U.S. Pat. No. 5,510,473. A bacteriophage T4 homologue of RecA, the UvsX protein, has been described. RecA mutants, having altered recombinase activities, have been described, for example, in U.S. Pat. Nos. 6,774,213; 7,176,007 and 7,294,494. Plant RecA homologues are described in, for example, U.S. Pat. Nos. 5,674,992; 6,388,169 and 6,809,183. RecA fragments containing recombinase activity have been described, for example, in U.S. Pat. No. 5,731,411. Mutant RecA proteins having enhanced recombinase activity such as, for example, RecA803 have been described. See, for example, Madiraju et al. (1988) Proc. Natl. Acad. Sci. USA 85:6592-6596.

A eukaryotic homologue of RecA, also possessing recombinase activity, is the Rad51 protein, first identified in the yeast Saccharomyces cerevisiae. See Bishop et al., (1992) Cell 69:439-56; Shinohara et al, (1992) Cell: 457-70; Aboussekhra, et al., (1992) Mol. Cell. Biol. 72, 3224-3234 and Basile et al., (1992) Mol. Cell. Biol. 12, 3235-3246. Plant Rad51 sequences are described in U.S. Pat. Nos. 6,541,684; 6,720,478; 6,905,857 and 7,034,117. Another yeast protein that is homologous to RecA is the Dmcl protein. RecA/Rad51 homologues in organisms other than E. coli and S. cerevisiae have been described. Morita et al. (1993) Proc. Natl. Acad. Sci. USA 90:6577-6580; Shinohara et al. (1993) Nature Genet. 4:239-243; Heyer (1994) Experientia 50:223-233; Maeshima et al. (1995) Gene 160:195-200; U.S. Pat. Nos. 6,541,684 and 6,905,857.

Additional descriptions of proteins having recombinase activity are found, for example, in Fugisawa et al. (1985) Nucl. Acids Res. 13:7473; Hsieh et al. (1986) Cell 44:885; Hsieh et al. (1989) J. Biol. Chem. 264:5089; Fishel et al. (1988) Proc. Natl. Acad. Sci. USA 85:3683; Cassuto et al. (1987) Mol. Gen. Genet. 208:10; Ganea et al. (1987) Mol. Cell Biol. 7:3124; Moore et al. (1990) J. Biol. Chem.: 11108; Keene et al. (1984) Nucl. Acids Res. 12:3057; Kimiec (1984) Cold Spring Harbor Symp. 48:675; Kimeic (1986) Cell 44:545; Kolodner et al. (1987) Proc. Natl. Acad. Sci. USA 84:5560; Sugino et al. (1985) Proc. Natl. Acad, Sci. USA 85:3683; Halbrook et al. (1989) J. Biol. Chem. 264:21403; Eisen et al. (1988) Proc. Natl. Acad. Sci. USA 85:7481; McCarthy et al. (1988) Proc. Natl. Acad. Sci. USA 85:5854; and Lowenhaupt et al. (1989) J. Biol. Chem. 264:20568, which are incorporated herein by reference. See also Brendel et al. (1997) J. Mol. Evol. 44:528.

Examples of proteins having recombinase activity include recA, recA803, uvsX, and other recA mutants and recA-like recombinases (Roca (1990) Crit. Rev. Biochem. Molec. Biol. 25:415), (Kolodner et al. (1987) Proc. Natl. Acad. Sci. U.S.A. 84:5560; Tishkoff et al. (1991) Molec. Cell. Biol. 11:2593), RuvC (Dunderdale et al. (1991) Nature 354:506), DST2, KEMI and XRN1 (Dykstra et al. (1991) Molec. Cell. Biol. 11:2583), STPa/DST1 (Clark et al. (1991) Molec. Cell. Biol. 11:2576), HPP-1 (Moore et al. (1991) Proc. Natl. Acad. Sci. U.S.A. 88:9067), other eukaryotic recombinases (Bishop et al. (1992) Cell 69:439; and Shinohara et al. (1992) Cell 69:457); incorporated herein by reference.

In vitro-evolved proteins having recombinase activity have been described in U.S. Pat. No. 6,686,515. Further publications relating to recombinases include, for example, U.S. Pat. Nos. 7,732,585, 7,361,641, 7,144,734. For a review of recombinases, see Cox (2001) Proc. Natl. Acad. Sci. USA 98:8173-8180.

A nucleoprotein filament, or “filament” may be formed. The term filament, in the context of forming a structure with a recombinase, is a term known to artisans in these fields. The nucleoprotein filament so formed can then be, e.g., contacted with another nucleic acid or introduced into a cell. Methods for forming nucleoprotein filaments, wherein the filaments comprise polypeptide sequences having recombinase activity and a nucleic acid, are well-known in the art. See, e.g., Cui et al. (2003) Marine Biotechnol. 5:174-184 and U.S. Pat. Nos. 4,888,274; 5,763,240; 5,948,653 and 7,199,281, the disclosures of which are incorporated by reference for the purposes of disclosing exemplary techniques for binding recombinases to nucleic acids to form nucleoprotein filaments.

In general, a molecule having recombinase activity is contacted with a linear, single-stranded nucleic acid. The linear, single-stranded nucleic acid may be a probe. The methods of preparation of such single stranded nucleic acids are known. The reaction mixture typically contains a magnesium ion. Optionally, the reaction mixture is buffered and optionally also contains ATP, dATP or a nonhydrolyzable ATP analogue, such as, for example, γ-thio-ATP (ATP-γ-S) or γ-thio-GTP (GTP-γ-S). Reaction mixtures can also optionally contain an ATP-generating system. Double-stranded DNA molecules can be denatured (e.g., by heat or alkali) either prior to, or during, filament formation. Optimization of the molar ratio of recombinase to nucleic acid is within the skill of the art. For example, a series of different concentrations of recombinase can be added to a constant amount of nucleic acid, and filament formation assayed by mobility in an agarose or acrylamide gel. Because bound protein retards the electrophoretic mobility of a polynucleotide, filament formation is evidenced by retarded mobility of the nucleic acid. Either maximum degree of retardation, or maximum amount of nucleic acid migrating with a retarded mobility, can be used to indicate optimal recombinase:nucleic acid ratios. Protein-DNA association can also be quantitated by measuring the ability of a polynucleotide to bind to nitrocellulose.

Provided herein, in other aspects, are additional methods of delivering a nucleic acid molecule encoding one or more human leukocyte antigens (HLAs) to a cell. In some embodiments, the method comprises delivery of a nucleic acid molecule encoding one or more HLAs via transcription activator-like effector nucleases (TALENs). In some embodiments, the method comprises delivery of a nucleic acid molecule encoding one or more HLAs via zinc finger nucleases (ZFNs). In some embodiments, the method comprises delivery of a nucleic acid molecule encoding one or more HLAs via clustered regularly interspaced short palindromic repeats/CRISPR associated endonuclease cas9 (CRISPR/Cas9).

In some embodiments, the nucleic acid molecule comprises a deletion in the endogenous HLA locus. In some embodiments, the deletion comprises a deletion in the endogenous HLA-A, HLA-B, or HLA-C locus, or any combination thereof. In some embodiments, the deletion is complete deletion of the endogenous HLA locus.

In some embodiments, the nucleic acid molecule further comprises a sequence encoding a human HLA class 1 heavy chain sequence. In some embodiments, the sequence encoding a human HLA class 1 heavy chain sequence comprises an HLA-A sequence, an HLA-B sequence, an HLA-C sequence, or any combination thereof. In some embodiments, the sequence encoding a human HLA class 1 heavy chain sequence comprises multiple alleles of an HLA-A sequence, an HLA-B sequence, an HLA-C sequence, or any combination thereof. In some embodiments, the sequence encoding a human HLA class 1 heavy chain sequence comprises an HLA-A sequence, wherein the HLA-A sequence is displaced between the HLA-B sequence and the HLA-C sequence.

In some embodiments, the sequence encoding a human HLA class 1 heavy chain sequence comprises fewer than 1700 base pairs (bp). In some embodiments, the sequence encoding a human HLA class 1 heavy chain sequence comprises fewer than 500 bp. In some embodiments, the sequence encoding a human HLA class 1 heavy chain sequence comprises fewer than 250 bp. In some embodiments, the sequence encoding a human HLA class 1 heavy chain sequence comprises fewer than 150 bp.

In some embodiments, the HLA-A sequence, HLA-B sequence, HLA-C sequences, or combination thereof comprises one or more flanking sequences. In some embodiments, the one or more flanking sequences comprise an endogenous HLA sequence. In some embodiments, the one or more flanking sequences are specific to one or more promoters. In some embodiments, the promoters comprise an HLA-A promoter, HLA-B promoter, HLA-C promoter, or combination thereof.

In some embodiments, the sequence encoding a human HLA class 1 heavy chain sequence does not comprise at least a portion of the HLA-A sequence, HLA-B sequence, HLA-C sequence, or combination thereof.

In some embodiments, the nucleic acid molecule encoding the human HLA class 1 heavy chain sequence comprises an HLA-E sequence or a fragment thereof, an HLA-F sequence or a fragment thereof, an HLA-G sequence or a fragment thereof, or any combination thereof. In some embodiments, at least one of the HLA-E sequence or the fragment thereof, HLA-F sequence or the fragment thereof, HLA-G sequence or the fragment thereof, or any combination thereof is inhibited from eliciting a T cell response when the construct is interrogated by one or more T cells.

In some embodiments, the nucleic acid molecule encoding a human HLA class 1 heavy chain sequence comprises one or more mutations, wherein a cell comprising the mutated human HLA class 1 heavy chain sequence comprising the one or more mutations does not elicit an immune response when the cell is interrogated by one or more CD8 cells. In some embodiments, the mutated human HLA class 1 heavy chain sequence encodes an HLA comprising one or more mutations at one or more of amino acid residues 115, 122, 128, 194, 197, 198, 212, 214, 222, 223, 224, 225, 226, 227, 228, 229, 230, 231, 232, 233, 243, 245, 248, 262, or any combination thereof.

### Methods of Treatment

Provided herein, in another aspect, is a method of treating a disease or disorder in a subject in need thereof, comprising administering a therapeutically effective amount of the nucleic acid molecule provided herein or the immune incompetent cell provided herein.

In some embodiments, the disease is an autoimmune disease. In some embodiments, the disease is type 1 diabetes. In some embodiments, the disease is rheumatoid arthritis. In some embodiments, the disease is psoriasis. In some embodiments, the disease is psoriatic arthritis. In some embodiments, the disease is multiple sclerosis. In some embodiments, the disease is systemic lupus erythematosus. In some embodiments, the disease is inflammatory bowel disease. In some embodiments, the disease is Addison's disease. In some embodiments, the disease is Graves' disease. In some embodiments, the disease is Sjögren's syndrome. In some embodiments, the disease is Hashimoto's thyroiditis. In some embodiments, the disease is Myasthenia gravis. In some embodiments, the disease is autoimmune vasculitis. In some embodiments, the disease is pernicious anemia. In some embodiments, the disease is celiac disease. In some embodiments, the disease is vasculitis.

In some embodiments, the disease is a cancer. In some embodiments, the disease is lung cancer. In some embodiments, the disease is breast cancer. In some embodiments, the disease is colorectal cancer. In some embodiments, the disease is prostate cancer. In some embodiments, the disease is skin cancer. In some embodiments, the disease is stomach cancer. In some embodiments, the disease is leukemia. In some embodiments, the disease is lymphoma. In some embodiments, the disease is bladder cancer. In some embodiments, the disease is renal cancer. In some embodiments, the disease is endometrial cancer. In some embodiments, the disease is pancreatic cancer. In some embodiments, the disease is thyroid cancer. In some embodiments, the disease is liver cancer. In some embodiments, the disease is ovarian cancer. In some embodiments, the disease is cervical cancer.

In some embodiments, the disease is a degenerative disease. In some embodiments, the disease is Alzheimer's disease. In some embodiments, the disease is amyotrophic lateral sclerosis. In some embodiments, the disease is Friedreich's ataxia. In some embodiments, the disease is Huntington's disease. In some embodiments, the disease is Lewy body disease. In some embodiments, the disease is Parkinson's disease. In some embodiments, the disease is spinal muscular atrophy. In some embodiments, the disease is multiple sclerosis. In some embodiments, the disease is muscular dystrophy. In some embodiments, the disease is cystic fibrosis. In some embodiments, the disease is Creutzfeldt-Jakob disease. In some embodiments, the disease is Tay-Sachs disease.

In some embodiments, the administration of the immune incompetent cells provided herein treats the disease or disorder without provoking an immune response.

When one or more constructs, nucleic acid molecules, or immune incompetent cells as provided herein are administered into a human subject, the dosage will normally be determined by a physician with the dosage generally varying according to the age, weight, and response of the individual subject, as well as the severity of the subject's symptoms.

The actual dosage employed may be varied depending upon the requirements of the subject and the severity of the condition being treated. Determination of the proper dosage for a particular situation is within the skill of the art. Generally, treatment is initiated with smaller dosages which are less than the optimum dose of the one or more constructs, nucleic acid molecules, or immune incompetent cells as provided herein. Thereafter, the dosage is increased by small amounts until the optimum effect under the circumstances is reached.

The amount and frequency of administration of the one or more constructs, nucleic acid molecules, or immune incompetent cells as provided herein, and if applicable other chemotherapeutic agents and/or radiation therapy, will be regulated according to the judgment of the attending clinician (physician) considering such factors as age, condition and size of the subject as well as severity of the disease being treated.

The chemotherapeutic agent and/or radiation therapy can be administered according to therapeutic protocols well known in the art. It will be apparent to those skilled in the art that the administration of the chemotherapeutic agent and/or radiation therapy can be varied depending on the disease being treated and the known effects of the chemotherapeutic agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., antineoplastic agent or radiation) on the subject, and in view of the observed responses of the disease to the administered therapeutic agents.

Also, in general, the one or more constructs, nucleic acid molecules, or immune incompetent cells as provided herein need not be administered in the same pharmaceutical composition as a chemotherapeutic agent, and may, because of different physical and chemical characteristics, be administered by a different route. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of the one or more constructs, nucleic acid molecules, or immune incompetent cells as provided herein (and where appropriate, chemotherapeutic agent and/or radiation) will depend upon the diagnosis of the attending physicians and their judgment of the condition of the subject and the appropriate treatment protocol.

The one or more constructs, nucleic acid molecules, or immune incompetent cells as provided herein (and where appropriate chemotherapeutic agent and/or radiation) may be administered concurrently (e.g., simultaneously, essentially simultaneously or within the same treatment protocol) or sequentially, depending upon the nature of the proliferative disease, the condition of the subject, and the actual choice of chemotherapeutic agent and/or radiation to be administered in conjunction (i.e., within a single treatment protocol) with the one or more constructs, nucleic acid molecules, or immune incompetent cells as provided herein.

In combinational applications and uses, the one or more constructs, nucleic acid molecules, or immune incompetent cells as provided herein and the chemotherapeutic agent and/or radiation need not be administered simultaneously or essentially simultaneously, and the initial order of administration of the one or more constructs, nucleic acid molecules, or immune incompetent cells as provided herein, and the chemotherapeutic agent and/or radiation, may not be important. Thus, the one or more constructs, nucleic acid molecules, or immune incompetent cells as provided herein may be administered first followed by the administration of the chemotherapeutic agent and/or radiation; or the chemotherapeutic agent and/or radiation may be administered first followed by the administration of the one or more constructs, nucleic acid molecules, or immune incompetent cells as provided herein. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the subject. For example, the chemotherapeutic agent and/or radiation may be administered first, and then the treatment continued with the administration of the one or more constructs, nucleic acid molecules, or immune incompetent cells as provided herein followed, where determined advantageous, by the administration of the chemotherapeutic agent and/or radiation, and so on until the treatment protocol is complete.

Thus, in accordance with experience and knowledge, the practicing physician can modify each protocol for the administration of one or more constructs, nucleic acid molecules, or immune incompetent cells as provided herein for treatment according to the individual subject's needs, as the treatment proceeds.

### Methods of Use

Provided herein, in another aspect, is a method of inhibiting a human leukocyte antigen (HLA) comprising contacting the HLA with a peptide that does not comprise T-cell receptor-binding residues or fragments.

In some embodiments, the peptide binds to one or more HLA binding groove domain residues of the HLA. In some embodiments, the peptide modulates a conformation of the HLA. In some embodiments, the conformation prevents a T cell from binding the HLA.

In some embodiments, the HLA is synthetic.

### Therapeutic Efficacy

The attending clinician, in judging whether treatment is effective at the dosage administered, will consider the general well-being of the subject as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. Relief of disease-related symptoms such as pain, and improvement in overall condition can also be used to help judge effectiveness of treatment.

In some embodiments, therapeutic efficacy is measured based on an effect of treating a proliferative disorder, such as cancer. In general, therapeutic efficacy of the methods and compositions of the invention, with regard to the treatment of a proliferative disorder (e.g. cancer, whether benign or malignant), may be measured by the degree to which the methods and compositions promote inhibition of tumor cell proliferation, the inhibition of tumor vascularization, the eradication of tumor cells, the reduction in the rate of growth of a tumor, and/or a reduction in the size of at least one tumor. Several parameters to be considered in the determination of therapeutic efficacy are discussed herein. The proper combination of parameters for a particular situation can be established by the clinician. The progress of the inventive method in treating cancer (e.g., reducing tumor size or eradicating cancerous cells) can be ascertained using any suitable method, such as those methods currently used in the clinic to track tumor size and cancer progress. The primary efficacy parameter used to evaluate the treatment of cancer by the inventive method and compositions preferably is a reduction in the size of a tumor. Tumor size can be figured using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, MRI, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

In some desirable embodiments, the growth of a tumor is stabilized (i.e., one or more tumors do not increase more than 1%, 5%, 10%, 15%, or 20% in size, and/or do not metastasize) as a result of the inventive method and compositions. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. Preferably, the inventive method reduces the size of a tumor at least about 5% (e.g., at least about 10%, 15%, 20%, or 25%). More preferably, tumor size is reduced at least about 30% (e.g., at least about 35%, 40%, 45%, 50%, 55%, 60%, or 65%). Even more preferably, tumor size is reduced at least about 70% (e.g., at least about 75%, 80%, 85%, 90%, or 95%). Most preferably, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

In some embodiments, the efficacy of the inventive method in reducing tumor size can be determined by measuring the percentage of necrotic (i.e., dead) tissue of a surgically resected tumor following completion of the therapeutic period. In some further embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is 100%, that is, no tumor tissue is present or detectable.

The efficacy of the inventive method can be determined by a number of secondary parameters. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA), prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also can efficiently distinguish small tumors from surrounding tissue (see, for example, U.S. Pat. No. 4,932,412).

In additional desirable embodiments, the treatment of cancer in a human patient in accordance with the inventive method is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, and (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period. While at least a 2% decrease in a tumor-associated antigen level is preferred, any decrease in the tumor-associated antigen level is evidence of treatment of a cancer in a patient by the inventive method. For example, with respect to unresectable, locally advanced pancreatic cancer, treatment can be evidenced by at least a 10% decrease in the CA19-9 tumor-associated antigen level at 4-12 weeks after completion of the therapeutic period as compared to the CA19-9 level before the therapeutic period. Similarly, with respect to locally advanced rectal cancer, treatment can be evidenced by at least a 10% decrease in the CEA tumor-associated antigen level at 4-12 weeks after completion of the therapeutic period as compared to the CEA level before the therapeutic period.

With respect to quality of life assessments, such as the Clinical Benefit Response Criteria, the therapeutic benefit of the treatment in accordance with the invention can be evidenced in terms of pain intensity, analgesic consumption, and/or the Karnofsky Performance Scale score. The treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Karnofsky Performance Scale score reported by a patient, such as for any consecutive four week period in the 12 weeks after completion of the therapeutic period as compared to the Karnofsky Performance Scale score reported by the patient before the therapeutic period.

The treatment of a proliferative disorder (e.g. cancer, whether benign or malignant) in a human patient desirably is evidenced by one or more (in any combination) of the foregoing results, although alternative or additional results of the referenced tests and/or other tests can evidence treatment efficacy.

In some embodiments, tumor size is reduced as a result of the inventive method preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. Desirably, the inventive method is associated with minimal adverse events, e.g. Grade 0, Grade 1, or Grade 2 adverse events, as graded by the CTEP/NCI. However, as discussed herein, reduction of tumor size, although preferred, is not required in that the actual size of tumor may not shrink despite the eradication of tumor cells. Eradication of cancerous cells is sufficient to realize a therapeutic effect. Likewise, any reduction in tumor size is sufficient to realize a therapeutic effect.

Detection, monitoring and rating of various cancers in a human are further described in Cancer Facts and Figures 2001, American Cancer Society, New York, N.Y., and International Patent Application WO 01/24684. Accordingly, a clinician can use standard tests to determine the efficacy of the various embodiments of the inventive method in treating cancer. However, in addition to tumor size and spread, the clinician also may consider quality of life and survival of the patient in evaluating efficacy of treatment.

In some embodiments, administration of one or more constructs, nucleic acid molecules, or immune incompetent cells as provided herein provides improved therapeutic efficacy. Improved efficacy may be measured using any method known in the art, including but not limited to those described herein. In some embodiments, the improved therapeutic efficacy is an improvement of at least about 10%, 20%, 30%, 40%, 50%, 60%, 70%, 75%, 80%, 90%, 95%, 100%, 110%, 120%, 150%, 200%, 300%, 400%, 500%, 600%, 700%, 1000% or more, using an appropriate measure (e.g. tumor size reduction, duration of tumor size stability, duration of time free from metastatic events, duration of disease-free survival). Improved efficacy may also be expressed as fold improvement, such as at least about 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 1000-fold, 10000-fold or more, using an appropriate measure (e.g. tumor size reduction, duration of tumor size stability, duration of time free from metastatic events, duration of disease-free survival).

### Pharmaceutical Compositions and Formulations

The disclosure provides compositions, including pharmaceutical compositions, comprising one or more constructs, nucleic acid molecules, or immune incompetent cells as provided herein.

In some embodiments, the one or more constructs, nucleic acid molecules, or immune incompetent cells as provided herein are formulated into pharmaceutical compositions. In specific embodiments, pharmaceutical compositions are formulated in a conventional manner using one or more physiologically acceptable carriers comprising excipients and auxiliaries which facilitate processing of the one or more constructs, nucleic acid molecules, or immune incompetent cells as provided herein into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising one or more constructs, nucleic acid molecules, or immune incompetent cells as provided herein and a pharmaceutically acceptable diluent(s), excipient(s), or carrier(s). In certain embodiments, the one or more constructs, nucleic acid molecules, or immune incompetent cells as provided herein are administered as pharmaceutical compositions in which the one or more constructs, nucleic acid molecules, or immune incompetent cells as provided herein are mixed with other active ingredients, as in combination therapy. In specific embodiments, the pharmaceutical compositions include one or more constructs, nucleic acid molecules, or immune incompetent cells as provided herein.

A pharmaceutical composition, as used herein, refers to a mixture of one or more constructs, nucleic acid molecules, or immune incompetent cells as provided herein with other chemical components, such as carriers, stabilizers, diluents, dispersing agents, suspending agents, thickening agents, and/or excipients. In certain embodiments, the pharmaceutical composition facilitates administration of the one or more constructs, nucleic acid molecules, or immune incompetent cells as provided herein to an organism. In some embodiments, in practicing the methods of treatment or use provided herein, therapeutically effective amounts of one or more constructs, nucleic acid molecules, or immune incompetent cells as provided herein are administered in a pharmaceutical composition to a mammal having a disease or condition to be treated. In specific embodiments, the mammal is a human. In certain embodiments, therapeutically effective amounts vary depending on the severity of the disease, the age and relative health of the subject and other factors. The one or more constructs, nucleic acid molecules, or immune incompetent cells as provided herein are used singly or in combination with one or more therapeutic agents as components of mixtures.

In one embodiment, one or more constructs, nucleic acid molecules, or immune incompetent cells as provided herein are formulated in an aqueous solution. In specific embodiments, the aqueous solution is selected from, by way of example only, a physiologically compatible buffer, such as Hank's solution, Ringer's solution, or physiological saline buffer. In other embodiments, one or more constructs, nucleic acid molecules, or immune incompetent cells as provided herein are formulated for transmucosal administration. In specific embodiments, transmucosal formulations include penetrants that are appropriate to the barrier to be permeated. In still other embodiments wherein the one or more constructs, nucleic acid molecules, or immune incompetent cells as provided herein are formulated for other parenteral injections, appropriate formulations include aqueous or nonaqueous solutions. In specific embodiments, such solutions include physiologically compatible buffers and/or excipients.

In still other embodiments, the one or more constructs, nucleic acid molecules, or immune incompetent cells as provided herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical composition of one or more constructs, nucleic acid molecules, or immune incompetent cells as provided herein is formulated in a form suitable for parenteral injection as sterile suspension, solution or emulsion in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the one or more constructs, nucleic acid molecules, or immune incompetent cells as provided herein in water-soluble form. In additional embodiments, suspensions of the one or more constructs, nucleic acid molecules, or immune incompetent cells as provided herein are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the one or more constructs, nucleic acid molecules, or immune incompetent cells as provided herein to allow for the preparation of highly concentrated solutions. Alternatively, in other embodiments, the active ingredient is in powder form for constitution with a suitable vehicle, e.g., sterile pyrogen-free water, before use.

In certain embodiments, pharmaceutical compositions are formulated in any conventional manner using one or more physiologically acceptable carriers comprising excipients and auxiliaries which facilitate processing of the one or more constructs, nucleic acid molecules, or immune incompetent cells as provided herein into preparations which can be used pharmaceutically. Proper formulation is dependent upon the route of administration chosen. Any pharmaceutically acceptable techniques, carriers, and excipients are optionally used as suitable. Pharmaceutical compositions comprising the one or more constructs, nucleic acid molecules, or immune incompetent cells as provided herein are manufactured in a conventional manner, such as, by way of example only, by means of conventional mixing, dissolving, granulating, dragee-making, levigating, emulsifying, encapsulating, entrapping or compression processes.

In some embodiments, a pharmaceutical composition comprising one or more constructs, nucleic acid molecules, or immune incompetent cells as provided herein illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, useful aqueous suspension contain one or more polymers as suspending agents. Useful polymers include water-soluble polymers such as cellulosic polymers, e.g., hydroxypropyl methylcellulose, and water-insoluble polymers such as cross-linked carboxyl-containing polymers. Certain pharmaceutical compositions described herein comprise a mucoadhesive polymer, selected for example from carboxymethylcellulose, carbomer (acrylic acid polymer), poly(methylmethacrylate), polyacrylamide, polycarbophil, acrylic acid/butyl acrylate copolymer, sodium alginate and dextran.

Useful pharmaceutical compositions also, optionally, include solubilizing agents to aid in the solubility of one or more constructs, nucleic acid molecules, or immune incompetent cells as provided herein. The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, useful pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, useful compositions also, optionally, include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other useful pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other useful compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other useful compositions include one or more antioxidants to enhance chemical stability where required. Suitable antioxidants include, by way of example only, ascorbic acid and sodium metabisulfite.

In certain embodiments, aqueous suspension compositions are packaged in single-dose non-reclosable containers. Alternatively, multiple-dose reclosable containers are used, in which case it is typical to include a preservative in the composition.

In alternative embodiments, other delivery systems are employed. Liposomes and emulsions are examples of delivery vehicles or carriers useful herein. In additional embodiments, the one or more constructs, nucleic acid molecules, or immune incompetent cells as provided herein are delivered using a sustained-release system, such as semipermeable matrices of solid hydrophobic polymers containing the therapeutic agent. Various sustained-release materials are useful herein. Depending on the chemical nature and the biological stability of the therapeutic reagent, additional strategies for protein stabilization are employed.

In certain embodiments, the formulations described herein comprise one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a composition comprising immune incompetent cells as provided herein is administered in a local rather than systemic manner, for example, via injection of the composition directly into an organ, often in a depot preparation or sustained release formulation. In specific embodiments, long acting formulations are administered by implantation (for example subcutaneously or intramuscularly) or by intramuscular injection. Furthermore, in other embodiments, the composition is delivered in a targeted drug delivery system, for example, in a liposome coated with an organ-specific antibody. In such embodiments, the liposomes are targeted to and taken up selectively by the organ. In yet other embodiments, a composition comprising immune incompetent cells as provided herein is provided in the form of a rapid release formulation, in the form of an extended release formulation, or in the form of an intermediate release formulation.

### Kits and Articles of Manufacture

For use in the therapeutic applications described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

The articles of manufacture provided herein contain packaging materials. Packaging materials for use in packaging pharmaceutical products Include those found in, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. For example, the container(s) includes one or more nucleic acid molecules or immune incompetent cells described herein, optionally in a composition. The container(s) optionally have a sterile access port (for example the container is an intravenous solution bag or a vial having a stopper pierceable by a hypodermic injection needle). Such kits optionally comprise a composition with an identifying description or label or instructions relating to its use in the methods described herein.

For example, a kit typically includes one or more additional containers, each with one or more of various materials (such as reagents, optionally in concentrated form, and/or devices) desirable from a commercial and user standpoint for use of a composition described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included. A label is optionally on or associated with the container. For example, a label is on a container when letters, numbers or other characters forming the label are attached, molded or etched into the container itself, a label is associated with a container when it is present within a receptacle or carrier that also holds the container, e.g., as a package insert. In addition, a label is used to indicate that the contents are to be used for a specific therapeutic application. In addition, the label indicates directions for use of the contents, such as in the methods described herein. In certain embodiments, the pharmaceutical composition is presented in a pack or dispenser device which contains one or more unit dosage forms comprising one or more constructs, nucleic acid molecules, immune incompetent cells, or pharmaceutical compositions thereof provided herein. The pack for example contains metal or plastic foil, such as a blister pack. Or, the pack or dispenser device is accompanied by instructions for administration. Or, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, is the labeling approved by the U.S. Food and Drug Administration for prescription drugs, or the approved product insert. In some embodiments, compositions comprising immune incompetent cells formulated in a compatible pharmaceutical carrier are prepared, placed in an appropriate container, and labeled for treatment of an indicated condition.

### EXAMPLES

### Example 1: Identification of Anchor Amino Acids of Peptide

Most HLA Class 1 alleles preferentially bind 9- to 12-mer peptides, and the majority of alleles accommodate peptides with anchor residues at the second and last positions, as these residues are buried in the peptide binding groove.

An analysis of 9-mer peptides was performed to determine the peptide binding preferences for all alleles of HLA-A, HLA-B, and HLA-C. The amino acid diversity was determined for the anchor residues at the second (e.g. P2) and last (e.g. P9 for a 9-mer) positions. Longer peptides also bind at these anchor positions, with the extra length accommodated by either bulging in the middle or overhanging outside of the peptide binding groove.

The HLA-A alleles investigated included HLA-A*01:01, HLA-A*02:01, HLA-A*02:02, HLA-A*02:03, HLA-A*02:04, HLA-A*02:05, HLA-A*02:06, HLA-A*02:07, HLA-A*02:11, HLA-A*03:01, HLA-A*11:01, HLA-A*11:02, HLA-A*23:01, HLA-A*24:02, HLA-A*24:07, HLA-A*25:01, HLA-A*26:01, HLA-A*29:02, HLA-A*30:01, HLA-A*30:02, HLA-A*31:01, HLA-A*32:01, HLA-A*33:01, HLA-A*33:03, HLA-A*34:01, HLA-A*34:02, HLA-A*36:01, HLA-A*66:01, HLA-A*68:01, HLA-A*68:02, and HLA-A*74:01.

The conserved anchor residues of each investigated HLA-A alleles are summarized in Table 4:

TABLE 4
Conserved Anchor Residues of Each HLA-A Allele

| | Second Amino Acid | Last Amino Acid |
|---|---|---|
| HLA Allele | Anchor Residue | Anchor Residue |
| HLA-A*01 | S/T | Y |
| HLA-A*02 | L/I/V/M | I/V/L |
| HLA-A*03 | L/V/I/T | K/Y |
| HLA-A*11 | T/V | K |
| HLA-A*23 | Y/F | F/L |
| HLA-A*24 | Y/F | F/L |
| HLA-A*25 | V/T | Y/W/F |
| HLA-A*26 | V/T | Y/F |
| HLA-A*29 | F/V | Y |
| HLA-A*30 | V/T | K/Y |
| HLA-A*31 | V/T/Y | R |
| HLA-A*32 | L/T/V | W/F/Y |
| HLA-A*33 | Y/V | R |
| HLA-A*34 | V/T | R/K |
| HLA-A*36 | T/S | Y |
| HLA-A*66 | V/T | R |
| HLA-A*68 | V/T | R/V |
| HLA-A*69 (n = 20) | V/S/H | Y |
| HLA-A*74 | L/V/T | R |

The frequency of second amino acid anchor residues amongst the investigated HLA-A alleles is summarized in Table 5:

TABLE 5
Frequency of Second Amino Acid Anchor Residues of HLA-A

| Second Amino | | Alleles |
|---|---|---|
| Acid Anchor | | Covered |
| Residue | HLA Alleles | (N = 19) |
| S | HLA-A*01, HLA-A*36, HLA-A*69 | 3 (16%) |
| T | HLA-A*01, HLA-A*03, HLA- | 13 (68%) |
| | A*11, HLA-A*25, HLA-A*26, | |
| | HLA-A*30, HLA-A*31, HLA- | |
| | A*32, HLA-A*34, HLA-A*36, | |
| | HLA-A*66, HLA-A*68, HLA-A*74 | |
| V | HLA-A*02, HLA-A*03, HLA- | 15 (79%) |
| | A*11, HLA-A*25, HLA- | |
| | A*26,HLA-A*29, HLA-A*30, | |
| | HLA-A*31, HLA-A*32, HLA- | |
| | A*33, HLA-A*34, HLA-A*66, | |
| | HLA-A*68, HLA-A*69, HLA-A*74 | |
| I | HLA-A*02, HLA-A*03 | 2 (11%) |
| L | HLA-A*02, HLA-A*03, HLA- | 4 (21%) |
| | A*32, HLA-A*74 | |
| M | HLA-A*02 | 1 (5%) |
| F | HLA-A*23, HLA-A*24, HLA-A*29 | 3 (16%) |
| Y | HLA-A*23, HLA-A*24, HLA- | 4 (21%) |
| | A*31, HLA-A*33 | |

The frequency of last amino acid anchor residues amongst the investigated HLA-A alleles is summarized in Table 6:

TABLE 6
Frequency of Last Amino Acid Anchor Residues of HLA-A

| Last Amino | | Alleles |
|---|---|---|
| Acid Anchor | | Covered |
| Residue | HLA Alleles | (N = 19) |
| I | HLA-A*02 | 1 (5%) |
| V | HLA-A*02, HLA-A*68 | 2 (11%) |
| L | HLA-A*02, HLA-A*23, HLA-A*24 | 3 (16%) |
| F | HLA-A*23, HLA-A*24, HLA- | 5 (26%) |
| | A*25, HLA-A*26, HLA-A*32 | |
| Y | HLA-A*01, HLA-A*03, HLA- | 9 (47%) |
| | A*25, HLA-A*26, HLA-A*29, | |
| | HLA-A*30, HLA-A*32, HLA- | |
| | A*36, HLA-A*69 | |
| R | HLA-A*31, HLA-A*33, HLA- | 6 (32%) |
| | A*34, HLA-A*66, HLA-A*68, | |
| | HLA-A*74 | |
| K | HLA-A*03, HLA-A*11, HLA- | 4 (21%) |
| | A*30, HLA-A*34 | |
| W | HLA-A*25, HLA-A*32 | 2 (11%) |

The HLA-B alleles investigated included HLA-B*07:02, HLA-B*07:04, HLA-B*08:01, HLA-B*13:01, HLA-B*13:02, HLA-B*14:02, HLA-B*15:01, HLA-B*15:02, HLA-B*15:03, HLA-B*15:10, HLA-B*15:17, HLA-B*18:01, HLA-B*27:05, HLA-B*35:01, HLA-B*35:03, HLA-B*35:07, HLA-B*37:01, HLA-B*38:01, HLA-B*38:02, HLA-B*40:01, HLA-B*40:02, HLA-B*40:06, HLA-B*42:01, HLA-B*44:02, HLA-B*44:03, HLA-B*45:01, HLA-B*46:01, HLA-B*49:01, HLA-B*50:01, HLA-B*51:01, HLA-B*52:01, HLA-B*53:01, HLA-B*54:01, HLA-B*55:01, HLA-B*55:02, HLA-B*56:01, HLA-B*57:01, HLA-B*57:03, HLA-B*58:01, and HLA-B*58:02.

The conserved anchor residues of each investigated HLA-B alleles are summarized in Table 7:

TABLE 7
Conserved Anchor Residues of Each HLA-B Allele

| | Second Amino Acid | Last Amino Acid |
|---|---|---|
| HLA Allele | Anchor Residue | Anchor Residue |
| HLA-B*07 | P | L |
| HLA-B*08 | L/P | L/V/F |
| HLA-B*13 | Q/L | L/I/F |
| HLA-B*14 | R | L/V |
| HLA-B*15 | Q/L/V/K/H/S/T | Y/F/L/V/W |
| HLA-B*18 | E | Y/F |
| HLA-B*27 | R | L/F/RY |
| HLA-B*35 | P/A | Y/F/L/M |
| HLA-B*37 | D/E | F/L/M/V |
| HLA-B*38 | H | L/I/F |
| HLA-B*40 | E | L/I/F/V/A/P |
| HLA-B*42 | P | L/F/V |
| HLA-B*44 | E | F/W/Y/L |
| HLA-B*45 | E | A/P/V |
| HLA-B*46 | A/L | Y/F/L |
| HLA-B*49 | E | V/I/L |
| HLA-B*50 | E | A/P/V |
| HLA-B*51 | P/A | I/V |
| HLA-B*52 | Q/M | I/V |
| HLA-B*53 | P/A | W/F/L |
| HLA-B*54 | P/A | I/A/V |
| HLA-B*55 | P | A/V |
| HLA-B*56 | P | A/V |
| HLA-B*57 | S/T/A | W/F/Y/L |
| HLA-B*58 | S/T/A | W/F/Y/L/I |

The frequency of second amino acid anchor residues amongst the investigated HLA-B alleles is summarized in Table 8:

TABLE 8
Frequency of Second Amino Acid Anchor Residues of HLA-B

| Second Amino | | Alleles |
|---|---|---|
| Acid Anchor | | Covered |
| Residue | HLA Alleles | (N = 25) |
| A | HLA-B*35, HLA-B*46, HLA- | 7 (28%) |
| | B*51, HLA-B*53, HLA-B*54, | |
| | HLA-B*57, HLA-B*58 | |
| S | HLA-B*15, HLA-B*57, HLA-B*58 | 3 (12%) |
| T | HLA-B*15, HLA-B*57, HLA-B*58 | 3 (12%) |
| V | HLA-B*15 | 1 (4%) |
| L | HLA-B*08, HLA-B*13, HLA- | 4 (16%) |
| | B*15, HLA-B*46 | |
| M | HLA-B*52 | 1 (4%) |
| H | HLA-B*15, HLA-B*38 | 2 (8%) |
| D | HLA-B*37 | 1 (4%) |
| E | HLA-B*18, HLA-B*37, HLA- | 7 (28%) |
| | B*40, HLA-B*44, HLA-B*45, | |
| | HLA-B*49, HLA-B*50 | |
| Q | HLA-B*13, HLA-B*15, HLA-B*52 | 3 (12%) |
| R | HLA-B*14, HLA-B*27 | 2 (8%) |
| K | HLA-B*15 | 1 (4%) |
| P | HLA-B*07, HLA-B*08, HLA- | 9 (36%) |
| | B*35, HLA-B*42, HLA-B*51, | |
| | HLA-B*53, HLA-B*54, HLA- | |
| | B*55, HLA-B*56 | |

The frequency of last amino acid anchor residues amongst the investigated HLA-B alleles is summarized in Table 9:

TABLE 9
Frequency of Last Amino Acid Anchor Residues of HLA-B

| Last Amino | | Alleles |
|---|---|---|
| Acid Anchor | | Covered |
| Residue | HLA Alleles | (N = 25) |
| A | HLA-B*40, HLA-B*45, HLA- | 6 (24%) |
| | B*50, HLA-B*54, HLA-B*55, | |
| | HLA-B*56 | |
| V | HLA-B*08, HLA-B*14, HLA- | 14 (56%) |
| | B*15, HLA-B*37, HLA-B*40, | |
| | HLA-B*42, HLA-B*45, HLA- | |
| | B*49, HLA-B*50, HLA-B*51, | |
| | HLA-B*52, HLA-B*54, HLA- | |
| | B*55, HLA-B*56 | |
| I | HLA-B*13, HLA-B*38, HLA- | 8 (32%) |
| | B*40, HLA-B*49, HLA-B*51, | |
| | HLA-B*52, HLA-B*54, HLA-B*58 | |
| L | HLA-B*07, HLA-B*08, HLA- | 17 (68%) |
| | B*13, HLA-B*14, HLA-B*15, | |
| | HLA-B*27, HLA-B*35, HLA- | |
| | B*37, HLA-B*38, HLA-B*40, | |
| | HLA-B*42, HLA-B*44, HLA- | |
| | B*46, HLA-B*49, HLA-B*53, | |
| | HLA-B*57, HLA-B*58 | |
| F | HLA-B*08, HLA-B*13, HLA- | 15 (60%) |
| | B*15, HLA-B*18, HLA-B*27, | |
| | HLA-B*35, HLA-B*37, HLA- | |
| | B*38, HLA-B*40, HLA-B*42, | |
| | HLA-B*44, HLA-B*46, HLA- | |
| | B*53, HLA-B*57, HLA-B*58 | |
| Y | HLA-B*15, HLA-B*18, HLA- | 8 (32%) |
| | B*27, HLA-B*35, HLA-B*44, | |
| | HLA-B*46, HLA-B*57, HLA-B*58 | |
| M | HLA-B*35, HLA-B*37, HLA-B*44 | 3 (12%) |
| W | HLA-B*15, HLA-B*53, HLA- | 4 (16%) |
| | B*57, HLA-B*58 | |
| P | HLA-B*40, HLA-B*45, HLA-B*50 | 3 (12%) |
| R | HLA-B*27 | 1 (4%) |

The HLA-C alleles investigated included HLA-C*01:02, HLA-C*02:02, HLA-C*03:02, HLA-C*03:03, HLA-C*03:04, HLA-C*04:01, HLA-C*04:03, HLA-C*05:01, HLA-C*06:02, HLA-C*07:01, HLA-C*07:02, HLA-C*07:04, HLA-C*08:01, HLA-C*08:02, HLA-C*12:02, HLA-C*12:03, HLA-C*14:02, HLA-C*14:03, HLA-C*15:02, HLA-C*16:01, and HLA-C*17:01.

The conserved anchor residues of each investigated HLA-C alleles are summarized in Table 10:

TABLE 10
Conserved Anchor Residues of Each HLA-C Allele

| | Second Amino Acid | Last Amino Acid |
|---|---|---|
| HLA Allele | Anchor Residue | Anchor Residue |
| HLA-C*01 | A/L/S | L/V/M |
| HLA-C*02 | A/S/T/Q/V | F/Y/L/V |
| HLA-C*03 | A/S/I | L/F/Y/M |
| HLA-C*04 | F/Y/W/A/T/V | L/F/V/M/I |
| HLA-C*05 | A/L/S/V | L/F/V/M |
| HLA-C*06 | R/A/Y | L/V/I |
| HLA-C*07 | R/T/N/Y | L/Y/F/M |
| HLA-C*08 | A/S/V | L/V |
| HLA-C*12 | A/I/S | F/Y/L/V |
| HLA-C*14 | Y/F | L/F/Y |
| HLA-C*15 | A/T/A | V/L |
| HLA-C*16 | A/S/T | L/Y/F |
| HLA-C*17 | A | L |

The frequency of second amino acid anchor residues amongst the investigated HLA-C alleles is summarized in Table 11:

TABLE 11
Frequency of Second Amino Acid Anchor Residues of HLA-C

| Second Amino | | Alleles |
|---|---|---|
| Acid Anchor | | Covered |
| Residue | HLA Alleles | (N = 13) |
| A | HLA-C*01, HLA-C*02, HLA- | 11 (85%) |
| | C*03, HLA-C*04, HLA-C*05, | |
| | HLA-C*06, HLA-C*08, HLA-C*12, | |
| | HLA-C*15, HLA-C*16, HLA- | |
| | C*17 | |
| S | HLA-C*01, HLA-C*02, HLA- | 7 (54%) |
| | C*03, HLA-C*05, HLA-C*08, | |
| | HLA-C*12, HLA-C*16 | |
| T | HLA-C*02, HLA-C*04, HLA- | 5 (38%) |
| | C*07, HLA-C*15, HLA-C*16 | |
| V | HLA-C*02, HLA-C*04, HLA- | 4 (31%) |
| | C*05, HLA-C*08 | |
| I | HLA-C*03, HLA-C*12 | 2 (15%) |
| L | HLA-C*01, HLA-C*05 | 2 (15%) |
| F | HLA-C*04, HLA-C*14 | 2 (15%) |
| Y | HLA-C*04, HLA-C*06, HLA- | 4 (31%) |
| | C*07, HLA-C*14 | |
| Q | HLA-C*02 | 1 (8%) |
| R | HLA-C*06, HLA-C*07 | 2 (15%) |
| N | HLA-C*07 | 1 (8%) |
| W | HLA-C*04 | 1 (8%) |

The frequency of last amino acid anchor residues amongst the investigated HLA-C alleles is summarized in Table 12:

TABLE 12
Frequency of Last Amino Acid Anchor Residues of HLA-C

| Last Amino | | Alleles |
|---|---|---|
| Acid Anchor | | Covered |
| Residue | HLA Alleles | (N = 13) |
| L | HLA-C*01, HLA-C*02, HLA- | 13 (100%) |
| | C*03, HLA-C*04, HLA-C*05, | |
| | HLA-C*06, HLA-C*07, HLA- | |
| | C*08, HLA-C*12, HLA-C*14, | |
| | HLA-C*15, HLA-C*16, HLA-C*17 | |
| V | HLA-C*01, HLA-C*02, HLA- | 8 (61%) |
| | C*04, HLA-C*05, HLA-C*06, | |
| | HLA-C*08, HLA-C*12, HLA-C*15 | |
| M | HLA-C*01, HLA-C*03, HLA- | 5 (38%) |
| | C*04, HLA-C*05, HLA-C*07 | |
| F | HLA-C*02, HLA-C*03, HLA- | 8 (61%) |
| | C*04, HLA-C*05, HLA-C*07, | |
| | HLA-C*12, HLA-C*14, HLA-C*16 | |
| Y | HLA-C*02, HLA-C*03, HLA- | 6 (46%) |
| | C*07, HLA-C*12, HLA-C*14, | |
| | HLA-C*16 | |
| I | HLA-C*04, HLA-C*06 | 2 (15%) |

### Example 2: Preparation of Nucleic Acid Molecule

A nucleic acid molecule comprising the synthetic HLA sequence is prepared according to known methods in the art. For example, the nucleic acid molecule is prepared by solid-phase oligonucleotide synthesis using nucleoside phosphoramidites. Alternatively, portions of the nucleic acid molecule are prepared by solid-phase oligonucleotide synthesis using nucleoside phosphoramidites, and the portions are assembled into the complete nucleic acid molecule according to known methods in the art. For example, the portions are assembled using endonuclease-mediated assembly, site-specific recombination, or long-overlap based assembly.

### Example 3: Preparation of Construct

A recombinant plasmid comprising the nucleic acid molecule comprising the synthetic HLA sequence is prepared according to known procedures for the preparation of recombinant plasmids. For example, the plasmid is cut with a restriction enzyme and the nucleic acid molecule comprising the synthetic HLA sequence is introduced into the plasmid via the use of DNA ligase.

The recombinant plasmid is subsequently transformed into a population of cells. The successfully transformed cells are selected according to known methods in the art, such as with the use of a selection antibiotic. The selected cells are cultured and induced to produce the construct. The cells are subsequently lysed and the construct is purified according to protein purification techniques known in the art, such as size exclusion chromatography, hydrophobic interaction chromatography, ion exchange chromatography, free-flow electrophoresis, immunoaffinity chromatography, immunoprecipitation, or high performance liquid chromatography.

Alternatively, the construct is preparing by solid phase peptide synthesis according to known methods in the art.

### Example 4: Preparation of Immune Incompetent Cells

An immune incompetent cell, such as an immune incompetent stem cell, is prepared by introducing the nucleic acid molecule of Example 2 into the cell's genome according to methods known in the art. For example, the nucleic acid molecule is delivered to the cell via a viral vector. Alternatively, the nucleic acid molecule is delivered to the cell via a non-viral method, such as the use of naked DNA injection, electroporation, gene gun, sonoporation, magnetofection, lipoplexes, dendrimers, inorganic nanoparticles, CRISPR, mRNA, or siRNA.

Alternatively, an immune incompetent cell, such as an immune incompetent stem cell, is prepared by incubating the cell with the construct of Example 3.

### Example 5: Immune Cell Proliferation Assay

Immune cells, for example T cells, are cultured and incubated with the immune incompetent cell of Example 4 and a radio-labelled nucleotide, such as ³H-thymidine. Following incubation, the immune cells are centrifugated and washed and the radioactivity is measured and compared to a control group of cells not treated with the immune incompetent cell of Example 4.

### Example 6: Treatment of Disease or Disorder

The immune incompetent cells of Example 4 are administered to a patient suffering from a disease or disorder. The patient's condition is monitored according to therapeutic standards of care according to the disease or disorder.

### Example 7: Transgenic Cloning of synHLA Constructs into B2Mnull EBV and K562 Cells

### Generation of B2M^{-/-} EBV-Transformed B-Cell Lines

CRISPR/Cas9 is used to mutate β2M in 2 EBV lines ((9031 and JK). EBV cells are transfected with Cas9 construct and HLA class I low/negative EBV cells are sorted. The cells are grown, and the absence of surface HLA class I is confirmed.

Generation of NK Cell Lines and Testing K562 and/or B2M^{-/-} EBV-Transformed B-Cell Lines for Susceptibility to NK Cell Mediated Killing

NK cells (CD3⁻, CD56⁺) are sorted and grown in the presence of IL-2, -15 and IL-21. NK cells from PBMC are sorted and expanded in cytokines (IL-2 and IL-15). The purity of NK cell lines may be confirmed by staining with CD3, CD4, CD8, CD56. The killing of K562 and/or WT and B2M/HLA class I^{-/-} EBV cells is compared.

Express synHLA Protein(s) in K562 and/or EBV-Transformed B-Cell Lines

A synHLA gene encoding a synHLA construct as described herein is cloned into either a lentiviral vector (pRRLSIN) or an EBV episomal vector (pCE). A gene block is constructed that encodes the synHLA codon-optimized for expression in mammalian cells. This gene may be cloned into pRRLSIN or pCE using, e.g., Infusion cloning. The construct may be sequenced to confirm that the sequence is correct and a plasmid is prepared. pRRLSIN may be used to make lentiviruses which are used to transduce K562 cells. pCE is electroporated in EBV cells and transfected cells selected by antibiotic selection. Transduced K562 are stained for synHLA construct and selected by FACS sorting. Transfected EBV cells (WT and B2MB2M/HLA class I^{-/-}) are selected in the presence of antibody and the expression of the synHLA construct(s) is assessed by flow cytometry with a monoclonal antibody. K562 or EBV cells expressing the synHLA construct(s) are tested for recognition and killing by NK cell lines generated as described above.

Determine if EBV Cells, or K562 Cells Expressing synHLA can Activate Antigen Specific CD8⁺ T Cell Clones.

Using either EBV-transformed B-cell lines: with or without HLA class I; and with or without synHLA, or K562 transduced with the appropriate HLA class I construct, the following is tested: Response of influenza MP₅₈₋₆₆ epitope specific CD8⁺ T cell clones, described above, pulsed with the cognate peptide. Proliferation of allogeneic primary CFSE-labelled CD8⁺ T cells, purified by magnetic beads or flow cytometry, in response to K562 and/or EBV-transformed B cell lines described above, using the method of Mannering, S. I. et al. A sensitive method for detecting proliferation of rare autoantigen-specific human T cells. J Immunol Methods, 2003, 283, 173-183. T cell killing of transduced cells will also be assessed by measuring interferon gamma secretion.

### Example 8: Recombinant Bacterial Expression of HLA Constructs

HLA constructs, such as synthetic HLA constructs (synHLA) and/or single chain dimers or trimers as described herein (e.g., those listed in Table 1) are recombinantly expressed in bacteria. Briefly, bacterial cells are transformed with nucleic acids encoding the sequence of a synHLA construct as described herein. In some cases, the protein may contain tags and additional sequence motifs for the purpose of, e.g., assisting purification, facilitating cleavage of purification tags or other purpose, including (but not limited to) a 6xHis tag, AviTag™ and/or TEV cleavage site. Optionally, the purification tag/s are removed by cleavage with TEV protease according to standard protocols. Optionally, proteins are biotinylated on their AviTag™ according to standard protocols.

The protein may be isolated and purified from the bacteria. If the protein is present as inclusion bodies, the protein may be refolded (e.g., by denaturing with a chaotropic agent and transferring to a dilute aqueous environment). Following refolding, the refolded protein may be purified (e.g., by immobilized metal-affinity chromatography (IMAC), anion exchange chromatography, size exclusion chromatography (SEC)). Isolated protein may be subjected to multiple quality control operations such as visualization by Coomassie Blue stained SDS-PAGE gels in the presence or absence of a reducing agent (e.g, dithiothreitol; DTT), or validated by techniques such as time of flight mass-spectrometry (TOF-MS) and dynamic light scattering (DLS).

### Example 9: Expression of Synthetic HLA Constructs in Bacteria

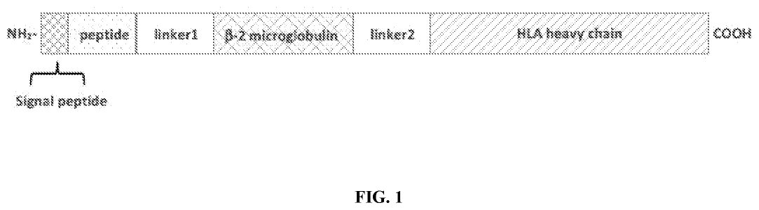

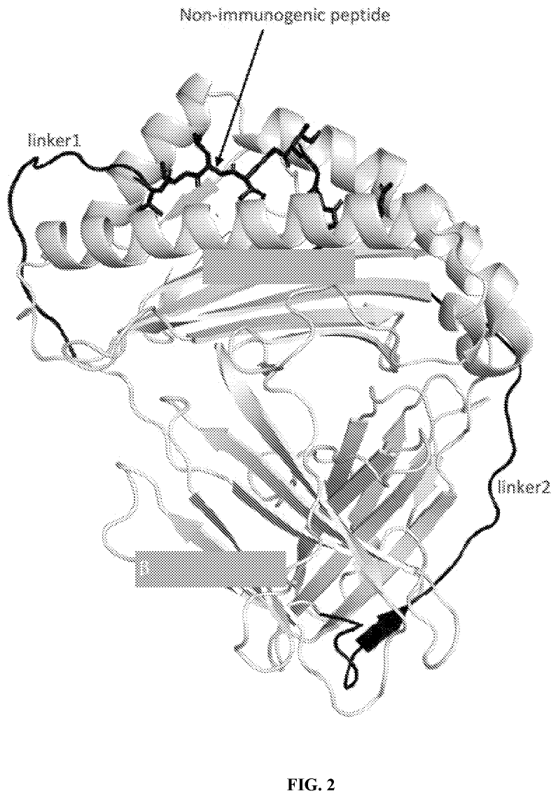

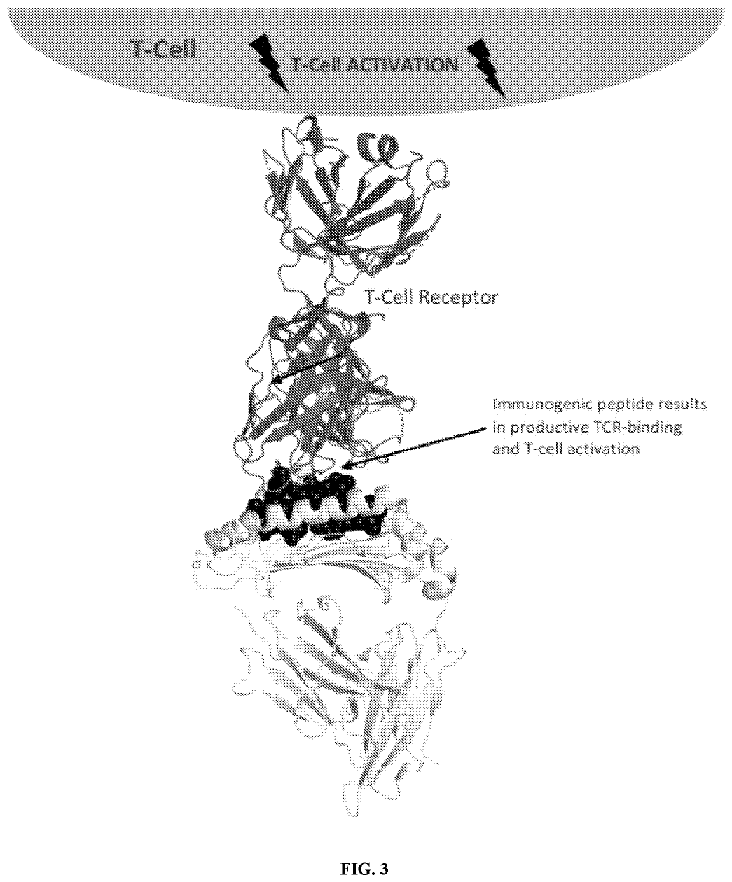

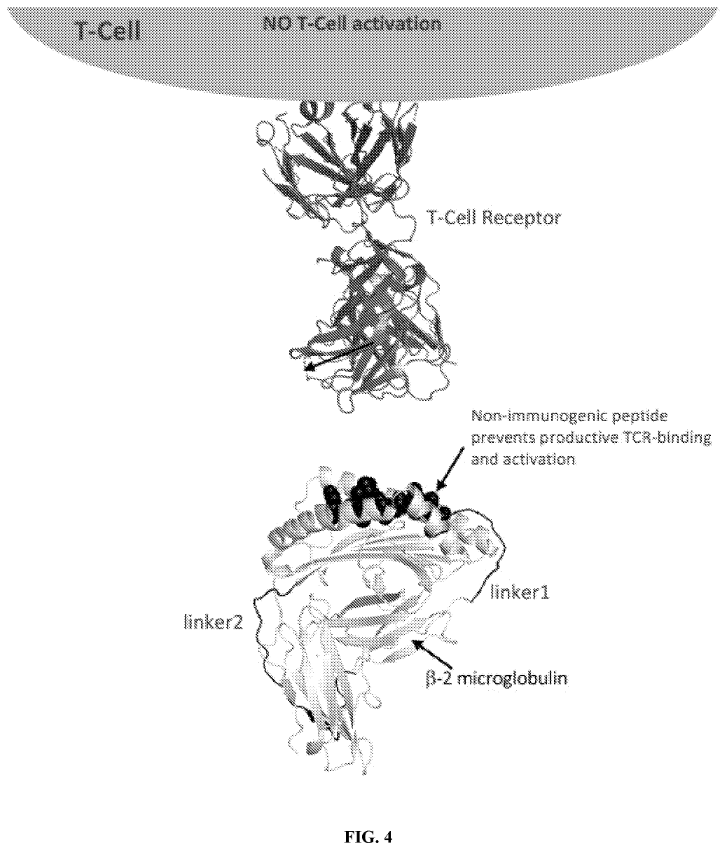

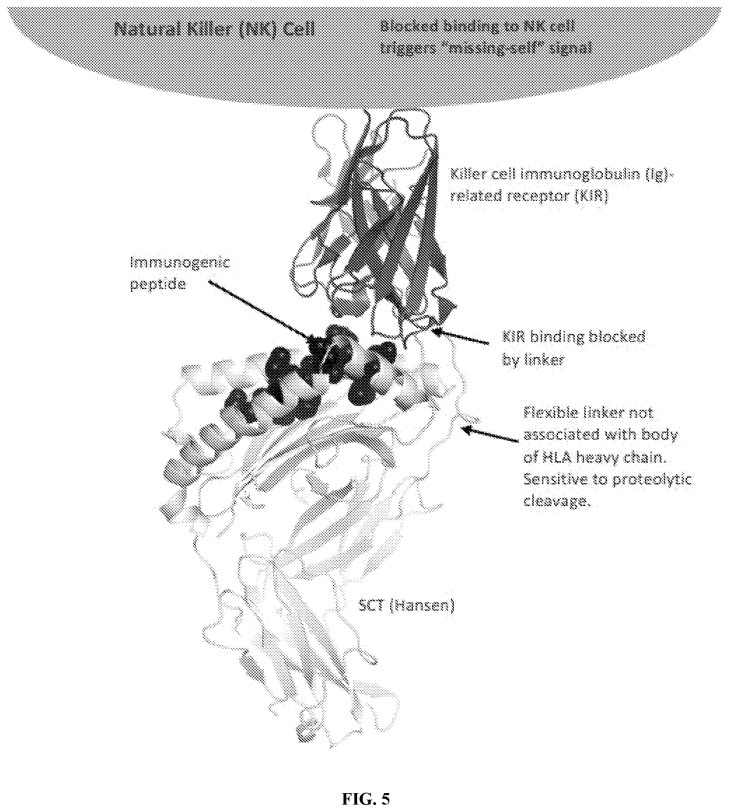

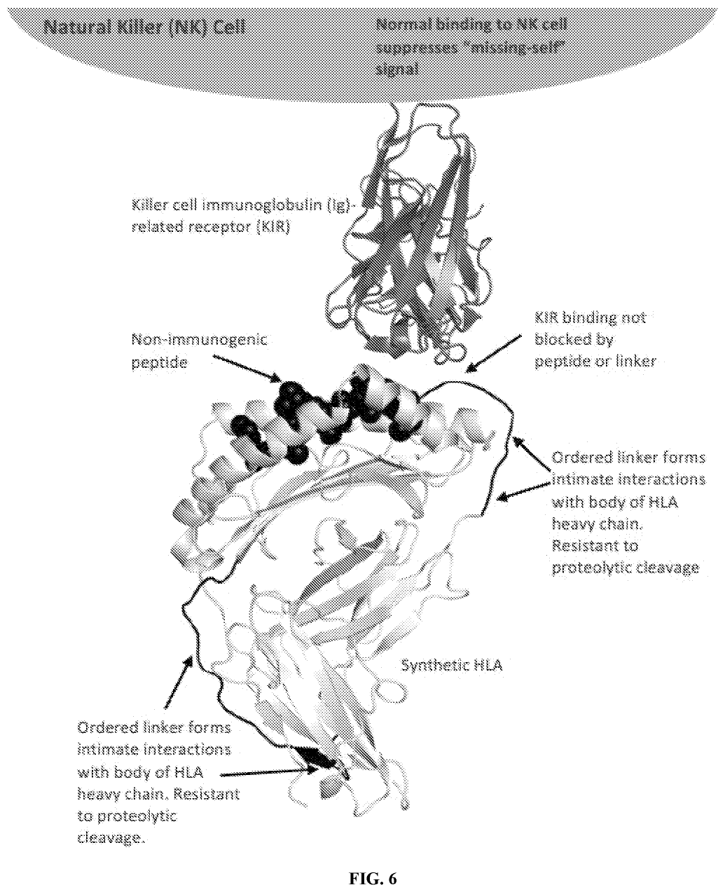

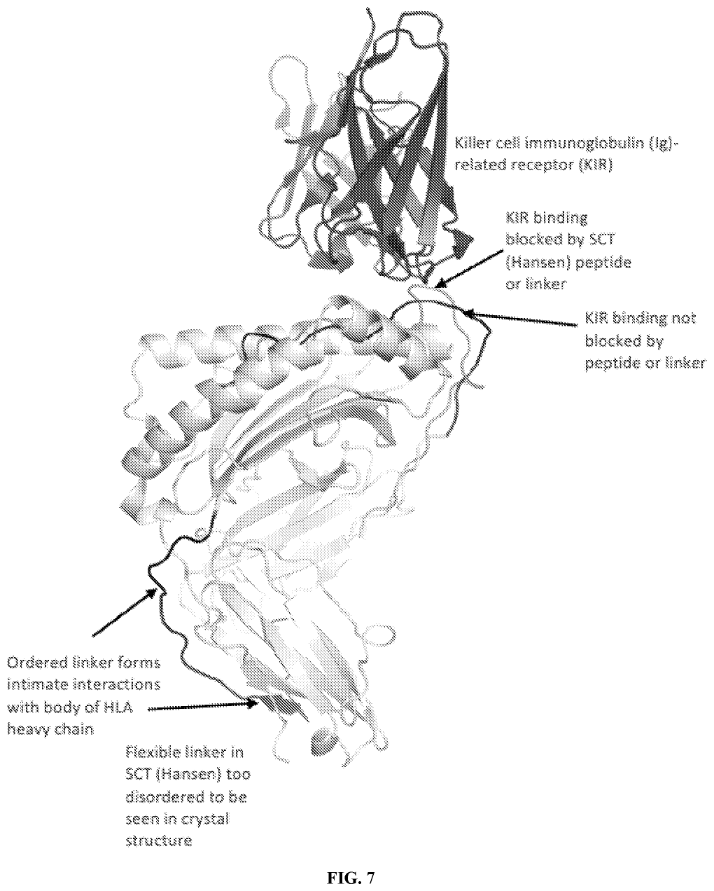

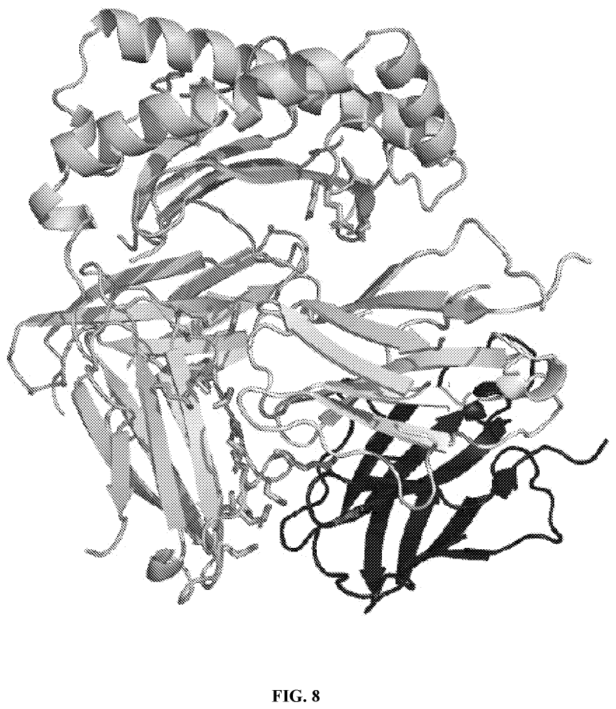

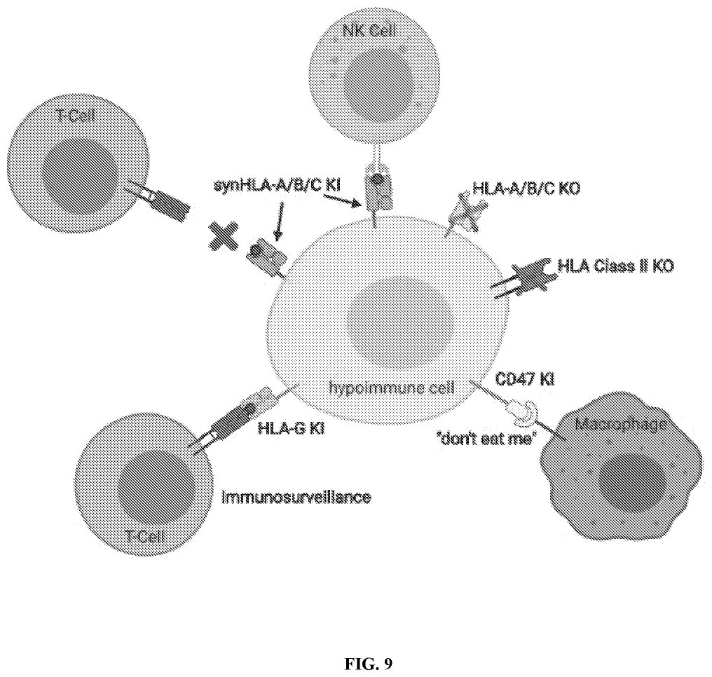

Synthetic HLA proteins as described herein (SYNC4-1, SEQ ID NO: 39; SYNA1-1, SEQ ID NO: 31; SYNC5-1, SEQ ID NO: 42; SYNC6-1, SEQ ID NO: 44; SCTC1-1, SEQ ID NO: 10) were expressed, isolated, refolded, purified, and analyzed as described in Example 8. Briefly, DNA encoding synthetic HLA proteins was inserted into an expression vector, then introduced into bacteria under antibiotic selection. The protein construct format was as described in FIG. 1, but with no N-terminal signal peptide and an additional C-terminal purification tags as detailed in Table 13. Cells harboring the appropriate sequences were grown to mid-log phase, and expression was induced with isopropyl β-D-1-thiogalactopyranoside (IPTG). Cells were harvested by centrifugation, lysed using a high-pressure homogenizer and centrifuged to isolate the soluble and insoluble fractions. Insoluble inclusion bodies containing synthetic HLA proteins were washed and resuspended in a denaturing buffer. Synthetic HLA proteins were refolded from the inclusion body preparation by dilution in a refold buffer, followed by incubation at 4° C. for up to 72 hours. The resulting protein was purified by a combination of IMAC, size exclusion and/or ion exchange chromatography

TABLE 13
Synthetic HLA proteins (test and control)
expressed in bacteria.

| | SEQ | | | | C- | Mutations | | |
|---|---|---|---|---|---|---|---|---|
| | ID | | Linker | Linker | term | | | Q226A, |
| Name | NO. | Peptide | L1* | L2^{&} | [symbol] | C1G^{#, $} | Y84^{$} | D227K |
| SYNA1- | 31 | GILGFVFTL | L | L | His | WT | A | WT$ |
| 1 | | (SEQ ID | | | | | | |
| | | NO: 158) | | | | | | |
| SCTC1- | 10 | GILGFVFTL | L | L | His | WT | WT | WT |
| 1 | | (SEQ ID | | | | | | |
| | | NO: 158) | | | | | | |
| SYNC4- | 39 | RYRPGTVAL | M | L | His | WT | A | WT |
| 1 | | (SEQ ID | | | | | | |
| | | NO: 159) | | | | | | |
| SYNC5- | 42 | RYRPGTVAL | M | L | His | WT | A | AK |
| 1 | | (SEQ ID | | | | | | |
| | | NO: 159) | | | | | | |
| SYNC6- | 44 | RYRPGTVAL | M + C | L | His | WT | C | AK |
| 1 | | (SEQ ID | | | | | | |
| | | NO: 159) | | | | | | |

*Linker 1 (L1) length, L = Long (GGGGSGGGGSGGGGS) (SEQ ID NO: 179), M = Medium (GGGGSGGGGSGS) (SEQ ID NO: 177), S = Short (GGGGSGGGGS) (SEQ ID NO: 175), + C = glycine in position 2 of the L1 linker mutated to cysteine; &Linker 2 (L2) length, L = Long (GGGGSGGGGSGGGGS; SEQ ID NO: 183);
^{$}WT = wild type sequence of Q226, D227, Y84 or C1.
Protein tags: His, Hexahis tag; AVI, AviTag ™.
A TEV protease cleavage site is included N-terminal to the Hexahis tag.
^{#}First position in the HLA domain.

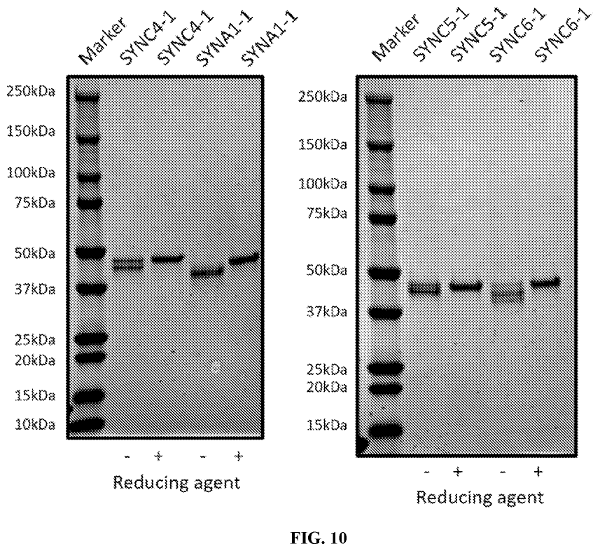

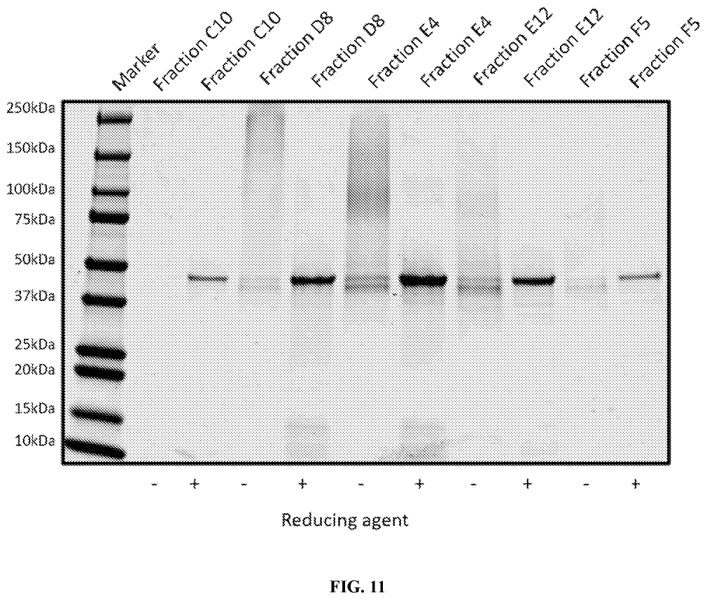

Results of the SDS-PAGE analysis of each construct are shown in FIGS. 10 and 11. Separation by SDS-PAGE followed by Coomassie Blue staining shows that SYNA1-1 migrates as a single band under non-reducing conditions suggesting homogeneity of disulfite bond formation (FIG. 10). SYNA1-1 contains the HLA-A*02 scaffold. The synthetic HLA proteins SCTC1-1, SYNC4-1, SYNC5-1 and SYNC6-1, which contain the HLA-C*07 scaffold, migrated as multiple species under non-reducing conditions (FIGS. 10 and 11). Both SYNC4-1 and SYNC5-1 migrated as 2 discreet species and SYNC6-1 migrated as 3 discreet species (FIG. 10). That these bands collapse into a single band under reducing conditions suggests that multiple species of disulfide-bonded proteins are present, perhaps as a result of an extra unpaired cysteine present in the HLA-C*07 domain, residue number 1 (C1) in the HLA domain.

Proteins were analyzed by TOF-MS to confirm their mass using standard instruments and protocols. Briefly, 10 μg of protein was heated to 50° C. for 5 minutes in the presence or absence of 10 mM reducing agent (e.g. TCEP, DTT) before being analyzed by TOF-MS. TOF-MS experiments revealed the SYNC4-1, SYNC5-1 and SYNC6-1 constructs were approximately 438 Da heavier than the expected mass (SYNC4-1, expected 47771.4 Da, observed 48209.9 Da; SYNC5-1 expected 47727.5 Da, observed 48165.9 Da; SYNC6-1 expected 47803.6 Da, observed 48242.2 Da.

### Example 10: Modulating Possibility of Disulfide Bond Formation

The cysteine present in the HLA-C*07 domain as described in Example 9 was mutated to glycine (C1G) to reduce the possibility or mis-paired disulfides forming and thus improving protein expression. This C1G mutation was introduced into two proteins, SYNC22-1 (SEQ ID NO: 80) and SCTC3-1 (SEQ ID NO: 14) (Table E).

In addition, in order to resolve the possible issue of first residue of the peptide prohibiting cleavage of the initiating methionine within the bacterial expression system, an R to S mutation was introduced into the first position of the peptide domain (R1S) in SYNC22-1 and SCTC3-1. It was predicted that this substitution would restore the ability of the bacterial machinery to remove the initiating methionine of the recombinant synthetic HLA proteins.

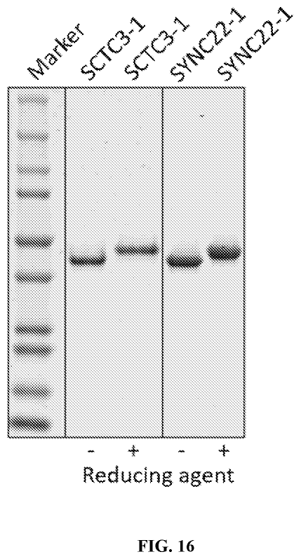

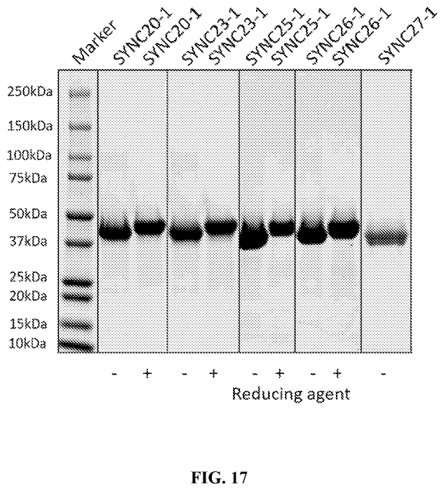

SYNC22-1 and SCTC3-1 were expressed, isolated, refolded, purified and analyzed as described in Examples 8 and 9. The protein construct format was as described in FIG. 1, but with no N-terminal signal peptide and with or without additional C-terminal purification tags (e.g., hexa-His tag or AviTag™) as detailed in Table E. The resulting recombinant proteins migrated as a single as a single species under non-reducing conditions suggesting homogeneity of disulfide bond formation (FIG. 15). Furthermore, when these proteins were analyzed by TOF-MS, the expected mass matched the observed mass (SYNC22-1, expected 47656.2 Da, observed 47657.5 Da; SCTC3-1, expected 47039.6 Da, observed 47040.6 Da) suggesting that the initiating methionine had been successfully cleaved. The results are summarized in Table 13. The masses observed are within the acceptable error range (±2 Da) for TOF-MS on the QTOF 5600 instrument.

Depending on the downstream application of recombinant proteins, they were subjected to TEV protease cleavage or biotinylation according to standard protocols. Briefly, TEV protease was incubated with the synthetic HLA proteins at a ratio of 1:100 w/w overnight at 4° C. and separated via size exclusion chromatography. Biotinylation of the AviTag™ was achieved by incubating the synthetic HLA proteins with bacterially expressed recombinant GST-labelled BirA in the presence of 10 mM ATP, 10 mM MgCl₂ and 50 μM D-biotin at 4° C. overnight. BirA was separated from the reaction mixture using Glutathione Sepharose 4b resin. Biotinylated synthetic HLA proteins were further purified by size exclusion chromatography.

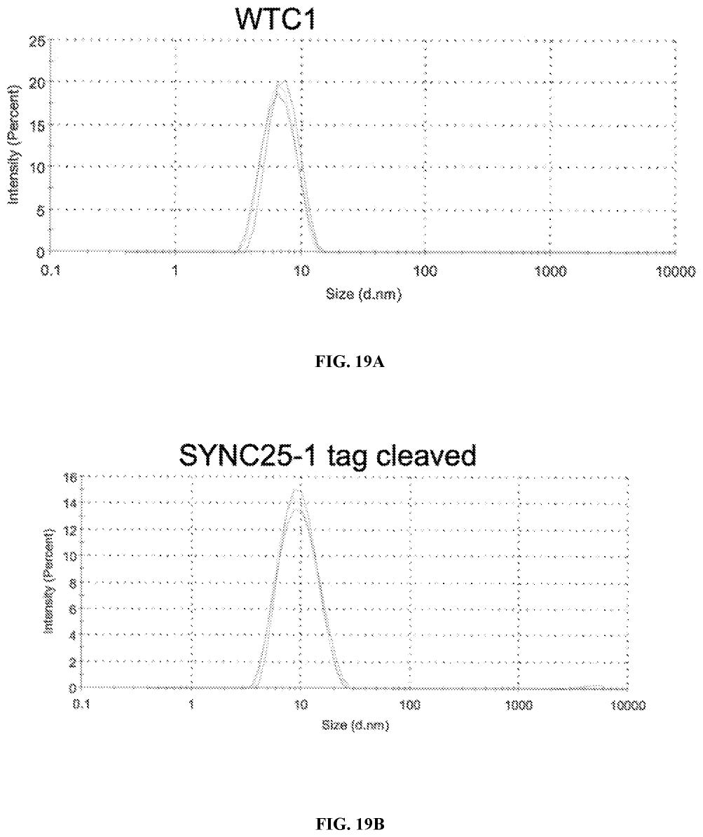

To assess the homogeneity of the protein sample, dynamic light scattering (DLS) was used to analyze SCTC3-1 according to standard protocols. Briefly, samples were added to the sample cuvette and analyzed in the Zetasizer. FIG. 18 shows that a double peak was observed for SCTC3-1, indicating more than one protein species may have been present in the sample, potentially a proportion of unfolded material. SCTC3-1 appeared homogenous by SDS-PAGE. The results are summarized in Table 16.

Subsequent synHLA constructs for recombinant protein expression were therefore all produced in the context of R1S in the peptide domain and CIG in the HLA domain.

TABLE 14
List of synthetic HLA proteins (control and test)
expressed in bacteria to determine the impact of
mutation of C1G (HLA domain) mutation of R1S
(peptide domain).

| | SEQ | | | | C- | Mutations | | |
|---|---|---|---|---|---|---|---|---|
| | ID | | Linker | Linker | term | | | Q226A, |
| Name | NO | Peptide | L1* | L2& | [symbol] | C1G^{#} | Y84^{$} | D227K^{$} |
| SCTC3-1 | 14 | SYRPGTVAL | L | L | His | G | WT | WT |
| | | (SEQ ID | | | | | | |
| | | NO: 160) | | | | | | |
| SYNC22- | 80 | SYRPGTVAL | M | L | His | G | A | WT |
| 1 | | (SEQ ID | | | | | | |
| | | NO: 160) | | | | | | |

*Linker 1 (L1) length, L = Long (GGGGSGGGGSGGGGS; SEQ ID NO: 179), M = Medium (GGGGSGGGGSGS; SEQ ID NO: 177), S = Short (GGGGSGGGGS; SEQ ID NO: 175). + C = glycine in position 2 of the L1 linker mutated to cysteine.
^{&}Linker 2 (L2) length, L = Long (GGGGSGGGGSGGGGS; SEQ ID NO: 183).
^{$}WT = wild type sequence of Q226, D227 or Y84. [symbol]
Protein tags: His, Hexa-his tag; AVI, AviTag ™.
A TEV protease cleavage site is included N-terminal to the Hexa-his tag.
^{#}First position in the HLA domain.

### Example 11: Expression of Further Synthetic HLA Proteins in Bacteria with R1S or C1G Mutations in the Peptide and HLA Domains

Synthetic HLA proteins harboring R1S and/or C1G mutations, along with other variations (Table 15) were expressed, isolated, refolded, purified, and analyzed as described in Examples 8 and 9. The protein construct format was as described in FIG. 1, but with no N-terminal signal peptide and with or without additional C-terminal purification tags (e.g. hexa-His tag or AviTag™) as detailed in Table 15.

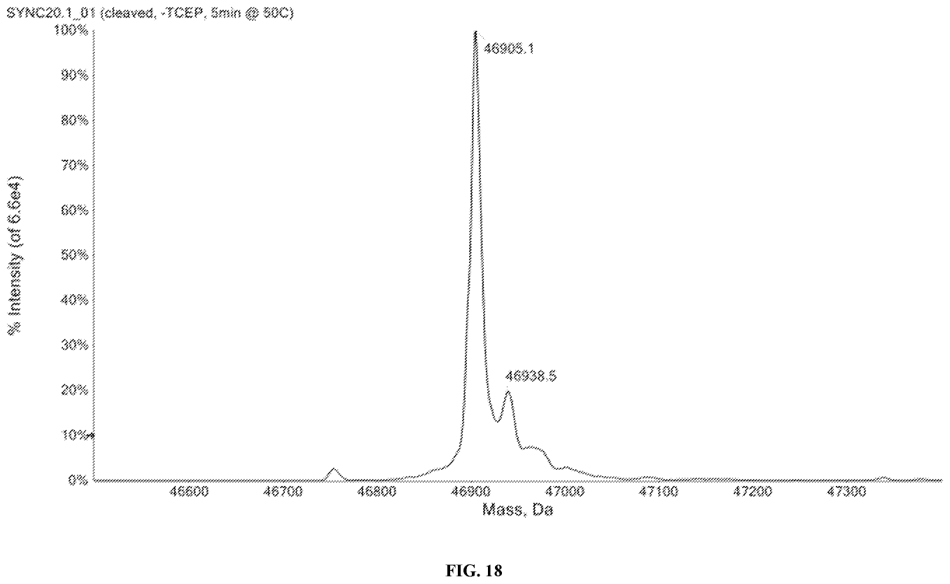

Separation by SDS-PAGE followed by Coomassie Blue staining showed that, with the exception of SYNC27-1, the synthetic HLA proteins tested migrate as a single band under non-reducing conditions suggesting homogeneity of disulfide bond formation for all proteins listed in Table 15. Results of the SDS-PAGE analysis for representative constructs are shown in FIG. 17. SYNC27-1 contains two extra cysteines which are predicted to create a disulfide staple between residue 84 of the HLA domain (Y84C) and the second residue of linker 1. It is predicted that the presence of these two extra cysteine residues have resulted in a proportion of the recombinant protein containing mis-paired disulfide bonds, evidenced in a doublet by SDS-PAGE under non-reducing conditions.

Depending on the downstream application of recombinant proteins, they were subjected to TEV protease cleavage or biotinylation according to standard protocols. Briefly, TEV protease was incubated with the synthetic HLA proteins at a ratio of 1:100 w/w overnight at 4° C. and separated via size exclusion chromatography. Biotinylation of the AviTag™ was achieved by incubating the synthetic HLA proteins with bacterially expressed recombinant GST-labelled BirA in the presence of 10 mM ATP, 10 mM MgCl2 and 50 μM D-biotin at 4° C. overnight. BirA was separated from the reaction mixture using Glutathione Sepharose 4B resin. Biotinylated synthetic HLA proteins were further purified by size exclusion chromatography.

TOF-MS experiments revealed that the observed masses matched the expected masses, confirming the cleavage of the initiator methionine and absence of the +305 Da S-glutathionylation after removal of the extra unpaired cysteine. The expected and observed masses are listed in Table 16 and a representative spectrum is shown in FIG. 18 (SYNC20-1, after cleavage of His-tag with TEV protease). The purification strategy, downstream processing, yields and analysis are summarized in Table 16.

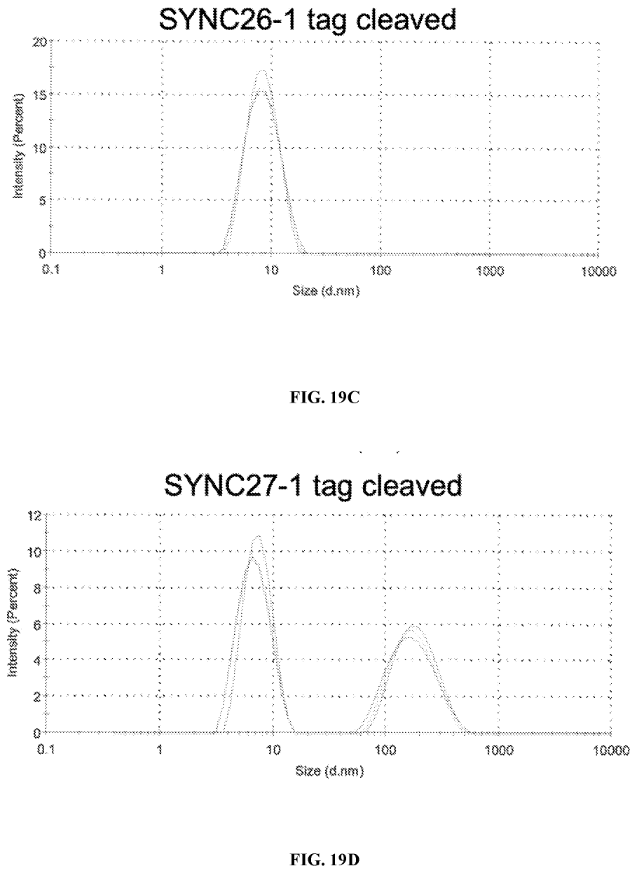

To assess the homogeneity of the protein sample, DLS was used to analyze SYNC25-1, SYNC26-1 and SYNC27-1 according to standard protocols. Briefly, samples were added to the sample cuvette and analyzed in the Zetasizer. FIGS. 19A-19F show single peaks indicative of a homogenous protein sample for SYNC25-1 and SYNC26-1, while double peaks were observed for SYNC27-1. This indicates there may be more than one protein species present in the sample, potentially a proportion of unfolded material, consistent with the doublet observed on a gel for SYNC27-1. The results are summarized in Table 16.

TABLE 15
List of synthetic HLA proteins (control and test) expressed
in bacteria harboring R1S and C1G mutations.

| | | | | | | Mutations | | |
|---|---|---|---|---|---|---|---|---|
| | SEQ ID | | Linker | Linker | | | | Q226A, |
| Name | NO | Peptide | L1* | L2^{&} | [symbol] | C1G^{#} | Y84^{$} | D227K^{$} |
| SCTC3-1 | 14 | SYRPGTVAL | L | L | His | G | WT | WT |
| | | (SEQ ID NO: | | | | | | |
| | | 160) | | | | | | |
| SYNC19- | 71 | SYRPGTVAL | L | L | His | G | A | WI |
| 1 | | (SEQ ID NO: | | | | | | |
| | | 160) | | | | | | |
| SYNC19- | 72 | SYRPGTVAL | L | L | His- | G | A | WT |
| 2 | | (SEQ ID NO: | | | AVI | | | |
| | | 160) | | | | | | |
| SYNC20- | 74 | SYRPGTVAL | L | L | His | G | A | AK |
| 1 | | (SEQ ID NO: | | | | | | |
| | | 160) | | | | | | |
| SYNC22- | 80 | SYRPGTVAL | M | L | His | G | A | WT |
| 1 | | (SEQ ID NO: | | | | | | |
| | | 160) | | | | | | |
| SYNC22- | 81 | SYRPGTVAL | M | L | His- | G | A | WT |
| 2 | | (SEQ ID NO: | | | AVI | | | |
| | | 160) | | | | | | |
| SYNC23- | 84 | SYRPGTVAL | M | L | His | G | A | AK |
| 1 | | (SEQ ID NO: | | | | | | |
| | | 160) | | | | | | |
| SYNC25- | 90 | SYRPGTVAL | S | L | His | G | A | WT |
| 1 | | (SEQ ID NO: | | | | | | |
| | | 160) | | | | | | |
| SYNC25- | 91 | SYRPGTVAL | S | L | His- | G | A | WT |
| 2 | | (SEQ ID NO: | | | AVI | | | |
| | | 160) | | | | | | |
| SYNC26- | 93 | SYRPGTVAL | S | L | His | G | A | AK |
| 1 | | (SEQ ID NO: | | | | | | |
| | | 160) | | | | | | |
| SYNC27- | 96 | SYRPGTVAL | S + C | L | His | G | C | WT |
| 1 | | (SEQ ID NO: | | | | | | |
| | | 160) | | | | | | |

*Linker 1 (L1) length, L = Long (GGGGSGGGGSGGGGS; SEQ ID NO: 179), M = Medium (GGGGSGGGGSGS; SEQ ID NO: 177), S = Short (GGGGSGGGGS; SEQ ID NO: 175), + C = glycine in position 2 of the LI linker mutated to cysteine.
*Linker 2 (L2) length, L = Long (GGGGSGGGGSGGGGS; SEQ ID NO: 183), M = Medium (GGGGSGGGGSGS; SEQ ID NO: 177)

^{S}WT=wild type sequence of Q226, D227 or Y84. \hat{ }Protein tags: His, Hexahis tag; AVI, AviTag™. A TEV protease cleavage site is included N-terminal to the Hexahis tag. ^{#}First position in the HLA domain.

TABLE 16
Expression, refold and purification of recombinant HLA proteins.

| | | | | | | | | | Purification | | | |
|---|---|---|---|---|---|---|---|---|---|---|---|---|
| | | | | | | | | Refold- | Strategy | | | DLS^{$} |
| | Culture | IB | Refold | | | Purification | | Purification | and | Mass Spec MW (Da) | | Single |
| | vol. | yield | Pre | Post | Recovery | Conc. | Total | efficiency | downstream | Non-Reduced | | Peak |
| Name | (L) | (mg) | (mg) | (mg) | (%) | (mg/ml) | (mg) | (%) | processing* | Expected | Observed | (Y/N/N.D.) |
| SYNA1-1 | 4 | 220 | 100 | 72 | 72 | 2.86 | 0.57 | 0.57 | Ni, MQ, S | 47937.8 | 47938.6 | N.D. |
| SCTC3-1 | 2 | 48 | 48 | 40 | 83 | 9.6 | 1.5 | 3.13 | D, S, C, | 47039.6 | 47040.6 | N |
| | | | | | | | | | Ni(r), S | | | (2 peaks) |
| SCTC3-2 | 2 | 92 | 92 | 72 | 78 | 11.8 | 2.6 | 2.83 | D, B, S | 50013.5 | 50015.6 | N.D. |
| SYNC19-1 | 2 | 78 | 78 | 54 | 69 | 1.9 | 0.53 | 0.68 | Ni, MQ, S | 47827.4 | 47828.7 | N.D. |
| SYNC1-2 | 2 | 96 | 96 | 57 | 59 | 16.5 | 2.48 | 2.60 | D, B, S | 49921.4 | 49923.4 | N.D. |
| SYNC20-1 | 2 | 74 | 74 | 62 | 84 | 7.7 | 1.5 | 2.03 | D, S, C, | 46903.5 | 46905.1 | N.D. |
| | | | | | | | | | Ni(r), S | | | |
| SYNC22-1 | 2 | 101 | 100 | 93 | 93 | 0.5 | 0.72 | 0.50 | Ni, MQ, S | 47656.2 | 47657.5 | N.D. |
| SYNC22-2 | 2 | 68 | 68 | 53 | 78 | 7.2 | 1.36 | 2.00 | D, B, S | 49759.3 | 49752.6 | N.D. |
| SYNC23-1 | 2 | 80 | 80 | 62 | 78 | 8.7 | 1.9 | 2.38 | D, S, C, | 46732.4 | 46733.5 | N.D. |
| | | | | | | | | | Ni(r), S | | | |
| SYNC25-1 | 3 | 127 | 100 | 93 | 93 | 14.3 | 2.3 | 2.30 | D, C, S, S | 46632.2 | 46633.7 | Y |
| SYNC25-2 | 2 | 92 | 92 | 57 | 62 | 8.7 | 1.48 | 1.60 | D, B, S | 49606.1 | 49608.2 | N.D. |
| SYNC26-1 | 2 | 95 | 95 | 72 | 76 | 16.7 | 3.6 | 3.79 | D, C, S, | 46588.2 | 46589.9 | Y |
| | | | | | | | | | Ni(r), S | | | |
| SYNC27-1 | 2 | 60 | 60 | 40 | 67 | 5.4 | 1.09 | 1.82 | D, S, C, | 46664.4 | 46665.8 | N |
| | | | | | | | | | Ni(r), S | | | (2 peaks) |
| SCD2-1/ | 2/2 | 163/99 | 100/50 | 105 | 70 | 19.6 | 3.2 | 2.13* | D, S, C, | 45718.5 | 45737.8 | Y |
| HHCC1-2 | | | | | | | | | Ni(r), S | | | |
| WTC1 | 2/2 | 110/500 | 50/65 | 99 | 94 | 14.5 | 8 | 7.6 | MQ, S, | 11860.3 | 11860.8 | Y |
| (RL9^{@}/HLA- | | | | | | | | | Ni(r) | (B2M) | (B2M) | |
| C*07:02/B2M) | | | | | | | | | | 31829.9 | 32135.6 | |
| | | | | | | | | | | (HLA- | (HLA- | |
| | | | | | | | | | | C*07:02) | C*07:02)^{#} | |

*Ni (NiNTA IMAC), Ni(r) (NiNTA resin clearing), D (DEAE AEX), MQ (monoQ AEX), DS (desalt), S (SEC), B (biotinylation) and C (protease tag-cleavage).
^{$}DLS, dynamic light scatter; N.D., not done.
^{@}RL9 = RL9 peptide, RYRPGTVAL (SEQ ID NO: 159).
^{#}+305 Da S-glutathionylation

### Example 12: Recombinant Bacterial Expression of Immune Receptors

Synthetic immune receptor proteins as described herein (as listed in Table 17) were expressed, isolated, refolded, purified, and analyzed as described in Examples 8 and 9. Briefly, DNA encoding immune receptors was inserted into an expression vector, then introduced into bacteria under antibiotic selection. The proteins contain tags described in Table 17. Cells harboring the appropriate sequences were grown to mid-log phase, and expression was induced with isopropyl B-D-1-thiogalactopyranoside (IPTG). Cells were harvested by centrifugation, lysed using a high-pressure homogenizer and centrifuged to isolate the soluble and insoluble fractions. Insoluble inclusion bodies containing immune receptor proteins were washed and resuspended in a denaturing buffer. Recombinant immune receptors proteins were refolded from the inclusion body preparation by dilution in a refold buffer, followed by incubation at 4° C. for up to 72 hours. The resulting protein was purified by a combination of IMAC, SEC, or anion exchange chromatography. Depending on the downstream application of recombinant immune receptors, they were subjected to PreScission protease cleavage or biotinylation according to standard protocols. Briefly, bacterially expressed recombinant GST-labelled PreScission protease was incubated with the immune receptors at a ratio of 1:100 w/w and incubated overnight at 4° C. The PreScission protease was separated from the reaction mixture using Glutathione Sepharose 4B resin. Cleaved immune receptors were further purified by size exclusion chromatography. Briefly, biotinylation of the AVI-tagged immune receptors was achieved by incubating with bacterially expressed recombinant GST-labelled BirA in the presence of 10 mM ATP, 10 mM MgCl2 and 50 μM D-biotin at 4° C. overnight. BirA was separated from the reaction mixture using Glutathione Sepharose 4B resin. Biotinylated immune receptors were further purified by size exclusion chromatography.

The purification strategy, downstream processing, yields and analysis are summarized in Table 18.

TABLE 17
Soluble immune receptor constructs.

| | SEQ ID | | | |
|---|---|---|---|---|
| Name | NO | Residues | Mutation | Tags^{\hat{ }} |
| CD8α_RP01 | 187 | 1-119 | WT | His- |
| | | | | AVI |
| CD8α_RP10 | 188 | 1-119 | C34S | His- |
| | | | | AVI |
| CD8α_RP11 | 189 | 1-119 | C34A | His- |
| | | | | AVI |
| KIR2DL2_RP02 | 190 | 1-206 | WT | His- |
| | | | | AVI |
| KIR2DL2_RP13 | 191 | 1-206 | C149A | His- |
| | | | | AVI |
| LILRB1_RP14 | 193 | 24-222 | WT | His- |
| | | | | AVI |

^{\hat{ }}Protein tags: His, Hexahis tag; Avi, AviTag ™. A PreScission protease site is included N-terminal to the Hexahis tag.

TABLE 18
Expression, refold and purification of soluble immune receptors.

| | | | | | | | | Refold- | | | | DLS |
|---|---|---|---|---|---|---|---|---|---|---|---|---|
| | Culture | IB | Refold | | | Purification | | Purification | | Mass Spec MW (Da) | | Single |
| | vol. | yield | Pre | Post | Recovery | Conc. | Total | efficiency | Purification | Non-Reduced | | Peak |
| Name | (L) | (mg) | (mg) | (mg) | (%) | (mg/ml) | (mg) | (%) | Strategy | Expected | Observed | (Y/N/N.D.) |
| CD8α_RP01 | 2 | 228 | 228 | 18 | 8 | 1.6 | 2.2 | 0.96 | Ni, S, B, S | 17456.4 | 17456.6 | N.D. |
| CD8α_RP10 | 2 | 97 | 97 | 16 | 17 | 3.3 | 1.0 | 1.03 | Ni, S, C, | 14078.0 | N.D. | N.D. |
| (cleaved) | | | | | | | | | Ni(r) | | | |
| CD8α_RP11 | 2 | 179 | 100 | 33 | 33 | 13.0 | 4.2 | 4.20 | Ni, S, C, | 14062.0 | 14062.6 | N.D. |
| (cleaved) | | | | | | | | | Ni(r), S | | | |
| CD8α_RP11 | 2 | 123 | 100 | 36 | 36 | 12.0 | 8.4 | 8.40 | Ni, S | 17198.3 | 17198.9 | N.D. |
| KIR2DL2_RP02 | 2 | 162 | 162 | 138 | 85 | 2.3 | 3.4 | 6.60 | Ni, S, | 26797.5 | 27103.3 | N.D. |
| | | | | | | | | | split(5.3 mg), | | | |
| | | | | | | | | | B, S | | | |
| KIR2DL2_RP02 | | | | | | 11.4 | 7.3 | | Ni, S, | 23435.2 | 23740.8 | Y |
| (cleaved) | | | | | | | | | split(15 mg), | | | |
| | | | | | | | | | C, S | | | |
| KIR2DL2_RP13 | 3 | 170 | 100 | 71 | 71 | 11.3 | 4.9 | 4.90 | Ni, S, C, S | 23403.2 | 23404.0 | N.D. |
| (cleaved) | | | | | | | | | | | | |
| KIR2DL2_RP13 | 3 | 187 | 100 | 66 | 66 | 18.0 | 5.8 | 5.80 | Ni, S | 26408.3 | 26540.2 | N.D. |
| KIR2DL3_RP03 | 2 | 138 | 138 | 185 | 134 | 2.2 | 2.2 | 6.67 | Ni, S, | 26778.5 | 26778.9 | Y |
| | | | | | | | | | split(4.4 mg), | | | (side peak) |
| | | | | | | | | | B, S | | | |
| KIR2DL3_RP03 | 3 | 187 | 100 | 66 | 66 | 17.5 | 7.0 | 5.80 | Ni, S, | 23416.2 | 23416.5 | N.D. |
| (cleaved) | | | | | | | | | split(13 mg), | | | |
| | | | | | | | | | C, S | | | |
| LILRB1_RP14 | 2 | 120 | 120 | 40 | 33 | 1.3 | 0.47 | 0.39 | Ni, S, S | 23911.8 | 23910.0 | N.D. |

Ni (NiNTA IMAC), Ni(r) (NiNTA resin clearing), D (DEAE AEX), MQ (monoQ AEX), DS (desalt), S (SEC), B (biotinylation) and C (protease tag-cleavage).

### Example 13: Thermal Stability Assay

A thermal stability assay was carried out to monitor the unfolding of proteins by applying a thermal gradient from 10-100° C. at 1.0° C. per minute on a Bio-Rad CFX96™ Real-Time System RT PCR. Protein unfolding was measured by monitoring the increase in signal from a fluorescent dye, SYPRO™ orange, which binds to hydrophobic regions of proteins as they unfold. Proteins were assayed at 5 μM and were measured individually and in combination as described below. Determination of the melting point, Tₘ (the temperature of the midpoint of the melt transition), of the proteins was done using the Bio-Rad CFX manager software. The first derivative (negative mode) of the protein melt curve identifies maxima that correspond to the Tₘ, a one-step protein unfolding event would have a single maximum, while a two-step unfolding event would have two maxima, and so on.

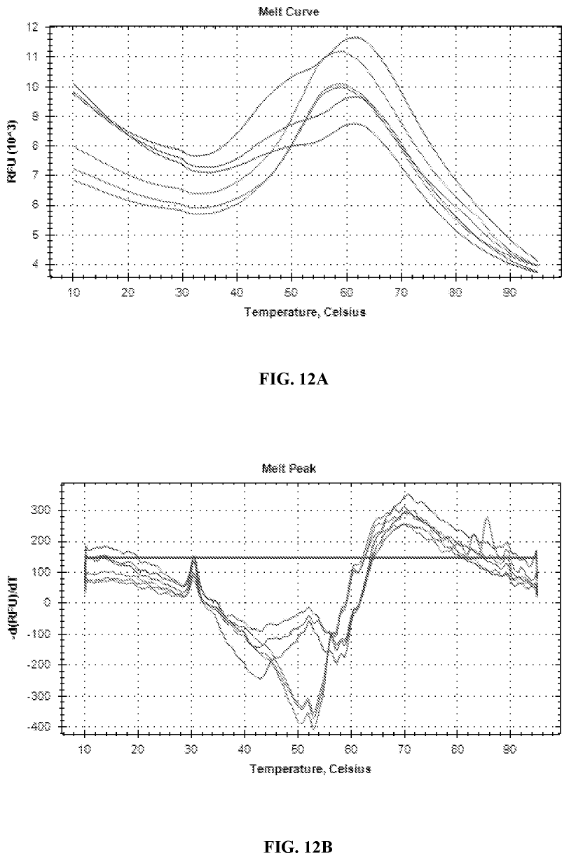

The melt curve of SYNC4-1 showed a pronounced two-step unfolding event (FIG. 12A), corresponding to a Tₘ of 42.6° C. & 57.2° C., while the combination of SYNC4-1 and KIR2DL2 results in a major shift to the right of the first maximum corresponding to a Tₘ of 54.8° C. and 57.4° C. The pronounced double maxima of SYNC4-1 alone became a condensed double peak (FIG. 12B). The shift in Tₘ to the right suggests the KIR2DL2 stabilized SYNC4-1 through protein-protein interaction.

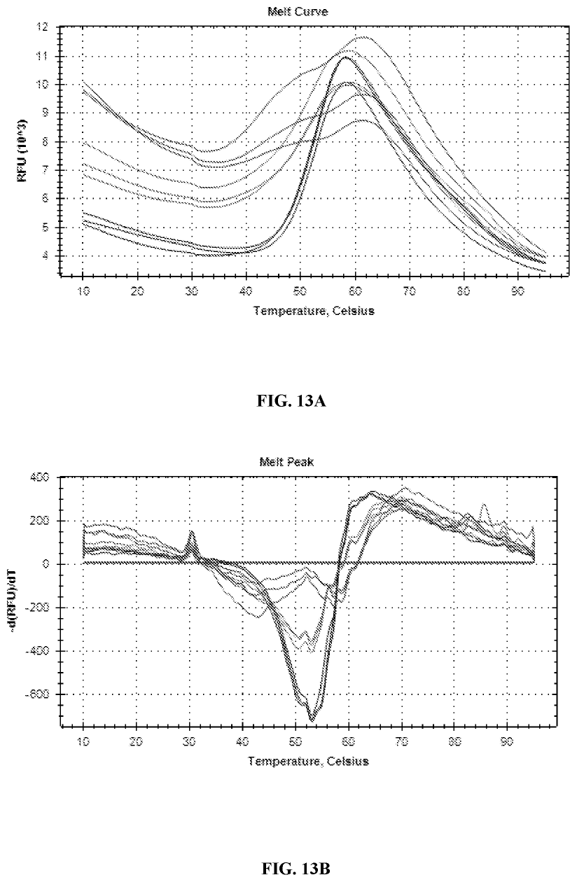

In comparison to the KIR2DL2 melt curve and first derivative (FIG. 13), the two maxima have further condensed to a single maximum with a slight shoulder on the left hand side, which correspond to a Tₘ 58.8° C. and 57.6° C. respectively. Once again, this strongly suggests there is a protein-protein interaction between SYNC4-1 and KIR2DL2.

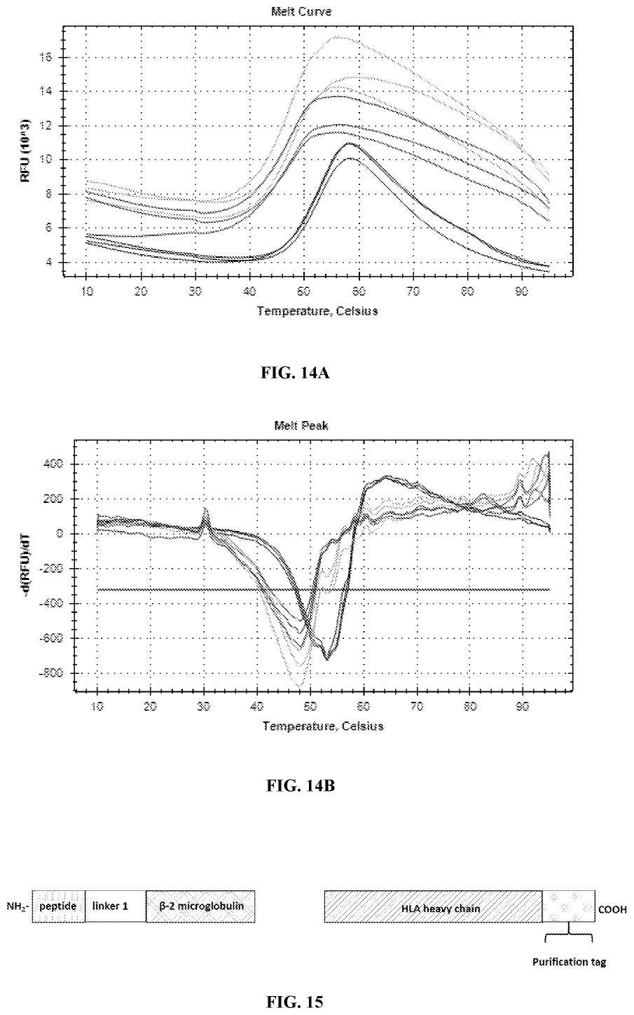

In contrast to SYNC1-1, SYNA1-1 which has the influenza peptide GILGFVFTL and a relatively long linker sequence that may interfere with KIR-binding, did not lead to a shift to the right when combined with KIR2DL2 (FIG. 14), indicating KIR2DL2 did not increase the thermal stability of SYNA1-1 (SYNA1-1 Tₘ=48° C., SYNA1-1+KIR2DL2 Tₘ=48.2° C.). Instead, the thermal stability of KIR2DL2 was reduced when combined with SYNA1-1 (FIG. 14), Tm of 58° C. and 48° C. respectively. These data suggest that there was no increase in thermal stability when combining SYNA1-1 with KIR2DL2, suggesting little to no interactions between the two proteins.

### Example 14: Interaction of Soluble Synthetic HLA Proteins with Immune Receptors

To investigate the ability of soluble synthetic HLA proteins to evade the immune system, their interaction with immune receptors is examined. Recombinant immune receptors such as the killer cell immunoglobulin-like receptor (KIR) on natural killer cells, and the T-cell receptor and CD8 co-receptor on T cells are tested. Standard protein-protein interaction techniques such as surface plasmon resonance, enzyme linked immunosorbent assay (ELISA), thermal melt assays, and circular dichroism are used to investigate this interaction. When compared to controls, soluble synthetic HLA proteins harboring specific mutations exhibit impaired binding to recombinant activating immune receptors and maintained or improved binding to inhibitory receptors.

To examine the interaction of soluble synthetic HLA proteins with immune receptors at the cell surface, competitive cellular assays are used. In these assays, soluble synthetic HLA proteins are incubated with mammalian cells expressing wild type Class I HLA molecules. The ability of the wild type cells to survive in the presence of immune cells (e.g. T cells and/or NK cells) is then measured. When compared to controls, soluble HLA proteins harboring specific mutations are less reactive with the activating receptors on immune cells and exhibit maintained or improved reactivity with inhibitory receptors, resulting in increased killing of the wild type mammalian cells.

### Example 15: Production of Recombinant HLA Proteins from Separate Transcriptional Units

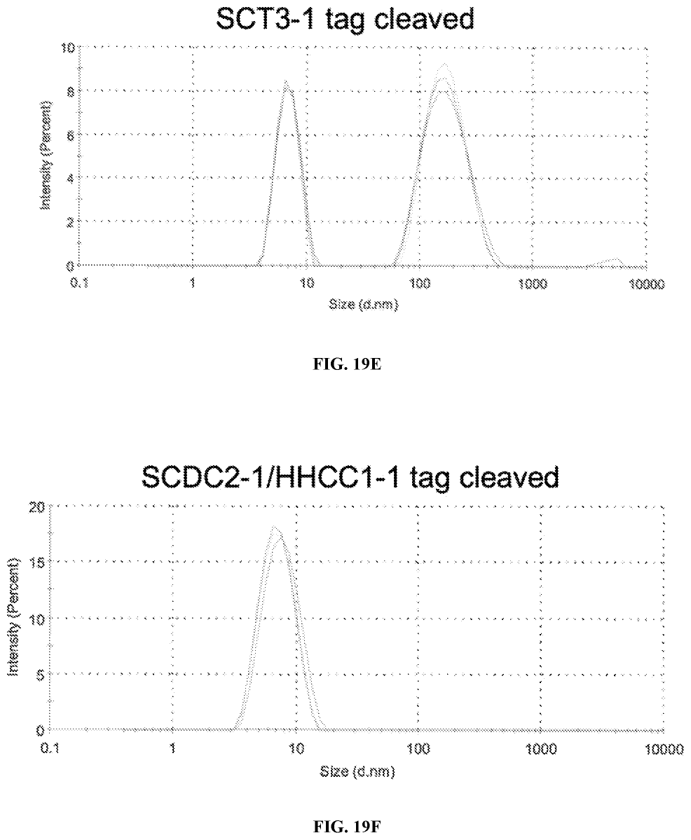

To potentially overcome the heterogeneity observed when a disulfide staple was introduced into SYNC27-1 (FIG. 17), synthetic HLA proteins were produced from separate transcriptional units to create the same protein lacking linker 2 (SCD2-1/HHCC1-1; SEQ ID NOs: 21/131). DNA encoding either the SCD2-1 or HHCC1-1 were inserted into a vector, then introduced into bacteria under antibiotic selection. The construct format was as described in FIG. 15. Briefly, the peptide and beta-2 microglobulin domains were expressed as a single protein joined by a linker (linker L1) (SCD2-1, SEQ ID NO: 21), and the HLA heavy chain domain (HHCC1-2, SEQ ID NO: 132) was produced as a separate protein. Cells harboring the appropriate constructs (SCD2-1 and HHCC1-2, described in Table 19) were grown to mid-log phase, and expression was induced with isopropyl B-D-1-thiogalactopyranoside (IPTG). Cells were harvested by centrifugation, lysed using a cell crusher and centrifuged to isolate the soluble and insoluble fractions. Insoluble inclusion bodies containing the appropriate domains of the synthetic HLA proteins were washed and resuspended in a denaturing buffer. The peptide and B-2 microglobulin protein was first refolded for 30 minutes before the HLA heavy chain domain was added to the refold solution. The resulting refolded protein was purified by a combination of IMAC, SEC and/or anion exchange chromatography. The purification strategy, yields, and analysis (TOF-MS, DLS) are summarized in Table 16. Coomassie Blue staining showed that the assembled final product separates into the two separate polypeptide chains only under reducing conditions and migrates as a single species under non-reducing conditions, suggesting it is correctly folded (FIG. 20).

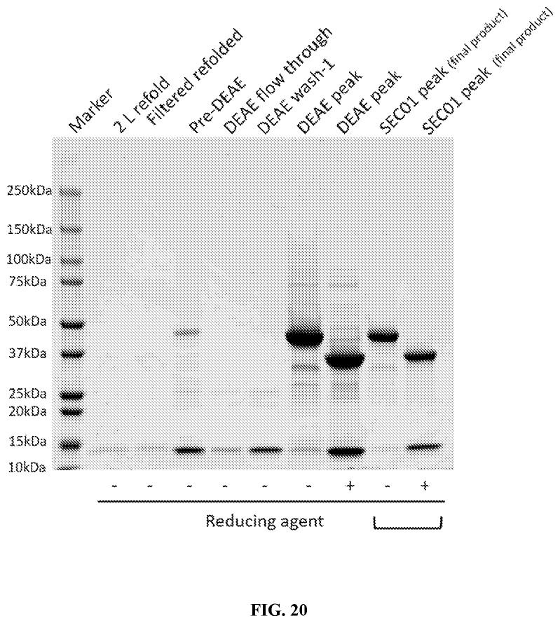

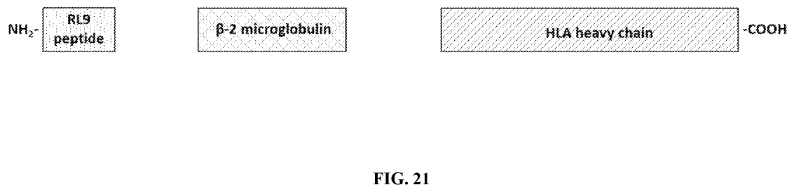

Additionally, a wild type class I HLA protein (WTC1) was using a similar approach to that described above, except the peptide B-2 microglobulin domain was produced without linker 1 and without the peptide domain. The construct format was as described in FIG. 21 and comprises a peptide domain (RL9 peptide, SEQ ID NO: 159), B-2 microglobulin domain (B2M, SEQ ID NO: 195) and HLA domain (HLA-C*07:02, SEQ ID NO: 194). Protein was produced as described above, but with the addition of 10 μM of the RYRPGTVAL (SEQ ID NOL 195) peptide (synthesized by Mimotopes, Melbourne, Australia) into the refold solution. Results of the SDS-PAGE analysis are shown in FIG. 22. Separation by SDS-PAGE followed by Coomassie blue staining shows that the assembled final product migrates as two species marginally smaller than the expected molecular weight under non-reducing conditions and at the expected molecular weight under reduced conditions, suggesting it is correctly folded (FIG. 22). The purification strategy, yields, and analysis (TOF-MS, DLS) are summarized in Table 16. The mass of the HLA domain was consistent with S-glutathionylation of a cysteine residue.

To assess the homogeneity of the protein samples, DLS was used to analyze SCD2-1/HHCC1-1 and WTC1 according to standard protocols. Briefly, samples were added to the sample cuvette and analyzed in the Zetasizer. FIGS. 19A and 19F show that a single peak indicative of a homogenous protein sample was observed for WTC1 and SCD2-1/HHCC1-1. The results are summarized in Table 16.

TABLE 19
List of recombinant HLA proteins (control and test)
expressed from separate transcriptional units.

| | SEQ ID | | Linker | Linker | | Mutations | | Q226A, |
|---|---|---|---|---|---|---|---|---|
| Name^{&} | NO. | Peptide | L1 | L2 | [symbol] | C1G^{#} | Y84^{$} | D227K^{$} |
| SCD2-1/ | 21/132 | SYRPGTVAL | S + C | none | His- | G | C | WT |
| HHCC1-2 | | | | | AVI | | | |
| WTC1 | 159/194/ | RYRPGTVAL | n/a | n/a | n/a | WT | WT | WT |
| (RL9^{@}/ | 195 | | | | | | | |
| HLA-C*07: | | | | | | | | |
| 02/B2M) | | | | | | | | |

*Linker 1 (L1) length, L = Long (GGGGSGGGGSGGGGS; SEQ ID NO: 179), M = Medium (GGGGSGGGGSGS; SEQ ID NO: 177), S = Short (GGGGSGGGGS; SEQ ID NO: 175), + C = glycine in position 2 of the LI linker mutated to cysteine.
*Linker 2 (L2) length, L = Long (GGGGSGGGGSGGGGS; SEQ ID NO: 183), M = Medium (GGGGSGGGGSGS; SEQ ID NO: 177).
^{$}WT = wild type sequence of Q226, D227 or Y84. [symbol]
Protein tags: His, Hexahis tag; AVI, AviTag ™.
A TEV protease cleavage site is included N-terminal to the Hexahis tag.
^{#}First position in the HLA domain.
^{@}RL9 = RL9 peptide, RYRPGTVAL (SEQ ID NO: 159).

### Example 16: Analysis of Interaction of Synthetic HLA Constructs and KIR Proteins by Surface Plasmon Resonance

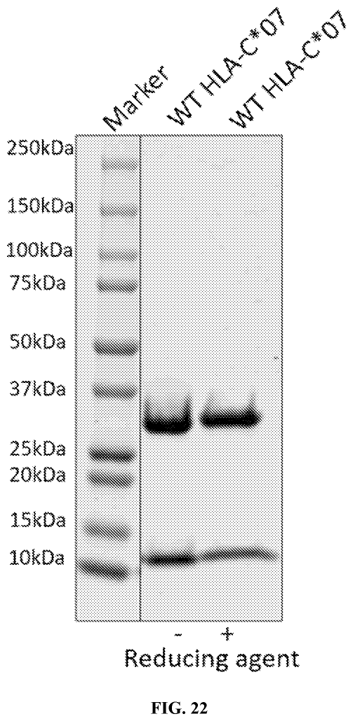

The interaction of synthetic HLA constructs with KIR immune proteins was examined by SPR according to standard protocols. Briefly, histidine-tagged KIR proteins (ligand) were immobilized via Nickel-NTA coupling with a nitrilotriacetic acid (NTA)-derivatized carboxymethyldextran sensor chip (NiD50L, XanTec Bioanalytics). Recombinant HLA proteins (analyte) (SYNC20-1, SYNC23-1, SYNC25-1, SYNC26-1, SCD2-1/HHCC1-2, and the wild type control protein WTC1) were then flowed over the surface producing a sensorgram which is a graph of time-traced SPR responses during a biomolecular interaction analysis (FIG. 23 shows a representative example). All experiments were performed at 25° C., where both analyte and ligand were diluted in the SPR buffer (20 mM HEPES pH 7.5, 100 mM NaCl, 50 μM EDTA, 0.05% (v/v) tween-20). The flow cells were enhanced with 500 μM NiCl₂ (10 μL×min⁻¹, 1 min).

Before use in SPR assays, synHLA proteins (SYNC20-1, SYNC23-1, SYNC25-1, SYNC26-1, SCD2-1/HHCC1-2) were modified by TEV cleavage as described in Example 4. Briefly, TEV protease was incubated with the synthetic HLA proteins at a ratio of 1:100 w/w overnight at 4° C. and separated via size exclusion chromatography.

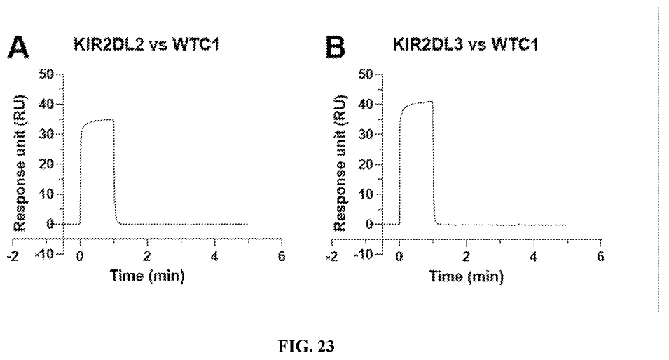

The response of the analyte injections at the end of the equilibrium phase were double referenced against reference cell and blank injection values before normalization corresponding to their respective ligand immobilization response. The relative response of each interaction are shown in FIG. 24. The SPR data shows all synHLA proteins tested bound to KIR2DL2 and KIR2DL3, albeit with a reduced relative response compared to WTC1 (FIG. 24).

The synHLA proteins with the highest relative responses to KIR2DL2 were SYNC26-1 and SYNC23-1. Both of these proteins contained four point mutations in the HLA heavy-chain (C1G, Y84A, Q226A, D227K), a 15-residue linker 2, and either a short (SYNC26-1) or medium (SYNC23-1) linker 1. When a longer linker 1 was introduced into the same protein backbone to create SYNC20-1, reduced binding to KIR2DL2 was observed. This suggests that the presence of a longer linker 1 may impair binding to KIR2DL2.

When SYNC26-1 was produced without the CD8 mutations (SYNC25-1, wt Q226, D227), reduced binding to KIR2DL2 was observed. Binding to KIR2DL2 was further decreased when a disulfide staple was introduced into the same construct lacking linker 2 (compare SYNC25-1 and SCD2-1/HHCC1-2), suggesting that the presence of this disulfide staple and/or lack of linker 2, may impair KIR2DL2 binding.

Similar overall trends to those described above were observed for binding of synHLA proteins to KIR2DL3; however, smaller differences between the proteins were generally observed and error bars were overlapping.

### Example 17: Generation of B2M^{-/-} EBV-Transformed B-Cell Lines

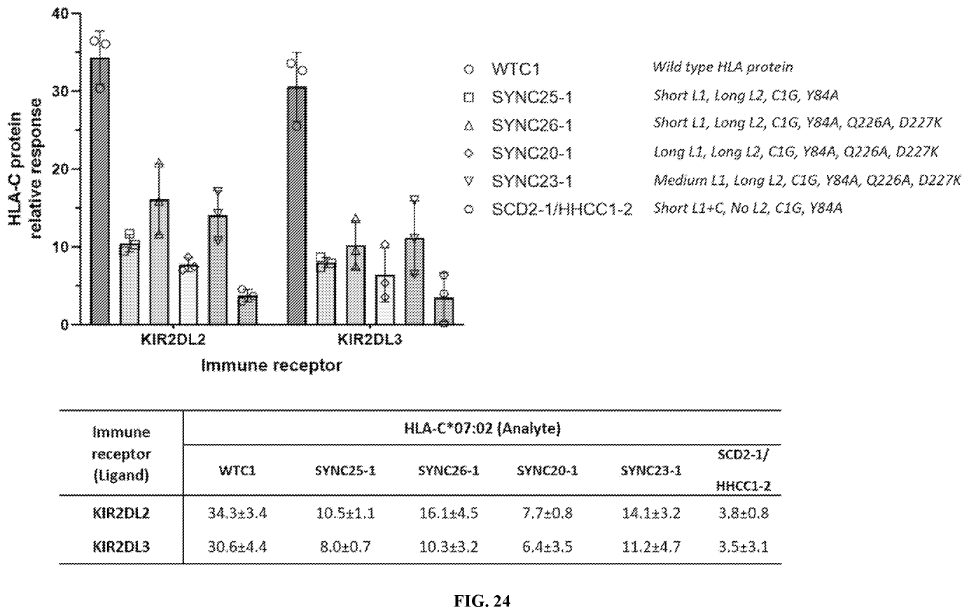

CRISPR/Cas9 was used to mutate B2M in 2 EBV lines (9031 and JK) such that B2M expression was ablated. EBV cells were transfected with Cas9 construct and HLA class I low/negative EBV cells were sorted. The cells were grown, and the absence of surface HLA class I was confirmed by antibody staining using anti-HLA antibody W6/32. Binding of W6/32 to the cell surface was detected with phycoerythrin labelled goat anti-mouse antibody (GAM-PE), as illustrated in FIG. 25. After three rounds of selection, homogeneous class I deficient lines were obtained. The left-hand column of FIG. 25 shows the class I and negative control staining of unmodified EBV cells (referred to as wild type). The right-hand column of FIG. 25 shows equivalent staining of B2M deficient cells. The numbers on the plots are the mean fluorescent intensities (MFI) of the staining. The negative control is staining in the absence of the primary antibody (W6/32) but in the presence of the secondary antibody alone (GAM-PE. The shaded plots indicate the staining profile negative control staining (no primary antibody). The open histograms with solid black borders represent the staining profile with W6/32 detected by GAM-PE.

### Example 18: Relative Expression of Synthetic HLA Proteins on the Surface of B2Mnull EBV Cells

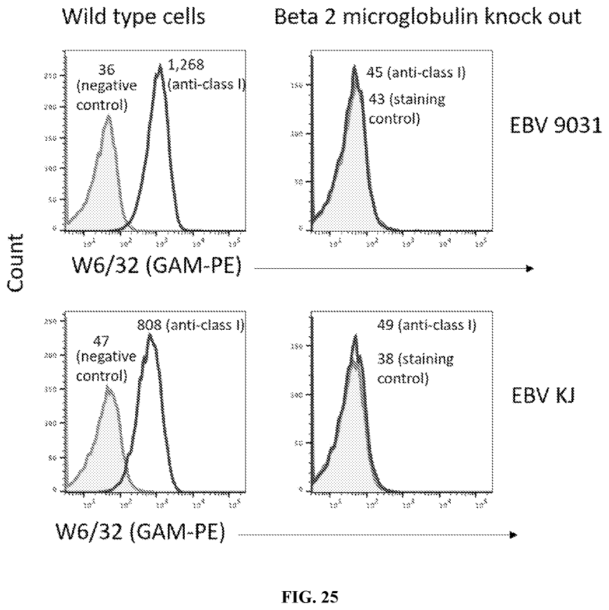

DNA encoding synthetic HLA protein controls as described herein (SCTA2-M1, SEQ ID NO: 5; SCDA1-M1, SEQ ID NO: 17) were cloned into the EBV episomal vector pCE. The vector harboring the constructs was introduced into B2M null EBV cells by electroporation according to standard protocols, followed by antibiotic selection. The expression of synthetic HLA protein controls at the cell surface was then examined by antibody staining and fluorescence activated cell sorting (FACS) (FIG. 26). Expression of SCTA2-M1 and SCTDA1-M1 was detected in approximately 20 and 40% of the cell population respectively, demonstrating that synthetic HLA protein controls can be expressed at the cell surface.

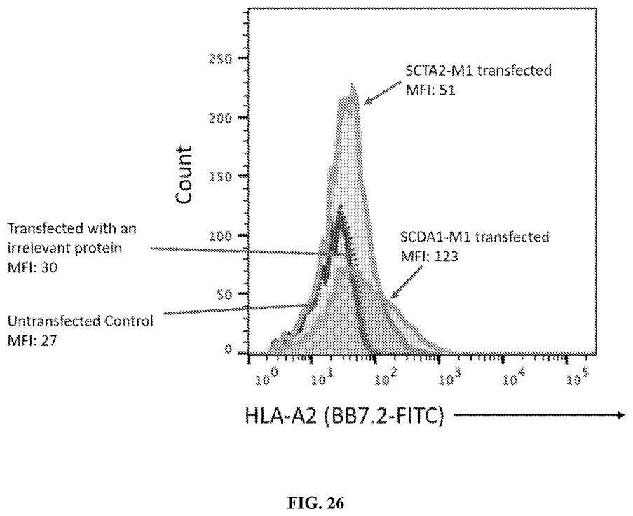

To facilitate more rapid reporting of successful transfection, the EBV episomal vector pCE (Addgene #41858) was modified to encode a fluorescent reporting protein (green fluorescent protein, GFP) within a multi-gene expression system (Lewis, Jo E., et al. “The use of a viral 2A sequence for the simultaneous over-expression of both the vgf gene and enhanced green fluorescent protein (eGFP) in vitro and in vivo.” Journal of neuroscience methods 256 (2015): 22-29, which is incorporated herein by reference in its entirety). The construct format and dominant products are as shown in FIG. 27, with GFP fluorescence providing a readout of successful transfection.

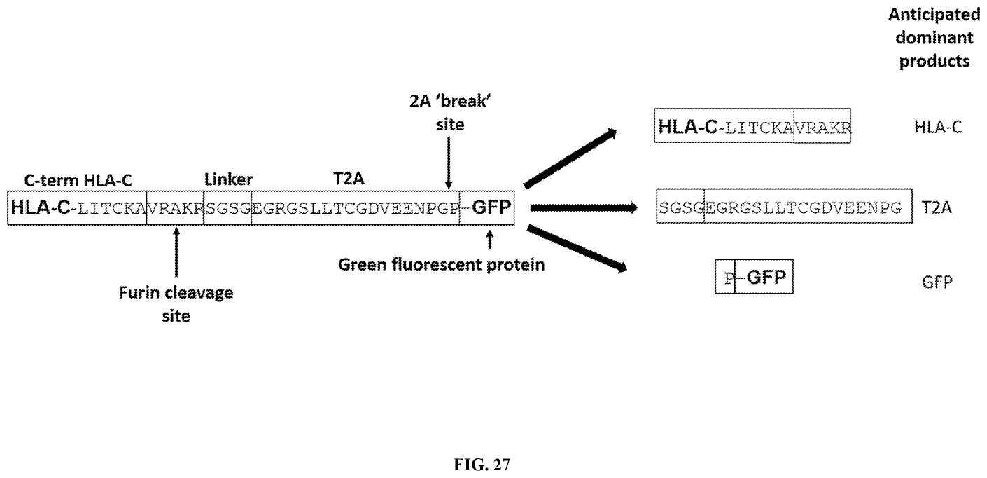

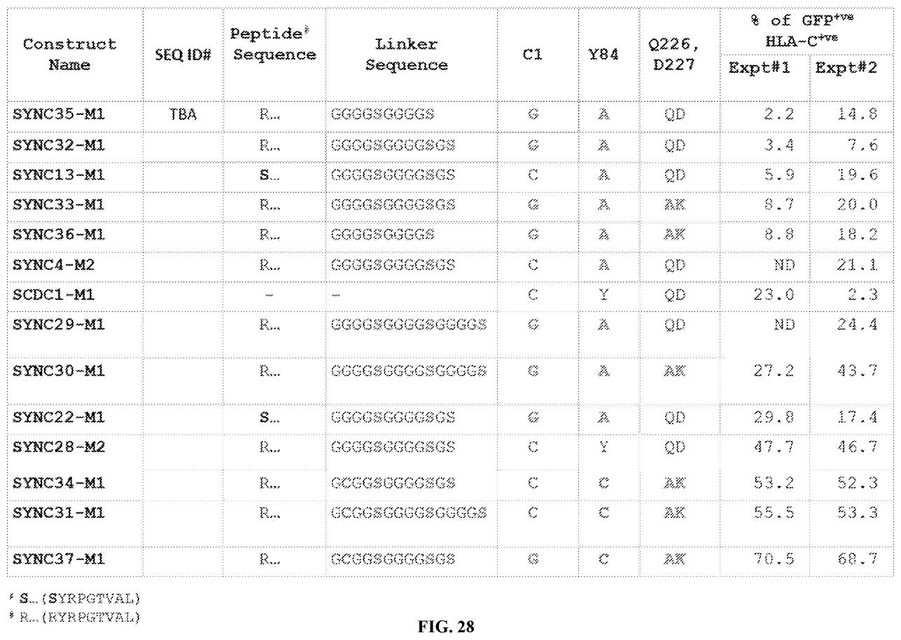

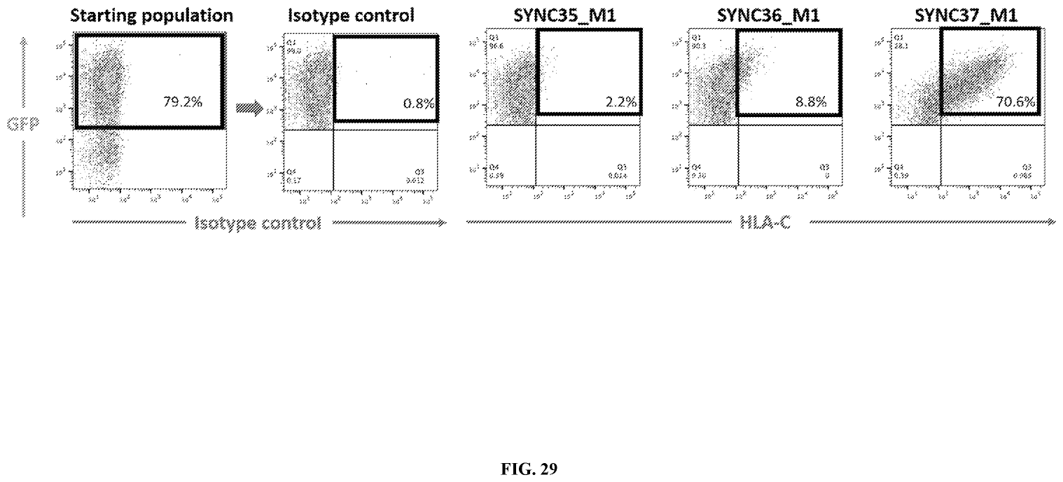

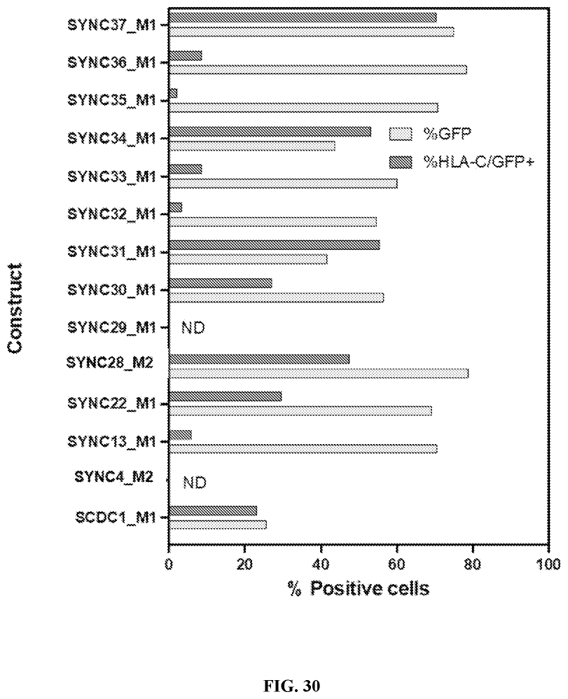

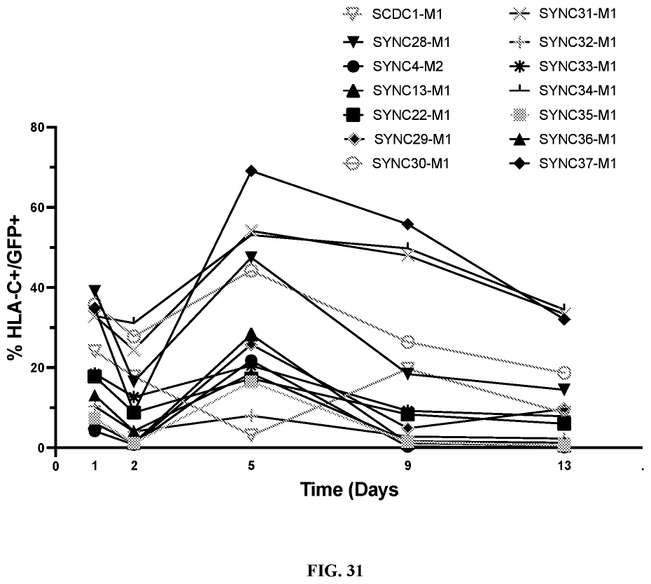

DNA encoding synHLA proteins (control and test) as described herein (FIG. 28) were cloned into the EBV episomal vector pCE modified for multi-gene expression (FIG. 27). The vector harboring the constructs was electroporated into B2M null EBV cells by electroporation and the cells were cultured according to standard protocols. After 1-13 days of culture the cells were stained for HLA-C expression using the antibody DT-9 (Mouse, IgG2b, AlexaFluor 647 labelled, Biolegend Cat #400330). Transfected cells were detected by their GFP expression, and HLA-C expression was detected within the GFP+ve cell population as expected (see FIGS. 29 and 30). The proportion of HLA-C expressing cells was determined and expressed as a percentage of transfected, GFP+, cells minus the staining from the isotype matched control antibody (Clone MCP-11, Mouse, IgG2b, AlexaFluor 647 labelled, Biolegend Cat #373307) staining (FIG. 28). The highest expressing proteins (detected in >50% of GFP positive cells) were SYNC37-M1 (SEQ ID NO: 118), SYNC31-M1 (SEQ ID NO: 106) and SYNC34-M1 (SEQ ID NO 112) (FIG. 28). Maximum cell surface expression of HLA-C was detected 5 days after transfection (FIG. 31). Western blotting analysis, using an antibody specific for GFP (mAb B34, supplied by Biolegend) revealed that GFP was predominantly in a free form, rather than linked to the synHLA protein (FIG. 32).

### Example 19: Preparation of NK Cells

NK cells were prepared in two ways: (1) NK cells were purified from peripheral blood mononuclear cells (PBMC) using a Miltenyi NK Cell Isolation Kit (Cat No. 130-092-657). Purified NK cells were expanded using a bead bound antibodies from a Miltenyi NK Activation/expansion kit (cat No. 130-094-483) in Miltenyi's NK media, supplemented with 5% pooled human serum and 500 U/ml of IL-2. Alternatively, PBMC were cultured as above until the NK cells were more than 90% of the cells present, which takes at least four weeks.

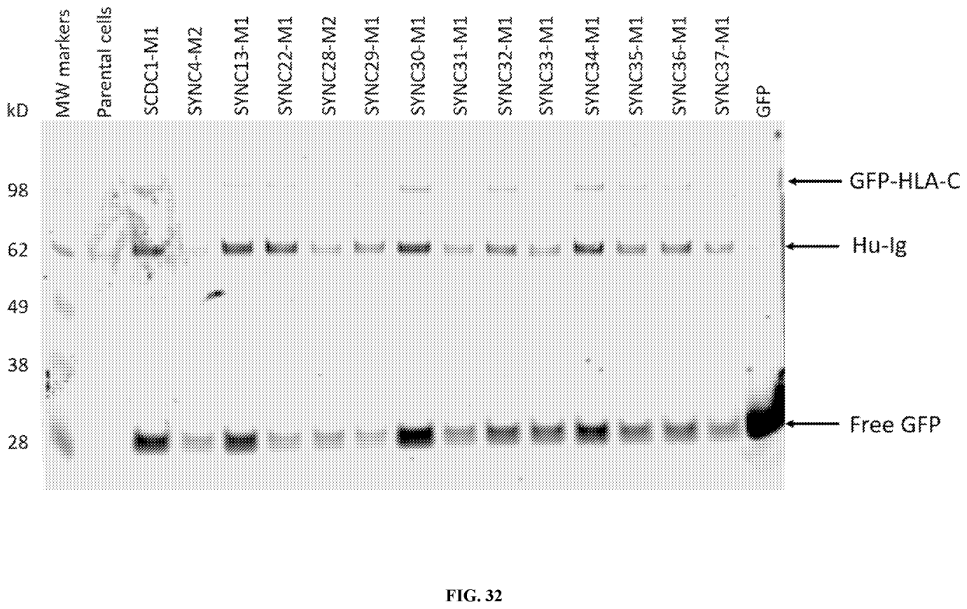

Following NK cell expansions, by either method, the purity of the NK cell population was determined by flow cytometry. This was done by staining the cells with fluorescently conjugated antibodies specific for CD3 and CD56. The proportion of CD3− and CD56+ cells, among all viable cells was determined (see FIG. 33). The purity of NK cells used in the experiments generally exceeded 95%, allowing highly pure NK cells to be obtained.

### Example 20: Relative Susceptibility of EBV Cells Expressing synHLA Proteins to NK-Mediated Lysis

To examine the susceptibility of cells expressing synthetic HLA molecules to NK cell-mediated killing, purified NK cells are incubated with cells expressing a subset of the synthetic HLA proteins described elsewhere herein. NK cells are prepared and analyzed as described elsewhere herein. To prepare the target cells, B2M deficient EBV cells are transfected with pCE plasmid encoding the expression of different HLA-C constructs. Five days after transfection GFP+, HLA-C+ (DT-9 staining) cells are sorted by flow cytometry. Target cells are loaded with ⁵¹Cr for an hour, then washed three times before 5,000 ⁵¹Cr labelled target cells are cultured with 100,000 NK cells, an effector to target cell ratio greater than 1:1. The chronic myeloid leukemia cell line, K562, is included as a positive control for NK mediated killing. After 5 hours of culture the supernatants are collected and the release of ⁵¹Cr is measured by measuring the gamma radiation in the supernatant. The results are expressed as the percentage net killing, relative to target cells cultured alone (background) and maximum lysis, target cells cultured with 2% Triton X-100. The lysis of cells expressing various synHLA constructs are compared to controls.

### Example 21: Relative Ability of EBV Cells Expressing synHLA Proteins to Stimulate CD8+ T Cell Responses

CD8⁺ T cells are purified from the peripheral blood of healthy volunteer donors using magnetic bead positive selection either by staining with a mAb specific for CD8α (clone SK1, phycoerythrin (PE) conjugated) followed by purification using Miltenyi anti-PE micro beads (Miltenyi Cat #130-048-801) or using Milteny REALease CD8 microbead kit (Cat No. 130-117-036). Following purification, the purity of CD8⁺ T cells is determined by staining the cells with antibodies specific for CD8alpha (clone SK1, phycoerythrin conjugated) for CD8.

To prepare the EBV cells expressing synHLA proteins, B2M deficient EBV cells are transfected with pCE plasmid encoding the expression of different HLA-C constructs. Five days after transfection GFP+, HLA-C+ (DT-9 staining) cells are sorted by flow cytometry.

To examine the ability of B2M^{-/-} EBV cells expressing synthetic HLA molecules to stimulate CD8+ T cell response, purified CD8⁺ T cells (50,000) and HLA-C expressing B2M^{-/-} EBV cells (50,000) are cultured together. After 3 days of culture the supernatant is collected and the concentration of IFNγ measured by ELISA. The ability of cells expressing various synHLA constructs to stimulated CD8+ T cell responses is compared to controls.

### Example 22. Engineering of Linker One Enhances Resistance to NK Killing and Enhances the Interaction with Inhibitory KIR Proteins

Inhibitory KIR proteins interact with peptide-bound HLA molecules via a conserved epitope in the peptide binding groove formed by alpha1 and alpha2 helices and bound peptide. Linker-1 sequences are engineered such that they do not sterically block KIR binding, therefore promoting inhibitory NK signaling and reducing killing by NK cells.

### Example 23. Engineering of Linker Two Enhances Resistance to NK Killing and Enhances the Interaction with LILR Proteins

The interaction of synthetic HLA constructs with LILR immune proteins is examined by SPR according to standard protocols. Briefly, a linker region between a B2M domain and an HLA heavy chain domain (linker 2) is engineered specifically to not block canonical/wildtype HLA-like interaction with LILR proteins.

When synHLA proteins with engineered linker 2 variants are examined for binding to recombinant LILR proteins, enhanced binding compared to controls is observed.

When cells expressing synHLA proteins with engineered linker 2 variants are examined for resistance to NK-mediated cell killing, diminished susceptibility to NK cell killing is observed.

### Example 24: Engineering of CD8 Binding Region

Deletion of residues in CD8 binding region allows increased expression in cells while ablating CD8 binding.

Mutation of Q226/D227 ablates CD8 binding. Deletion of residues T225, Q226, D227, and E232 abrogates CD8-binding and improves stability and folding of synHLA constructs as described herein.

### Example 25: Engineering of Peptide

Engineering of peptide weakens the interaction between synHLA constructs as described herein and T-cell receptor and T cell activation and immunogenicity, while maintaining the interaction with inhibitory KIR molecules and avoiding NK cell activation and subsequent killing.

### Example 26: Engineering of Position of a Disulfide Staple

Altering position of disulfide staples allows improved KIR binding and enhanced resistance to NK killing when compared to original disulfide staple (Y84 and position 2 of linker 1). Disulfide staples are also expected to increase the stability and folding of the construct and levels of expression on the cell surface. Staples also serve to lock the peptide in the HLA peptide-binding groove, preventing exchange with potentially immunogenic endogenous peptide.

### Example 27: Combining synHLA and CD47 Expression

Combining synHLA and CD47 (WT) expression in cells increases resistance to NK killing and to killing by macrophages (phagocytosis).

### Example 28: Modulation of Binding to NK Receptors by Engineering of HLA-E

synHLA-E molecules are analogous to synHLA-C, comprising the synHLA domain structure and peptide sequences derived from signal peptides of HLA-A, B, C, and G. Heterodimeric receptors CD94/NKG2A or CD94/NKG2B expressed on the surface of NK cells recognize the synHLA-E molecule, producing an inhibitory effect on the cytotoxic activity of the NK cell to prevent cell lysis. The synHLA-E is engineered in order to maintain or enhance binding to these inhibitory receptors, and/or reduce binding to the activating receptors CD94/NKG2C or CD94/NKG2E, therefore decreasing NK cell activation.

### Example 29: Combining synHLA Proteins Comprised of Polymorphic HLAs with HLA-E

Combinations of synHLA molecules based upon HLA-C (and all alleles thereof) and HLA-E are expressed and found to have an additive or synergistic effect on NK-killing resistance compared to either HLA-C or HLA-E expression individually.

### Example 30: Thermal Stability of synHLA Constructs

A thermal stability assay was carried out to monitor the unfolding of proteins by applying a thermal gradient from 10-100° C. at 1.0° C. per minute on a Bio-Rad CFX96™ Real-Time System RT PCR. Protein unfolding was measured by monitoring the increase in signal from a fluorescent dye, SYPRO™ orange, which binds to hydrophobic regions of proteins as they unfold. Proteins were assayed at 5 μM and were measured individually and in combination as described below. Determination of the melting point, Tₘ (the temperature of the midpoint of the melt transition), of the proteins was done using the Bio-Rad CFX manager software. The first derivative (negative mode) of the protein melt curve identifies maxima that correspond to the Tₘ, a one-step protein unfolding event would have a single maximum, while a two-step unfolding event would have two maxima, and so on.

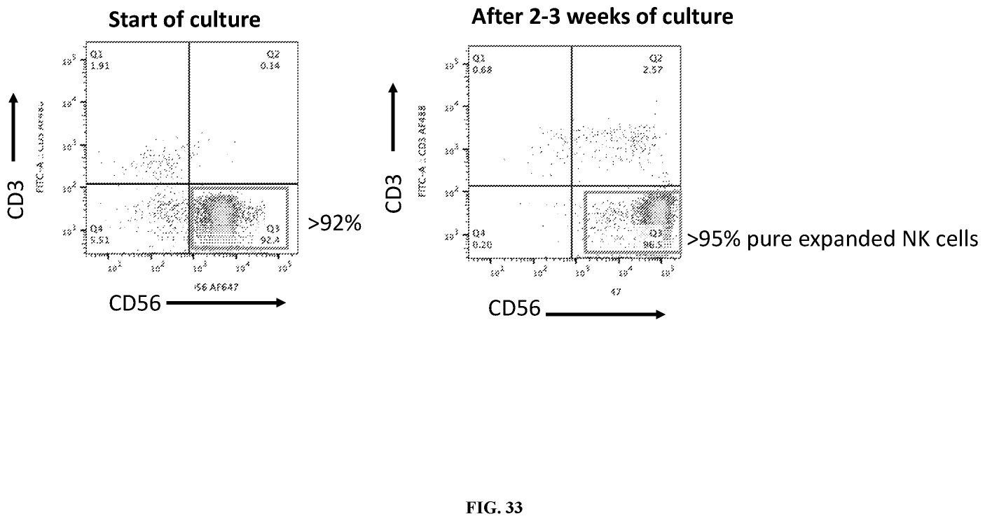

The relevant are shown in Table 20 below. Wild-type HLA-C*07 (SEQ ID NO: 194) with a Histone H3 peptide bound (SEQ ID NO: 159) and a beta-2 microglobulin with R19 peptide construct (SEQ ID NO: 22) were used as controls. FIG. 34 shows Tₘ data for the constructs in Table 20.

TABLE 20
Thermal stability of synHLA constructs

| Construct | SEQ ID NO(s): | Linker 1 | Linker 2 |
|---|---|---|---|
| WT HLA- | 159 and 194 | N/A | N/A |
| C*07 | | | |
| RYR | | | |
| SCTC3.0 | 13 | SEQ ID NO: | N/A |
| | | 179 | |
| SYNC19.1 | 71 | SEQ ID NO: | SEQ ID NO: |
| | | 179 | 183 |
| SYNC20.0 | 73 | SEQ ID NO: | SEQ ID NO: |
| | | 179 | 183 |
| SYNC21.0-L2 | 76 (residues | SEQ ID NO: | N/A |
| | 1-123, 139-415) | 179 | |
| SYNC22.1 | 80 | SEQ ID NO: | SEQ ID NO: |
| | | 177 | 183 |
| SYNC22.1-L2 | 80 (residues | SEQ ID NO: | N/A |
| | 1-120 and | 177 | |
| | 136-425) | | |
| SYNC23.0 | 83 | SEQ ID NO: | SEQ ID NO: |
| | | 177 | 183 |
| SYNC24.0 | 86 | SEQ ID NO: | SEQ ID NO: |
| | | 178 | |
| SYNC24.0-L2 | 86 (1-120, | SEQ ID NO: | N/A |
| | 135-412) | 178 | |
| SYNC25.1 | 90 (1-118, | SEQ ID NO: | N/A |
| | 134-423) | 177 | |
| SYNC26.0 | 93 | SEQ ID NO: | SEQ ID NO: |
| | | 175 | 183 |
| SYNC27.0 | 95 | SEQ ID NO: | SEQ ID NO: |
| | | 180 | 183 |
| SYNC27.0-L2 | 95 (residues | SEQ ID NO: | N/A |
| | 1-118, 134-410) | 176 | |
| SYNC38.0 | 119 | SEQ ID NO: | SEQ ID NO: |
| | | 175 | 183 |
| SYNC40.0 | 121 | SEQ ID NO: | SEQ ID NO: |
| | | 175 | 183 |
| SYNC41.0 | 122 | SEQ ID NO: | SEQ ID NO: |
| | | 175 | 183 |
| SYNC42.0 | 123 | SEQ ID NO: | SEQ ID NO: |
| | | 181 | 183 |
| SYNC44.0 | 125 | SEQ ID NO: | SEQ ID NO: |
| | | 175 | 183 |
| SYNC44.0-L2 | 125 (residues | SEQ ID NO: | N/A |
| | 1-118, 134-415) | 175 | |
| SYNC45.0 | 126 | SEQ ID NO: | N/A |
| | | 175 | |
| SYNC47.0-L2 | 128 (residues | SEQ ID NO: | N/A |
| | 1-118 and | 175 | |
| | 134-410) | | |
| SCD3.0 | 22 | SEQ ID NO: | N/A |
| | | 177 | |

In general, constructs with a disulfide staple (e.g., SYNC21.0-L2, SYNC24.0-L2, SYNC27.0-L2, SYNC24.0, SYNC27.0) showed a much higher thermal stability, regardless of linker length. Among constructs without disulfide staples, those with the shortest linker 1 regions (e.g., SYNC42.0) showed the lowest thermal stability overall. The highest thermal stability among unstapled constructs was those with the next shortest linker 1 regions (e.g., SYNC25.1 and SYNC26), followed by those with a long linker 1 region (e.g., SYNC19.1 and SYNC20), and finally those with a medium length linker 1 region (E.g., SYNC22.1 and SYNC23).

### Example 31: Analysis of Interaction of Synthetic HLA Constructs and Killer-Cell Immunoglobulin-Like Receptor Proteins by Surface Plasmon Resonance (SPR)

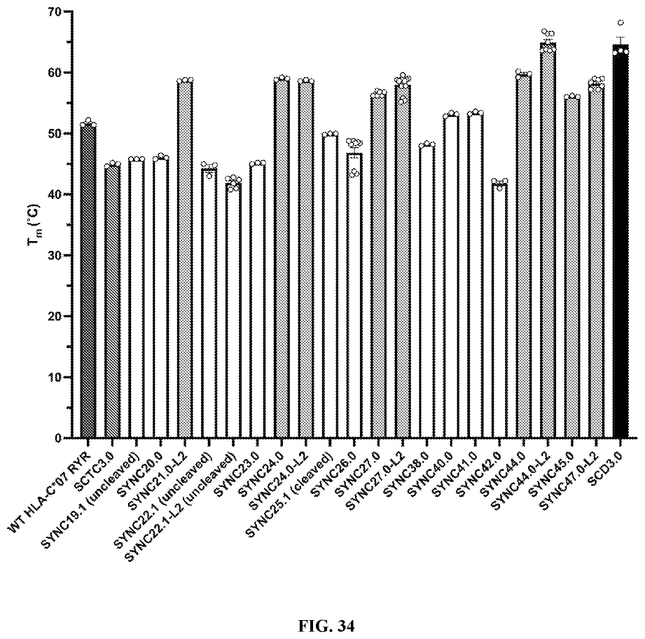

The interaction of synthetic HLA constructs with KIR immune proteins was examined by SPR according to standard protocols. Briefly, histidine-tagged KIR proteins (ligand) were immobilized via Nickel-NTA coupling with a nitrilotriacetic acid (NTA)-derivatized carboxymethyldextran sensor chip (NiD50L, XanTec Bioanalytics). Recombinant HLA proteins as listed in Table 21 were then flowed over the surface, producing a sensorgram. Representative sensorgrams for SYNC21 (top right), SYNC27 (top left), SYNC109 (bottom right), and SYNC110 (bottom left) are illustrated in FIG. 35. All experiments were performed at 25° C. where both analyte and ligand were diluted in the SPR buffer (20 mM HEPES pH 7.5, 100 mM NaCl, 50 μM EDTA, 0.05% (v/v) tween-20). The flow cells were enhanced with 500 μM NiCl₂ (10 μL×min⁻¹, 1 min).

The response of the analyte injections at the end of the equilibrium phase were double referenced against reference cell and blank injection values before normalization corresponding to their respective ligand immobilization response. The relative response of each interaction is shown in Table 21. Relative to wild-type, SYNC26, SYNC42, and SYNC44 showed superior binding to KIR2DL2 while SYNC21 showed comparable binding to KIR2DL3 as WT.

TABLE 21
Measured binding of synHLA constructs to KIRs

Measured K_{D} (μM)

| Analyte | SEQ ID NO: | KIR2DL2 | KIR2DL3 |
|---|---|---|---|
| WT HLA-C* | 194 | 28.9 | 5.9 |
| | | 18 | 22 |
| | | 66 | 175 |
| SCTC3 | 13 | | 2.9 |
| SYNC20 | 73 | 2000 | 15 |
| | | | 27 |
| SYNC23 | 83 | 124 | |
| SYNC26-2 | 94 | 2.9 | |
| SYNC42 | 123 | 4.7 | 19 |
| SYNC21 | 76 | 105 | 5.9 |
| SYNC24 | 86 | 244 | |
| SYNC27 | 95 | 688 | 88 |
| SYNC44-L2 | 125 (residues 1-118, | 7.4 | 67 |
| | 134-415) | | |
| SYNC47-L2 | 128 (residues 1-118 | 1602 | 1.8 |
| | and 134-410) | | |
| SYNC108-L2 | 246 | | 12.5 |
| SYNC109-L2 | 247 | | 4144 |
| SYNC110-L2 | 248 | | 4.8 |

The synHLA proteins with the highest relative responses to KIR2DL2 were SYNC26-1 and SYNC23-1. Both of these proteins contained four point mutations in the HLA heavy-chain (CIG, Y84A, Q226A, D227K), a 15-residue linker 2, and either a short (SYNC26-1) or medium (SYNC23-1) linker 1. When a longer linker 1 was introduced into the same protein backbone to create SYNC20-1, reduced binding to KIR2DL2 was observed. This suggests that the presence of a longer linker 1 may impair binding to KIR2DL2.

### Example 32: synHLA Constructs Afford Protection from NK Cell Mediated Killing

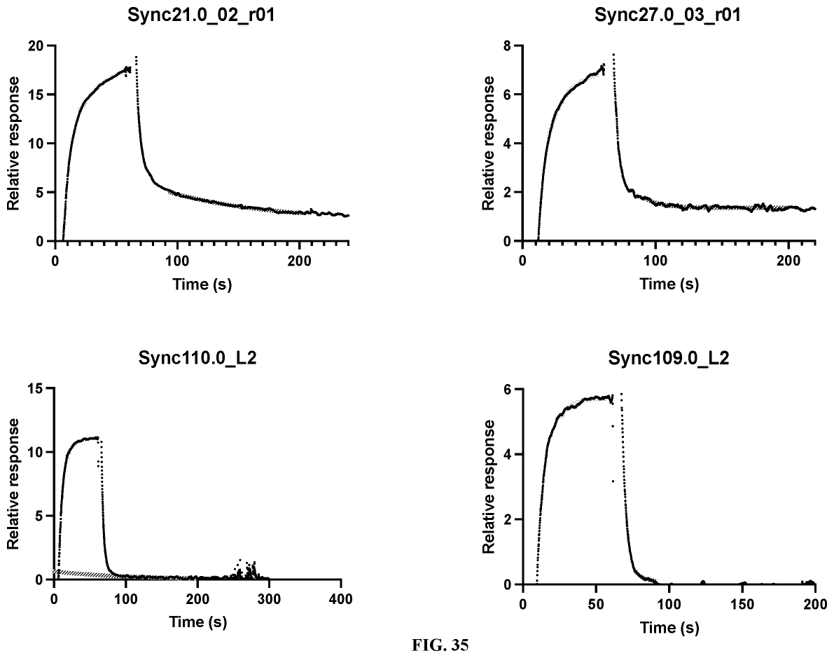

In order to test protection from NK cell-mediated killing, synHLA-C complexes as described herein were expressed in K562 cell lines by lentiviral transduction. The target complexes listed in Table 22 were cloned into a pRRLSIN vector along with GFP and transduced into K562 cells. Following sorting and confirmation of HLA expression (79.5% or greater for all constructs), target cells were loaded with ⁵¹Cr as described in Example 20. Wild-type K562, GFP-transduced cells, CD47-transduced cells, and mCherry transduced cells were used as negative controls while HLA-E transduced cells were used as a positive control. After 5 hours of culture, supernatants were collected and the release of ⁵¹Cr was measured. The results are depicted in FIG. 36 as the percentage of net killing, relative to WT K562 cultured alone and maximum lysis, target cells cultured with 2% TritonX-100.

As illustrated in FIG. 36, constructs comprising SYNC13, SYNC22, SYNC28, SYNC30, SYNC31, SYNC32, SYNC33, SYNC34, SYNC35, and SYNC36 showed protection from NK cell killing, releasing less ⁵¹Cr relative to background.

TABLE 22
Constructs evaluated for NK cell killing in Example 32

| Construct | SEQ ID NO: |
|---|---|
| SYNC4 | 38 |
| SYNC13 | 57 |
| SYNC22 | 79 |
| SYNC28 | 98 |
| SYNC29 | 101 |
| SYNC30 | 103 |
| SYNC31 | 105 |
| SYNC32 | 107 |
| SYNC33 | 109 |
| SYNC34 | 111 |
| SYNC35 | 113 |
| SYNC36 | 115 |
| SYNC37 | 117 |

### Example 33: Combining synHLA Proteins Comprising Polymorphic HLAs with HLA-E

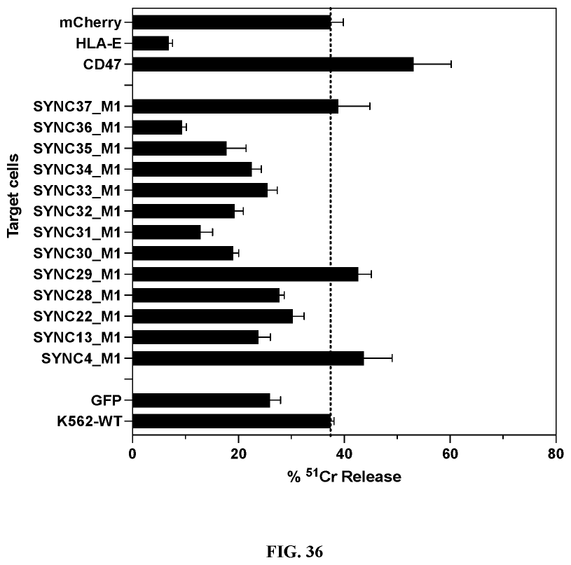

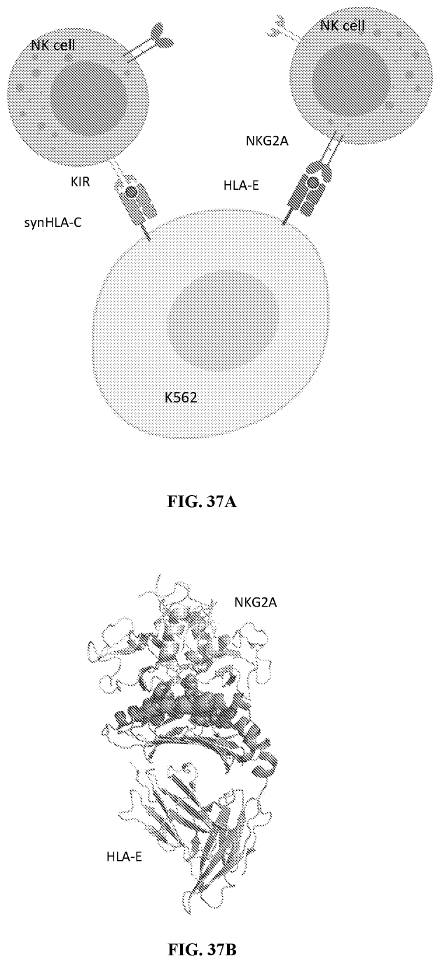

Combinations of synHLA-C molecules and HLA-E molecules were co-expressed and found to have an additive or synergistic effect on NK-killing resistance compared to either HLA-C or HLA-E expression individually. synHLA-C constructs were expressed in K562 cell lines by lentiviral transduction. The target complexes listed in Table 22 were cloned into a pRRLSIN vector along with GFP and HLA-E and transduced into K562 cells. A schematic showing synergistic blocking of NK-cell killing in cells co-expressing synHLA (e.g., synHLA-C) molecules and HLA-E molecules is shown in FIG. 37A.As illustrated in FIGS. 37A and 37B, HLA-E interacts with the inhibitory CD94/NKG2A receptor on NK cells to further inhibit an NK-cell response beyond the inhibition of KIRs by HLA-C (illustrated in FIG. 37A for example purposes as a synHLA construct as described herein). Following sorting and confirmation of HLA expression, target cells were loaded with ⁵¹Cr as described in Example 20. Wild-type K562, were used as a negative control while HLA-E transduced cells (both with GFP and without) were used as positive controls. After 5 hours of culture, supernatants were collected and the release of ⁵¹Cr was measured. The results are depicted in FIG. 38 as the percentage of net killing, relative to WT K562 cultured alone and maximum lysis, target cells cultured with 2% TritonX-100.

As illustrated in FIG. 38, co-expression of HLA-E with SYNC4, SYNC13, SYNC22, SYNC30, and SYNC32 shows synergistic inhibition of NK cell killing, releasing less chromium than cells expressing only HLA-E.

### Example 34: A Luciferase Assay for Measuring NK Cell Cytotoxicity

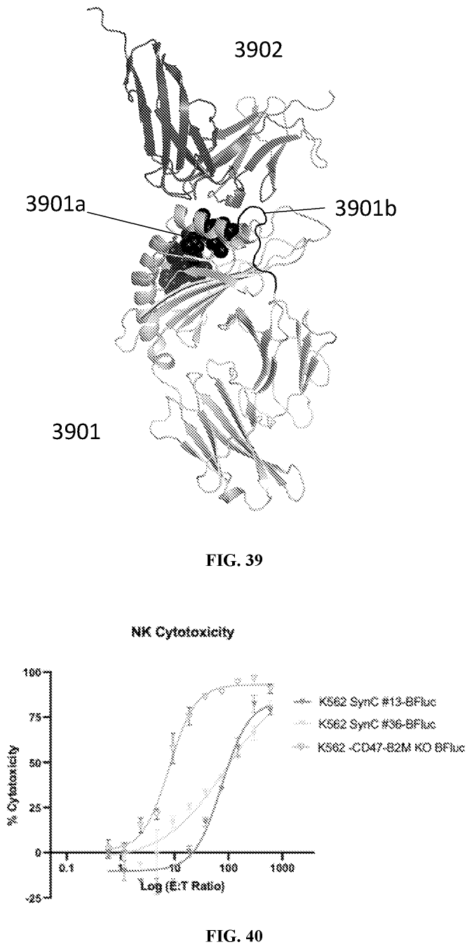

A CD47/B2M KO K562 cell line was generated for cytotoxic assays. A luciferase cassette was delivered by lentivirus transduction to generate a K562-CD47-B2M KO BFluc cell line that constitutively expresses luciferase. Engineered synHLA-C constructs (SEQ ID NOs: 58 and 116) were individually transduced into the K562-CD47-B2M KO BFluc cell line. To assess protection efficacy, an assay was implemented where IL-2 activated NK cells from healthy donors were co-cultured with K562-CD47-B2M KO BFluc, K562-CD47-B2M KO BFluc #13 and K562-CD47-B2M KO BFluc #36. At high ratios of NK cells (effector cells) m cells susceptible to cytotoxic killing (K562-CD47-B2M KO BFluc-target cells) would kill 100% of the target cells and no luminescence signal would be observed from the Luciferase containing target cells. Titration of effector to target cells (E:T) results in less killing of target cells and luciferase signal. The data would be expressed as a percentage of cell killing for each construct to enable comparison between different K562-CD47-B2M KO BFluc constructs. Representative data for the assay are shown in FIG. 40. The rightward shift of the response curved for the two synHLA constructs suggests they successfully protected their respective target cells from NK-cell killing.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A construct comprising:
a. one or more targeting moieties;
b. one or more major histocompatibility complex (MHC) regions, wherein at least one of said one or more MHC regions comprise a cluster of differentiation 8 (CD8) binding site; and
c. one or more linker regions.

2. The construct of claim 1, wherein said CD8 binding site comprises one or more mutations.

3. The construct of claim 1 or 2, further comprising one or more disulfide staple pairs.

4. The construct of any one of claims 1-3, wherein said one or more MHC regions are inhibited from eliciting a T cell response when said construct is interrogated by one or more T cells or an NK cell response when said construct is interrogated by one or more NK cells.

5. The construct of any one of claims 1-3, wherein said one or more MHC regions comprise one or more mutated residues relative to the wild-type version of said one or more MHC regions, wherein said one or more mutated residues are located at a corresponding position to the tyrosine 84 (Y84) residue of the human leukocyte antigen (HLA) protein HLA-C.

6. The construct of claim 5, wherein said one or more mutated residues comprise alanine.

7. The construct of claim 5 or 6, wherein said one or more mutated residues comprise cysteine.

8. The construct of any one of claims 1-7, wherein said CD8 binding site comprises a mutation to the residue corresponding to the Q226 residue of HLA-C.

9. The construct of any one of claims 1-8, wherein said CD8 binding site comprises a mutation to the residue corresponding to the D227K residue of HLA-C.

10. The construct of any one of claims 1-9, wherein the T225 residue of said CD8 binding site is deleted.

11. The construct of any one of claims 1-10, wherein the Q226 residue of said CD8 binding site is deleted.

12. The construct of any one of claims 1-11, wherein the T225 residue of said CD8 binding site is deleted.

13. The construct of any one of claims 1-12, wherein the E232 residue of said CD8 binding site is deleted.

14. The construct of any one of claims 1-13, wherein said one or more MHC regions further comprise one or more mutations to the residue corresponding to the C1 residue of HLA-C.

15. The construct of claim 14, wherein said residue is a glycine.

16. The construct of any one of claims 1-15, wherein said disulfide staple pair is formed between the residue of said one or more MHC regions corresponding to the Y84 residue of an HLA-C and a residue in said one or more linker regions.

17. The construct of any one of claims 1-16, wherein said disulfide staple pair is formed between the residue of said one or more MHC regions corresponding to the R69 residue of an HLA-C and a residue in said one or more targeting moieties.

18. The construct of any one of claims 1-17, wherein said disulfide staple pair is formed between the residue of said one or more MHC regions corresponding to the A150 residue of an HLA-C and a residue in said one or more targeting moieties.

19. The construct of any one of claims 1-18, wherein said disulfide staple pair is formed between the residue of said one or more MHC regions corresponding to the A73 residue of an HLA-C and a residue in said one or more targeting moieties.

20. The construct of any one of claims 1-19, wherein said construct is soluble.

21. The construct of any one of claims 1-20, wherein said construct is insoluble.

22. The construct of any one of claims 1-21, wherein said construct comprises a beta-2 microglobulin (B2M) leader sequence.

23. The construct of any one of claims 1-22, wherein said construct further comprises an N-terminal signal sequence.

24. The construct of any one of claims 1-23, wherein said construct further comprises a C-terminal signal sequence.

25. The construct of any one of claims 1-24, wherein said construct is a single chain trimer (SCT).

26. The construct of any one of claims 1-25, wherein said construct comprises a single chain dimer (SCD).

27. The construct of any one of claims 1-26, wherein said one or more MHC regions comprise one or more human HLA class 1 heavy chain sequences.

28. The construct of claim 27, wherein said one or more human HLA class 1 heavy chain sequences are derived from HLA-A, HLA-B, HLA-C, HLA-D, HLA-E, HLA-F, HLA-G, or some combination thereof.

29. The construct of any one of claims 1-28, wherein said one or more targeting moieties comprises a peptide.

30. The construct of claim 29, wherein said peptide comprises a sequence at least about 50%, about 60%, about 70%, about 80%, about 90%, about 95%, or about 99% identical to any one of SEQ ID NOs: 158-174.

31. The construct of any one of claims 1-30, wherein a linker region of said one or more linker regions comprises a sequence at least about 50%, about 60%, about 70%, about 80%, about 90%, about 95%, or about 99% identical to any one of SEQ ID NOs 175-186:

32. The construct of any one of claims 1-31, comprising a first linker region and a second linker region, wherein said first linker region comprises a sequence at least about 50%, about 60%, about 70%, about 80%, about 90%, about 95%, or about 99% identical to any one of SEQ ID NOs: 175-186; and wherein said second linker region comprises a sequence at least about 50%, about 60%, about 70%, about 80%, about 90%, about 95%, or about 99% identical to any one of SEQ ID NOs: 175-186.

33. The construct of any one of claims 1-32, comprising a sequence at least about 50%, about 60%, about 70%, about 80%, about 90%, about 95%, or about 99% identical to any one of SEQ ID NOs: 30-129 and 142-157.

34. The construct of any one of claims 1-33, wherein said construct comprises in N-terminus to C-terminus order:
a. a targeting moiety of said one or more targeting moieties;
b. a first linker of said one or more linkers; and
c. a MHC region of said one or more MHC regions; and
d. a disulfide stable pair configured to associate said targeting moiety and said MHC region or configured to associate said first linker and said MHC region.

35. The construct of claim 34, wherein said targeting moiety of said one or more targeting moieties comprises a sequence at least about 50%, 60%, 70%, 80%, 90%, 95%, or 99% identical to any one of SEQ ID NOs: 158-174.

36. The construct of claim 34 or 35, wherein said first linker comprises a sequence at least about 50%, 60%, 70%, 80%, 90%, 95%, or 99% identical to any one of SEQ ID NOs: 175-186.

37. The construct of any one of claims 34-36, wherein said MHC region comprises a sequence at least about 50%, 60%, 70%, 80%, 90%, 95%, or 99% identical to any one of SEQ ID NOs: 130-141.

38. The construct of any one of claims 34-37, further comprising a B2M leader sequence between said first linker and said MHC region.

39. The construct of claim 38, further comprising a second linker between said B2M leader sequence and said MHC region.

40. The construct of claim 39, wherein said second linker comprises a sequence at least about 50%, 60%, 70%, 80%, 90%, 95%, or 99% identical to any one of SEQ ID NOS: 175-186.

41. A nucleic acid encoding the construct of any one of any one of claims 1-40.

42. An engineered vector encoding the nucleic acid of claim 41.

43. The engineered vector of claim 42, wherein said vector is a plasmid, a minicircle, an adeno-associated virus (AAV) derived virion, a lentivirus, an adenovirus, or a herpes simplex virus (HSV).

44. A method of generating a hypo-immunogenic cell comprising administering to a cell said vector of claim 42 or 43.

45. A hypo-immunogenic pluripotent stem cell comprising a construct comprising:
a. one or more targeting moieties;
b. one or more major histocompatibility complex (MHC) regions, wherein at least one of said one or more MHC regions comprise a cluster of differentiation 8 (CD8) binding site; and
c. one or more linker regions;

46. The hypo-immunogenic pluripotent stem cell of claim 45, wherein said CD8 binding site comprises one or more mutations.

47. The hypo-immunogenic pluripotent stem cell of claim 45 or 46, further comprising one or more disulfide staple pairs.

48. The hypo-immunogenic pluripotent stem cell of claim 47, wherein said one or more MHC regions are inhibited from eliciting a T cell response when said complex is interrogated by one or more T cells.

49. The hypo-immunogenic pluripotent stem cell of claim 47 or 48, wherein said one or more MHC regions comprise one or more mutated residues relative to the wild-type version of said one or more MHC regions, wherein said one or more mutated residues are located at a corresponding position to the tyrosine 84 (Y84) residue of the human leukocyte antigen (HLA) protein HLA-C.

50. The hypo-immunogenic pluripotent stem cell of claim 49, wherein said one or more mutated residues comprise alanine.

51. The hypo-immunogenic pluripotent stem cell of claim 49 or 50, wherein said one or more mutated residues comprise cysteine.

52. The hypo-immunogenic pluripotent stem cell of any one of claims 47-51, wherein said CD8 binding site comprises a mutation to the residue corresponding to the Q226 residue of HLA-C.

53. The hypo-immunogenic pluripotent stem cell of any one of claims 47-52, wherein said CD8 binding site comprises a mutation to the residue corresponding to the D227K residue of HLA-C.

54. The hypo-immunogenic pluripotent stem cell of any one of claims 47-53, wherein the T225 residue of said CD8 binding site is deleted.

55. The hypo-immunogenic pluripotent stem cell of any one of claims 47-54, wherein the Q226 residue of said CD8 binding site is deleted.

56. The hypo-immunogenic pluripotent stem cell of any one of claims 47-55, wherein the T22D2275 residue of said CD8 binding site is deleted.

57. The hypo-immunogenic pluripotent stem cell of any one of claims 47-56, wherein the E232 residue of said CD8 binding site is deleted.

58. The hypo-immunogenic pluripotent stem cell of any one of claims 47-57, wherein said one or more MHC regions further comprise one or more mutations to the residue corresponding to the C1 residue of HLA-C.

59. The hypo-immunogenic pluripotent stem cell of claim 58, wherein said residue is a glycine.

60. The hypo-immunogenic pluripotent stem cell of any one of claims 47-59, wherein said disulfide staple pair is formed between the residue of said one or more MHC regions corresponding to the Y84 residue of an HLA-C and a residue in said one or more linker regions.

61. The hypo-immunogenic pluripotent stem cell of any one of claims 47-60, wherein said disulfide staple pair is formed between the residue of said one or more MHC regions corresponding to the R69 residue of an HLA-C and a residue in said one or more targeting moieties.

62. The hypo-immunogenic pluripotent stem cell of any one of claims 47-61, wherein said disulfide staple pair is formed between the residue of said one or more MHC regions corresponding to the A150 residue of an HLA-C and a residue in said one or more targeting moieties.

63. The hypo-immunogenic pluripotent stem cell of any one of claims 47-62, wherein said disulfide staple pair is formed between the residue of said one or more MHC regions corresponding to the A73 residue of an HLA-C and a residue in said one or more targeting moieties.

64. The hypo-immunogenic pluripotent stem cell of any one of claims 47-63, wherein said construct is soluble.

65. The hypo-immunogenic pluripotent stem cell of any one of claims 47-64, wherein said construct is insoluble.

66. The hypo-immunogenic pluripotent stem cell of any one of claims 47-65, wherein said construct comprises a beta-2 microglobulin (B2M) leader sequence.

67. The hypo-immunogenic pluripotent stem cell of any one of claims 47-66, wherein said construct further comprises an N-terminal signal sequence.

68. The hypo-immunogenic pluripotent stem cell of any one of claims 47-67, wherein said construct further comprises a C-terminal signal sequence.

69. The hypo-immunogenic pluripotent stem cell of any one of claims 47-68, wherein said construct is a single chain trimer (SCT).

70. The hypo-immunogenic pluripotent stem cell of claim 47-69, wherein said construct is a single chain dimer (SCD).

71. The hypo-immunogenic pluripotent stem cell of claim 47-70, wherein said one or more MHC regions comprise one or more human HLA class 1 heavy chain sequences.

72. The hypo-immunogenic pluripotent stem cell of claim 71, wherein said one or more human HLA class 1 heavy chain sequences are derived from HLA-A, HLA-B, HLA-C, HLA-D, HLA-E, HLA-F, HLA-G, or some combination thereof.

73. The hypo-immunogenic pluripotent stem cell of any one of claims 47-62, wherein said one or more targeting moieties comprises a peptide.

74. The construct of claim 73, wherein said peptide comprises a sequence at least about 50%, 60%, 70%, 80%, 90%, 95%, or 99% identical to any one of SEQ ID NOs: 158-174.

75. The hypo-immunogenic pluripotent stem cell of any one of claims 45-74, wherein a linker region of said one or more linker regions comprises a sequence at least about 50%, 60%, 70%, 80%, 90%, 95%, or 99% identical to any one of SEQ ID NOs: 175-186.

76. The hypo-immunogenic pluripotent stem cell of any one of claims 47-75, comprising a first linker region and a second linker region, wherein said first linker region comprises a sequence at least about 50%, 60%, 70%, 80%, 90%, 95%, or 99% identical to any one of SEQ ID NOs: 175-186; and wherein said second linker region comprises a sequence at least about 50%, 60%, 70%, 80%, 90%, 95%, or 99% identical to any one of SEQ ID NOs: 175-186.

77. The hypo-immunogenic pluripotent stem cell of any one of claims 47-76, comprising a sequence at least 50%, 60%, 70%, 80%, 90%, 95%, or 99% identical to any one of SEQ ID NOs: 30-129 and 142-157.

78. The hypo-immunogenic pluripotent stem cell of any one of claims 47-77, wherein said construct comprises in N-terminus to C-terminus order:
a. a targeting moiety of said one or more targeting moieties;
b. a first linker of said one or more linkers; and
c. a MHC region of said one or more MHC regions; and
d. a disulfide stable pair configured to associate said targeting moiety and said MHC region or configured to associate said first linker and said MHC region.

79. The hypo-immunogenic pluripotent stem cell of claim 78, wherein said targeting moiety of said one or more targeting moieties comprises a sequence at least about 50%, 60%, 70%, 80%, 90%, 95%, or 99% identical to any one of SEQ ID NOs: 158-174.

80. The hypo-immunogenic pluripotent stem cell of claim 78 or 79, wherein said first linker comprises a sequence at least about 50%, 60%, 70%, 80%, 90%, 95%, or 99% identical to any one of SEQ ID NOs: 175-186.

81. The hypo-immunogenic pluripotent stem cell of any one of claims 78-80, wherein said MHC region comprises a sequence at least about 50%, 60%, 70%, 80%, 90%, 95%, or 99% identical to any one of SEQ ID NOs: 130-141.

82. The hypo-immunogenic pluripotent stem cell of any one of claims 78-81, further comprising a B2M leader sequence between said first linker and said MHC region.

83. The hypo-immunogenic pluripotent stem cell of claim 82, further comprising a second linker between said B2M leader sequence and said MHC region.

84. The hypo-immunogenic pluripotent stem cell of claim 83, wherein said second linker comprises a sequence at least about 50%, 60%, 70%, 80%, 90%, 95%, or 99% identical to any one of SEQ ID NOs: 175-186.

85. A method of generating a hypo-immunogenic pluripotent stem cell comprising:
a. generating a construct comprising one or more targeting moieties, one or more linker regions, and one or more major histocompatibility complex (MHC) regions, wherein at least one of said one or more MHC regions comprise a cluster of differentiation 8 (CD8) binding site;
b. providing said construct in a pluripotent stem cell (PSC); and
c. expressing said construct in said (PSC).

86. The method of claim 85, wherein said CD8 binding site comprises one or more mutations

87. The method of claim 85 or 86, wherein said one or more MHC regions are inhibited from eliciting a T cell response when said complex is interrogated by one or more T cells.

88. The method of any one of claims 85-87, wherein said one or more MHC regions comprise one or more mutated residues relative to the wild-type version of said one or more MHC regions, wherein said one or more mutated residues are located at a corresponding position to the tyrosine 84 (Y84) residue of the human leukocyte antigen (HLA) protein HLA-C.

89. The method of claim 88, wherein said one or more mutated residues comprise alanine.

90. The method of claim 88 or 89, wherein said one or more mutated residues comprise cysteine.

91. The method of any one of claims 86-90, wherein said CD8 binding site comprises a mutation to the residue corresponding to the Q226 residue of HLA-C.

92. The method of any one of claims 86-91, wherein said CD8 binding site comprises a mutation to the residue corresponding to the D227K residue of HLA-C.

93. The method of any one of claims 86-92, wherein the T225 residue of said CD8 binding site is deleted.

94. The method of any one of claims 86-93, wherein the Q226 residue of said CD8 binding site is deleted.

95. The method of any one of claims 86-94, wherein the T22D2275 residue of said CD8 binding site is deleted.

96. The method of any one of claims 86-95, wherein the E232 residue of said CD8 binding site is deleted.

97. The method of any one of claims 86-96, wherein said one or more MHC regions further comprise one or more mutations to the residue corresponding to the C1 residue of HLA-C.

98. The method of claim 97, wherein said residue is a glycine.

99. The method of any one of claims 86-98, wherein said disulfide staple pair is formed between the residue of said one or more MHC regions corresponding to the Y84 residue of an HLA-C and a residue in said one or more linker regions.

100. The method of any one of claims 86-99, wherein said disulfide staple pair is formed between the residue of said one or more MHC regions corresponding to the R69 residue of an HLA-C and a residue in said one or more targeting moieties.

101. The method of any one of claims 86-100, wherein said disulfide staple pair is formed between the residue of said one or more MHC regions corresponding to the A150 residue of an HLA-C and a residue in said one or more targeting moieties.

102. The method of any one of claims 86-101, wherein said disulfide staple pair is formed between the residue of said one or more MHC regions corresponding to the A73 residue of an HLA-C and a residue in said one or more targeting moieties.

103. The method of any one of claims 86-102, wherein said construct is soluble.

104. The method of any one of claims 86-103, wherein said construct is insoluble.

105. The method of any one of claims 86-104, wherein said construct comprises a beta-2 microglobulin (B2M) leader sequence.

106. The method of any one of claims 86-105, wherein said construct further comprises an N-terminal signal sequence.

107. The method of any one of claims 86-106, wherein said construct further comprises a C-terminal signal sequence.

108. The method of any one of claims 86-107, wherein said construct is a single chain trimer (SCT).

109. The method of any one of claims 86-108, wherein said construct is a single chain dimer (SCD).

110. The method of any one of claims 86-109, wherein said one or more MHC regions comprise one or more human HLA class 1 heavy chain sequences.

111. The method of claim 110, wherein said one or more human HLA class 1 heavy chain sequences are derived from HLA-A, HLA-B, HLA-C, HLA-D, HLA-E, HLA-F, HLA-G, or some combination thereof.

112. The method of any one of claims 86-111, wherein said one or more targeting moieties comprises a peptide.

113. The method of claim 112, wherein said peptide comprises a sequence at least about 50%, 60%, 70%, 80%, 90%, 95%, or 99% identical to any one of SEQ ID NOs: 158-174.

114. The method of any one of claims 86-113, wherein a linker region of said one or more linker regions comprises a sequence at least about 50%, 60%, 70%, 80%, 90%, 95%, or 99% identical to any one of SEQ ID NOs 175-186:

115. The method of any one of claims 86-114, comprising a first linker region and a second linker region, wherein said first linker region comprises a sequence at least about 50%, 60%, 70%, 80%, 90%, 95%, or 99% identical to any one of SEQ ID NOs: 175-186; and wherein said second linker region comprises a sequence at least about 50%, 60%, 70%, 80%, 90%, 95%, or 99% identical to any one of SEQ ID NOs: 175-186.

116. The method of any one of claims 86-115, comprising a sequence at least 50%, 60%, 70%, 80%, 90%, 95%, or 99% identical to any one of SEQ ID NOs: 30-129 and 142-157.

117. The method of any one of claims 86-116, wherein said construct comprises in N-terminus to C-terminus order:
a. a targeting moiety of said one or more targeting moieties;
b. a first linker of said one or more linkers; and
c. a MHC region of said one or more MHC regions; and
d. a disulfide stable pair configured to associate said targeting moiety and said MHC region or configured to associate said first linker and said MHC region.

118. The method of claim 117, wherein said targeting moiety of said one or more targeting moieties comprises a sequence at least about 50%, 60%, 70%, 80%, 90%, 95%, or 99% identical to any one of SEQ ID NOs: 158-174.

119. The method of claim 117 or 118, wherein said first linker comprises a sequence at least about 50%, 60%, 70%, 80%, 90%, 95%, or 99% identical to any one of SEQ ID NOs: 175-186.

120. The method of any one of claims 117-119, wherein said MHC region comprises a sequence at least about 50%, 60%, 70%, 80%, 90%, 95%, or 99% identical to any one of SEQ ID NOs: 130-141.

121. The method of any one of claims 86-120, further comprising a B2M leader sequence between said first linker and said MHC region.

122. The method of claim 121, further comprising a second linker between said B2M leader sequence and said MHC region.

123. The method of claim 122, wherein said second linker comprises a sequence at least about 50%, 60%, 70%, 80%, 90%, 95%, or 99% identical to any one of SEQ ID NOs: 175-186.

124. A construct comprising:
a. a targeting moiety;
b. a major histocompatibility complex (MHC) region; and
c. a linker region disposed between said targeting moiety and said MHC region;
wherein one of said targeting moiety, said MHC region, and said linker region comprises a first cysteine residue and another of said targeting moiety, said MHC region, and said linker region comprises a second cysteine residue, wherein said first cysteine residue and said second cysteine residue are configured to form a disulfide bond with one another when said construct is expressed on a surface of a cell.

125. The construct of claim 124, wherein said MHC region comprises an MHC class I heavy chain.

126. The construct of claim 125, wherein said MHC region is derived from an HLA-A, HLA-B, or HLA-C sequence.

127. The construct of any one of claims 124-126, wherein said MHC class I heavy chain comprises a sequence at least about 50%, 60%, 70%, 80%, 90%, 95%, or 99% identical to any one of SEQ ID NOs 130-141 and 279-288.

128. The construct of any one of claims 126-127, wherein said MHC class I heavy chain comprises a mutation corresponding to the C1 residue of HLA-C (e.g., SEQ ID NO: 194).

129. The construct of claim 128, wherein said mutation comprises a glycine residue.

130. The construct of any one of claims 126-129, further comprising a second MHC region.

131. The construct of claim 130, wherein said second MHC region is derived from an HLA-A, HLA-B, HLA-C, HLA-E, HLA-F, or HLA-G sequence.

132. The construct of any one of claims 124-131, further comprising a beta-2 microglobulin (B2M) region.

133. The construct of claim 132, wherein said B2M region is disposed between said targeting moiety and said MHC region.

134. The construct of claim 132 or 133, wherein said B2M region comprises a sequence at least about 50%, 60%, 70%, 80%, 90%, 95%, or 99% identical to SEQ ID NO: 195.

135. The construct of any one of claims 132-134, further comprising a second linker region disposed between said B2M region and said targeting moiety or said MHC region.

136. The construct of claim 135, wherein said linker region is disposed between said targeting moiety and said B2M region and said second linker region is disposed between said B2M region and said MHC region.

137. The construct of claim 136, wherein said linker region is less than fifteen amino acid residues in length.

138. The construct of claim 136, wherein said linker region is less than fourteen amino acid residues in length.

139. The construct of claim 136, wherein said linker region is less than thirteen amino acid residues in length.

140. The construct of any one of claims 136-139, wherein said linker region is at least eight amino acid residues in length.

141. The construct of any one of claims 136-140, wherein said linker region comprises at least one cysteine residue.

142. The construct of claim 141, wherein said linker region comprises amino acids selected from the group consisting of: glycine, serine, and cysteine.

143. The construct of any one of claims 124-136, wherein said linker region comprises a sequence that is at least about 50%, 60%, 70%, 80%, 90%, 95%, or 99% identical to any one of SEQ ID NOs 175-186.

144. The construct of any one of claims 124-136, wherein said linker region comprises a sequence selected from SEQ ID NOs: 175-186 and 198.

145. The construct of claim 136, wherein said second linker region comprises a sequence that is at least about 50%, 60%, 70%, 80%, 90%, 95%, or 99% identical to any one of SEQ ID NOs 175-186.

146. The construct of claim 136, wherein said second linker region comprises a sequence selected from SEQ ID NOs: 175-186 and 198.

147. The construct of claim 136, wherein said linker region and said second linker region are independently selected from SEQ ID NOs: 175-186 and 198.

148. The construct of claim 147, wherein said linker region comprises SEQ ID NO: 175 or 176 and said second linker region comprises SEQ ID NO: 183.

149. The construct of any one claims 124-148, wherein said targeting moiety comprises said first cysteine residue and said MHC region comprises said second cysteine residue.

150. The construct of claim 149, wherein said first cysteine residue is located any one of positions 1-9 of said targeting moiety.

151. The construct of claim 149, wherein said first cysteine residue is a C5, C7, or C8 residue of said targeting moiety.

152. The construct of claim 149, wherein said second cysteine residue corresponds to a Y84 residue an HLA-C heavy chain.

153. The construct of claim 149, wherein said second cysteine residue corresponds to an R69 residue an HLA-C heavy chain.

154. The construct of claim 149, wherein said second cysteine residue corresponds to an A73 residue of an HLA-C heavy chain.

155. The construct of claim 149, wherein said second cysteine residue corresponds to an A150 residue of an HLA-C heavy chain.

156. The construct of any one of claims 124-155, wherein said linker region comprises said first cysteine and said MHC region comprises said second cysteine residue.

157. The construct of claim 156, wherein said first cysteine residue is a C2 of said linker.

158. The construct of any one of claims 124-157, wherein said targeting moiety comprises a peptide configured to form a complex with said MHC region.

159. The construct of claim 158, wherein said peptide comprises a sequence at least about 50%, about 60%, about 70%, about 80%, about 90%, about 95%, or about 99% identical to any one of SEQ ID NOs: 158-174.

160. The construct of claim 158 or 159, wherein said peptide comprises a second amino acid residue selected form L, M, S, I, F, T, V, and Y.

161. The construct of claim 160, wherein said second amino acid residue is selected from T, V, and Y.

162. The construct of claim 160 or 161, wherein said peptide comprises a last amino acid residue selected from V, I, F, W, Y, L, R, and K.

163. The construct of claim 162, wherein said last amino acid residue is selected from Y, L, R, and K.

164. The construct of claim 158 or 159, wherein said peptide comprises a second amino acid residue selected from E, P, L, Q, A, R, H, S, T, V, M, D, and K.

165. The construct of claim 164, wherein said second amino acid residue is selected from E, P, L, Q, A, R, and H.

166. The construct of claim 164 or 165, wherein said peptide comprises a last amino acid residue selected from V, L, F, A, I, Y, M, W, P, and R.

167. The construct of claim 166, wherein said last amino acid residue is selected from V, L, and F.

168. The construct of claim 158 or 159, wherein said peptide comprises a second amino acid residue selected from A, Y, S, T, V, I, L, F, Q, R, N, and W.

169. The construct of claim 168, wherein said second amino acid residue is selected from A and Y.

170. The construct of claim 168 or 169, wherein said peptide comprises a last amino acid residue selected from L, V, M, F, Y, and I.

171. The construct of claim 170, wherein said last amino acid residue is L.

172. The construct of any one of claim 162-163, 166-167, or 170-171, wherein said last amino acid residue is a ninth, tenth, eleventh, twelfth reside of said peptide.

173. The construct of any one of claims 124-172, wherein said construct comprises a sequence at least about 50%, 60%, 70%, 80%, 90%, 95%, or 99% identical to any one of SEQ ID NOs 30-129, 142-157, and 203-278.

174. The construct of any one of claims 124-173, wherein said construct is inhibited form eliciting an NK cell response when said construct is interrogated by one or more NK cells.

175. A construct comprising:
a. a targeting moiety;
b. a major histocompatibility complex (MHC) region; and
c. a linker region disposed between said targeting moiety and said MHC region;
wherein said linker region comprises fewer than fifteen amino acid residues.

176. The construct of claim 175, wherein said MHC region comprises an MHC class 1 heavy chain.

177. The construct of claim 176, wherein said MHC class I heavy chain is derived from an HLA-A, HLA-B, or HLA-C sequence.

178. The construct of any one of claims 175-177, wherein said MCH region comprises a sequence at least about 50%, 60%, 70%, 80%, 90%, 95%, or 99% identical to any one of SEQ ID NOs 130-141 and 279-288.

179. The construct of claim 177 or 178, wherein said MHC class I heavy chain comprises a mutation corresponding to the C1 residue of HLA-C (e.g., SEQ ID NO: 194).

180. The construct of claim 179, wherein said mutation comprises a glycine residue.

181. The construct of any one of claims 175-180, further comprising a second MHC region.

182. The construct of claim 181, wherein said second MHC region is derived from an HLA-A, BLA-B, HLA-C, HLAE, HLA-F, or HLA-G sequence.

183. The construct of any one of claims 175-182, further comprising a beta-2 microglobulin (B2M) region.

184. The construct of claim 183, wherein said B2M region is disposed between said targeting moiety and said MHC region.

185. The construct of claim 183 or 184, wherein said B2M region comprises a sequence at least about 50%, 60%, 70%, 80%, 90%, 95%, or 99% identical to SEQ ID NO: 195.

186. The construct of claim 184 or 185, further comprising a second linker region disposed between said B2M region and said targeting moiety or said MHC region.

187. The construct of claim 186, wherein said linker region is disposed between said targeting moiety and said B2M region and said second linker region is disposed between said B2M region and said MHC region.

188. The construct of any one of claims 175-187, wherein said linker region is less than fourteen amino acid residues in length.

189. The construct of any one of claims 175-187, wherein said linker region is less than thirteen amino acid residues in length.

190. The construct of any one of claims 175-187, wherein said linker region is at least eight amino acid residues in length.

191. The construct of any one of claims 175-190, wherein said linker comprises a first cysteine residue configured to form a disulfide staple pair with a second cysteine residue of said MHC region.

192. The construct of claim 191, wherein said first cysteine residue is a C2 of said linker.

193. The construct of claim 191, wherein said linker comprises amino acids selected from the group consisting of: glycine, serine, and cysteine.

194. The construct of any one of claims 175-187, wherein said linker comprises a sequence that is at least about 50%, 60%, 70%, 80%, 90%, 95%, or 99% identical to any one of SEQ ID NOs 175-186 and 198.

195. The construct of any one of claims 175-187, wherein said linker comprises a sequence selected from SEQ ID NOs: 175-186 and 198.

196. The construct of claim 186 or 187, wherein said second linker region comprises a sequence that is at least about 50%, 60%, 70%, 80%, 90%, 95%, or 99% identical to any one of SEQ ID NOs 175-186.

197. The construct of claim 186 or 187, wherein said second linker region comprises a sequence selected from SEQ ID NOs: 175-186 and 198.

198. The construct of claim 186 or 187, wherein said linker region and said second linker region are independently selected from SEQ ID NOs: 175-186 and 198.

199. The construct of claim 198, wherein said linker region comprises SEQ ID NO: 175 or 176 and said second linker region comprises SEQ ID NO: 183.

200. The construct of any one of claims 175-199, wherein said targeting moiety comprises a peptide configured to form a complex with said MHC region.

201. The construct of claim 200, wherein said peptide comprises a sequence at least about 50%, about 60%, about 70%, about 80%, about 90%, about 95%, or about 99% identical to any one of SEQ ID NOs: 158-174.

202. The construct of claim 200 or 201, wherein said peptide comprises a second amino acid residue selected form L, M, S, I, F, T, V, and Y.

203. The construct of claim 202, wherein said second amino acid residue is selected from T, V, and Y.

204. The construct of claim 202 or 203, wherein said peptide comprises a last amino acid residue selected from V, I, F, W, Y, L, R, and K.

205. The construct of claim 204, wherein said last amino acid residue is selected from Y, L, R, and K.

206. The construct of claim 205, wherein said peptide comprises a second amino acid residue selected from E, P, L, Q, A, R, H, S, T, V, M, D, and K.

207. The construct of claim 206, wherein said second amino acid residue is selected from E, P, L, Q, A, R, and H.

208. The construct of claim 206 or 207, wherein said peptide comprises a last amino acid residue selected from V, L, F, A, I, Y, M, W, P, and R.

209. The construct of claim 208, wherein said last amino acid residue is selected from V, L, and F.

210. The construct of claim 200 or 201, wherein said peptide comprises a second amino acid residue selected from A, Y, S, T, V, I, L, F, Q, R, N, and W.

211. The construct of claim 210, wherein said second amino acid residue is selected from A and Y.

212. The construct of claim 210 or 211, wherein said peptide comprises a last amino acid residue selected from L, V, M, F, Y, and I.

213. The construct of claim 212, wherein said last amino acid residue is L.

214. The construct of any one of claim 204-205, 208-209, or 212-213, wherein said last amino acid residue is a ninth, tenth, eleventh, twelfth reside of said peptide.

215. A nucleic acid encoding the construct of any one of claims 124-214.

216. An engineered vector encoding the nucleic acid of claim 215.

217. The engineered vector of claim 216, wherein said vector is a plasmid, a minicircle, a CELiD, an adeno-associated virus (AAV) derived virion, a lentivirus, an adenovirus, or a herpes simplex virus (HSV).

218. A method of generating a hypo-immunogenic cell comprising administering to a cell said vector of claim 216 or 217.

219. A hypo-immunogenic cell comprising the construct of any one of claims 124-214.

220. The hypo-immunogenic cell of claim 219, wherein said cell is a stem cell.

221. The hypo-immunogenic cell of claim 220, wherein said stem cell is an embryonic stem cell (ESC), a mesenchymal stem cell (MSC), an induced pluripotent stem cell (iPSC), or a hematopoietic stem cell (HSC).
